SEQUENCE LISTING (102 sequences)

SEQ ID NO: 1 - DNA, 24 bp, Artificial Sequence
  Primer 1 (Allele X)
aaagctgcgtgatgatgaaatcga

SEQ ID NO: 2 - DNA, 24 bp, Artificial Sequence
  Primer 2 (Allele Y)
aaagctgcgtgatgatgaaatcgg

SEQ ID NO: 3 - DNA, 24 bp, Artificial Sequence
  Common Primer
ttgaggctgacctgaagcacttga

SEQ ID NO: 4 - DNA, 101 bp, Homo sapiens
gagctttgaggctgacctgaagcacttgaaggagaaggtgtctgcgggagycgatttcatcatcacgcagcttttctttgaggctgacacattcttccgct

SEQ ID NO: 5 - DNA, 20 bp, Artificial Sequence
  Primer 1 (Allele X)
ccgccctgggttcggctatt

SEQ ID NO: 6 - DNA, 19 bp, Artificial Sequence
  Primer 2 (Allele Y)
cgccctgggttcggctatc

SEQ ID NO: 7 - DNA, 21 bp, Artificial Sequence
  Common Primer
tcccgctctggcccagcaatt

SEQ ID NO: 8 - DNA, 101 bp, Homo sapiens
gcggcctgaaggatgctgataaccgggagccccgccctgggttcggctatyccgggcaccccgggccggcggggcgaggctctccaattgctgggccagag

SEQ ID NO: 9 - DNA, 20 bp, Artificial Sequence
  Primer 1 (Allele X)
ggagcccagataccccaaaa

SEQ ID NO: 10 - DNA, 20 bp, Artificial Sequence
  Primer 2 (Allele Y)
ggagcccagataccccaaag

SEQ ID NO: 11 - DNA, 22 bp, Artificial Sequence
  Common Primer
ccgggctgtgctttctcgtctt

SEQ ID NO: 12 - DNA, 101 bp, Homo sapiens
gaccgggctgtgctttctcgtcttcagcaccagcaggcagctggctccggytttggggtatctgggctccaggcagaagcacagcctccccgacctgccct

SEQ ID NO: 13 - DNA, 29 bp, Artificial Sequence
  Primer 1 (Allele X)
aattagagagctaagcactttttagatac

SEQ ID NO: 14 - DNA, 30 bp, Artificial Sequence
  Primer 2 (Allele Y)
caattagagagctaagcactttttagatat

SEQ ID NO: 15 - DNA, 29 bp, Artificial Sequence
  Common Primer
gggtgcctcatacggcaattaaattatat

SEQ ID NO: 16 - DNA, 101 bp, Homo sapiens
gcacagctgttatttactgaacaattagagagctaagcactttttagataytatataatttaattgccgtatgaggcacccttagttttcagacgagaaac

SEQ ID NO: 17 - DNA, 22 bp, Artificial Sequence
  Primer 1 (Allele X)
cattttaccacatggtcctcac

SEQ ID NO: 18 - DNA, 26 bp, Artificial Sequence
  Primer 2 (Allele Y)
caytcattttaccacatggtcctcat

SEQ ID NO: 19 - DNA, 25 bp, Artificial Sequence
  Common Primer
gacacatatgaggcagctgagagta

SEQ ID NO: 20 - DNA, 101 bp, Homo sapiens
agtccagcaggctgaatgccttcaacaytcattttaccacatggtcctcayttactctcagctgcctcatatgtgtcacctcacaartaatcaaataaaat

SEQ ID NO: 21 - DNA, 24 bp, Artificial Sequence
  Primer 1 (Allele X)
atcagtgaaggaatcgctttctgg

SEQ ID NO: 22 - DNA, 24 bp, Artificial Sequence
  Primer 2 (Allele Y)
atcagtgaaggaatcgctttctgc

SEQ ID NO: 23 - DNA, 26 bp, Artificial Sequence
  Common Primer
ccctattccatgctgttatgggtgaa

SEQ ID NO: 24 - DNA, 101 bp, Homo sapiens
ccctattccatgctgttatgggtgaaactctgggagattctcctattgacscagaaagcgattccttcactgatacactgtctgcaaacatatcacaaggt

SEQ ID NO: 25 - DNA, 21 bp, Artificial Sequence
  Primer 1 (Allele X)
ccaggaactggagcgaaagtg

SEQ ID NO: 26 - DNA, 22 bp, Artificial Sequence
  Primer 2 (Allele Y)
cccaggaactggagcgaaagta

SEQ ID NO: 27 - DNA, 26 bp, Artificial Sequence
  Common Primer
cagagaagatctgagcatttgggctt

SEQ ID NO: 28 - DNA, 101 bp, Homo sapiens
  misc_feature (19)..(19): n is a, c, g, or t
  misc_feature (70)..(70): n is a, c, g, or t
  misc_feature (74)..(74): n is a, c, g, or t
  misc_feature (87)..(87): n is a, c, g, or t
aagaggcatctgggccagngactctgagccccaggaactggagcgaaagtragatttgccccatgagganaagntgaactccactcncagggcctctgagg

SEQ ID NO: 29 - DNA, 25 bp, Artificial Sequence
  Primer 1 (Allele X)
acagagactatccaagtgcatcact

SEQ ID NO: 30 - DNA, 25 bp, Artificial Sequence
  Primer 2 (Allele Y)
acagagactatccaagtgcatcaca

SEQ ID NO: 31 - DNA, 30 bp, Artificial Sequence
  Common Primer
ctgaattattattctaggttccttgcgact

SEQ ID NO: 32 - DNA, 101 bp, Homo sapiens
ttagaatgtctgaattattattctaggttccttgcgactgctgtgaatttwgtgatgcacttggatagtctctgttactctaaagttttaataggtaacag

SEQ ID NO: 33 - DNA, 23 bp, Artificial Sequence
  Primer 1 (Allele X)
cagagcctgagtattgggaatgt

SEQ ID NO: 34 - DNA, 23 bp, Artificial Sequence
  Primer 2 (Allele Y)
cagagcctgagtattgggaatgc

SEQ ID NO: 35 - DNA, 27 bp, Artificial Sequence
  Common Primer
attctgaggaactagataagcagggtt

SEQ ID NO: 36 - DNA, 101 bp, Homo sapiens
attctgaggaactagataagcagggttcctggggccacagacaggcctgcrcattcccaatactcaggctctgctcttgcgtgaactgggctcaacattcc

SEQ ID NO: 37 - DNA, 19 bp, Artificial Sequence
  Primer 1 (Allele X)
gtcatcgtggccatcgccc

SEQ ID NO: 38 - DNA, 20 bp, Artificial Sequence
  Primer 2 (Allele Y)
ggtcatcgtggccatcgcct

SEQ ID NO: 39 - DNA, 22 bp, Artificial Sequence
  Common Primer
ggtcatggtctggagtctcgga

SEQ ID NO: 40 - DNA, 101 bp, Homo sapiens
cggtgctggccaccgtgggaggcaacctgctggtcatcgtggccatcgccyggactccgagactccagaccatgaccaacgtgttcgtgacttcgctggcc

SEQ ID NO: 41 - DNA, 22 bp, Artificial Sequence
  Primer 1 (Allele X)
agagttcctttgaggccaggga

SEQ ID NO: 42 - DNA, 21 bp, Artificial Sequence
  Primer 2 (Allele Y)
gagttcctttgaggccagggg

SEQ ID NO: 43 - DNA, 25 bp, Artificial Sequence
  Common Primer
ccctacaatgtactagtaggcctct

SEQ ID NO: 44 - DNA, 101 bp, Homo sapiens
  misc_feature (24)..(24): n is a, c, g, or t
  misc_feature (46)..(46): n is a, c, g, or t
acaatgtactagtaggcctctgcnctggcaatacagataagataangtagyccctggcctcaaaggaactctcctccttaggttgcatttgtataatgttt

SEQ ID NO: 45 - DNA, 21 bp, Artificial Sequence
  Primer 1 (Allele X)
cacccacacctcgtccctttc

SEQ ID NO: 46 - DNA, 21 bp, Artificial Sequence
  Primer 2 (Allele Y)
cacccacacctcgtccctttg

SEQ ID NO: 47 - DNA, 19 bp, Artificial Sequence
  Common Primer
gaagccatgcgccggacca

SEQ ID NO: 48 - DNA, 101 bp, Homo sapiens
  misc_feature (16)..(16): n is a, c, g, or t
  misc_feature (18)..(18): n is a, c, g, or t
  misc_feature (38)..(38): n is a, c, g, or t
tcttgctggcacccantngaagccatgcgccggaccangacgtcacgcagsaaagggacgaggtgtgggtggtgggcatgggcatcgtcatgtctctcatc

SEQ ID NO: 49 - DNA, 23 bp, Artificial Sequence
  Primer 1 (Allele X)
gaagcaaaggtagcacacgaggt

SEQ ID NO: 50 - DNA, 22 bp, Artificial Sequence
  Primer 2 (Allele Y)
aagcaaaggtagcacacgaggc

SEQ ID NO: 51 - DNA, 19 bp, Artificial Sequence
  Common Primer
gaccccgagctccggtcct

SEQ ID NO: 52 - DNA, 101 bp, Homo sapiens
gcaggtgcccgtggaacctgggcctgaccccgagctccggtcctggcggcrcctcgtgtgctacctttgcttctacggcttcatggcgcagatacggccag

SEQ ID NO: 53 - DNA, 25 bp, Artificial Sequence
  Primer 1 (Allele X)
gcaacaatctgggctatgagatcaa

SEQ ID NO: 54 - DNA, 24 bp, Artificial Sequence
  Primer 2 (Allele Y)
caacaatctgggctatgagatcag

SEQ ID NO: 55 - DNA, 29 bp, Artificial Sequence
  Common Primer
ttttgctgcttcttttggctctgacttta

SEQ ID NO: 56 - DNA, 101 bp, Homo sapiens
ctgcttgagttgtagaaagaaccgctgcaacaatctgggctatgagatcartaaagtcagagccaaaagaagcagcaaaatgtacctgaagactcgttctc

SEQ ID NO: 57 - DNA, 18 bp, Artificial Sequence
  Primer 1 (Allele X)
cggtcctggatggcctcg

SEQ ID NO: 58 - DNA, 19 bp, Artificial Sequence
  Primer 2 (Allele Y)
gcggtcctggatggcctca

SEQ ID NO: 59 - DNA, 20 bp, Artificial Sequence
  Common Primer
gtgcaggacgccgrgctgat

SEQ ID NO: 60 - DNA, 101 bp, Homo sapiens
tctatccccgtgcccacagatcgtcctggggtgcaggacgccgrgctgatygaggccatccaggaccgcctgtccaacayactgcagacgtayatccgstg

SEQ ID NO: 61 - DNA, 22 bp, Artificial Sequence
  Primer 1 (Allele X)
gggakgagctgaccagtgaaga

SEQ ID NO: 62 - DNA, 21 bp, Artificial Sequence
  Primer 2 (Allele Y)
ggakgagctgaccagtgaagc

SEQ ID NO: 63 - DNA, 30 bp, Artificial Sequence
  Common Primer
aggtaaagaacraagacttcaaagacactt

SEQ ID NO: 64 - DNA, 101 bp, Homo sapiens
ccccaaggaggagctgctgaagatgtggggggakgagctgaccagtgaagmaagtgtctttgaagtcttygttctttacctctcgggagaaccaaaccgga

SEQ ID NO: 65 - DNA, 19 bp, Artificial Sequence
  Primer 1 (Allele X)
gttcccagttctgccccag

SEQ ID NO: 66 - DNA, 21 bp, Artificial Sequence
  Primer 2 (Allele Y)
ctgttcccagttctgccccac

SEQ ID NO: 67 - DNA, 25 bp, Artificial Sequence
  Common Primer
cctcactctatcaccagttcctcat

SEQ ID NO: 68 - DNA, 101 bp, Homo sapiens
ctctctctcttcctcactctatcaccagttcctcatgacttcccccatgcstggggcagaactgggaacagcatgtctcaaggcgagggttgctttgctgg

SEQ ID NO: 69 - DNA, 18 bp, Artificial Sequence
  Primer 1 (Allele X)
cgccctaggcacagctgg

SEQ ID NO: 70 - DNA, 19 bp, Artificial Sequence
  Primer 2 (Allele Y)
gcgccctaggcacagctga

SEQ ID NO: 71 - DNA, 24 bp, Artificial Sequence
  Common Primer
gacatcgaagccctgctgtctcaa

SEQ ID NO: 72 - DNA, 101 bp, Homo sapiens
ccagaccattgacatcgagcctgacatcgaagccctgctgtctcaagggcycagctgtgcctagggcgcccctcctaccccggctgcttggcagttgcagt

SEQ ID NO: 73 - DNA, 22 bp, Artificial Sequence
  Primer 1 (Allele X)
gccttcttgctggcacccaata

SEQ ID NO: 74 - DNA, 21 bp, Artificial Sequence
  Primer 2 (Allele Y)
ccttcttgctggcacccaatg

SEQ ID NO: 75 - DNA, 20 bp, Artificial Sequence
  Common Primer
cgtggtccggcgcatggctt

SEQ ID NO: 76 - DNA, 101 bp, Homo sapiens
gcgccatggggcaacccgggaacggcagcgccttcttgctggcacccaatrgaagccatgcgccggaccacgacgtcacgcaggaaagggacgaggtgtgg

SEQ ID NO: 77 - DNA, 20 bp, Artificial Sequence
  Primer 1 (Allele X)
ccatctaccatgcgtcctgt

SEQ ID NO: 78 - DNA, 20 bp, Artificial Sequence
  Primer 2 (Allele Y)
ccatctaccatgcgtcctgg

SEQ ID NO: 79 - DNA, 26 bp, Artificial Sequence
  Common Primer
cagctctcagattctgtgatgctcaa

SEQ ID NO: 80 - DNA, 101 bp, Homo sapiens
cctttccagctctcagattctgtgatgctcaaagggtgagctctgtgggcmcaggacgcatggtagatggagcttagtctttctggtatccagctgggagc

SEQ ID NO: 81 - DNA, 21 bp, Artificial Sequence
  Primer 1 (Allele X)
attgcaggacccaaggagctc

SEQ ID NO: 82 - DNA, 18 bp, Artificial Sequence
  Primer 2 (Allele Y)
gcaggacccaaggagctg

SEQ ID NO: 83 - DNA, 23 bp, Artificial Sequence
  Common Primer
cccaagtccacctgcctatccat

SEQ ID NO: 84 - DNA, 101 bp, Homo sapiens
tttaagcaacctacaggggcagccctggagattgcaggacccaaggagctsgcaggatggataggcaggtggacttggggtattgaggtctcaggcagcca

SEQ ID NO: 85 - DNA, 20 bp, Artificial Sequence
  Primer 1 (Allele X)
ggcaggaggcccagctcatt

SEQ ID NO: 86 - DNA, 20 bp, Artificial Sequence
  Primer 2 (Allele Y)
ggcaggaggcccagctcata

SEQ ID NO: 87 - DNA, 28 bp, Artificial Sequence
  Common Primer
atattctcaagatggcaaccgcatacat

SEQ ID NO: 88 - DNA, 101 bp, Homo sapiens
cagcctttcaggttggatattctcaagatggcaaccgcatacatccggagwatgagctgggcctcctgcctggctttccacagggaggagaaggtgaggtc

SEQ ID NO: 89 - DNA, 26 bp, Artificial Sequence
  Primer 1 (Allele X)
gtgggcacaaatttaatcaaagtggc

SEQ ID NO: 90 - DNA, 27 bp, Artificial Sequence
  Primer 2 (Allele Y)
agtgggcacaaatttaatcaaagtggt

SEQ ID NO: 91 - DNA, 22 bp, Artificial Sequence
  Common Primer
cagggccgtggctgttgtagat

SEQ ID NO: 92 - DNA, 101 bp, Homo sapiens
aactgaattttttcagaatgcagaagtgggcacaaatttaatcaaagtggyatctacaacagccacggccctgaaggaagaagatggccaagtctactgcc

SEQ ID NO: 93 - DNA, 25 bp, Artificial Sequence
  Primer 1 (Allele X)
cctgctgcctatacagtcactttta

SEQ ID NO: 94 - DNA, 25 bp, Artificial Sequence
  Primer 2 (Allele Y)
cctgctgcctatacagtcacttttt

SEQ ID NO: 95 - DNA, 29 bp, Artificial Sequence
  Common Primer
agctcagagaatttcagagctggaataaa

SEQ ID NO: 96 - DNA, 23 bp, Artificial Sequence
  Common Primer
gacagagcgagactccgtctcaa

SEQ ID NO: 97 - DNA, 288 bp, Homo sapiens
atacagtcactttttttttttttttgagacggagtctcgctctgtcgcccaggctggagtgcagtggcgggatctcggctcactgcaacgtccgcctcccgggttcacgccattctcctgcctcagcctcccaagtagctgggaccacagcgcccgccactacgcccggctaattttttgtatttttagtagagacggggtttcaccgttttagccgggatggtctcgatctcctgacctcgtgatccgcccgcctcggcctcccaaagtgctgggattacaggcgtg

SEQ ID NO: 98 - DNA, 388 bp, Homo sapiens
tgctggagaccactcccatcctttctcccatttctctagacctgctgcctatacagtcactttttttttttttttgagacggagtctcgctctgtcgcccaggctggagtgcagtggcgggatctcggctcactgcaacgtccgcctcccgggttcacgccattctcctgcctcagcctcccaagtagctgggaccacagcgcccgccactacgcccggctaattttttgtatttttagtagagacggggtttcaccgttttagccgggatggtctcgatctcctgacctcgtgatccgcccgcctcggcctcccaaagtgctgggattacaggcgtgatacagtcacttttatgtggtttcgccaattttattccagctctgaaatt

SEQ ID NO: 99 - DNA, 100 bp, Homo sapiens
tgctggagaccactcccatcctttctcccatttctctagacctgctgcctatacagtcacttttatgtggtttcgccaattttattccagctctgaaatt

SEQ ID NO: 100 - DNA, 188 bp, Homo sapiens
  misc_feature (101)..(101): n is SEQ ID NO 101 or SEQ ID NO 102
tgcaggtgtctgcagcatgtggccccaggccggggactctgtaagccactgctggagagccactcccatcctttctcccatttctctagacctgctgcctnatgtggtttcgccaattttattccagctctgaaattctctgagctccccttacaagcagaggtgagctaagggctggagctcaaggc

SEQ ID NO: 101 - DNA, 14 bp, Homo sapiens
atacagtcactttt

SEQ ID NO: 102 - DNA, 64 bp, Homo sapiens
atacagtcactttttttttttttttgagacggagtctcgctctgtcgcccatacagtcactttt

(19) United States
(12) Patent Application Publication
(10) Pub. No.: US 2025/0000816 A1
(43) Pub. Date: Jan. 2, 2025

(54) NUTRITIONAL COMPOSITIONS

(71) Applicant: Nutri-Gentix Limited, Brighton (GB)

(72) Inventors: Jeremy Duncan Poland, Brighton (GB); Alexander William Blyth, Brighton (GB)

(21) Appl. No.: 18/709,380

(22) PCT Filed: Nov. 11, 2021
(86) PCT No.: PCT/GB2021/052917

§ 371 Date: May 10, 2024

(51) Int. Cl. A61K 31/07; A23L 33/15; A23L 33/155; A23L 33/16; A61K 31/355; A61K 31/375; A61K 31/4415; A61K 31/519; A61K 31/593; A61K 31/714; A61K 33/04; A61K 33/06

(52) U.S. Cl. CPC A61K 31/07; A23L 33/15; A23L 33/155; A23L 33/16; A61K 31/355; A61K 31/375; A61K 31/4415; A61K 31/519; A61K 31/593; A61K 31/714; A61K 33/04; A61K 33/06

(57) ABSTRACT

The present invention relates to a nutritional composition comprising one or more micronutrients, wherein the one or more micronutrients comprise vitamin A, pyridoxine (vitamin B6), folic acid (vitamin B9), cyanocobalamin (vitamin B12), ascorbic acid (vitamin C), cholecalciferol (vitamin D3), vitamin E, calcium, and/or selenium. Also provided are corresponding kits, methods of manufacturing and using the same, and methods of determining a subject's nutritional requirements.

### CROSS REFERENCE TO RELATED APPLICATION

This application is a U.S. National Phase Application based on International Patent Application No. PCT/GB2021/052917 filed on Nov. 11, 2021, the contents of which are incorporated herein by reference as if fully set forth herein.

### REFERENCE TO SEQUENCE LISTING

The Sequence Listing associated with this application is provided in text format in lieu of a paper copy, and is hereby incorporated by reference into the specification. The name of the text file containing the Sequence Listing is 3831966_ST25.txt. The text file is 22,160 bytes, was created on May 9, 2024, and is being submitted electronically via Patent Center.

The present invention relates to nutrition.

Nutritional compositions are currently formulated based on recommended daily allowances of the various micronutrient and macronutrient components (e.g. the European Union's Nutritional Reference Value (NRVs)).

Despite this, the health effects of nutrients and nutriomes (nutrient combinations) are significantly influenced by variations in genes that alter the uptake and metabolism of nutrients of a subject. Furthermore, manufacturers of foodstuffs, food supplements, and vitamin supplements formulate their products based on such recommended daily allowances, as this provides the necessary economies of scale together with increased profit margins versus formulating products based on an individual's specific nutritional requirements.

While tests have been developed that can provide a subject with a Personal Daily Allowance (PDA; i.e. their individual nutritional requirements), it is currently uneconomical to manufacture such personalised nutritional supplements on an individual basis.

The present invention overcomes one or more of the above-mentioned problems.

The present inventors surprisingly found unexpected correlations across subjects for certain nutrient needs. Utilising the knowledge of specific correlations between the uptake and/or metabolism of various micronutrients (and optionally macronutrients), together with a plurality of subjects' genetic profiles, the present inventors have, surprisingly, been able to stratify the population into specific nutritional groups (as represented by the nutritional reference standards herein). In more detail, the identification of a set of determined nutritional reference standards allows for the production of a set of nutritional compositions where one of the nutritional compositions corresponds to a subject's genetic requirement for the one or more micronutrients comprised therein. Indeed, surprisingly, the inventors found that the majority of the population can be grouped into a small number of different categories (corresponding to the reference standards herein) and that a small number of nutritional compositions can be produced that meet the nutritional requirements of the majority of the population. The inventors were able to group the population into far fewer nutritional groups that had common nutritional needs than was expected. This “semi-personalised” approach allows for the nutritional needs of the subject to be closely met, but in an economical and/or commercially scalable manner. The nutritional compositions that encompass the vast majority of the population are thus provided herein.

In one aspect of the present invention, there is provided a nutritional composition comprising one or more micronutrients, wherein the one or more micronutrients comprise vitamin A, pyridoxine (vitamin B6), folic acid (vitamin B9), cyanocobalamin (vitamin B12), ascorbic acid (vitamin C), cholecalciferol (vitamin D3), vitamin E, calcium, and/or selenium.

In one aspect of the present invention, there is provided a nutritional composition comprising one or more micronutrients, wherein an amount of the one or more micronutrients in the composition is based on a subject's nutritional requirement for the one or more micronutrients, wherein the subject's nutritional requirement for the one or more micronutrients has been determined using the subject's genetic profile, and wherein the one or more micronutrients comprise vitamin A, pyridoxine (vitamin B6) (e.g. pyridoxine HCl), folic acid (vitamin B9), cyanocobalamin (vitamin B12), ascorbic acid (vitamin C), cholecalciferol (vitamin D3), vitamin E, calcium, and/or selenium.

In a related aspect, the invention provides a kit comprising a nutritional composition, wherein the nutritional composition comprises one or more micronutrients, wherein the kit comprises:
(a) a container comprising at least a daily unit dose of the one or more micronutrients; or
(b) a plurality of containers each comprising a fraction of the daily unit dose of the one or more micronutrients; and
(c) optionally instructions for use of the same;
   wherein the kit comprises at least one daily unit dose of the one or more micronutrients, and wherein the one or more micronutrients comprise vitamin A, pyridoxine (vitamin B6), folic acid (vitamin B9), cyanocobalamin (vitamin B12), ascorbic acid (vitamin C), cholecalciferol (vitamin D3), vitamin E, calcium, and/or selenium.

The term “container” as used in the context of a kit of the invention is intended to encompass any form of packaging of the composition and is not intended to imply a particular structure.

An amount of the one or more micronutrients in the composition or comprised in the kit may be based on a subject's nutritional requirement for the one or more micronutrients, wherein the subject's nutritional requirement for the one or more micronutrients has been determined using the subject's genetic profile, preferably using a method of the invention.

A “subject” as used herein may be a mammal, such as a human or other mammal. Preferably “subject” means a human subject. The compositions and kits of the invention are thus preferably suitable for administration to a human subject.

A micronutrient may encompass vitamins and/or minerals which are beneficial or essential to human health. A nutritional composition according to the invention comprises at least one of: vitamin A, pyridoxine (vitamin B6), folic acid (vitamin B9), cyanocobalamin (vitamin B12), ascorbic acid (vitamin C), cholecalciferol (vitamin D3), vitamin E, calcium, and/or selenium. For example, a nutritional composition of the invention may comprise at least two, three, four, five, six, seven or eight of vitamin A, pyridoxine (vitamin B6), folic acid (vitamin B9), cyanocobalamin (vitamin B12), ascorbic acid (vitamin C), cholecalciferol (vitamin D3), vitamin E, calcium, and/or selenium. Preferably, a nutritional composition of the invention comprises vitamin A, pyridoxine (vitamin B6), folic acid (vitamin B9), cyanocobalamin (vitamin B12), ascorbic acid (vitamin C), cholecalciferol (vitamin D3), vitamin E, calcium, and selenium.

A reference to a micronutrient herein preferably encompasses any pharmaceutically acceptable salt thereof (e.g. a salt that is suitable for human consumption). In other words, a reference to a micronutrient may be a reference to any pharmaceutically acceptable salt of a micronutrient. However, certain forms of the micronutrients are preferred, as detailed herein. The composition (or kit) of the invention may comprise (e.g. additionally comprise) a pharmaceutically acceptable salt of any of the micronutrients described herein.

Vitamin A may be a group of unsaturated nutritional organic compounds that includes retinol, retinal, and several provitamin A carotenoids (such as beta-carotene). The skilled person will understand which compounds are encompassed by the term “vitamin A”. The term “vitamin A” as used herein is intended to encompass any vitamin A compounds, or any combination of vitamin A compounds. By way of non-limiting example, a composition of the invention may comprise retinol. Alternatively, a composition of the invention may comprise retinol and beta-carotene. Preferably, a composition of the invention comprises retinol (e.g. retinol acetate). Vitamin E may be a group of eight fat soluble compounds that includes, for example, alpha-Tocopherol, beta-Tocopherol, alpha-Tocotrienol and gamma-Tocotrienol. The skilled person will understand which compounds are encompassed by the term “vitamin E”. The term “vitamin E” as used herein is intended to encompass any vitamin E compounds, or any combination of vitamin E compounds. By way of non-limiting example, a composition of the invention may comprise alpha-Tocopherol. Alternatively, a composition of the invention may comprise beta-Tocopherol and alpha-Tocotrienol. Preferably, a composition of the invention comprises alpha-Tocopherol (e.g. D-Alpha Tocopheryl Succinate). The skilled person will understand which forms of calcium are suitable for inclusion in nutritional compositions. For example, the calcium may be in the form of a salt, such as, calcium carbonate, calcium citrate or calcium lactate. The skilled person will understand which forms of selenium are suitable for inclusion in nutritional compositions. For example, the selenium may be in the form of a salt, such as, selenomethionine, selenocysteine, selenite or selenate. Preferably, a composition of the invention comprises selenomethionine (e.g. L-Selenomethionine).

Preferably, a composition of the invention comprises a daily unit dose of the one or more micronutrients or a fraction of a daily unit dose of the one or more micronutrients. For example, the fraction of a daily unit dose of the one or more micronutrients may be at least 10%, 20%, 30%, 40%, 50%, 60%, 70%, 80%, 90% or 95% of a daily unit dose of the one or more micronutrients. Preferably, a fraction of the daily unit dose of the one or more micronutrients is 50% of a daily unit dose of the one or more micronutrients. This is particularly useful where (for example), the composition is provided as a single serving and wherein it is intended that a subject consumes two servings per day. In this regard, a kit of the invention may comprise a container comprising a fraction of the daily unit dose of the one or more micronutrients of the invention. Such containers may comprise a pre-packaged single serving of the one or more micronutrients.

In some embodiments, the composition of the invention comprises more than a daily unit dose of the one or more micronutrients, e.g. 2, 3, 4, 5, 10, 15, 20 or 30 or more daily unit doses of the one or more micronutrients, preferably 7 or 14 daily unit doses of the one or more micronutrients. This is particularly useful where (for example), the composition is provided as a double serving (for consuming over the course of two days) and wherein it is intended that a subject divides the serving in two (or more parts, e.g. four parts). Preferably, a kit of the invention may comprise a container comprising at least one (e.g. 2, 3, 4, 5, 10, 15, 20 or 30 or more) daily unit doses of the one or more micronutrients of the invention, preferably 7 or 14 daily unit doses of the one or more micronutrients. The kit may further comprise a container comprising at least one (e.g. 2, 3, 4, 5, 10, 15, 20 or 30 or more) daily unit doses of one or more macronutrients, preferably 7 or 14 daily unit doses of the one or more micronutrients.

In one embodiment, the composition or kit of the invention comprises 1-20, 2-19, 3-18, 4-17 or 5-16 daily unit doses of the one or more micronutrients, preferably 5-20 (more preferably 7 or 14) daily unit doses of the one or more micronutrients.

In one embodiment, the kit of the invention comprises at least 100 g, 200 g, 300 g, 400 g or 500 g of the composition of the invention, preferably 500 g or 1000 g of the composition of the invention. Said composition may be a dry composition. In such embodiments, preferably said composition comprises the one or more micronutrients, one or more further micronutrients, and one or more macronutrients.

A composition or kit of the invention preferably comprises at least two (e.g. at least three, four, five, six, seven or eight, or more) micronutrients, wherein a weight ratio between the at least two (e.g. at least three, four, five, six, seven or eight, or more) micronutrients is the same as the weight ratio between the at least two (e.g. at least three, four, five, six, seven or eight, or more) micronutrients in a daily unit dose described herein.

Instructions may be present in a kit of the invention and said instructions may explain how to divide up a composition into single servings and/or how to prepare the composition for consumption, e.g. by hydration with an aqueous solution. The instructions may include instructions detailing the amount (weight) of a micronutrient composition and the amount (weight) of a macronutrient composition to be mixed to provide a nutritional composition.

A preferred serving size of a composition comprising micronutrients (and no macronutrients) is 1-3.5 g, more preferably 1.7-2.7 g. A preferred serving size of a composition comprising macronutrients (and no micronutrients) may be 25-34 or 31.5-34 g, more preferably 32.3-33.3 g. A preferred daily unit dose of a composition comprising micronutrients (and no macronutrients) is 2-7 g, more preferably 4-5.4 g. A preferred serving size of a composition comprising macronutrients (and no micronutrients) may be 50-68 g or 63-68 g, more preferably 64.6-66 g.

Suitable daily unit doses of the one or more micronutrients are provided below. The skilled person will readily determine how much of each micronutrient is present in the daily dose as well as any fraction thereof. For example, where a daily unit dose of vitamin A is 130% of a reference vitamin A daily dose of 800 μg, the skilled person will appreciate that a composition comprising a daily unit dose comprises 1,040 μg of vitamin A. Likewise, where a daily unit dose of vitamin A is 130% of a reference vitamin A daily dose of 800 μg, the skilled person will appreciate that a composition comprising a 50% fraction of a daily unit dose comprises 520 μg of vitamin A.

A daily unit dose may be a daily unit dose of vitamin A, pyridoxine (vitamin B6), folic acid (vitamin B9), cyanocobalamin (vitamin B12), ascorbic acid (vitamin C), cholecalciferol (vitamin D3), vitamin E, calcium, and/or selenium.

The daily unit doses may be as defined below.

A daily unit dose of vitamin A may refer to a dose of vitamin A that is greater than 100% to 375% of a reference vitamin A daily dose, wherein the reference vitamin A daily dose is 800 μg. A lower end of the range may be at least 105%, preferably 110%. An upper limit of the range may be 300% or 250%, preferably 200%. Preferably, a daily unit dose of vitamin A may refer to a dose of vitamin A that is 110% to 200% of a reference vitamin A daily dose.

A daily unit dose of pyridoxine may refer to a dose of pyridoxine that is greater than 100% to 7143% of a reference pyridoxine daily dose, wherein the reference pyridoxine daily dose is 1.4 mg. A lower end of the range may be at least 130%, preferably 150%. An upper limit of the range may be 5000% or 2500%, preferably 1000%. Preferably, a daily unit dose of pyridoxine may refer to a dose of pyridoxine that is 150% to 1000% of a reference pyridoxine daily dose.

A daily unit dose of folic acid may refer to a dose of folic acid that is greater than 100% to 500% of a reference folic acid daily dose, wherein the reference folic acid daily dose is 200 μg. A lower end of the range may be at least 105%, preferably 110%. An upper limit of the range may be 400% or 300%, preferably 250%. Preferably, a daily unit dose of folic acid may refer to a dose of folic acid that is 110% to 250% of a reference folic acid daily dose.

A daily unit dose of cyanocobalamin may refer to a dose of cyanocobalamin that is greater than 100% to 40000% of a reference cyanocobalamin daily dose, wherein the reference cyanocobalamin daily dose is 2.5 μg. A lower end of the range may be at least 130%, preferably 150%. An upper limit of the range may be 10000% or 5000%, preferably 1000%. Preferably, a daily unit dose of cyanocobalamin may refer to a dose of cyanocobalamin that is 150% to 1000% of a reference cyanocobalamin daily dose.

A daily unit dose of ascorbic acid may refer to a dose of ascorbic acid that is greater than 100% to 2500% of a reference ascorbic acid daily dose, wherein the reference ascorbic acid daily dose is 80 mg. A lower end of the range may be at least 140%, preferably 160%. An upper limit of the range may be 1000%, preferably 750%. Preferably, a daily unit dose of ascorbic acid may refer to a dose of ascorbic acid that is 160% to 750% of a reference ascorbic acid daily dose.

A daily unit dose of cholecalciferol may refer to a dose of cholecalciferol that is greater than 100% to 2000% of a reference cholecalciferol daily dose, wherein the reference cholecalciferol daily dose is 5 μg. A lower end of the range may be at least 140%, preferably 160%. An upper limit of the range may be 1000%, preferably 750%. Preferably, a daily unit dose of ascorbic acid may refer to a dose of cholecalciferol that is 160% to 750% of a reference cholecalciferol daily dose.

A daily unit dose of vitamin E may refer to a dose of vitamin E that is greater than 100% to 9167% of a reference vitamin E daily dose, wherein the reference vitamin E daily dose is 12 mg. A lower end of the range may be at least 105%, preferably 115%. An upper limit of the range may be 7500% or 5000%, preferably 2000%. Preferably, a daily unit dose of vitamin E may refer to a dose of vitamin E that is 115% to 2000% of a reference vitamin E daily dose.

A daily unit dose of calcium may refer to a dose of calcium that is greater than 100% to 313% of a reference calcium daily dose, wherein the reference calcium daily dose is 800 mg. A lower end of the range may be at least 105%, preferably 107%. An upper limit of the range may be 250% or 200%, preferably 175%. Preferably, a daily unit dose daily unit dose of calcium may refer to a dose of calcium that is 105% to 175% of a reference calcium daily dose.

A daily unit dose of selenium may refer to a dose of selenium that is greater than 100% to 727% of a reference selenium daily dose, wherein the reference selenium daily dose is 55 μg. A lower end of the range may be at least 105% or 110%, preferably 115%. An upper limit of the range may be 500% or 300%, preferably 250%. Preferably, a daily unit dose of selenium may refer to a dose of selenium that is 115% to 250% of a reference selenium daily dose.

The term “greater than” when used together with a range herein (e.g. greater than 100% to 375%) refers to the lower end of the range. In other words, by way of example, greater than 100% to 375% means that the lower end of the range is more than 100%, it is not intended to mean that the amount is greater than 375%.

A daily unit dose may refer to (or may comprise):
(a) a dose of vitamin A that is: (i) greater than 100% to 127.5% (e.g. a lower end of the range may be at least 105%, 110%, 115% or 120%, preferably 110% and/or an upper end of the range may be less than or equal to 125%, 120%, 115% or 110%) of a reference vitamin A daily dose; (ii) greater than 127.5% to 158.5% of a reference vitamin A daily dose (e.g. a lower end of the range may be at least 130%, 135%, 140%, 145% or 150% and/or an upper end of the range may be less than or equal to 155%, 150%, 145%, 140%); or (iii) greater than 158.5% to 375% (e.g. a lower end of the range may be at least 160%, 165%, 170%, 175% or 180% and/or an upper limit of the range may be less than or equal to 350%, 325%, 300%, 275% or 250%, preferably 200%) of a reference vitamin A daily dose, wherein the reference vitamin A daily dose is 800 μg;
(b) a dose of pyridoxine that is: (i) greater than 100% to 305% (e.g. a lower end of the range may be at least 130%, 135%, 140%, 145% or 150%, preferably 150% and/or an upper end of the range may be less than or equal to 300%, 295%, 290%, 285% or 280%) of a reference pyridoxine daily dose; (ii) greater than 305% to 590% of a reference pyridoxine daily dose (e.g. a lower end of the range may be at least 310%, 315%, 320%, 325% or 330% and/or an upper end of the range may be less than or equal to 585%, 580%, 575%, 570% or 565%); or (iii) greater than 590% to 7143% (e.g. a lower end of the range may be at least 600%, 620%, 640%, 660% or 680% and/or an upper limit of the range may be less than or equal to 5000%, 4500%, 4000%, 3500%, 3000% or 2500%, preferably 1000%) of a reference pyridoxine daily dose, wherein the reference pyridoxine daily dose is 1.4 mg;
(c) a dose of folic acid that is: (i) greater than 100% to 145% (e.g. a lower end of the range may be at least 105%, 110%, 115%, 120% or 125%, preferably 110% and/or an upper end of the range may be less than or equal to 140%, 135%, 130%, 125% or 120%) of a reference folic acid daily dose; (ii) greater than 145% to 200% of a reference folic acid daily dose (e.g. a lower end of the range may be at least 150%, 155%, 160%, 165% or 170% and/or an upper end of the range may be less than or equal to 195%, 190%, 185%, 180% or 175%); or (iii) greater than 200% to 500% (e.g. a lower end of the range may be at least 205%, 210%, 215%, 220% or 225% and/or an upper limit of the range may be less than or equal to 450%, 400%, 350%, or 300%, preferably 250%) of a reference folic acid daily dose, wherein the reference folic acid daily dose is 200 μg;
(d) a dose of cyanocobalamin that is: (i) greater than 100% to 305% (e.g. a lower end of the range may be at least 130%, 135%, 140%, 145% or 150%, preferably 150% and/or an upper end of the range may be less than or equal to 300%, 295%, 290%, 285% or 280%) of a reference cyanocobalamin daily dose; (ii) greater than 305% to 590% of a reference cyanocobalamin daily dose (e.g. a lower end of the range may be at least 310%, 315%, 320%, 325% or 330% and/or an upper end of the range may be less than or equal to 585%, 580%, 575%, 570% or 565%); or (iii) greater than 590% to 40000% (e.g. a lower end of the range may be at least 600%, 605%, 610%, 620% or 625% and/or an upper limit of the range may be less than or equal to 10000%, 9000%, 8000%, 7000%, 6000% or 5000%, preferably 1000%) of a reference cyanocobalamin daily dose, wherein the reference cyanocobalamin daily dose is 2.5 μg;
(e) a dose of ascorbic acid that is: (i) greater than 100% to 275% (e.g. a lower end of the range may be at least 140%, 145%, 150%, 155% or 160%, preferably 160% and/or an upper end of the range may be less than or equal to 270%, 265%, 260%, 255% or 250%) of a reference ascorbic acid daily dose; (ii) greater than 275% to 460% of a reference ascorbic acid daily dose (e.g. a lower end of the range may be at least 280%, 285%, 290%, 295% or 300% and/or an upper end of the range may be less than or equal to 455%, 450%, 445%, 440% or 435%); or (iii) greater than 460% to 2500% (e.g. a lower end of the range may be at least 465%, 470%, 475%, 480% or 485% and/or an upper limit of the range may be less than or equal to 1000%, 950%, 900%, 850% or 800%, preferably 750%) of a reference ascorbic acid daily dose, wherein the reference ascorbic acid daily dose is 80 mg;
(f) a dose of cholecalciferol that is: (i) greater than 100% to 320% (e.g. a lower end of the range may be at least 140%, 145%, 150%, 155% or 160%, preferably 160% and/or an upper end of the range may be less than or equal to 315%, 310%, 305%, 300% or 295%) of a reference cholecalciferol daily dose; (ii) greater than 320% to 545% of a reference cholecalciferol daily dose (e.g. a lower end of the range may be at least 325%, 330%, 335%, 340% or 345% and/or an upper end of the range may be less than or equal to 540%, 535%, 530%, 525% or 520%); or (iii) greater than 545% to 2000% (e.g. a lower end of the range may be at least 550%, 560%, 570%, 580% or 590% and/or an upper limit of the range may be less than or equal to 1000%, 950%, 900%, 850% or 800%, preferably 750%) of a reference cholecalciferol daily dose, wherein the reference cholecalciferol daily dose is 5 μg;
(g) a dose of vitamin E that is: (i) greater than 100% to 465% (e.g. a lower end of the range may be at least 105%, 110%, 115%, 120% or 125%, preferably 115% and/or an upper end of the range may be less than or equal to 460%, 455%, 450%, 445% or 440%) of a reference vitamin E daily dose; (ii) greater than 465% to 1090% of a reference vitamin E daily dose (e.g. a lower end of the range may be at least 470%, 475%, 480%, 485% or 490% and/or an upper end of the range may be less than or equal to 1000%, 900%, 800%, 700% or 600%); or (iii) greater than 1090% to 9167% (e.g. a lower end of the range may be at least 1100%, 1200%, 1300%, 1400% or 1500% and/or an upper limit of the range may be less than or equal to 7500%, 7000%, 6500%, 6000%, 5500% or 5000%, preferably 2000%) of a reference vitamin E daily dose, wherein the reference vitamin E daily dose is 12 mg;
(h) a dose of calcium that is: (i) greater than 100% to 122.5% (e.g. a lower end of the range may be at least 105%, 107%, 109%, 111% or 113%, preferably 107% and/or an upper end of the range may be less than or equal to 120%, 118%, 116%, 114% or 112%) of a reference calcium daily dose; (ii) greater than 122.5% to 143% of a reference calcium daily dose (e.g. a lower end of the range may be at least 124%, 126%, 128%, 130% or 132% and/or an upper end of the range may be less than or equal to 140%, 138%, 136%, 134% or 132%); or (iii) greater than 143% to 313% (e.g. a lower end of the range may be at least 150%, 155%, 160%, 165% or 170% and/or upper limit of the range may be less than or equal to 300%, 275%, 250%, 225% or 200%, preferably 175%) of a reference calcium daily dose, wherein the reference calcium daily dose is 800 mg; and/or
(i) a dose of selenium that is: (i) greater than 100% to 160% (e.g. a lower end of the range may be at least 105%, 110%, 115%, 120% or 125%, preferably 115% and/or an upper end of the range may be less than or equal to 155%, 150%, 145%, 140% or 135%) of a reference selenium daily dose; or (ii) greater than 160% to 727% (e.g. a lower end of the range may be at least 170%, 180%, 190%, 200% or 210% and/or an upper limit of the range may be less than or equal to 500%, 450%, 400%, 350% or 300%, preferably 250%) of a reference selenium daily dose, wherein the reference selenium daily dose is 55 μg.

A daily unit dose of the one or more micronutrients may refer to (or may comprise):
(a) a dose of vitamin A that is: (i) 115% to 125% of a reference vitamin A daily dose; (ii) 130% to 157% of a reference vitamin A daily dose; or (iii) 160% to 178% of a reference vitamin A daily dose, wherein the reference vitamin A daily dose is 800 μg;
(b) a dose of pyridoxine that is: (i) 200% to 260% of a reference pyridoxine daily dose; (ii) 350% to 530% of a reference pyridoxine daily dose; or (iii) 650% to 720% of a reference pyridoxine daily dose, wherein the reference pyridoxine daily dose is 1.4 mg;
(c) a dose of folic acid that is: (i) 120% to 140% of a reference folic acid daily dose; (ii) 150% to 195% of a reference folic acid daily dose; or (iii) 205% to 210% of a reference folic acid daily dose, wherein the reference folic acid daily dose is 200 μg;
(d) a dose of cyanocobalamin that is: (i) 200% to 260% of a reference cyanocobalamin daily dose; (ii) 350% to 530% of a reference cyanocobalamin daily dose; or (iii) 650% to 720% of a reference cyanocobalamin daily dose, wherein the reference cyanocobalamin daily dose is 2.5 μg;
(e) a dose of ascorbic acid that is: (i) 190% to 210% (preferably 200%) of a reference ascorbic acid daily dose; (ii) 350% to 420% of a reference ascorbic acid daily dose; or (iii) 500% to 570% of a reference ascorbic acid daily dose, wherein the reference ascorbic acid daily dose is 80 mg;
(f) a dose of cholecalciferol that is: (i) 180% to 240% of a reference cholecalciferol daily dose; (ii) 400% to 490% of a reference cholecalciferol daily dose; or (iii) 600% to 660% of a reference cholecalciferol daily dose, wherein the reference cholecalciferol daily dose is 5 μg;
(g) a dose of vitamin E that is: (i) 125% to 135% (preferably 130%) of a reference vitamin E daily dose; (ii) 800% to 880% of a reference vitamin E daily dose; or (iii) 1300% to 1370% of a reference vitamin E daily dose, wherein the reference vitamin E daily dose is 12 mg;
(h) a dose of calcium that is: (i) 110% to 120% of a reference calcium daily dose; (ii) 125% to 141% of a reference calcium daily dose; or (iii) 145% to 147% of a reference calcium daily dose, wherein the reference calcium daily dose is 800 mg; and/or
(i) a dose of selenium that is: (i) 120% to 155% of a reference selenium daily dose; or (ii) 165% to 205% of a reference selenium daily dose, wherein the reference selenium daily dose is 55 μg.

The daily unit doses for each of the micronutrients above preferably correspond to base/normal (i.e. where the daily unit dose is preceded by a (i)), medium (i.e. where the daily unit dose is preceded by a (ii)), or high levels of the micronutrients (i.e. where the daily unit dose is preceded by a (iii)), or base/normal (i.e. where the daily unit dose is preceded by a (i)) or high levels (i.e. where the daily unit dose is preceded by a (ii)) in the context of selenium. Similarly, the daily unit doses for each of the micronutrients above preferably correspond to either base/normal (i.e. where the daily unit dose is preceded by a (i)), medium (i.e. where the daily unit dose is preceded by a (ii)), or high need of the micronutrients (i.e. where the daily unit dose is preceded by a (iii)), or base/normal (i.e. where the daily unit dose is preceded by a (i)) or high need (i.e. where the daily unit dose is preceded by a (ii)) in the context of selenium. Said need is preferably a need for the indicated daily unit dose of the micronutrient.

As explained in more detail in the Examples section herein, the present inventors discovered 17 nutritional reference standards (a.k.a. archetypes) corresponding to the genetically-determined nutritional requirements of the majority of the population. Based on said nutritional reference standards, 17 nutritional compositions were prepared, each corresponding to a nutritional reference standard and thus each tailored to a portion of the population.

The tailored daily unit doses of one or more micronutrients corresponding to each nutritional reference standard (and thus nutritional compositions comprising a daily unit dose [or a plurality of daily unit doses or a fraction of daily unit dose] of the one or more micronutrients corresponding to the nutritional reference standard) are provided below as parts (a) to (q). The embodiments above further defining the lower and upper limits of the micronutrient ranges apply equally to each of (a) to (q) below.

A daily unit dose of the one or more micronutrients may comprise:
(a) vitamin A present at greater than 127.5% to 158.5% of a reference vitamin A daily dose, wherein the reference vitamin A daily dose is 800 μg; pyridoxine present at greater than 305% to 590% of a reference pyridoxine daily dose, wherein the reference pyridoxine daily dose is 1.4 mg; folic acid present at greater than 100% to 145% of a reference folic acid daily dose, wherein the reference folic acid daily dose is 200 μg; cyanocobalamin present at greater than 305% to 590% of a reference cyanocobalamin daily dose, wherein the reference cyanocobalamin daily dose is 2.5 μg; ascorbic acid present at greater than 275% to 460% of a reference ascorbic acid daily dose, wherein the reference ascorbic acid daily dose is 80 mg; cholecalciferol present at greater than 545% to 2000% of a reference cholecalciferol daily dose, wherein the reference cholecalciferol daily dose is 5 μg; vitamin E present at greater than 465% to 1090% of a reference vitamin E daily dose, wherein the reference vitamin E daily dose is 12 mg; calcium present at greater than 100% to 122.5% of a reference calcium daily dose, wherein the reference calcium daily dose is 800 mg; and/or (preferably and) selenium present at greater than 160% to 727% of a reference selenium daily dose, wherein the reference selenium daily dose is 55 μg; or
(b) vitamin A present at greater than 158.5% to 375% of a reference vitamin A daily dose, wherein the reference vitamin A daily dose is 800 μg; pyridoxine present at greater than 305% to 590% of a reference pyridoxine daily dose, wherein the reference pyridoxine daily dose is 1.4 mg; folic acid present at greater than 100% to 145% of a reference folic acid daily dose, wherein the reference folic acid daily dose is 200 μg; cyanocobalamin present at greater than 305% to 590% of a reference cyanocobalamin daily dose, wherein the reference cyanocobalamin daily dose is 2.5 μg; ascorbic acid present at greater than 275% to 460% of a reference ascorbic acid daily dose, wherein the reference ascorbic acid daily dose is 80 mg; cholecalciferol present at greater than 320% to 545% of a reference cholecalciferol daily dose, wherein the reference cholecalciferol daily dose is 5 μg; vitamin E present at greater than 465% to 1090% of a reference vitamin E daily dose, wherein the reference vitamin E daily dose is 12 mg; calcium present at greater than 100% to 122.5% of a reference calcium daily dose, wherein the reference calcium daily dose is 800 mg; and/or (preferably and) selenium present at greater than 100% to 160% of a reference selenium daily dose, wherein the reference selenium daily dose is 55 μg; or
(c) vitamin A present at greater than 127.5% to 158.5% of a reference vitamin A daily dose, wherein the reference vitamin A daily dose is 800 μg; pyridoxine present at greater than 590% to 7143% of a reference pyridoxine daily dose, wherein the reference pyridoxine daily dose is 1.4 mg; folic acid present at greater than 200% to 500% of a reference folic acid daily dose, wherein the reference folic acid daily dose is 200 μg; cyanocobalamin present at greater than 590% to 40000% of a reference cyanocobalamin daily dose, wherein the reference cyanocobalamin daily dose is 2.5 μg; ascorbic acid present at greater than 275% to 460% of a reference ascorbic acid daily dose, wherein the reference ascorbic acid daily dose is 80 mg; cholecalciferol present at greater than 545% to 2000% of a reference cholecalciferol daily dose, wherein the reference cholecalciferol daily dose is 5 μg; vitamin E present at greater than 465% to 1090% of a reference vitamin E daily dose, wherein the reference vitamin E daily dose is 12 mg; calcium present at greater than 122.5% to 143% of a reference calcium daily dose, wherein the reference calcium daily dose is 800 mg; and/or (preferably and) selenium present at greater than 100% to 160% of a reference selenium daily dose, wherein the reference selenium daily dose is 55 μg; or
(d) vitamin A present at greater than 158.5% to 375% of a reference vitamin A daily dose, wherein the reference vitamin A daily dose is 800 μg; pyridoxine present at greater than 305% to 590% of a reference pyridoxine daily dose, wherein the reference pyridoxine daily dose is 1.4 mg; folic acid present at greater than 100% to 145% of a reference folic acid daily dose, wherein the reference folic acid daily dose is 200 μg; cyanocobalamin present at greater than 305% to 590% of a reference cyanocobalamin daily dose, wherein the reference cyanocobalamin daily dose is 2.5 μg; ascorbic acid present at greater than 275% to 460% of a reference ascorbic acid daily dose, wherein the reference ascorbic acid daily dose is 80 mg; cholecalciferol present at greater than 545% to 2000% of a reference cholecalciferol daily dose, wherein the reference cholecalciferol daily dose is 5 μg; vitamin E present at greater than 465% to 1090% of a reference vitamin E daily dose, wherein the reference vitamin E daily dose is 12 mg; calcium present at greater than 143% to 313% of a reference calcium daily dose, wherein the reference calcium daily dose is 800 mg; and/or (preferably and) selenium present at greater than 100% to 160% of a reference selenium daily dose, wherein the reference selenium daily dose is 55 μg; or
(e) vitamin A present at greater than 158.5% to 375% of a reference vitamin A daily dose, wherein the reference vitamin A daily dose is 800 μg; pyridoxine present at greater than 590% to 7143% of a reference pyridoxine daily dose, wherein the reference pyridoxine daily dose is 1.4 mg; folic acid present at greater than 100% to 145% of a reference folic acid daily dose, wherein the reference folic acid daily dose is 200 μg; cyanocobalamin present at greater than 590% to 40000% of a reference cyanocobalamin daily dose, wherein the reference cyanocobalamin daily dose is 2.5 μg; ascorbic acid present at greater than 460% to 2500% of a reference ascorbic acid daily dose wherein the reference ascorbic acid daily dose is 80 mg; cholecalciferol present at greater than 545% to 2000% of a reference cholecalciferol daily dose, wherein the reference cholecalciferol daily dose is 5 μg; vitamin E present at greater than 1090% to 9167% of a reference vitamin E daily dose, wherein the reference vitamin E daily dose is 12 mg; calcium present at greater than 122.5% to 143% of a reference calcium daily dose, wherein the reference calcium daily dose is 800 mg; and/or (preferably and) selenium present at greater than 160% to 727% of a reference selenium daily dose, wherein the reference selenium daily dose is 55 μg; or
(f) vitamin A present at greater than 127.5% to 158.5% of a reference vitamin A daily dose, wherein the reference vitamin A daily dose is 800 μg; pyridoxine present at greater than 590% to 7143% of a reference pyridoxine daily dose, wherein the reference pyridoxine daily dose is 1.4 mg; folic acid present at greater than 145% to 200% of a reference folic acid daily dose, wherein the reference folic acid daily dose is 200 μg; cyanocobalamin present at greater than 590% to 40000% of a reference cyanocobalamin daily dose, wherein the reference cyanocobalamin daily dose is 2.5 μg; ascorbic acid present at greater than 275% to 460% of a reference ascorbic acid daily dose, wherein the reference ascorbic acid daily dose is 80 mg; cholecalciferol present at greater than 100% to 320% of a reference cholecalciferol daily dose, wherein the reference cholecalciferol daily dose is 5 μg; vitamin E present at greater than 465% to 1090% of a reference vitamin E daily dose, wherein the reference vitamin E daily dose is 12 mg; calcium present at greater than 100% to 122.5% of a reference calcium daily dose, wherein the reference calcium daily dose is 800 mg; and/or (preferably and) selenium present at greater than 160% to 727% of a reference selenium daily dose, wherein the reference selenium daily dose is 55 μg; or
(g) vitamin A present at greater than 127.5% to 158.5% of a reference vitamin A daily dose, wherein the reference vitamin A daily dose is 800 μg; pyridoxine present greater than 100% to 305% of a reference pyridoxine daily dose, wherein the reference pyridoxine daily dose is 1.4 mg; folic acid present at greater than 145% to 200% of a reference folic acid daily dose, wherein the reference folic acid daily dose is 200 μg; cyanocobalamin present at greater than 100% to 305% of a reference cyanocobalamin daily dose, wherein the reference cyanocobalamin daily dose is 2.5 μg; ascorbic acid present at greater than 460% to 2500% of a reference ascorbic acid daily dose wherein the reference ascorbic acid daily dose is 80 mg; cholecalciferol present at greater than 320% to 545% of a reference cholecalciferol daily dose, wherein the reference cholecalciferol daily dose is 5 μg; vitamin E present at greater than 1090% to 9167% of a reference vitamin E daily dose, wherein the reference vitamin E daily dose is 12 mg; calcium present at greater than 122.5% to 143% of a reference calcium daily dose, wherein the reference calcium daily dose is 800 mg; and/or (preferably and) selenium present at greater than 160% to 727% of a reference selenium daily dose, wherein the reference selenium daily dose is 55 μg; or
(h) vitamin A present at greater than 127.5% to 158.5% of a reference vitamin A daily dose, wherein the reference vitamin A daily dose is 800 μg; pyridoxine present at greater than 305% to 590% of a reference pyridoxine daily dose, wherein the reference pyridoxine daily dose is 1.4 mg; folic acid present at greater than 145% to 200% of a reference folic acid daily dose, wherein the reference folic acid daily dose is 200 μg; cyanocobalamin present at greater than 305% to 590% of a reference cyanocobalamin daily dose, wherein the reference cyanocobalamin daily dose is 2.5 μg; ascorbic acid present at greater than 275% to 460% of a reference ascorbic acid daily dose, wherein the reference ascorbic acid daily dose is 80 mg; cholecalciferol present at greater than 100% to 320% of a reference cholecalciferol daily dose, wherein the reference cholecalciferol daily dose is 5 μg; vitamin E present at greater than 465% to 1090% of a reference vitamin E daily dose, wherein the reference vitamin E daily dose is 12 mg; calcium present at greater than 122.5% to 143% of a reference calcium daily dose, wherein the reference calcium daily dose is 800 mg; and/or (preferably and) selenium present at greater than 160% to 727% of a reference selenium daily dose, wherein the reference selenium daily dose is 55 μg; or
(i) vitamin A present at greater than 127.5% to 158.5% of a reference vitamin A daily dose, wherein the reference vitamin A daily dose is 800 μg; pyridoxine present at greater than 305% to 590% of a reference pyridoxine daily dose, wherein the reference pyridoxine daily dose is 1.4 mg; folic acid present at greater than 145% to 200% of a reference folic acid daily dose, wherein the reference folic acid daily dose is 200 μg; cyanocobalamin present at greater than 305% to 590% of a reference cyanocobalamin daily dose, wherein the reference cyanocobalamin daily dose is 2.5 μg; ascorbic acid present at greater than 100% to 275% of a reference ascorbic acid daily dose, wherein the reference ascorbic acid daily dose is 80 mg; cholecalciferol present at greater than 320% to 545% of a reference cholecalciferol daily dose, wherein the reference cholecalciferol daily dose is 5 μg; vitamin E present at greater than 100% to 465% of a reference vitamin E daily dose, wherein the reference vitamin E daily dose is 12 mg; calcium present at greater than 122.5% to 143% of a reference calcium daily dose, wherein the reference calcium daily dose is 800 mg; and/or (preferably and) selenium present at greater than 100% to 160% of a reference selenium daily dose, wherein the reference selenium daily dose is 55 μg; or
(j) vitamin A present at greater than 158.5% to 375% of a reference vitamin A daily dose, wherein the reference vitamin A daily dose is 800 μg; pyridoxine present at greater than 305% to 590% of a reference pyridoxine daily dose, wherein the reference pyridoxine daily dose is 1.4 mg; folic acid present at greater than 100% to 145% of a reference folic acid daily dose, wherein the reference folic acid daily dose is 200 μg; cyanocobalamin present at greater than 305% to 590% of a reference cyanocobalamin daily dose, wherein the reference cyanocobalamin daily dose is 2.5 μg; ascorbic acid present at greater than 460% to 2500% of a reference ascorbic acid daily dose wherein the reference ascorbic acid daily dose is 80 mg; cholecalciferol present at greater than 320% to 545% of a reference cholecalciferol daily dose, wherein the reference cholecalciferol daily dose is 5 μg; vitamin E present at greater than 1090% to 9167% of a reference vitamin E daily dose, wherein the reference vitamin E daily dose is 12 mg; calcium present at greater than 122.5% to 143% of a reference calcium daily dose, wherein the reference calcium daily dose is 800 mg; and/or (preferably and) selenium present at greater than 160% to 727% of a reference selenium daily dose, wherein the reference selenium daily dose is 55 μg; or
(k) vitamin A present at greater than 158.5% to 375% of a reference vitamin A daily dose, wherein the reference vitamin A daily dose is 800 μg; pyridoxine present at greater than 305% to 590% of a reference pyridoxine daily dose, wherein the reference pyridoxine daily dose is 1.4 mg; folic acid present at greater than 145% to 200% of a reference folic acid daily dose, wherein the reference folic acid daily dose is 200 μg; cyanocobalamin present at greater than 305% to 590% of a reference cyanocobalamin daily dose, wherein the reference cyanocobalamin daily dose is 2.5 μg; ascorbic acid present at greater than 460% to 2500% of a reference ascorbic acid daily dose wherein the reference ascorbic acid daily dose is 80 mg; cholecalciferol present at greater than 320% to 545% of a reference cholecalciferol daily dose, wherein the reference cholecalciferol daily dose is 5 μg; vitamin E present at greater than 1090% to 9167% of a reference vitamin E daily dose, wherein the reference vitamin E daily dose is 12 mg; calcium present at greater than 122.5% to 143% of a reference calcium daily dose, wherein the reference calcium daily dose is 800 mg; and/or (preferably and) selenium present at greater than 100% to 160% of a reference selenium daily dose, wherein the reference selenium daily dose is 55 μg; or
(l) vitamin A present at greater than 127.5% to 158.5% of a reference vitamin A daily dose, wherein the reference vitamin A daily dose is 800 μg; pyridoxine present at greater than 305% to 590% of a reference pyridoxine daily dose, wherein the reference pyridoxine daily dose is 1.4 mg; folic acid present at greater than 145% to 200% of a reference folic acid daily dose, wherein the reference folic acid daily dose is 200 μg; cyanocobalamin present at greater than 305% to 590% of a reference cyanocobalamin daily dose, wherein the reference cyanocobalamin daily dose is 2.5 μg; ascorbic acid present at greater than 460% to 2500% of a reference ascorbic acid daily dose wherein the reference ascorbic acid daily dose is 80 mg; cholecalciferol present at greater than 100% to 320% of a reference cholecalciferol daily dose, wherein the reference cholecalciferol daily dose is 5 μg; vitamin E present at greater than 1090% to 9167% of a reference vitamin E daily dose, wherein the reference vitamin E daily dose is 12 mg; calcium present at greater than 100% to 122.5% of a reference calcium daily dose, wherein the reference calcium daily dose is 800 mg; and/or (preferably and) selenium present at greater than 160% to 727% of a reference selenium daily dose, wherein the reference selenium daily dose is 55 μg; or
(m) vitamin A present at greater than 158.5% to 375% of a reference vitamin A daily dose, wherein the reference vitamin A daily dose is 800 μg; pyridoxine present at greater than 100% to 305% of a reference pyridoxine daily dose, wherein the reference pyridoxine daily dose is 1.4 mg; folic acid present at greater than 200% to 500% of a reference folic acid daily dose, wherein the reference folic acid daily dose is 200 μg; cyanocobalamin present at greater than 100% to 305% of a reference cyanocobalamin daily dose, wherein the reference cyanocobalamin daily dose is 2.5 μg; ascorbic acid present at greater than 275% to 460% of a reference ascorbic acid daily dose, wherein the reference ascorbic acid daily dose is 80 mg; cholecalciferol present at greater than 320% to 545% of a reference cholecalciferol daily dose, wherein the reference cholecalciferol daily dose is 5 μg; vitamin E present at greater than 465% to 1090% of a reference vitamin E daily dose, wherein the reference vitamin E daily dose is 12 mg; calcium present at greater than 122.5% to 143% of a reference calcium daily dose, wherein the reference calcium daily dose is 800 mg; and/or (preferably and) selenium present at greater than 100% to 160% of a reference selenium daily dose, wherein the reference selenium daily dose is 55 μg; or
(n) vitamin A present at greater than 127.5% to 158.5% of a reference vitamin A daily dose, wherein the reference vitamin A daily dose is 800 μg; pyridoxine present at greater than 590% to 7143% of a reference pyridoxine daily dose, wherein the reference pyridoxine daily dose is 1.4 mg; folic acid present greater than 145% to 200% of a reference folic acid daily dose, wherein the reference folic acid daily dose is 200 μg; cyanocobalamin present at greater than 590% to 40000% of a reference cyanocobalamin daily dose, wherein the reference cyanocobalamin daily dose is 2.5 μg; ascorbic acid present at greater than 460% to 2500% of a reference ascorbic acid daily dose wherein the reference ascorbic acid daily dose is 80 mg; cholecalciferol present at greater than 320% to 545% of a reference cholecalciferol daily dose, wherein the reference cholecalciferol daily dose is 5 μg; vitamin E present at greater than 1090% to 9167% of a reference vitamin E daily dose, wherein the reference vitamin E daily dose is 12 mg; calcium present at greater than 122.5% to 143% of a reference calcium daily dose, wherein the reference calcium daily dose is 800 mg; and/or (preferably and) selenium present at greater than 160% to 727% of a reference selenium daily dose, wherein the reference selenium daily dose is 55 μg; or
(o) vitamin A present at greater than 158.5% to 375% of a reference vitamin A daily dose, wherein the reference vitamin A daily dose is 800 μg; pyridoxine present at greater than 100% to 305% of a reference pyridoxine daily dose, wherein the reference pyridoxine daily dose is 1.4 mg; folic acid present at greater than 145% to 200% of a reference folic acid daily dose, wherein the reference folic acid daily dose is 200 μg; cyanocobalamin present at greater than 100% to 305% of a reference cyanocobalamin daily dose, wherein the reference cyanocobalamin daily dose is 2.5 μg; ascorbic acid present at greater than 460% to 2500% of a reference ascorbic acid daily dose, wherein the reference ascorbic acid daily dose is 80 mg; cholecalciferol present at greater than 100% to 320% of a reference cholecalciferol daily dose, wherein the reference cholecalciferol daily dose is 5 μg; vitamin E present at greater than 1090% to 9167% of a reference vitamin E daily dose, wherein the reference vitamin E daily dose is 12 mg; calcium present at greater than 100% to 122.5% of a reference calcium daily dose, wherein the reference calcium daily dose is 800 mg; and/or (preferably and) selenium present at greater than 100% to 160% of a reference selenium daily dose, wherein the reference selenium daily dose is 55 μg; or
(p) vitamin A present at greater than 127.5% to 158.5% of a reference vitamin A daily dose, wherein the reference vitamin A daily dose is 800 μg; pyridoxine present at greater than 100% to 305% of a reference pyridoxine daily dose, wherein the reference pyridoxine daily dose is 1.4 mg; folic acid present at greater than 145% to 200% of a reference folic acid daily dose, wherein the reference folic acid daily dose is 200 μg; cyanocobalamin present at greater than 100% to 305% of a reference cyanocobalamin daily dose, wherein the reference cyanocobalamin daily dose is 2.5 μg; ascorbic acid present at greater than 275% to 460% of a reference ascorbic acid daily dose, wherein the reference ascorbic acid daily dose is 80 mg; cholecalciferol present at greater than 100% to 320% of a reference cholecalciferol daily dose, wherein the reference cholecalciferol daily dose is 5 μg; vitamin E present at greater than 465% to 1090% of a reference vitamin E daily dose, wherein the reference vitamin E daily dose is 12 mg; calcium present at greater than 100% to 122.5% of a reference calcium daily dose, wherein the reference calcium daily dose is 800 mg; and/or (preferably and) selenium present at greater than 160% to 727% of a reference selenium daily dose, wherein the reference selenium daily dose is 55 μg; or
(q) vitamin A present at greater than 127.5% to 158.5% of a reference vitamin A daily dose, wherein the reference vitamin A daily dose is 800 μg; pyridoxine present at greater than 305% to 590% of a reference pyridoxine daily dose, wherein the reference pyridoxine daily dose is 1.4 mg; folic acid present at greater than 145% to 200% of a reference folic acid daily dose, wherein the reference folic acid daily dose is 200 μg; cyanocobalamin present at greater than 305% to 590% of a reference cyanocobalamin daily dose, wherein the reference cyanocobalamin daily dose is 2.5 μg; ascorbic acid present at greater than 460% to 2500% of a reference ascorbic acid daily dose, wherein the reference ascorbic acid daily dose is 80 mg; cholecalciferol present at greater than 545% to 2000% of a reference cholecalciferol daily dose, wherein the reference cholecalciferol daily dose is 5 μg; vitamin E present at greater than 1090% to 9167% of a reference vitamin E daily dose, wherein the reference vitamin E daily dose is 12 mg; calcium present at greater than 143% to 313% of a reference calcium daily dose, wherein the reference calcium daily dose is 800 mg; and/or (preferably and) selenium present at greater than 100% to 160% of a reference selenium daily dose, wherein the reference selenium daily dose is 55 μg.

Again, a nutritional composition corresponding to each nutritional reference standard may comprise a fraction of a daily unit dose of the one or more micronutrients.

For example, a daily unit dose of the one or more micronutrients may comprise:
(a) vitamin A present at 130% to 157% (preferably 130%) of a reference vitamin A daily dose, wherein the reference vitamin A daily dose is 800 μg; pyridoxine present at 350% to 530% (preferably 350%) of a reference pyridoxine daily dose, wherein the reference pyridoxine daily dose is 1.4 mg; folic acid present at 120% to 140% (preferably 120%) of a reference folic acid daily dose, wherein the reference folic acid daily dose is 200 μg; cyanocobalamin present at 350% to 530% (preferably 350%) of a reference cyanocobalamin daily dose, wherein the reference cyanocobalamin daily dose is 2.5 μg; ascorbic acid present at 350% to 420% (preferably 350%) of a reference ascorbic acid daily dose, wherein the reference ascorbic acid daily dose is 80 mg; cholecalciferol present at 600% to 660% (preferably 600%) of a reference cholecalciferol daily dose, wherein the reference cholecalciferol daily dose is 5 μg; vitamin E present at 800% to 880% (preferably 800%) of a reference vitamin E daily dose, wherein the reference vitamin E daily dose is 12 mg; calcium present at 110% to 120% (preferably 110%) of a reference calcium daily dose, wherein the reference calcium daily dose is 800 mg; and/or (preferably and) selenium present at 165% to 205% (preferably 165%) of a reference selenium daily dose, wherein the reference selenium daily dose is 55 μg; or
(b) vitamin A present at 160% to 178% (preferably 160%) of a reference vitamin A daily dose, wherein the reference vitamin A daily dose is 800 μg; pyridoxine present at 350% to 530% (preferably 370%) of a reference pyridoxine daily dose, wherein the reference pyridoxine daily dose is 1.4 mg; folic acid present at 120% to 140% (preferably 125%) of a reference folic acid daily dose, wherein the reference folic acid daily dose is 200 μg; cyanocobalamin present at 350% to 530% (preferably 370%) of a reference cyanocobalamin daily dose, wherein the reference cyanocobalamin daily dose is 2.5 μg; ascorbic acid present at 350% to 420% (preferably 360%) of a reference ascorbic acid daily dose, wherein the reference ascorbic acid daily dose is 80 mg; cholecalciferol present at 400% to 490% (preferably 400%) of a reference cholecalciferol daily dose, wherein the reference cholecalciferol daily dose is 5 μg; vitamin E present at 800% to 880% (preferably 810%) of a reference vitamin E daily dose, wherein the reference vitamin E daily dose is 12 mg; calcium present at 110% to 120% (preferably 112%) of a reference calcium daily dose, wherein the reference calcium daily dose is 800 mg; and/or (preferably and) selenium present at 120% to 155% (preferably 120%) of a reference selenium daily dose, wherein the reference selenium daily dose is 55 μg; or
(c) vitamin A present at 130% to 157% (preferably 133%) of a reference vitamin A daily dose, wherein the reference vitamin A daily dose is 800 μg; pyridoxine present at 650% to 720% (preferably 650%) of a reference pyridoxine daily dose, wherein the reference pyridoxine daily dose is 1.4 mg; folic acid present at 205% to 210% (preferably 205%) of a reference folic acid daily dose, wherein the reference folic acid daily dose is 200 μg; cyanocobalamin present at 650% to 720% (preferably 650%) of a reference cyanocobalamin daily dose, wherein the reference cyanocobalamin daily dose is 2.5 μg; ascorbic acid present at 350% to 420% (preferably 370%) of a reference ascorbic acid daily dose, wherein the reference ascorbic acid daily dose is 80 mg; cholecalciferol present at 600% to 660% (preferably 615%) of a reference cholecalciferol daily dose, wherein the reference cholecalciferol daily dose is 5 μg; vitamin E present at 800% to 880% (preferably 820%) of a reference vitamin E daily dose, wherein the reference vitamin E daily dose is 12 mg; calcium present at 125% to 141% (preferably 125%) of a reference calcium daily dose, wherein the reference calcium daily dose is 800 mg; and/or (preferably and) selenium present at 120% to 155% (preferably 125%) of a reference selenium daily dose, wherein the reference selenium daily dose is 55 μg; or
(d) vitamin A present at 160% to 178% (preferably 163%) of a reference vitamin A daily dose, wherein the reference vitamin A daily dose is 800 μg; pyridoxine present at 350% to 530% (preferably 410%) of a reference pyridoxine daily dose, wherein the reference pyridoxine daily dose is 1.4 mg; folic acid present at 120% to 140% (preferably 130%) of a reference folic acid daily dose, wherein the reference folic acid daily dose is 200 μg; cyanocobalamin present at 350% to 530% (preferably 410%) of a reference cyanocobalamin daily dose, wherein the reference cyanocobalamin daily dose is 2.5 μg; ascorbic acid present at 350% to 420% (preferably 380%) of a reference ascorbic acid daily dose, wherein the reference ascorbic acid daily dose is 80 mg; cholecalciferol present at 600% to 660% (preferably 630%) of a reference cholecalciferol daily dose, wherein the reference cholecalciferol daily dose is 5 μg; vitamin E present at 800% to 880% (preferably 830%) of a reference vitamin E daily dose, wherein the reference vitamin E daily dose is 12 mg; calcium present at 145% to 147% (preferably 145%) of a reference calcium daily dose, wherein the reference calcium daily dose is 800 mg; and/or (preferably and) selenium present at 120% to 155% (preferably 130%) of a reference selenium daily dose, wherein the reference selenium daily dose is 55 μg; or
(e) vitamin A present at 160% to 178% (preferably 166%) of a reference vitamin A daily dose, wherein the reference vitamin A daily dose is 800 μg; pyridoxine present at 650% to 720% (preferably 670%) of a reference pyridoxine daily dose, wherein the reference pyridoxine daily dose is 1.4 mg; folic acid present at 120% to 140% (preferably 135%) of a reference folic acid daily dose, wherein the reference folic acid daily dose is 200 μg; cyanocobalamin present at 650% to 720% (preferably 670%) of a reference cyanocobalamin daily dose, wherein the reference cyanocobalamin daily dose is 2.5 μg; ascorbic acid present at 500% to 570% (preferably 500%) of a reference ascorbic acid daily dose, wherein the reference ascorbic acid daily dose is 80 mg; cholecalciferol present at: 600% to 660% (preferably 645%) of a reference cholecalciferol daily dose, wherein the reference cholecalciferol daily dose is 5 μg; vitamin E present at 1300% to 1370% (preferably 1300%) of a reference vitamin E daily dose, wherein the reference vitamin E daily dose is 12 mg; calcium present at 125% to 141% (preferably 127%) of a reference calcium daily dose, wherein the reference calcium daily dose is 800 mg; and/or (preferably and) selenium present at 165% to 205% (preferably 170%) of a reference selenium daily dose, wherein the reference selenium daily dose is 55 μg; or
(f) vitamin A present at 130% to 157% (preferably 136%) of a reference vitamin A daily dose, wherein the reference vitamin A daily dose is 800 μg; pyridoxine present at 650% to 720% (preferably 690%) of a reference pyridoxine daily dose, wherein the reference pyridoxine daily dose is 1.4 mg; folic acid present at 150% to 195% (preferably 150%) of a reference folic acid daily dose, wherein the reference folic acid daily dose is 200 μg; cyanocobalamin present at 650% to 720% (preferably 690%) of a reference cyanocobalamin daily dose, wherein the reference cyanocobalamin daily dose is 2.5 μg; ascorbic acid present at 350% to 420% (preferably 390%) of a reference ascorbic acid daily dose, wherein the reference ascorbic acid daily dose is 80 mg; cholecalciferol present at 180% to 240% (preferably 180%) of a reference cholecalciferol daily dose, wherein the reference cholecalciferol daily dose is 5 μg; vitamin E present at 800% to 880% (preferably 840%) of a reference vitamin E daily dose, wherein the reference vitamin E daily dose is 12 mg; calcium present at 110% to 120% (preferably 114%) of a reference calcium daily dose, wherein the reference calcium daily dose is 800 mg; and/or (preferably and) selenium present at 165% to 205% (preferably 175%) of a reference selenium daily dose, wherein the reference selenium daily dose is 55 μg; or
(g) vitamin A present at 130% to 157% (preferably 139%) of a reference vitamin A daily dose, wherein the reference vitamin A daily dose is 800 μg; pyridoxine present at 200% to 260% (preferably 200%) of a reference pyridoxine daily dose, wherein the reference pyridoxine daily dose is 1.4 mg; folic acid present at 150% to 195% (preferably 155%) of a reference folic acid daily dose, wherein the reference folic acid daily dose is 200 μg; cyanocobalamin present at 200% to 260% (preferably 200%) of a reference cyanocobalamin daily dose, wherein the reference cyanocobalamin daily dose is 2.5 μg; ascorbic acid present at 500% to 570% (preferably 510%) of a reference ascorbic acid daily dose, wherein the reference ascorbic acid daily dose is 80 mg; cholecalciferol present at 400% to 490% (preferably 415%) of a reference cholecalciferol daily dose, wherein the reference cholecalciferol daily dose is 5 μg; vitamin E present at 1300% to 1370% (preferably 1310%) of a reference vitamin E daily dose, wherein the reference vitamin E daily dose is 12 mg; calcium present at 125% to 141% (preferably 129%) of a reference calcium daily dose, wherein the reference calcium daily dose is 800 mg; and/or (preferably and) selenium present at 165% to 205% (preferably 180%) of a reference selenium daily dose, wherein the reference selenium daily dose is 55 μg; or
(h) vitamin A present at 130% to 157% (preferably 142%) of a reference vitamin A daily dose, wherein the reference vitamin A daily dose is 800 μg; pyridoxine present at 350% to 530% (preferably 430%) of a reference pyridoxine daily dose, wherein the reference pyridoxine daily dose is 1.4 mg; folic acid present at 150% to 195% (preferably 160%) of a reference folic acid daily dose, wherein the reference folic acid daily dose is 200 μg; cyanocobalamin present at 350% to 530% (preferably 430%) of a reference cyanocobalamin daily dose, wherein the reference cyanocobalamin daily dose is 2.5 μg; ascorbic acid present at 350% to 420% (preferably 400%) of a reference ascorbic acid daily dose, wherein the reference ascorbic acid daily dose is 80 mg; cholecalciferol present at 180% to 240% (preferably 195%) of a reference cholecalciferol daily dose, wherein the reference cholecalciferol daily dose is 5 μg; vitamin E present at 800% to 880% (preferably 850%) of a reference vitamin E daily dose, wherein the reference vitamin E daily dose is 12 mg; calcium present at 125% to 141% (preferably 131%) of a reference calcium daily dose, wherein the reference calcium daily dose is 800 mg; and/or (preferably and) selenium present at 165% to 205% (preferably 185%) of a reference selenium daily dose, wherein the reference selenium daily dose is 55 μg; or
(i) vitamin A present at 130% to 157% (preferably 145%) of a reference vitamin A daily dose, wherein the reference vitamin A daily dose is 800 μg; pyridoxine present at 350% to 530% (preferably 450%) of a reference pyridoxine daily dose, wherein the reference pyridoxine daily dose is 1.4 mg; folic acid present at 150% to 195% (preferably 165%) of a reference folic acid daily dose, wherein the reference folic acid daily dose is 200 μg; cyanocobalamin present at 350% to 530% (preferably 450%) of a reference cyanocobalamin daily dose, wherein the reference cyanocobalamin daily dose is 2.5 μg; ascorbic acid present at 190% to 210% (preferably 200%) of a reference ascorbic acid daily dose, wherein the reference ascorbic acid daily dose is 80 mg; cholecalciferol present at 400% to 490% (preferably 430%) of a reference cholecalciferol daily dose, wherein the reference cholecalciferol daily dose is 5 μg; vitamin E present at 125% to 135% (preferably 130%) of a reference vitamin E daily dose, wherein the reference vitamin E daily dose is 12 mg; calcium present at 125% to 141% (preferably 133%) of a reference calcium daily dose, wherein the reference calcium daily dose is 800 mg; and/or (preferably and) selenium present at 120% to 155% (preferably 135%) of a reference selenium daily dose, wherein the reference selenium daily dose is 55 μg; or
(j) vitamin A present at 160% to 178% (preferably 169%) of a reference vitamin A daily dose, wherein the reference vitamin A daily dose is 800 μg; pyridoxine present at 350% to 530% (preferably 470%) of a reference pyridoxine daily dose, wherein the reference pyridoxine daily dose is 1.4 mg; folic acid present at 120% to 140% (preferably 140%) of a reference folic acid daily dose, wherein the reference folic acid daily dose is 200 μg; cyanocobalamin present at 350% to 530% (preferably 470%) of a reference cyanocobalamin daily dose, wherein the reference cyanocobalamin daily dose is 2.5 μg; ascorbic acid present at 500% to 570% (preferably 520%) of a reference ascorbic acid daily dose, wherein the reference ascorbic acid daily dose is 80 mg; cholecalciferol present at 400% to 490% (preferably 445%) of a reference cholecalciferol daily dose, wherein the reference cholecalciferol daily dose is 5 μg; vitamin E present at 1300% to 1370% (preferably 1320%) of a reference vitamin E daily dose, wherein the reference vitamin E daily dose is 12 mg; calcium present at 125% to 141% (preferably 135%) of a reference calcium daily dose, wherein the reference calcium daily dose is 800 mg; and/or (preferably and) selenium present at 165% to 205% (preferably 190%) of a reference selenium daily dose, wherein the reference selenium daily dose is 55 μg; or
(k) vitamin A present at 160% to 178% (preferably 172%) of a reference vitamin A daily dose, wherein the reference vitamin A daily dose is 800 μg; pyridoxine present at 350% to 530% (preferably 490%) of a reference pyridoxine daily dose, wherein the reference pyridoxine daily dose is 1.4 mg; folic acid present at 150% to 195% (preferably 170%) of a reference folic acid daily dose, wherein the reference folic acid daily dose is 200 μg; cyanocobalamin present at 350% to 530% (preferably 490%) of a reference cyanocobalamin daily dose, wherein the reference cyanocobalamin daily dose is 2.5 μg; ascorbic acid present at 500% to 570% (preferably 530%) of a reference ascorbic acid daily dose wherein the reference ascorbic acid daily dose is 80 mg; cholecalciferol present at 400% to 490% (preferably 460%) of a reference cholecalciferol daily dose, wherein the reference cholecalciferol daily dose is 5 μg; vitamin E present at 1300% to 1370% (preferably 1330%) of a reference vitamin E daily dose, wherein the reference vitamin E daily dose is 12 mg; calcium present at 125% to 141% (preferably 137%) of a reference calcium daily dose, wherein the reference calcium daily dose is 800 mg; and/or (preferably and) selenium present at 120% to 155% (preferably 140%) of a reference selenium daily dose, wherein the reference selenium daily dose is 55 μg; or
(l) vitamin A present at 130% to 157% (preferably 148%) of a reference vitamin A daily dose, wherein the reference vitamin A daily dose is 800 μg; pyridoxine present at 350% to 530% (preferably 510%) of a reference pyridoxine daily dose, wherein the reference pyridoxine daily dose is 1.4 mg; folic acid present at 150% to 195% (preferably 175%) of a reference folic acid daily dose, wherein the reference folic acid daily dose is 200 μg; cyanocobalamin present at 350% to 530% (preferably 510%) of a reference cyanocobalamin daily dose, wherein the reference cyanocobalamin daily dose is 2.5 μg; ascorbic acid present at 500% to 570% (preferably 540%) of a reference ascorbic acid daily dose wherein the reference ascorbic acid daily dose is 80 mg; cholecalciferol present at 180% to 240% (preferably 210%) of a reference cholecalciferol daily dose, wherein the reference cholecalciferol daily dose is 5 μg; vitamin E present 1300% to 1370% (preferably 1340%) of a reference vitamin E daily dose, wherein the reference vitamin E daily dose is 12 mg; calcium present at 110% to 120% (preferably 116%) of a reference calcium daily dose, wherein the reference calcium daily dose is 800 mg; and/or (preferably and) selenium present at 165% to 205% (preferably 195%) of a reference selenium daily dose, wherein the reference selenium daily dose is 55 μg; or
(m) vitamin A present at 160% to 178% (preferably 175%) of a reference vitamin A daily dose, wherein the reference vitamin A daily dose is 800 μg; pyridoxine present at 200% to 260% (preferably 220%) of a reference pyridoxine daily dose, wherein the reference pyridoxine daily dose is 1.4 mg; folic acid present at 205% to 210% (preferably 210%) of a reference folic acid daily dose, wherein the reference folic acid daily dose is 200 μg; cyanocobalamin present at 200% to 260% (preferably 220%) of a reference cyanocobalamin daily dose, wherein the reference cyanocobalamin daily dose is 2.5 μg; ascorbic acid present at 350% to 420% (preferably 410%) of a reference ascorbic acid daily dose, wherein the reference ascorbic acid daily dose is 80 mg; cholecalciferol present at 400% to 490% (preferably 475%) of a reference cholecalciferol daily dose, wherein the reference cholecalciferol daily dose is 5 μg; vitamin E present at 800% to 880% (preferably 870%) of a reference vitamin E daily dose, wherein the reference vitamin E daily dose is 12 mg; calcium present at 125% to 141% (preferably 139%) of a reference calcium daily dose, wherein the reference calcium daily dose is 800 mg; and/or (preferably and) selenium present at 120% to 155% (preferably 145%) of a reference selenium daily dose, wherein the reference selenium daily dose is 55 μg; or
(n) vitamin A present at 130% to 157% (preferably 151%) of a reference vitamin A daily dose, wherein the reference vitamin A daily dose is 800 μg; pyridoxine present at 650% to 720% (preferably 720%) of a reference pyridoxine daily dose, wherein the reference pyridoxine daily dose is 1.4 mg; folic acid present at 150% to 195% (preferably 180%) of a reference folic acid daily dose, wherein the reference folic acid daily dose is 200 μg; cyanocobalamin present at 650% to 720% (preferably 720%) of a reference cyanocobalamin daily dose, wherein the reference cyanocobalamin daily dose is 2.5 μg; ascorbic acid present at 500% to 570% (preferably 550%) of a reference ascorbic acid daily dose, wherein the reference ascorbic acid daily dose is 80 mg; cholecalciferol present at 400% to 490% (preferably 490%) of a reference cholecalciferol daily dose, wherein the reference cholecalciferol daily dose is 5 μg; vitamin E present at 1300% to 1370% (preferably 1350%) of a reference vitamin E daily dose, wherein the reference vitamin E daily dose is 12 mg; calcium present at 125% to 141% (preferably 141%) of a reference calcium daily dose, wherein the reference calcium daily dose is 800 mg; and/or (preferably and) selenium present at 165% to 205% (preferably 200%) of a reference selenium daily dose, wherein the reference selenium daily dose is 55 μg; or
(o) vitamin A present at 160% to 178% (preferably 178%) of a reference vitamin A daily dose, wherein the reference vitamin A daily dose is 800 μg; pyridoxine present at 200% to 260% (preferably 240%) of a reference pyridoxine daily dose, wherein the reference pyridoxine daily dose is 1.4 mg; folic acid present at 150% to 195% (preferably 185%) of a reference folic acid daily dose, wherein the reference folic acid daily dose is 200 μg; cyanocobalamin present at 200% to 260% (preferably 240%) of a reference cyanocobalamin daily dose, wherein the reference cyanocobalamin daily dose is 2.5 μg; ascorbic acid present at 500% to 570% (preferably 560%) of a reference ascorbic acid daily dose, wherein the reference ascorbic acid daily dose is 80 mg; cholecalciferol present at 180% to 240% (preferably 225%) of a reference cholecalciferol daily dose, wherein the reference cholecalciferol daily dose is 5 μg; vitamin E present at 1300% to 1370% (preferably 1360%) of a reference vitamin E daily dose, wherein the reference vitamin E daily dose is 12 mg; calcium present at 110% to 120% (preferably 118%) of a reference calcium daily dose, wherein the reference calcium daily dose is 800 mg; and/or (preferably and) selenium present at 120% to 155% (preferably 150%) of a reference selenium daily dose, wherein the reference selenium daily dose is 55 μg; or
(p) vitamin A present at 130% to 157% (preferably 154%) of a reference vitamin A daily dose, wherein the reference vitamin A daily dose is 800 μg; pyridoxine present at 200% to 260% (preferably 260%) of a reference pyridoxine daily dose, wherein the reference pyridoxine daily dose is 1.4 mg; folic acid present at 150% to 195% (preferably 190%) of a reference folic acid daily dose, wherein the reference folic acid daily dose is 200 μg; cyanocobalamin present at 200% to 260% (preferably 260%) of a reference cyanocobalamin daily dose, wherein the reference cyanocobalamin daily dose is 2.5 μg; ascorbic acid present at 350% to 420% (preferably 420%) of a reference ascorbic acid daily dose, wherein the reference ascorbic acid daily dose is 80 mg; cholecalciferol present at 180% to 240% (preferably 240%) of a reference cholecalciferol daily dose, wherein the reference cholecalciferol daily dose is 5 μg; vitamin E present at 800% to 880% (preferably 880%) of a reference vitamin E daily dose, wherein the reference vitamin E daily dose is 12 mg; calcium present at 110% to 120% (preferably 120%) of a reference calcium daily dose, wherein the reference calcium daily dose is 800 mg; and/or (preferably and) selenium present at 165% to 205% (preferably 205%) of a reference selenium daily dose, wherein the reference selenium daily dose is 55 μg; or
(q) vitamin A present at 130% to 157% (preferably 157%) of a reference vitamin A daily dose, wherein the reference vitamin A daily dose is 800 μg; pyridoxine present at 350% to 530% (preferably 530%) of a reference pyridoxine daily dose, wherein the reference pyridoxine daily dose is 1.4 mg; folic acid present at 150% to 195% (preferably 195%) of a reference folic acid daily dose, wherein the reference folic acid daily dose is 200 μg; cyanocobalamin present at 350% to 530% (preferably 530%) of a reference cyanocobalamin daily dose, wherein the reference cyanocobalamin daily dose is 2.5 μg; ascorbic acid present at 500% to 570% (preferably 570%) of a reference ascorbic acid daily dose, wherein the reference ascorbic acid daily dose is 80 mg; cholecalciferol present at 600% to 660% (preferably 660%) of a reference cholecalciferol daily dose, wherein the reference cholecalciferol daily dose is 5 μg; vitamin E present at 1300% to 1370% (preferably 1370%) of a reference vitamin E daily dose, wherein the reference vitamin E daily dose is 12 mg; calcium present at 145% to 147% (preferably 147%) of a reference calcium daily dose, wherein the reference calcium daily dose is 800 mg; and/or (preferably and) selenium present at 120% to 155% (preferably 155%) of a reference selenium daily dose, wherein the reference selenium daily dose is 55 μg.

An amount of one or more ingredients present in a composition of the invention may be defined by way of a weight % (wt. %). The term “weight percentage” and “wt. %” are used synonymously herein. The term “weight percentage” as used herein may mean a weight percentage of the total composition (e.g. dry composition), wherein the total weight of the composition is 100 wt. %, unless context dictates otherwise. For example, for a 100 g composition, 10 g of an ingredient corresponds to 10 wt. % of the composition. Preferably, wt. % as used herein refers to a dry composition.

Preferably, a composition of the invention is a dry composition. The term “dry composition” or “dry nutritional composition” as used herein refers to a composition in which no, or substantially no, water is present. In some embodiments the term “dry composition” or “dry nutritional composition” refers to a composition in which the only water present is that associated with, or absorbed by, an component of the composition. In one embodiment the term “dry composition” or “dry nutritional composition” refers to a composition having a water content of less than 5 wt. %, such as less than 1 or 0.1 wt. %. Preferably the term “dry” refers to a composition having a water content of less than 0.01 wt. %.

For example, a composition of the invention (or kit) may comprise:
(a) vitamin A present at greater than 0.017 wt. % to 0.065 wt. % (preferably 0.018-0.050 wt. % or 0.019-0.045 wt. %) of the total micronutrients present in the composition;
(b) pyridoxine present at greater than 0.030 wt. % to 2.174 wt. % (preferably 0.040-1 wt. % or 0.05-0.5 wt. %) of the total micronutrients present in the composition;
(c) folic acid present at greater than 0.004 wt. % to 0.022 wt. % (preferably 0.0045-0.015 wt. % or 0.0047-0.010 wt. %) of the total micronutrients present in the composition;
(d) cyanocobalamin present at greater than 0.00005 wt. % to 0.02174 wt. % (preferably 0.00007-0.005 wt. % or 0.00009-0.001 wt. %) of the total micronutrients present in the composition;
(e) ascorbic acid present at greater than 1.739 wt. % to 43.478 wt. % (preferably 2-20 wt. % or 2.5-12 wt. %) of the total micronutrients present in the composition;
(f) cholecalciferol present at greater than 0.00011 wt. % to 0.00217 wt. % (preferably 0.00015-0.001 wt. %) of the total micronutrients present in the composition;
(g) vitamin E present at greater than 0.26 wt. % to 23.91 wt. % (preferably 0.27-15 wt. % or 0.29-5 wt. %) of the total micronutrients present in the composition;
(h) calcium present at greater than 17.39 wt. % to 54.43 wt. % (preferably 18-40 wt. % or 18.5-30 wt. %) of the total micronutrients present in the composition; and/or
(i) selenium present at greater than 0.001 wt. % to 0.009 wt. % (preferably 0.0012-0.0050 wt. %) of the total micronutrients present in the composition.

In the above example, the wt. % is not necessarily the wt. % of the total composition (e.g. where other ingredients, such as macronutrients are present) but is the wt. % of total micronutrients present. For example, while a composition may comprise 5 wt. % of micronutrients (and 95 wt. % of macronutrients), that 5 wt. % may correspond to 100 wt. % of total micronutrients present; thus, a wt. % might be expressed as a wt. % of that 100 wt. % of total micronutrients.

In one embodiment:
(a) vitamin A is present at: (i) greater than 0.017 wt. % to 0.022 wt. % (e.g. 0.0172-0.021 wt. %, 0.0174-0.020 wt. %, 0.0176-0.019 wt. % or 0.0178-0.018 wt. %, preferably 0.018-0.022 wt. % or 0.019-0.022 wt. %, e.g. a lower end of the range may be at least 0.0172 wt. %, 0.0174 wt. %, 0.0176 wt. %, 0.0178 wt. % or 0.018 wt. %, preferably 0.018 wt. % or 0.019 wt. % and/or an upper end of the range may be less than or equal to 0.022 wt. %, 0.0215 wt. %, 0.0210 wt. %, 0.0205 wt. % or 0.020 wt. %) of the total micronutrients present in the composition; (ii) greater than 0.022 wt. % to 0.028 wt. % of the total micronutrients present in the composition (e.g. a lower end of the range may be at least 0.0225 wt. %, 0.023 wt. %, 0.0235 wt. %, 0.024 wt. % or 0.0245 wt. % and/or an upper end of the range may be less than or equal to 0.0275 wt. %, 0.027 wt. %, 0.0265 wt. %, 0.0260 wt. % or 0.0255 wt. %); or (iii) greater than 0.028 wt. % to 0.065 wt. % (preferably greater than 0.028-0.050 wt. % or greater than 0.028-0.045 wt. %, e.g. a lower end of the range may be at least 0.029 wt. %, 0.030 wt. %, 0.031 wt. %, 0.032 wt. % or 0.032 wt. % and/or an upper end of the range may be less than or equal to 0.06 wt. %, 0.055 wt. %, 0.05 wt. %, 0.045 wt. % or 0.04 wt. %) of the total micronutrients present in the composition;
(b) pyridoxine is present at: (i) greater than 0.030 wt. % to 0.093 wt. % (e.g. 0.032-0.091 wt. %, 0.034-0.089 wt. %, 0.036-0.087 wt. %, 0.038-0.085 wt. % or 0.040-0.083 wt. %, preferably 0.040-0.093 wt. % or 0.05-0.093 wt. %, e.g. a lower end of the range may be at least 0.032 wt. %, 0.034 wt. %, 0.036 wt. %, 0.038 wt. % or 0.040 wt. %, preferably 0.040 wt. % or 0.05 wt. % and/or an upper end of the range may be less than or equal to 0.093 wt. %, 0.090 wt. %, 0.088 wt. %, 0.086 wt. %, 0.084 wt. % or 0.082 wt. %, preferably 0.093 wt. %) of the total micronutrients present in the composition; (ii) greater than 0.093 wt. % to 0.180 wt. % of the total micronutrients present in the composition (e.g. a lower end of the range may be at least 0.095 wt. %, 0.100 wt. %, 0.105 wt. %, 0.110 wt. % or 0.115 wt. % and/or an upper end of the range may be less than or equal to 0.175 wt. %, 0.170 wt. %, 0.165 wt. %, 0.160 wt. %, 0.155 wt. % or 0.150 wt. %); or (iii) greater than 0.180 wt. % to 2.174 wt. % (preferably greater than 0.180-1 wt. % or greater than 0.180-0.5 wt. %, e.g. a lower end of the range may be at least 0.200 wt. %, 0.400 wt. %, 0.600 wt. %, 0.800 wt. % or 1.0 wt. % and/or an upper end of the range may be less than or equal to 2.0 wt. %, 1.8 wt. %, 1.6 wt. %, 1.4 wt. %, 1.2 wt. % or 1.0 wt. %) of the total micronutrients present in the composition;
(c) folic acid is present at: (i) greater than 0.004 wt. % to 0.006 wt. % (e.g. 0.004-0.0058 wt. %, 0.0041-0.0056 wt. %, 0.0042-0.0054 wt. %, 0.0043-0.0052 wt. %, 0.0044-0.0050 wt. % or 0.0045-0.0048 wt. %, preferably 0.0045-0.006 wt. % or 0.0047-0.006 wt. %, e.g. a lower end of the range may be at least 0.004 wt. %, 0.0041 wt. %, 0.0042 wt. %, 0.0043 wt. %, 0.0044 wt. % or 0.0045 wt. %, preferably 0.0045 wt. % or 0.0047 wt. % and/or an upper end of the range may be less than or equal to 0.006 wt. %, 0.0058 wt. %, 0.0056 wt. %, 0.0054 wt. %, 0.0052 wt. % or 0.0050 wt. %, preferably 0.006 wt. %) of the total micronutrients present in the composition; (ii) greater than 0.006 wt. % to 0.009 wt. % of the total micronutrients present in the composition (e.g. a lower end of the range may be at least 0.0062 wt. %, 0.0064 wt. %, 0.0066 wt. %, 0.0068 wt. %, 0.0070 wt. % or 0.0072 wt. % and/or an upper end of the range may be less than or equal to 0.0088 wt. %, 0.0086 wt. %, 0.0084 wt. %, 0.0082 wt. %, 0.0080 wt. % or 0.0078 wt. %); or (iii) greater than 0.009 wt. % to 0.022 wt. % (preferably greater than 0.009-0.015 wt. % or greater than 0.009-0.01 wt. %, e.g. a lower end of the range may be at least 0.010 wt. %, 0.011 wt. %, 0.012 wt. %, 0.013 wt. %, 0.014 wt. % or 0.015 wt. % and/or an upper end of the range may be less than or equal to 0.021 wt. %, 0.020 wt. %, 0.019 wt. %, 0.018 wt. %, 0.017 wt. % or 0.016 wt. %) of the total micronutrients present in the composition;
(d) cyanocobalamin is present at: (i) greater than 0.00005 wt. % to 0.00017 wt. % (e.g. 0.000055-0.00016 wt. %, 0.00006-0.00015 wt. %, 0.000065-0.00014 wt. % or 0.00007-0.00013 wt. %, preferably 0.00007-0.00017 wt. % or 0.00009-0.00017 wt. %, e.g. a lower end of the range may be at least 0.000055 wt. %, 0.00006 wt. %, 0.000065 wt. % or 0.00007 wt. %, preferably 0.00007 wt. % or 0.00009 wt. % and/or an upper end of the range may be less than or equal to 0.00017 wt. %, 0.00016 wt. %, 0.00015 wt. % or 0.00014 wt. %, preferably 0.00017 wt. %) of the total micronutrients present in the composition; (ii) greater than 0.00017 wt. % to 0.00032 wt. % of the total micronutrients present in the composition (e.g. a lower end of the range may be at least 0.00020 wt. %, 0.00022 wt. %, 0.00024 wt. % or 0.00026 wt. %, and/or an upper end of the range may be less than or equal to 0.00030 wt. %, 0.00028 wt. %, 0.00026 wt. % or 0.00024 wt. %); or (iii) greater than 0.00032 wt. % to 0.02174 wt. % (preferably greater than 0.00032-0.005 wt. % or greater than 0.00032-0.001 wt. %, e.g. a lower end of the range may be at least 0.00050 wt. %, 0.001 wt. %, 0.005 wt. % or 0.01 wt. %, and/or an upper end of the range may be less than or equal to 0.02 wt. %, 0.015 wt. %, 0.01 wt. % or 0.005 wt. %) of the total micronutrients present in the composition;
(e) ascorbic acid is present at: (i) greater than 1.739 wt. % to 4.783 wt. % (e.g. 1.8-4.5 wt. %, 1.85-4.3 wt. %, 1.9-4.1 wt. %, 1.95-3.9 wt. % or 2-3.7 wt. %, preferably 2-4.783 wt. % or 2.5-4.783 wt. %, e.g. a lower end of the range may be at least 1.8 wt. %, 1.85 wt. %, 1.9 wt. %, 1.95 wt. % or 2 wt. %, preferably 2 wt. % or 2.5 wt. % and/or an upper end of the range may be less than or equal to 4.783 wt. % 4.7 wt. %, 4.5 wt. %, 4.3 wt. % or 4.1 wt. %, preferably 4.783 wt. %) of the total micronutrients present in the composition; (ii) greater than 4.783 wt. % to 8.000 wt. % of the total micronutrients present in the composition (e.g. a lower end of the range may be at least 5 wt. %, 5.2 wt. %, 5.4 wt. %, 5.6 wt. % or 5.8 wt. % and/or an upper end of the range may be less than or equal to 7.8 wt. %, 7.6 wt. %, 7.4 wt. %, 7.2 wt. % or 7 wt. %); or (iii) greater than 8.000 wt. % to 43.478 wt. % (preferably greater than 8-20 wt. % or greater than 8-12 wt. %, e.g. a lower end of the range may be at least 10 wt. %, 12 wt. %, 14 wt. %, 16 wt. % or 18 wt. % and/or an upper end of the range may be less than or equal to 40 wt. %, 38 wt. %, 36 wt. %, 34 wt. % or 32 wt. %) of the total micronutrients present in the composition;
(f) cholecalciferol is present at: (i) greater than 0.00011 wt. % to 0.00035 wt. % (e.g. 0.00012-0.00034 wt. %, 0.00013-0.00033 wt. %, 0.00014-0.00032 wt. % or 0.00015-0.00031 wt. %, preferably 0.00015-0.00035 wt. %, e.g. a lower end of the range may be at least 0.00012 wt. %, 0.00013 wt. %, 0.00014 wt. % or 0.00015 wt. %, preferably 0.00015 wt. % and/or an upper end of the range may be less than or equal to 0.00035 wt. %, 0.00034 wt. %, 0.00033 wt. %, 0.00032 wt. % or 0.00031 wt. %, preferably 0.00035 wt. %) of the total micronutrients present in the composition; (ii) greater than 0.00035 wt. % to 0.00059 wt. % of the total micronutrients present in the composition (e.g. a lower end of the range may be at least 0.00036 wt. %, 0.00038 wt. %, 0.00040 wt. % or 0.00042 wt. % and/or an upper end of the range may be less than or equal to 0.00058 wt. %, 0.00056 wt. %, 0.00054 wt. %, 0.00052 wt. % or 0.00050 wt. %); or (iii) greater than 0.00059 wt. % to 0.00217 wt. % (preferably greater than 0.00059-0.001 wt. %, e.g. a lower end of the range may be at least 0.00060 wt. %, 0.00070 wt. %, 0.00080 wt. % or 0.00090 wt. % and/or an upper end of the range may be less than or equal to 0.00200 wt. %, 0.00190 wt. %, 0.00180 wt. %, 0.00170 wt. % or 0.00160 wt. %) of the total micronutrients present in the composition;
(g) vitamin E is present at: (i) greater than 0.26 wt. % to 1.21 wt. % (e.g. 0.262-1.19 wt. %, 0.264-1.17 wt. %, 0.266-1.15 wt. %, 0.268-1.13 wt. % or 0.27-1.11 wt. %, preferably 0.27-1.21 wt. % or 0.29-1.21 wt. %, e.g. a lower end of the range may be at least 0.262 wt. %, 0.264 wt. %, 0.266 wt. %, 0.268 wt. % or 0.27 wt. %, preferably 0.27 wt. % or 0.29 wt. % and/or an upper end of the range may be less than or equal to 1.21 wt. %, 1.19 wt. %, 1.17 wt. %, 1.15 wt. % or 1.13 wt. %, preferably 1.21 wt. %) of the total micronutrients present in the composition; (ii) greater than 1.21 wt. % to 2.84 wt. % of the total micronutrients present in the composition (e.g. a lower end of the range may be at least 1.25 wt. %, 1.3 wt. %, 1.35 wt. %, 1.4 wt. % or 1.45 wt. % and/or an upper end of the range may be less than or equal to 2.8 wt. %, 2.75 wt. %, 2.7 wt. %, 2.65 wt. % or 2.6 wt. %); or (iii) greater than 2.84 wt. % to 23.91 wt. % (preferably greater than 2.84-15 wt. % or greater than 2.84-5 wt. %, e.g. a lower end of the range may be at least 3 wt. %, 3.5 wt. %, 4 wt. %, 4.5 wt. % or 5 wt. % and/or an upper end of the range may be less than or equal to 14.5 wt. %, 14 wt. %, 13.5 wt. %, 13 wt. % or 12.5 wt. %) of the total micronutrients present in the composition;
(h) calcium is present at: (i) greater than 17.39 wt. % to 21.30 wt. % (e.g. 17.4-21 wt. %, 17.6-20.8 wt. %, 17.8-20.6 wt. % or 18-20.4 wt. %, preferably 18-21.30 wt. % or 18.5-21.30 wt. %, e.g. a lower end of the range may be at least 17.4 wt. %, 17.6 wt. %, 17.8 wt. % or 18 wt. %, preferably 18 wt. % or 18.5 wt. % and/or an upper end of the range may be less than or equal to 21.30 wt. %, 21 wt. %, 19 wt. %, 17 wt. % or 15 wt. %, preferably 21.30 wt. %) of the total micronutrients present in the composition; (ii) greater than 21.30 wt. % to 24.87 wt. % of the total micronutrients present in the composition (e.g. a lower end of the range may be at least 21.5 wt. %, 21.7 wt. %, 21.9 wt. % or 22.1 wt. % and/or an upper end of the range may be less than or equal to 24.5 wt. %, 24.3 wt. %, 24.1 wt. %, 23.9 wt. % or 23.7 wt. %); or (iii) greater than 24.87 wt. % to 54.43 wt. % (preferably greater than 24.87-40 wt. % or greater than 24.87-30 wt. %, e.g. a lower end of the range may be at least 25 wt. %, 27 wt. %, 29 wt. % or 31 wt. % and/or an upper end of the range may be less than or equal to 54 wt. %, 52 wt. %, 50 wt. %, 48 wt. % or 46 wt. %) of the total micronutrients present in the composition; and/or
(i) selenium is present at: (i) greater than 0.001 wt. % to 0.002 wt. % (e.g. 0.0011-0.0019 wt. %, 0.0012-0.0018 wt. %, 0.0013-0.0017 wt. % or 0.0014-0.0016 wt. %, preferably 0.0012-0.002 wt. %, e.g. a lower end of the range may be at least 0.0012 wt. %, 0.0014 wt. %, 0.0016 wt. % or 0.0018 wt. %, preferably 0.0012 wt. % and/or an upper end of the range may be less than or equal to 0.002 wt. %, 0.0018 wt. %, 0.0016 wt. % or 0.0014 wt. %, preferably 0.002 wt. %) of the total micronutrients present in the composition; or (ii) greater than 0.002 wt. % to 0.009 wt. % (preferably greater than 0.002-0.0050 wt. %, e.g. a lower end of the range may be at least 0.0025 wt. %, 0.003 wt. %, 0.0035 wt. % or 0.004 wt. % and/or an upper end of the range may be less than or equal to 0.0085 wt. %, 0.008 wt. %, 0.0075 wt. % or 0.007 wt. %) of the total micronutrients present in the composition.

In one embodiment:
(a) vitamin A is present at: (i) 0.020 wt. % to 0.022 wt. % of the total micronutrients present in the composition; (ii) 0.023 wt. % to 0.027 wt. % of the total micronutrients present in the composition; or (iii) 0.028 wt. % to 0.031 wt. % of the total micronutrients present in the composition;
(b) pyridoxine is present at: (i) 0.061 wt. % to 0.079 wt. % of the total micronutrients present in the composition; (ii) 0.107 wt. % to 0.161 wt. % of the total micronutrients present in the composition; or (iii) 0.198 wt. % to 0.219 wt. % of the total micronutrients present in the composition;
(c) folic acid is present at: (i) 0.0052 wt. % to 0.0061 wt. % of the total micronutrients present in the composition; (ii) 0.0065 wt. % to 0.0085 wt. % of the total micronutrients present in the composition; or (iii) 0.0089 wt. % to 0.0091 wt. % of the total micronutrients present in the composition;
(d) cyanocobalamin is present at: (i) 0.00011 wt. % to 0.00014 wt. % of the total micronutrients present in the composition; (ii) 0.00019 wt. % to 0.00029 wt. % of the total micronutrients present in the composition; or (iii) 0.00035 wt. % to 0.00039 wt. % of the total micronutrients present in the composition;
(e) ascorbic acid is present at: (i) 3.304 wt. % to 3.652 wt. % of the total micronutrients present in the composition; (ii) 6.087 wt. % to 7.304 wt. % of the total micronutrients present in the composition; or (iii) 8.696 wt. % to 9.913 wt. % of the total micronutrients present in the composition;
(f) cholecalciferol is present at: (i) 0.00020 wt. % to 0.00026 wt. % of the total micronutrients present in the composition; (ii) 0.00043 wt. % to 0.00053 wt. % of the total micronutrients present in the composition; or (iii) 0.00065 wt. % to 0.00072 wt. % of the total micronutrients present in the composition;
(g) vitamin E is present at: (i) 0.33 wt. % to 0.35 wt. % of the total micronutrients present in the composition; (ii) 2.09 wt. % to 2.30 wt. % of the total micronutrients present in the composition; or (iii) 3.39 wt. % to 3.57 wt. % of the total micronutrients present in the composition;
(h) calcium is present at: (i) 19.13 wt. % to 20.87 wt. % of the total micronutrients present in the composition; (ii) 21.74 wt. % to 24.52 wt. % of the total micronutrients present in the composition; or (iii) 25.22 wt. % to 25.57 wt. % of the total micronutrients present in the composition; and/or
(i) selenium is present at: (i) 0.0014 wt. % to 0.0019 wt. % of the total micronutrients present in the composition; or (ii) 0.0020 wt. % to 0.0025 wt. % of the total micronutrients present in the composition.

The wt. % s for each of the micronutrients above preferably correspond to base/normal (i.e. where the wt. % is preceded by a (i)), medium (i.e. where the wt. % is preceded by a (ii)), or high levels of the micronutrients (i.e. where the wt. % is preceded by a (iii)), or base/normal (i.e. where the wt. % is preceded by a (i)) or high levels (i.e. where the wt. % is preceded by a (ii)) in the context of selenium. Similarly, the wt. % s for each of the micronutrients above preferably correspond to either base/normal (i.e. where the wt. % is preceded by a (i)), medium (i.e. where the wt. % is preceded by a (ii)), or high need of the micronutrients (i.e. where the wt. % is preceded by a (iii)), or base/normal (i.e. where the wt. % is preceded by a (i)) or high need (i.e. where the wt. % is preceded by a (ii)) in the context of selenium. Said need is preferably a need for the indicated wt. % of the micronutrient.

The nutritional compositions corresponding to each nutritional reference standard may be expressed in corresponding wt. % s. The embodiments above further defining the wt. % ranges of the micronutrients apply equally to each of (a) to (q) below.

In one embodiment:
(a) vitamin A is present at greater than 0.022 wt. % to 0.028 wt. % of the total micronutrients present in the composition; pyridoxine is present at greater than 0.093 wt. % to 0.180 wt. % of the total micronutrients present in the composition; folic acid is present at greater than 0.004 wt. % to 0.006 wt. % of the total micronutrients present in the composition; cyanocobalamin is present at greater than 0.00017 wt. % to 0.00032 wt. % of the total micronutrients present in the composition; ascorbic acid is present at greater than 4.783 wt. % to 8.000 wt. % of the total micronutrients present in the composition; cholecalciferol is present at greater than 0.00059 wt. % to 0.00217 wt. % of the total micronutrients present in the composition; vitamin E is present at greater than 1.21 wt. % to 2.84 wt. % of the total micronutrients present in the composition; calcium is present at greater than 17.39 wt. % to 21.30 wt. % of the total micronutrients present in the composition; and/or (preferably and) selenium is present at greater than 0.002 wt. % to 0.009 wt. % of the total micronutrients present in the composition; or
(b) vitamin A is present at greater than 0.028 wt. % to 0.065 wt. % of the total micronutrients present in the composition; pyridoxine is present at greater than 0.093 wt. % to 0.180 wt. % of the total micronutrients present in the composition; folic acid is present at greater than 0.004 wt. % to 0.006 wt. % of the total micronutrients present in the composition; cyanocobalamin is present at greater than 0.00017 wt. % to 0.00032 wt. % of the total micronutrients present in the composition; ascorbic acid is present at greater than 4.783 wt. % to 8.000 wt. % of the total micronutrients present in the composition; cholecalciferol is present at greater than 0.00035 wt. % to 0.00059 wt. % of the total micronutrients present in the composition; vitamin E is present at greater than 1.21 wt. % to 2.84 wt. % of the total micronutrients present in the composition; calcium is present at greater than 17.39 wt. % to 21.30 wt. % of the total micronutrients present in the composition; and/or (preferably and) selenium is present at greater than 0.001 wt. % to 0.002 wt. % of the total micronutrients present in the composition; or
(c) vitamin A is present at greater than 0.022 wt. % to 0.028 wt. % of the total micronutrients present in the composition; pyridoxine is present at greater than 0.180 wt. % to 2.174 wt. % of the total micronutrients present in the composition; folic acid is present at greater than 0.009 wt. % to 0.022 wt. % of the total micronutrients present in the composition; cyanocobalamin is present at greater than 0.00032 wt. % to 0.02174 wt. % of the total micronutrients present in the composition; ascorbic acid is present at greater than 4.783 wt. % to 8.000 wt. % of the total micronutrients present in the composition; cholecalciferol is present at greater than 0.00059 wt. % to 0.00217 wt. % of the total micronutrients present in the composition; vitamin E is present at greater than 1.21 wt. % to 2.84 wt. % of the total micronutrients present in the composition; calcium is present at greater than 21.30 wt. % to 24.87 wt. % of the total micronutrients present in the composition; and/or (preferably and) selenium is present at greater than 0.001 wt. % to 0.002 wt. % of the total micronutrients present in the composition; or
(d) vitamin A is present at greater than 0.028 wt. % to 0.065 wt. % of the total micronutrients present in the composition; pyridoxine is present at greater than 0.093 wt. % to 0.180 wt. % of the total micronutrients present in the composition; folic acid is present at greater than 0.004 wt. % to 0.006 wt. % of the total micronutrients present in the composition; cyanocobalamin is present at greater than 0.00017 wt. % to 0.00032 wt. % of the total micronutrients present in the composition; ascorbic acid is present at greater than 4.783 wt. % to 8.000 wt. % of the total micronutrients present in the composition; cholecalciferol is present at greater than 0.00059 wt. % to 0.00217 wt. % of the total micronutrients present in the composition; vitamin E is present at greater than 1.21 wt. % to 2.84 wt. % of the total micronutrients present in the composition; calcium is present at greater than 24.87 wt. % to 54.43 wt. % of the total micronutrients present in the composition; and/or (preferably and) selenium is present at greater than 0.001 wt. % to 0.002 wt. % of the total micronutrients present in the composition; or
(e) vitamin A is present at greater than 0.028 wt. % to 0.065 wt. % of the total micronutrients present in the composition; pyridoxine is present at greater than 0.180 wt. % to 2.174 wt. % of the total micronutrients present in the composition; folic acid is present at greater than 0.004 wt. % to 0.006 wt. % of the total micronutrients present in the composition; cyanocobalamin is present at greater than 0.00032 wt. % to 0.02174 wt. % of the total micronutrients present in the composition; ascorbic acid is present at greater than 8.000 wt. % to 43.478 wt. % of the total micronutrients present in the composition; cholecalciferol is present at greater than 0.00059 wt. % to 0.00217 wt. % of the total micronutrients present in the composition; vitamin E is present at greater than 2.84 wt. % to 23.91 wt. % of the total micronutrients present in the composition; calcium is present at greater than 21.30 wt. % to 24.87 wt. % of the total micronutrients present in the composition; and/or (preferably and) selenium is present at greater than 0.002 wt. % to 0.009 wt. % of the total micronutrients present in the composition; or
(f) vitamin A is present at greater than 0.022 wt. % to 0.028 wt. % of the total micronutrients present in the composition; pyridoxine is present at greater than 0.180 wt. % to 2.174 wt. % of the total micronutrients present in the composition; folic acid is present at greater than 0.006 wt. % to 0.009 wt. % of the total micronutrients present in the composition; cyanocobalamin is present at greater than 0.00032 wt. % to 0.02174 wt. % of the total micronutrients present in the composition; ascorbic acid is present at greater than 4.783 wt. % to 8.000 wt. % of the total micronutrients present in the composition; cholecalciferol is present at greater than 0.00011 wt. % to 0.00035 wt. % of the total micronutrients present in the composition; vitamin E is present at greater than 1.21 wt. % to 2.84 wt. % of the total micronutrients present in the composition; calcium is present at greater than 17.39 wt. % to 21.30 wt. % of the total micronutrients present in the composition; and/or (preferably and) selenium is present at greater than 0.002 wt. % to 0.009 wt. % of the total micronutrients present in the composition; or
(g) vitamin A is present at greater than 0.022 wt. % to 0.028 wt. % of the total micronutrients present in the composition; pyridoxine is present at greater than 0.030 wt. % to 0.093 wt. % of the total micronutrients present in the composition; folic acid is present at greater than 0.006 wt. % to 0.009 wt. % of the total micronutrients present in the composition; cyanocobalamin is present at greater than 0.00005 wt. % to 0.00017 wt. % of the total micronutrients present in the composition; ascorbic acid is present at greater than 8.000 wt. % to 43.478 wt. % of the total micronutrients present in the composition; cholecalciferol is present at greater than 0.00035 wt. % to 0.00059 wt. % of the total micronutrients present in the composition; vitamin E is present at greater than 2.84 wt. % to 23.91 wt. % of the total micronutrients present in the composition; calcium is present at greater than 21.30 wt. % to 24.87 wt. % of the total micronutrients present in the composition; and/or (preferably and) selenium is present at greater than 0.002 wt. % to 0.009 wt. % of the total micronutrients present in the composition; or
(h) vitamin A is present at greater than 0.022 wt. % to 0.028 wt. % of the total micronutrients present in the composition; pyridoxine is present at greater than 0.093 wt. % to 0.180 wt. % of the total micronutrients present in the composition; folic acid is present at greater than 0.006 wt. % to 0.009 wt. % of the total micronutrients present in the composition; cyanocobalamin is present at greater than 0.00017 wt. % to 0.00032 wt. % of the total micronutrients present in the composition; ascorbic acid is present at greater than 4.783 wt. % to 8.000 wt. % of the total micronutrients present in the composition; cholecalciferol is present at greater than 0.00011 wt. % to 0.00035 wt. % of the total micronutrients present in the composition; vitamin E is present at greater than 1.21 wt. % to 2.84 wt. % of the total micronutrients present in the composition; calcium is present at greater than 21.30 wt. % to 24.87 wt. % of the total micronutrients present in the composition; and/or (preferably and) selenium is present at greater than 0.002 wt. % to 0.009 wt. % of the total micronutrients present in the composition; or
(i) vitamin A is present at greater than 0.022 wt. % to 0.028 wt. % of the total micronutrients present in the composition; pyridoxine is present at greater than 0.093 wt. % to 0.180 wt. % of the total micronutrients present in the composition; folic acid is present at greater than 0.006 wt. % to 0.009 wt. % of the total micronutrients present in the composition; cyanocobalamin is present at greater than 0.00017 wt. % to 0.00032 wt. % of the total micronutrients present in the composition; ascorbic acid is present at greater than 1.739 wt. % to 4.783 wt. % of the total micronutrients present in the composition; cholecalciferol is present at greater than 0.00035 wt. % to 0.00059 wt. % of the total micronutrients present in the composition; vitamin E is present at greater than 0.26 wt. % to 1.21 wt. % of the total micronutrients present in the composition; calcium is present at greater than 21.30 wt. % to 24.87 wt. % of the total micronutrients present in the composition; and/or (preferably and) selenium is present at greater than 0.001 wt. % to 0.002 wt. % of the total micronutrients present in the composition; or
(j) vitamin A is present at greater than 0.028 wt. % to 0.065 wt. % of the total micronutrients present in the composition; pyridoxine is present at greater than 0.093 wt. % to 0.180 wt. % of the total micronutrients present in the composition; folic acid is present at greater than 0.004 wt. % to 0.006 wt. % of the total micronutrients present in the composition; cyanocobalamin is present at greater than 0.00017 wt. % to 0.00032 wt. % of the total micronutrients present in the composition; ascorbic acid is present at greater than 8.000 wt. % to 43.478 wt. % of the total micronutrients present in the composition; cholecalciferol is present at greater than 0.00035 wt. % to 0.00059 wt. % of the total micronutrients present in the composition; vitamin E is present at greater than 2.84 wt. % to 23.91 wt. % of the total micronutrients present in the composition; calcium is present at greater than 21.30 wt. % to 24.87 wt. % of the total micronutrients present in the composition; and/or (preferably and) selenium is present at greater than 0.002 wt. % to 0.009 wt. % of the total micronutrients present in the composition; or
(k) vitamin A is present at greater than 0.028 wt. % to 0.065 wt. % of the total micronutrients present in the composition; pyridoxine is present at greater than 0.093 wt. % to 0.180 wt. % of the total micronutrients present in the composition; folic acid is present at greater than 0.006 wt. % to 0.009 wt. % of the total micronutrients present in the composition; cyanocobalamin is present at greater than 0.00017 wt. % to 0.00032 wt. % of the total micronutrients present in the composition; ascorbic acid is present at greater than 8.000 wt. % to 43.478 wt. % of the total micronutrients present in the composition; cholecalciferol is present at greater than 0.00035 wt. % to 0.00059 wt. % of the total micronutrients present in the composition; vitamin E is present at greater than 2.84 wt. % to 23.91 wt. % of the total micronutrients present in the composition; calcium is present at greater than 21.30 wt. % to 24.87 wt. % of the total micronutrients present in the composition; and/or (preferably and) selenium is present at greater than 0.001 wt. % to 0.002 wt. % of the total micronutrients present in the composition; or
(l) vitamin A is present at greater than 0.022 wt. % to 0.028 wt. % of the total micronutrients present in the composition; pyridoxine is present at greater than 0.093 wt. % to 0.180 wt. % of the total micronutrients present in the composition; folic acid is present at greater than 0.006 wt. % to 0.009 wt. % of the total micronutrients present in the composition; cyanocobalamin is present at greater than 0.00017 wt. % to 0.00032 wt. % of the total micronutrients present in the composition; ascorbic acid is present at greater than 8.000 wt. % to 43.478 wt. % of the total micronutrients present in the composition; cholecalciferol is present at greater than 0.00011 wt. % to 0.00035 wt. % of the total micronutrients present in the composition; vitamin E is present at greater than 2.84 wt. % to 23.91 wt. % of the total micronutrients present in the composition; calcium is present at greater than 17.39 wt. % to 21.30 wt. % of the total micronutrients present in the composition; and/or (preferably and) selenium is present at greater than 0.002 wt. % to 0.009 wt. % of the total micronutrients present in the composition; or
(m) vitamin A is present at greater than 0.028 wt. % to 0.065 wt. % of the total micronutrients present in the composition; pyridoxine is present at greater than 0.030 wt. % to 0.093 wt. % of the total micronutrients present in the composition; folic acid is present at greater than 0.009 wt. % to 0.022 wt. % of the total micronutrients present in the composition; cyanocobalamin is present at greater than 0.00005 wt. % to 0.00017 wt. % of the total micronutrients present in the composition; ascorbic acid is present at greater than 4.783 wt. % to 8.000 wt. % of the total micronutrients present in the composition; cholecalciferol is present at greater than 0.00035 wt. % to 0.00059 wt. % of the total micronutrients present in the composition; vitamin E is present at greater than 1.21 wt. % to 2.84 wt. % of the total micronutrients present in the composition; calcium is present at greater than 21.30 wt. % to 24.87 wt. % of the total micronutrients present in the composition; and/or (preferably and) selenium is present at greater than 0.001 wt. % to 0.002 wt. % of the total micronutrients present in the composition; or
(n) vitamin A is present at greater than 0.022 wt. % to 0.028 wt. % of the total micronutrients present in the composition; pyridoxine is present at greater than 0.180 wt. % to 2.174 wt. % of the total micronutrients present in the composition; folic acid is present at greater than 0.006 wt. % to 0.009 wt. % of the total micronutrients present in the composition; cyanocobalamin is present at greater than 0.00032 wt. % to 0.02174 wt. % of the total micronutrients present in the composition; ascorbic acid is present at greater than 8.000 wt. % to 43.478 wt. % of the total micronutrients present in the composition; cholecalciferol is present at greater than 0.00035 wt. % to 0.00059 wt. % of the total micronutrients present in the composition; vitamin E is present at greater than 2.84 wt. % to 23.91 wt. % of the total micronutrients present in the composition; calcium is present at greater than 21.30 wt. % to 24.87 wt. % of the total micronutrients present in the composition; and/or (preferably and) selenium is present at greater than 0.002 wt. % to 0.009 wt. % of the total micronutrients present in the composition; or
(o) vitamin A is present at greater than 0.028 wt. % to 0.065 wt. % of the total micronutrients present in the composition; pyridoxine is present at greater than 0.030 wt. % to 0.093 wt. % of the total micronutrients present in the composition; folic acid is present at greater than 0.006 wt. % to 0.009 wt. % of the total micronutrients present in the composition; cyanocobalamin is present at greater than 0.00005 wt. % to 0.00017 wt. % of the total micronutrients present in the composition; ascorbic acid is present at greater than 8.000 wt. % to 43.478 wt. % of the total micronutrients present in the composition; cholecalciferol is present at greater than 0.00011 wt. % to 0.00035 wt. % of the total micronutrients present in the composition; vitamin E is present at greater than 2.84 wt. % to 23.91 wt. % of the total micronutrients present in the composition; calcium is present at greater than 17.39 wt. % to 21.30 wt. % of the total micronutrients present in the composition; and/or (preferably and) selenium is present at greater than 0.001 wt. % to 0.002 wt. % of the total micronutrients present in the composition; or
(p) vitamin A is present at greater than 0.022 wt. % to 0.028 wt. % of the total micronutrients present in the composition; pyridoxine is present at greater than 0.030 wt. % to 0.093 wt. % of the total micronutrients present in the composition; folic acid is present at greater than 0.006 wt. % to 0.009 wt. % of the total micronutrients present in the composition; cyanocobalamin is present at greater than 0.00005 wt. % to 0.00017 wt. % of the total micronutrients present in the composition; ascorbic acid is present at greater than 4.783 wt. % to 8.000 wt. % of the total micronutrients present in the composition; cholecalciferol is present at greater than 0.00011 wt. % to 0.00035 wt. % of the total micronutrients present in the composition; vitamin E is present at greater than 1.21 wt. % to 2.84 wt. % of the total micronutrients present in the composition; calcium is present at greater than 17.39 wt. % to 21.30 wt. % of the total micronutrients present in the composition; and/or (preferably and) selenium is present at greater than 0.002 wt. % to 0.009 wt. % of the total micronutrients present in the composition; or
(q) vitamin A is present at greater than 0.022 wt. % to 0.028 wt. % of the total micronutrients present in the composition; pyridoxine is present at greater than 0.093 wt. % to 0.180 wt. % of the total micronutrients present in the composition; folic acid is present at greater than 0.006 wt. % to 0.009 wt. % of the total micronutrients present in the composition; cyanocobalamin is present at greater than 0.00017 wt. % to 0.00032 wt. % of the total micronutrients present in the composition; ascorbic acid is present at greater than 8.000 wt. % to 43.478 wt. % of the total micronutrients present in the composition; cholecalciferol is present at greater than 0.00059 wt. % to 0.00217 wt. % of the total micronutrients present in the composition; vitamin E is present at greater than 2.84 wt. % to 23.91 wt. % of the total micronutrients present in the composition; calcium is present at greater than 24.87 wt. % to 54.43 wt. % of the total micronutrients present in the composition; and/or (preferably and) selenium is present at greater than 0.001 wt. % to 0.002 wt. % of the total micronutrients present in the composition.

In one embodiment:
(a) vitamin A is present at 0.023 wt. % to 0.027 wt. % of the total micronutrients present in the composition; pyridoxine is present at 0.107 wt. % to 0.161 wt. % of the total micronutrients present in the composition; folic acid is present at 0.0052 wt. % to 0.0061 wt. % of the total micronutrients present in the composition; cyanocobalamin is present at 0.00019 wt. % to 0.00029 wt. % of the total micronutrients present in the composition; ascorbic acid is present at 6.087 wt. % to 7.304 wt. % of the total micronutrients present in the composition; cholecalciferol is present at 0.00065 wt. % to 0.00072 wt. % of the total micronutrients present in the composition; vitamin E is present at 2.09 wt. % to 2.30 wt. % of the total micronutrients present in the composition; calcium is present at 19.13 wt. % to 20.87 wt. % of the total micronutrients present in the composition; and/or (preferably and) selenium is present at 0.0020 wt. % to 0.0025 wt. % of the total micronutrients present in the composition; or
(b) vitamin A is present at 0.028 wt. % to 0.031 wt. % of the total micronutrients present in the composition; pyridoxine is present at 0.107 wt. % to 0.161 wt. % of the total micronutrients present in the composition; folic acid is present at 0.0052 wt. % to 0.0061 wt. % of the total micronutrients present in the composition; cyanocobalamin is present at 0.00019 wt. % to 0.00029 wt. % of the total micronutrients present in the composition; ascorbic acid is present at 6.087 wt. % to 7.304 wt. % of the total micronutrients present in the composition; cholecalciferol is present at 0.00043 wt. % to 0.00053 wt. % of the total micronutrients present in the composition; vitamin E is present at 2.09 wt. % to 2.30 wt. % of the total micronutrients present in the composition; calcium is present at 19.13 wt. % to 20.87 wt. % of the total micronutrients present in the composition; and/or (preferably and) selenium is present at 0.0014 wt. % to 0.0019 wt. % of the total micronutrients present in the composition; or
(c) vitamin A is present at 0.023 wt. % to 0.027 wt. % of the total micronutrients present in the composition; pyridoxine is present at 0.198 wt. % to 0.219 wt. % of the total micronutrients present in the composition; folic acid is present at 0.0089 wt. % to 0.0091 wt. % of the total micronutrients present in the composition; cyanocobalamin is present at 0.00035 wt. % to 0.00039 wt. % of the total micronutrients present in the composition; ascorbic acid is present at 6.087 wt. % to 7.304 wt. % of the total micronutrients present in the composition; cholecalciferol is present at 0.00065 wt. % to 0.00072 wt. % of the total micronutrients present in the composition; vitamin E is present at 2.09 wt. % to 2.30 wt. % of the total micronutrients present in the composition; calcium is present at 21.74 wt. % to 24.52 wt. % of the total micronutrients present in the composition; and/or (preferably and) selenium is present at 0.0014 wt. % to 0.0019 wt. % of the total micronutrients present in the composition; or
(d) vitamin A is present at 0.028 wt. % to 0.031 wt. % of the total micronutrients present in the composition; pyridoxine is present at 0.107 wt. % to 0.161 wt. % of the total micronutrients present in the composition; folic acid is present at 0.0052 wt. % to 0.0061 wt. % of the total micronutrients present in the composition; cyanocobalamin is present at 0.00019 wt. % to 0.00029 wt. % of the total micronutrients present in the composition; ascorbic acid is present at 6.087 wt. % to 7.304 wt. % of the total micronutrients present in the composition; cholecalciferol is present at 0.00065 wt. % to 0.00072 wt. % of the total micronutrients present in the composition; vitamin E is present at 2.09 wt. % to 2.30 wt. % of the total micronutrients present in the composition; calcium is present at 25.22 wt. % to 25.57 wt. % of the total micronutrients present in the composition; and/or (preferably and) selenium is present at 0.0014 wt. % to 0.0019 wt. % of the total micronutrients present in the composition; or
(e) vitamin A is present at 0.028 wt. % to 0.031 wt. % of the total micronutrients present in the composition; pyridoxine is present at 0.198 wt. % to 0.219 wt. % of the total micronutrients present in the composition; folic acid is present at 0.0052 wt. % to 0.0061 wt. % of the total micronutrients present in the composition; cyanocobalamin is present at 0.00035 wt. % to 0.00039 wt. % of the total micronutrients present in the composition; ascorbic acid is present at 8.696 wt. % to 9.913 wt. % of the total micronutrients present in the composition; cholecalciferol is present at 0.00065 wt. % to 0.00072 wt. % of the total micronutrients present in the composition; vitamin E is present at 3.39 wt. % to 3.57 wt. % of the total micronutrients present in the composition; calcium is present at 21.74 wt. % to 24.52 wt. % of the total micronutrients present in the composition; and/or (preferably and) selenium is present at 0.0020 wt. % to 0.0025 wt. % of the total micronutrients present in the composition; or
(f) vitamin A is present at 0.023 wt. % to 0.027 wt. % of the total micronutrients present in the composition; pyridoxine is present at 0.198 wt. % to 0.219 wt. % of the total micronutrients present in the composition; folic acid is present at 0.0065 wt. % to 0.0085 wt. % of the total micronutrients present in the composition; cyanocobalamin is present at 0.00035 wt. % to 0.00039 wt. % of the total micronutrients present in the composition; ascorbic acid is present at 6.087 wt. % to 7.304 wt. % of the total micronutrients present in the composition; cholecalciferol is present at 0.00020 wt. % to 0.00026 wt. % of the total micronutrients present in the composition; vitamin E is present at 2.09 wt. % to 2.30 wt. % of the total micronutrients present in the composition; calcium is present at 19.13 wt. % to 20.87 wt. % of the total micronutrients present in the composition; and/or (preferably and) selenium is present at 0.0020 wt. % to 0.0025 wt. % of the total micronutrients present in the composition; or
(g) vitamin A is present at 0.023 wt. % to 0.027 wt. % of the total micronutrients present in the composition; pyridoxine is present at 0.061 wt. % to 0.079 wt. % of the total micronutrients present in the composition; folic acid is present at 0.0065 wt. % to 0.0085 wt. % of the total micronutrients present in the composition; cyanocobalamin is present at 0.00011 wt. % to 0.00014 wt. % of the total micronutrients present in the composition; ascorbic acid is present at 8.696 wt. % to 9.913 wt. % of the total micronutrients present in the composition; cholecalciferol is present at 0.00043 wt. % to 0.00053 wt. % of the total micronutrients present in the composition; vitamin E is present at 3.39 wt. % to 3.57 wt. % of the total micronutrients present in the composition; calcium is present at 21.74 wt. % to 24.52 wt. % of the total micronutrients present in the composition; and/or (preferably and) selenium is present at 0.0020 wt. % to 0.0025 wt. % of the total micronutrients present in the composition; or
(h) vitamin A is present at 0.023 wt. % to 0.027 wt. % of the total micronutrients present in the composition; pyridoxine is present at 0.107 wt. % to 0.161 wt. % of the total micronutrients present in the composition; folic acid is present at 0.0065 wt. % to 0.0085 wt. % of the total micronutrients present in the composition; cyanocobalamin is present at 0.00019 wt. % to 0.00029 wt. % of the total micronutrients present in the composition; ascorbic acid is present at 6.087 wt. % to 7.304 wt. % of the total micronutrients present in the composition; cholecalciferol is present at 0.00020 wt. % to 0.00026 wt. % of the total micronutrients present in the composition; vitamin E is present at 2.09 wt. % to 2.30 wt. % of the total micronutrients present in the composition; calcium is present at 21.74 wt. % to 24.52 wt. % of the total micronutrients present in the composition; and/or (preferably and) selenium is present at 0.0020 wt. % to 0.0025 wt. % of the total micronutrients present in the composition; or
(i) vitamin A is present at 0.023 wt. % to 0.027 wt. % of the total micronutrients present in the composition; pyridoxine is present at 0.107 wt. % to 0.161 wt. % of the total micronutrients present in the composition; folic acid is present at 0.0065 wt. % to 0.0085 wt. % of the total micronutrients present in the composition; cyanocobalamin is present at 0.00019 wt. % to 0.00029 wt. % of the total micronutrients present in the composition; ascorbic acid is present at 3.304 wt. % to 3.652 wt. % of the total micronutrients present in the composition; cholecalciferol is present at 0.00043 wt. % to 0.00053 wt. % of the total micronutrients present in the composition; vitamin E is present at 0.33 wt. % to 0.35 wt. % of the total micronutrients present in the composition; calcium is present at 21.74 wt. % to 24.52 wt. % of the total micronutrients present in the composition; and/or (preferably and) selenium is present at 0.0014 wt. % to 0.0019 wt. % of the total micronutrients present in the composition; or
(j) vitamin A is present at 0.028 wt. % to 0.031 wt. % of the total micronutrients present in the composition; pyridoxine is present at 0.107 wt. % to 0.161 wt. % of the total micronutrients present in the composition; folic acid is present at 0.0052 wt. % to 0.0061 wt. % of the total micronutrients present in the composition; cyanocobalamin is present at 0.00019 wt. % to 0.00029 wt. % of the total micronutrients present in the composition; ascorbic acid is present at 8.696 wt. % to 9.913 wt. % of the total micronutrients present in the composition; cholecalciferol is present at 0.00043 wt. % to 0.00053 wt. % of the total micronutrients present in the composition; vitamin E is present at 3.39 wt. % to 3.57 wt. % of the total micronutrients present in the composition; calcium is present at: 21.74 wt. % to 24.52 wt. % of the total micronutrients present in the composition; and/or (preferably and) selenium is present at 0.0020 wt. % to 0.0025 wt. % of the total micronutrients present in the composition; or
(k) vitamin A is present at 0.028 wt. % to 0.031 wt. % of the total micronutrients present in the composition; pyridoxine is present at 0.107 wt. % to 0.161 wt. % of the total micronutrients present in the composition; folic acid is present at 0.0065 wt. % to 0.0085 wt. % of the total micronutrients present in the composition; cyanocobalamin is present at 0.00019 wt. % to 0.00029 wt. % of the total micronutrients present in the composition; ascorbic acid is present at 8.696 wt. % to 9.913 wt. % of the total micronutrients present in the composition; cholecalciferol is present at 0.00043 wt. % to 0.00053 wt. % of the total micronutrients present in the composition; vitamin E is present at 3.39 wt. % to 3.57 wt. % of the total micronutrients present in the composition; calcium is present at 21.74 wt. % to 24.52 wt. % of the total micronutrients present in the composition; and/or (preferably and) selenium is present at 0.0014 wt. % to 0.0019 wt. % of the total micronutrients present in the composition; or
(l) vitamin A is present at 0.023 wt. % to 0.027 wt. % of the total micronutrients present in the composition; pyridoxine is present at 0.107 wt. % to 0.161 wt. % of the total micronutrients present in the composition; folic acid is present at 0.0065 wt. % to 0.0085 wt. % of the total micronutrients present in the composition; cyanocobalamin is present at 0.00019 wt. % to 0.00029 wt. % of the total micronutrients present in the composition; ascorbic acid is present at 8.696 wt. % to 9.913 wt. % of the total micronutrients present in the composition; cholecalciferol is present at 0.00020 wt. % to 0.00026 wt. % of the total micronutrients present in the composition; vitamin E is present at 3.39 wt. % to 3.57 wt. % of the total micronutrients present in the composition; calcium is present at 19.13 wt. % to 20.87 wt. % of the total micronutrients present in the composition; and/or (preferably and) selenium is present at 0.0020 wt. % to 0.0025 wt. % of the total micronutrients present in the composition; or
(m) vitamin A is present at 0.028 wt. % to 0.031 wt. % of the total micronutrients present in the composition; pyridoxine is present at 0.061 wt. % to 0.079 wt. % of the total micronutrients present in the composition; folic acid is present at 0.0089 wt. % to 0.0091 wt. % of the total micronutrients present in the composition; cyanocobalamin is present at 0.00011 wt. % to 0.00014 wt. % of the total micronutrients present in the composition; ascorbic acid is present at 6.087 wt. % to 7.304 wt. % of the total micronutrients present in the composition; cholecalciferol is present at 0.00043 wt. % to 0.00053 wt. % of the total micronutrients present in the composition; vitamin E is present at 2.09 wt. % to 2.30 wt. % of the total micronutrients present in the composition; calcium is present at 21.74 wt. % to 24.52 wt. % of the total micronutrients present in the composition; and/or selenium (preferably and) is present at 0.0014 wt. % to 0.0019 wt. % of the total micronutrients present in the composition; or
(n) vitamin A is present at 0.023 wt. % to 0.027 wt. % of the total micronutrients present in the composition; pyridoxine is present at 0.198 wt. % to 0.219 wt. % of the total micronutrients present in the composition; folic acid is present at 0.0065 wt. % to 0.0085 wt. % of the total micronutrients present in the composition; cyanocobalamin is present at 0.00035 wt. % to 0.00039 wt. % of the total micronutrients present in the composition; ascorbic acid is present at 8.696 wt. % to 9.913 wt. % of the total micronutrients present in the composition; cholecalciferol is present at 0.00043 wt. % to 0.00053 wt. % of the total micronutrients present in the composition; vitamin E is present at 3.39 wt. % to 3.57 wt. % of the total micronutrients present in the composition; calcium is present at 21.74 wt. % to 24.52 wt. % of the total micronutrients present in the composition; and/or selenium (preferably and) is present at 0.0020 wt. % to 0.0025 wt. % of the total micronutrients present in the composition; or
(o) vitamin A is present at 0.028 wt. % to 0.031 wt. % of the total micronutrients present in the composition; pyridoxine is present at 0.061 wt. % to 0.079 wt. % of the total micronutrients present in the composition; folic acid is present at 0.0065 wt. % to 0.0085 wt. % of the total micronutrients present in the composition; cyanocobalamin is present at 0.00011 wt. % to 0.00014 wt. % of the total micronutrients present in the composition; ascorbic acid is present at 8.696 wt. % to 9.913 wt. % of the total micronutrients present in the composition; cholecalciferol is present at 0.00020 wt. % to 0.00026 wt. % of the total micronutrients present in the composition; vitamin E is present at 3.39 wt. % to 3.57 wt. % of the total micronutrients present in the composition; calcium is present at 19.13 wt. % to 20.87 wt. % of the total micronutrients present in the composition; and/or (preferably and) selenium is present at 0.0014 wt. % to 0.0019 wt. % of the total micronutrients present in the composition; or
(p) vitamin A is present at 0.023 wt. % to 0.027 wt. % of the total micronutrients present in the composition; pyridoxine is present at 0.061 wt. % to 0.079 wt. % of the total micronutrients present in the composition; folic acid is present at 0.0065 wt. % to 0.0085 wt. % of the total micronutrients present in the composition; cyanocobalamin is present at 0.00011 wt. % to 0.00014 wt. % of the total micronutrients present in the composition; ascorbic acid is present at 6.087 wt. % to 7.304 wt. % of the total micronutrients present in the composition; cholecalciferol is present at 0.00020 wt. % to 0.00026 wt. % of the total micronutrients present in the composition; vitamin E is present at 2.09 wt. % to 2.30 wt. % of the total micronutrients present in the composition; calcium is present at 19.13 wt. % to 20.87 wt. % of the total micronutrients present in the composition; and/or (preferably and) selenium is present at 0.0020 wt. % to 0.0025 wt. % of the total micronutrients present in the composition; or
(q) vitamin A is present at 0.023 wt. % to 0.027 wt. % of the total micronutrients present in the composition; pyridoxine is present at 0.107 wt. % to 0.161 wt. % of the total micronutrients present in the composition; folic acid is present at 0.0065 wt. % to 0.0085 wt. % of the total micronutrients present in the composition; cyanocobalamin is present at 0.00019 wt. % to 0.00029 wt. % of the total micronutrients present in the composition; ascorbic acid is present at 8.696 wt. % to 9.913 wt. % of the total micronutrients present in the composition; cholecalciferol is present at 0.00065 wt. % to 0.00072 wt. % of the total micronutrients present in the composition; vitamin E is present at 3.39 wt. % to 3.57 wt. % of the total micronutrients present in the composition; calcium is present at 25.22 wt. % to 25.57 wt. % of the total micronutrients present in the composition; and/or (preferably and) selenium is present at 0.0014 wt. % to 0.0019 wt. % of the total micronutrients present in the composition.

A composition of the invention may comprise one or more further micronutrients, e.g. selected from thiamin (B1) (e.g. thiamine HCL), riboflavin (B2), nicotinic acid (B3) (e.g. niacin or nicotinamide, preferably nicotinamide), pantothenic acid (B5) (e.g. calcium pantothenate), biotin (B7) (e.g. D-biotin), para-aminobenzoic acid (PABA) (B10) (e.g. derived from and/or extracted from yeast), vitamin K1 (e.g. phylloquinone), inositol, choline (Vitamin J) (e.g. L-choline bitartrate), chloride, chromium (e.g. chromium picolinate), phosphorus (e.g. dicalcium phosphate), iodine (e.g. potassium iodide), iron, molybdenum (e.g. molybdenum bisglycinate), manganese (e.g. manganese bisglycinate), magnesium (e.g. magnesium oxide), fluoride, potassium (e.g. potassium gluconate), copper (e.g. copper gluconate), sodium (e.g. sodium fluoride) and/or zinc (e.g. zinc oxide). Preferably, a nutritional composition of the invention further comprises thiamin (B1), riboflavin (B2), nicotinic acid (B3), pantothenic acid (B5), biotin (B7), para-aminobenzoic acid (PABA) (B10), vitamin K1, inositol, choline (Vitamin J), chloride, chromium, phosphorus, iodine, iron, molybdenum, manganese, magnesium, fluoride, potassium, copper, and/or zinc. For example, a composition of the invention may further comprise at least 1, 2, 3, 4, 5, 6, 7, 8, 9, 10, 11, 12, 13, 14, 15, 16, 17, 18, 19 or 20 of thiamin (B1), riboflavin (B2), nicotinic acid (B3), pantothenic acid (B5), biotin (B7), para-aminobenzoic acid (PABA) (B10), vitamin K1, inositol, choline (Vitamin J), chloride, chromium, phosphorus, iodine, iron, molybdenum, manganese, magnesium, fluoride, potassium, copper, and/or zinc. Preferably, a nutritional composition of the invention further comprises thiamin (B1), riboflavin (B2), nicotinic acid (B3), pantothenic acid (B5), biotin (B7), para-aminobenzoic acid (PABA) (B10), vitamin K1, inositol, choline (Vitamin J), chloride, chromium, phosphorus, iodine, iron, molybdenum, manganese, magnesium, fluoride, potassium, copper, and zinc.

The micronutrients of the invention may consist of:
(i) vitamin A, pyridoxine (vitamin B6), folic acid (vitamin B9), cyanocobalamin (vitamin B12), ascorbic acid (vitamin C), cholecalciferol (vitamin D3), vitamin E, calcium, and selenium; and
(ii) at least 1, 2, 3, 4, 5, 6, 7, 8, 9, 10, 11, 12, 13, 14, 15, 16, 17, 18, 19 or 20 of thiamin (B1), riboflavin (B2), nicotinic acid (B3), pantothenic acid (B5), biotin (B7), para-aminobenzoic acid (PABA) (B10), vitamin K1, inositol, choline (Vitamin J), chloride, chromium, phosphorus, iodine, iron, molybdenum, manganese, magnesium, fluoride, potassium, copper, and/or zinc.

For example, the micronutrients of the invention may consist of:
(i) vitamin A, pyridoxine (vitamin B6), folic acid (vitamin B9), cyanocobalamin (vitamin B12), ascorbic acid (vitamin C), cholecalciferol (vitamin D3), vitamin E, calcium, and selenium; and
(ii) thiamin (B1), riboflavin (B2), nicotinic acid (B3), pantothenic acid (B5), biotin (B7), para-aminobenzoic acid (PABA) (B10), vitamin K1, inositol, choline (Vitamin J), chloride, chromium, phosphorus, iodine, iron, molybdenum, manganese, magnesium, fluoride, potassium, copper, and zinc.

The one or more further micronutrients may be present at a daily unit dose of the one or more further micronutrients or a fraction of a daily unit dose of the one or more further micronutrients. For example, the fraction of a daily unit dose of the one or more further micronutrients may be at least 10%, 20%, 30%, 40%, 50%, 60%, 70%, 80%, 90% or 95% of a daily unit dose of the one or more further micronutrients. Preferably, a fraction of the daily unit dose of the one or more further micronutrients is 50% of a daily unit dose of the one or more micronutrients. This is particularly useful where (for example), the composition is provided as a single serving and wherein it is intended that a subject consumes two servings per day. In this regard, a kit of the invention may comprise a container comprising a fraction of the daily unit dose of the one or more further micronutrients. Such containers may comprise a pre-packaged single serving of the one or more further micronutrients (optionally in combination with the one or more micronutrients of the invention).

In some embodiments, the composition of the invention comprises more than a daily unit dose of the one or more further micronutrients, e.g. two, three, four, five or more daily unit doses of the one or more further micronutrients. In this regard, a kit of the invention may comprise a container comprising at least one (e.g. two, three or more) daily unit doses of the one or more further micronutrients (optionally in combination with the one or more micronutrients of the invention).

A composition or kit of the invention preferably comprises at least two (e.g. at least three, four, five, six, seven or eight, or more) further micronutrients, wherein a weight ratio between the at least two (e.g. at least three, four, five, six, seven or eight, or more) further micronutrients is the same as the weight ratio between the at least two (e.g. at least three, four, five, six, seven or eight, or more) further micronutrients in a daily unit dose described herein.

Suitable daily unit doses of the one or more further micronutrients are provided below.

A daily unit dose of the one or more further micronutrients may comprise:
(a) a dose of thiamin (B1) that is 100% to 120% (preferably 108%) of a reference thiamin (B1) daily dose, wherein the reference thiamin (B1) daily dose is 1.1 mg;
(b) a dose of riboflavin (B2) that is 90% to 110% (preferably 100%) of a reference riboflavin (B2) daily dose, wherein the reference riboflavin (B2) daily dose is 1.4 mg;
(c) a dose of nicotinic acid (B3) that is 100% to 120% (preferably 112%) of a reference nicotinic acid (B3) daily dose, wherein the reference nicotinic acid (B3) daily dose is 16 mg;
(d) a dose of pantothenic acid (B5) that is 90% to 110% (preferably 100%) of a reference pantothenic acid (B5) daily dose, wherein the reference pantothenic acid (B5) daily dose is 6 mg;
(e) a dose of biotin (B7) that is 90% to 110% (preferably 100%) of a reference biotin (B7) daily dose, wherein the reference biotin (B7) daily dose is 50 μg;
(f) a dose of para-aminobenzoic acid (PABA) (B10) that is 90% to 110% (preferably 100%) of a reference para-aminobenzoic acid (PABA) (B10) daily dose, wherein the reference para-aminobenzoic acid (PABA) (B10) daily dose is 25 mg;
(g) a dose of vitamin K1 that is 150% to 220% (preferably 187%) of a reference vitamin K1 daily dose, wherein the reference vitamin K1 daily dose is 75 μg;
(h) a dose of inositol that is 90% to 110% (preferably 100%) of a reference inositol daily dose, wherein the reference inositol daily dose is 30 mg;
(i) a dose of choline (Vitamin J) that is 120% to 150% (preferably 137%) of a reference choline (Vitamin J) daily dose, wherein the reference choline (Vitamin J) daily dose is 400 mg;
(j) a dose of chloride that is 90% to 110% (preferably 100%) of a reference chloride daily dose, wherein the reference chloride daily dose is 800 mg;
(k) a dose of chromium that is 90% to 110% (preferably 100%) of a reference chromium daily dose, wherein the reference chromium daily dose is 40 μg;
(l) a dose of phosphorus that is 90% to 110% (preferably 100%) of a reference phosphorus daily dose, wherein the reference phosphorus daily dose is 700 mg;
(m) a dose of iodine that is 90% to 110% (preferably 100%) of a reference iodine daily dose, wherein the reference iodine daily dose is 150 μg;
(n) a dose of iron that is 90% to 110% (preferably 100%) of a reference iron daily dose, wherein the reference iron daily dose is 14 μg;
(o) a dose of molybdenum that is 90% to 110% (preferably 100%) of a reference molybdenum daily dose, wherein the reference molybdenum daily dose is 50 μg;
(p) a dose of manganese that is 100% to 120% (preferably 116%) of a reference manganese daily dose, wherein the reference manganese daily dose is 2 μg;
(q) a dose of magnesium that is 90% to 110% (preferably 100%) of a reference magnesium daily dose, wherein the reference magnesium daily dose is 375 mg;
(r) a dose of fluoride that is 90% to 110% (preferably 100%) of a reference fluoride daily dose, wherein the reference fluoride daily dose is 3.5 mg;
(s) a dose of potassium that is 10% to 20% (preferably 15%) of a reference potassium daily dose, wherein the reference potassium daily dose is 2000 mg;
(t) a dose of copper that is 90% to 110% (preferably 100%) of a reference copper daily dose, wherein the reference copper daily dose is 1 mg; and/or
(u) a dose of zinc that is 100% to 120% (preferably 110%) of a reference zinc daily dose, wherein the reference zinc daily dose is 10 mg.

A macronutrient may be any nutrient that provides energy (or calories). The composition (or kit) of the invention preferably comprises one or more macronutrients. In some embodiments, the macronutrient may be a carbohydrate, a protein, a fat and/or fibre. Preferably, a macronutrient is at least a protein.

A preferred serving size of a composition comprising micronutrients and macronutrients is 30-60 g, more preferably 35 g or 50 g.

A kit of the invention may comprise a further container comprising one or more macronutrients. Alternatively, the one or more macronutrients may be present in a container comprising the one or more micronutrients. The instructions present with a kit may explain how to admix the micronutrients and macronutrients.

The composition of the invention may comprise at least one carbohydrate. The carbohydrate may be a digestible carbohydrate or an indigestible carbohydrate. In one embodiment, an indigestible carbohydrate may be a fibre. Carbohydrates suitable for inclusion in nutritional compositions will be well known to the skilled person and include, for example, sugars and starches. Carbohydrates may include monosaccharides, disaccharides, oligosaccharides, and polysaccharides. Preferred carbohydrates include sucrose, glucose, fructose, lactose, galactose, maltose, starch and maltodextrin. The at least one carbohydrate of the composition is preferably sucrose. A carbohydrate may be present at 0.5-5 wt. % (e.g. 1-4 wt. %) (e.g. where the composition is a low carbohydrate composition). A carbohydrate may be present at 6-17 wt. % (e.g. 14-16.5 wt. %) (e.g. where the composition is a medium carbohydrate composition). A carbohydrate may be present at 18-35 wt. % (e.g. 19-26 wt. %) (e.g. where the composition is a high carbohydrate composition).

The composition of the invention may comprise at least one fat. Fats suitable for inclusion in nutritional compositions will be well known to the skilled person. The at least one fat of the composition is preferably a vegetable fat, such as coconut oil, rape seed oil, sunflower oil, soy lecithin, palm oil or flaxseed, preferably flaxseed (e.g. wherein the composition comprises flaxseed powder, such as milled flaxseed powder). A fat may be present at 5-13 wt. % (e.g. 7-12 wt. %) (e.g. where the composition is a low fat composition). A fat may be present at 14-19 wt. % (e.g. 15-17 wt. %) (e.g. where the composition is a medium fat composition). A fat may be present at 20-40 wt. % (e.g. 26-31 wt. %) (e.g. where the composition is a high fat composition).

The composition of the invention (or kit) may comprise a fibre. The fibre may be any suitable dietary fibre and may be present at 1-20 wt. %, for example 2-15 wt. %. Preferably, the fibre is flaxseed (e.g. wherein the composition comprises flaxseed powder, such as milled flaxseed powder).

Preferably, a composition of the invention (or kit) comprises a protein. The protein may be a plant-derived protein, an animal-derived protein or a synthetic protein (or combinations thereof). Protein sources suitable for inclusion in nutritional compositions will be well known to the skilled person and include, for example, soy protein and whey protein. A preferred protein source may be pea protein and/or brown rice protein isolate (e.g. a vegan protein blend comprising pea protein and brown rice protein). A protein may be present at 20-80 wt. %, for example 40-80 wt. %, or 55-65 wt. % (preferably 63 wt. %). The amount of protein in the composition may be based on a lifestyle goal of a subject. By way of non-limiting example, the lifestyle goal of the subject may be to build lean muscle mass.

The one or more macronutrients may be present at a daily unit dose of the one or more macronutrients or a fraction of a daily unit dose of the one or more macronutrients. For example, the fraction of a daily unit dose of the one or more macronutrients may be at least 10%, 20%, 30%, 40%, 50%, 60%, 70%, 80%, 90% or 95% of a daily unit dose of the one or more macronutrients. Preferably, a fraction of the daily unit dose of the one or more macronutrients is 50% of a daily unit dose of the one or more macronutrients. This is particularly useful where (for example), the composition is provided as a single serving and wherein it is intended that a subject consumes two servings per day. In this regard, a kit of the invention may comprise a container comprising a fraction of the daily unit dose of the one or more macronutrients. Such containers may comprise a pre-packaged single serving of the one or more macronutrients (optionally in combination with the one or more micronutrients and/or one or more further micronutrients of the invention).

In some embodiments, the composition of the invention comprises more than a daily unit dose of the one or more macronutrients, e.g. two, three, four, five or more daily unit doses of the one or more macronutrients. In this regard, a kit of the invention may comprise a container comprising at least one (e.g. two, three or more) daily unit doses of the one or more macronutrients (optionally in combination with the one or more micronutrients and/or one or more further micronutrients of the invention).

Suitable daily unit doses of the one or more macronutrients are provided below.

A daily unit dose of protein may refer to a dose of protein that is 1-500% of a reference protein daily dose, wherein the reference protein daily dose is 44 g. A lower end of the range may be at least 20% or 50%, preferably 75%. An upper limit of the range may be 400%, 300%, 200%, or 150% preferably 125%. Preferably, a daily unit dose of protein may refer to a dose of protein that is 75% to 125% of a reference protein daily dose.

A daily unit dose of carbohydrate may refer to a dose of carbohydrate that is 0.1-200% of a reference carbohydrate daily dose, wherein the reference carbohydrate daily dose is 260 g.

Where the daily unit dose is a low carbohydrate daily unit dose a lower end of the range may be at least 0.25% or 0.5%, preferably 1%. An upper limit of the range may be 4% or 3%, preferably 2.5%. Preferably, a daily unit dose of carbohydrate may refer to a dose of carbohydrate that is 1% to 2.5% of a reference carbohydrate daily dose.

Where the daily unit dose is a medium carbohydrate daily unit dose a lower end of the range may be at least 4.1% or 4.5%, preferably 5%. An upper limit of the range may be 12.9%, 10%, or 8%, preferably 7%. Preferably, daily unit dose of carbohydrate may refer to a dose of carbohydrate that is 5% to 7% of a reference carbohydrate daily dose.

Where the daily unit dose is a high carbohydrate daily unit dose a lower end of the range may be at least 13.0% or 14%, preferably 15%. An upper limit of the range may be 150%, 100%, 50%, or 20%, preferably 17%. Preferably, daily unit dose of carbohydrate may refer to a dose of carbohydrate that is 15% to 17% of a reference carbohydrate daily dose.

A daily unit dose of fat may refer to a dose of fat that is 1-150% of a reference fat daily dose, wherein the reference fat daily dose is 70 g.

Where the daily unit dose is a low fat daily unit dose a lower end of the range may be at least 1% or 3%, preferably 5%. An upper limit of the range may be 15.3%, 14%, or 12%, preferably 10%. Preferably, a daily unit dose of fat may refer to a dose of fat that is 5% to 10% of a reference fat daily dose.

Where the daily unit dose is a medium fat daily unit dose a lower end of the range may be at least 15.4% or 17%, preferably 20%. An upper limit of the range may be 31.4% or 30%, preferably 25%. Preferably, a daily unit dose of fat may refer to a dose of fat that is 20% to 25% of a reference fat daily dose.

Where the daily unit dose is a high fat daily unit dose a lower end of the range may be at least 31.5%, 35%, or 37%, preferably 39%. An upper limit of the range may be 125%, 100%, or 75%, or 50%, preferably 42%. Preferably, a daily unit dose of fat may refer to a dose of fat that is 39% to 42% of a reference fat daily dose.

A daily unit dose of fibre may refer to a dose of fibre that is 1-500% of a reference fibre daily dose, wherein the reference fibre daily dose is 30 g. A lower end of the range may be at least 2%, 10% or 20%. An upper limit of the range may be 250%, 200%, 100% or 75%, preferably 50%. Preferably, a daily unit dose of fibre may refer to a dose of fibre that is 1% to 50% of a reference fibre daily dose.

A composition of the invention may further comprise:
(i) low carbohydrate and low fat;
(ii) low carbohydrate and medium fat;
(iii) medium carbohydrate and low fat;
(iv) medium carbohydrate and medium fat;
(v) high carbohydrate and low fat;
(vi) high carbohydrate and medium fat;
(vii) high carbohydrate and high fat;
(viii) low carbohydrate and high fat; or
(ix) medium carbohydrate and high fat.

Preferably said composition further comprises protein and/or fibre.

The amount of a micronutrient present in a composition (or kit) of the invention may be expressed as a weight ratio to protein.

A composition or kit of the invention preferably comprises at least two (e.g. at least three, or four) macronutrients, wherein a weight ratio between the at least two (e.g. at least three, or four) macronutrients is the same as the weight ratio between the at least two (e.g. at least three, or four) macronutrients in a daily unit dose described herein.

The composition (or kit) may comprise:
(a) vitamin A present at a weight ratio of vitamin A to protein of greater than 1:55,000 to 1:14,667 (preferably 1:50,000 to 1:25,000);
(b) pyridoxine present at a weight ratio of pyridoxine to protein of greater than 1:31,429 to 1:440 (preferably 1:20,000 to 1:2,000);
(c) folic acid present at a weight ratio of folic acid to protein of greater than 1:220,000 to 1:44,000 (preferably 1:200,000 to 1:75,000);
(d) cyanocobalamin present at a weight ratio of cyanocobalamin to protein of greater than 1:17,600,000 to 1:44,000 (preferably 1:12,000,000 to 1:1,000,000);
(e) ascorbic acid present at a weight ratio of ascorbic acid to protein of greater than 1:550 to 1:22 (preferably 1:350 to 1:50);
(f) cholecalciferol present at a weight ratio of cholecalciferol to protein of greater than 1:8,800,000 to 1:440,000 (preferably 1:6,000,000 to 1:750,000);
(g) vitamin E present at a weight ratio of vitamin E to protein of greater than 1:3,667 to 1:40 (preferably 1:3,200 to 1:150);
(h) calcium present at a weight ratio of calcium to protein of greater than 1:55 to 1:18 (preferably 1:52 to 1:1.25); and/or
(i) selenium present at a weight ratio of selenium to protein of greater than 1:800,000 to 1:110,041 (preferably 1:720,000 to 1:250,000).

The composition (or kit) may comprise:
(a) vitamin A present at a weight ratio of vitamin A to protein of: (i) greater than 1:55,000 to 1:43,137 (e.g. 1:54,000 to 1:44,000, 1:53,000 to 1:45,000, 1:52,000 to 1:46,000, 1:51,000 to 1:47,000 or 1:50,000 to 1:48,000 preferably 1:50,000 to 1:43,137, e.g. a lower end of the range may be at least 1:54,000, 1:53,000, 1:52,000, 1:51,000 or 1:50,000, preferably 1:50,000 and/or an upper end of the range may be less than or equal to 1:44,000, 1:46,000, 1:48,000, 1:50,000 or 1:52,000, preferably 1:43,137); (ii) greater than 1:43,137 to 1:34,700 (e.g. a lower end of the range may be at least 1:43,000, 1:42,000, 1:41,000, 1:40,000 or 1:39,000 and/or an upper end of the range may be less than or equal to 1:35,000, 1:36,000, 1:37,000, 1:38,000 or 1:39,000); or (iii) greater than 1:34,700 to 1:14,667 (preferably greater than 1:34,700 to 1:25,000, e.g. a lower end of the range may be at least 1:34,000, 1:33,000, 1:32,000, 1:31,000 or 1:30,000 and/or an upper end of the range may be less than or equal to 1:15,000, 1:16,000, 1:17,000, 1:18,000 or 1:19,000);
(b) pyridoxine present at a weight ratio of pyridoxine to protein of: (i) greater than 1:31,429 to 1:10,304 (e.g. 1:30,000 to 1:11,000, 1:29,000 to 1:12000, 1:28,000 to 1:13,000, 1:27,000 to 1:14,000, 1:26,000 to 1:15,000 or 1:25,000 to 1:16,000, preferably 1:20,000 to 1:10,304, e.g. a lower end of the range may be at least 1:30,000, 1:29,000, 1:28,000, 1:27,000 or 1:26,000, preferably 1:20,000 and/or an upper end of the range may be less than or equal to 1:11,000, 1:12,000, 1:13,000, 1:14,000 or 1:15,000, preferably 1:10,304); (ii) greater than 1:10,304 to 1:5,327 (e.g. a lower end of the range may be at least 1:10,000, 1:9,500, 1:9,000, 1:8,500 or 1:8,000 and/or an upper end of the range may be less than or equal to 1:5,500, 1:6,000, 1:6,500, 1:7,000 or 1:7,500); or (iii) greater than 1:5,327 to 1:440 (preferably greater than 1:5,327 to 1:2,000, e.g. a lower end of the range may be at least 1:5,000, 1:4,500, 1:4,000, 1:3,500 or 1:3,000 and/or an upper end of the range may be less than or equal to 1:450, 1:500, 1:550, 1:600 or 1:750);
(c) folic acid present at a weight ratio of folic acid to protein of: (i) greater than 1:220,000 to 1:151,724 (e.g. 1:215,000 to 1:152,000, 1:210,000 to 1:154,000, 1:205,000 to 1:156,000 or 1:200,000 to 1:158,000, preferably 1:200,000 to 1:151,724, e.g. a lower end of the range may be at least 1:215,000, 1:210,000, 1:205,000, 1:200,000 or 1:195,000, preferably 1:200,000 and/or an upper end of the range may be less than or equal to 1:155,000, 1:160,000, 1:165,000, 1:170,000 or 1:175,000, preferably 1:151,724); (ii) greater than 1:151,724 to 1:110,000 e.g. a lower end of the range may be at least 1:150,000, 1:145,000, 1:140,000, 1:135,000 or 1:130,000 and/or an upper end of the range may be less than or equal to 1:115,000, 1:120,000, 1:125,000, 1:130,000 or 1:140,000; or (iii) greater than 1:110,000 to 1:44,000 (preferably greater than 1:110,000 to 1:75,000, e.g. a lower end of the range may be at least 1:105,000, 1:100,000, 1:95,000, 1:90,000 or 1:85,000 and/or an upper end of the range may be less than or equal to 1:46,000, 1:48,000, 1:50,000, 1:52,000 or 1:54,000);
(d) cyanocobalamin present at a weight ratio of cyanocobalamin to protein of: (i) greater than 1:17,600,000 to 1:5,770,492 (e.g. 1:16,000,000 to 1:6,000,000, 1:15,000,000 to 1:7,000,000, 1:14,000,000 to 1:8,000,000, 1:13,000,000 to 1:9,000,000 or 1:12,000,000 to 1:10,000,000, preferably 1:12,000,000 to 1:5,770,492, e.g. a lower end of the range may be at least 1:16,000,000, 1:15,000,000, 1:14,000,000, 1:13,000,000 or 1:12,000,000, preferably 1:12,000,000 and/or an upper end of the range may be less than or equal to 1:6,000,000, 1:6,500,000, 1:7,000,000, 1:7,500,000 or 1:8,000,000, preferably 1:5,770,492); (ii) greater than 1:5,770,492 to 1:2,983,051 (e.g. a lower end of the range may be at least 1:5,500,000, 1:5,000,000, 1:4,500,000, 1:4,000,000 or 1:3,500,000 and/or an upper end of the range may be less than or equal to 1:3,000,000, 1:3, 100,000, 1:3,200,000, 1:3,300,000 or 1:3,400,000); or (iii) greater than 1:2,983,051 to 1:44,000 (preferably greater than 1:2,983,051 to 1:1,000,000, e.g. a lower end of the range may be at least 1:2,500,000, 1:2,000,000, 1:1,500,000, 1:1,000,000 or 1:500,000 and/or an upper end of the range may be less than or equal to 1:50,000, 1:100,000, 1:100,500, 1:200,000 or 1:250,000);
(e) ascorbic acid present at a weight ratio of ascorbic acid to protein of: (i) greater than 1:550 to 1:200 (e.g. 1:500 to 1:220, 1:450 to 1:240, 1:400 to 1:260 or 1:350 to 1:280, preferably 1:350 to 1:200, e.g. a lower end of the range may be at least 1:500, 1:450, 1:400 or 1:350, preferably 1:350 and/or an upper end of the range may be less than or equal to 1:220, 1:240, 1:260, 1:280 or 1:300, preferably 1:200); (ii) greater than 1:200 to 1:120 (e.g. a lower end of the range may be at least 1:195, 1:190, 1:185 or 1:180 and/or an upper end of the range may be less than or equal to 1:125, 1:130, 1:135, 1:140 or 1:145); or (iii) greater than 1:120 to 1:22 (preferably greater than 1:120 to 1:50, e.g. a lower end of the range may be at least 1:115, 1:110, 1:105 or 1:100 and/or an upper end of the range may be less than or equal to 1:55, 1:60, 1:65, 1:70 or 1:75);
(f) cholecalciferol present at a weight ratio of cholecalciferol to protein of: (i) greater than 1:8,800,000 to 1:2,750,000 (e.g. 1:8,500,000 to 1:3,000,000, 1:8,000,000 to 1:3,500,000, 1:7,500,000 to 1:4,000,000, 1:7,000,000′ to 1:4,500,000 or 1:6,500,000 to 1:5,000,000, preferably 1:6,000,000 to 1:2,750,000, e.g. a lower end of the range may be at least 1:8,500,000, 1:8,000,000, 1:7,500,000 or 1:7,000,000, preferably 1:6,000,000 and/or an upper end of the range may be less than or equal to 1:3,000,000 1:3,500,000, 1:4,000,000, 1:4,500,000 or 1:5,000,000, preferably 1:2,750,000); (ii) greater than 1:2,750,000 to 1:1,614,679 (e.g. a lower end of the range may be at least 1:2,500,000, 1:2,300,000, 1:2, 100,000 or 1:1,900,000 and/or an upper end of the range may be less than or equal to 1:1,700,000, 1:1,800,000, 1:1,900,000 or 1:2,000,000); or (iii) greater than 1:1,614,679 to 1:440,000 (preferably greater than 1:1,614,679 to 1:750,000, e.g. a lower end of the range may be at least 1:1,500,000, 1:1,300,000, 1:1,100,000 or 1:900,000 and/or an upper end of the range may be less than or equal to 1:500,000, 1:550,000, 1:600,000 or 1:650,000);
(g) vitamin E present at a weight ratio of vitamin E to protein of: (i) greater than 1:3,667 to 1:789 (e.g. 1:3,500 to 1:800, 1:3,400 to 1:850, 1:3,300 to 1:900 or 1:3,200 to 1:950, preferably 1:3,200 to 1:789, e.g. a lower end of the range may be at least 1:3,500, 1:3,000, 1:2,500 or 1:2,000, preferably 1:3,200 and/or an upper end of the range may be less than or equal to 1:800, 1:1000, 1:1200, 1:1400 or 1:1600, preferably 1:789); (ii) greater than 1:789 to 1:336 (e.g. a lower end of the range may be at least 1:750, 1:700, 1:650 or 1:600 and/or an upper end of the range may be less than or equal to 1:350, 1:400, 1:450, 1:500 or 1:550); or (iii) greater than 1:336 to 1:40 (preferably greater than 1:336 to 1:150, e.g. a lower end of the range may be at least 1:300, 1:280, 1:260 or 1:240 and/or an upper end of the range may be less than or equal to 1:50, 1:100, 1:150 or 1:200);
(h) calcium present at a weight ratio of calcium to protein of: (i) greater than 1:55 to 1:45 (e.g. 1:54 to 1:46, 1:53 to 1:47 or 1:52 to 1:48, preferably 1:52 to 1:45, e.g. a lower end of the range may be at least 1:54, 1:53, 1:52 or 1:51, preferably 1:52 and/or an upper end of the range may be less than or equal to 1:46, 1:47, 1:48 or 1:49, preferably 1:45); (ii) greater than 1:45 to 1:38 (e.g. a lower end of the range may be at least 1:44, 1:43, 1:42 or 1:41 and/or an upper end of the range may be less than or equal to 1:39, 1:40, 1:41 or 1:42); or (iii) greater than 1:38 to 1:18 (preferably greater than 1:38 to 1:1.25, e.g. a lower end of the range may be at least 1:36, 1:34, 1:32 or 1:30 and/or an upper end of the range may be less than or equal to 1:20, 1:22, 1:24 or 1:26); and/or
(i) selenium present at a weight ratio of selenium to protein of: (i) greater than 1:800,000 to 1:500,000 (e.g. 1:780,000 to 1:520,000, 1:760,000 to 1:540,000, 1:740,000 to 1:560,000 or 1:720,000 to 1:580,000, preferably 1:720,000 to 1:500,000, e.g. a lower end of the range may be at least 1:780,000, 1:760,000, 1:740,000 or 1:720,000, preferably 1:720,000 and/or an upper end of the range may be less than or equal to 1:520,000, 1:540,000, 1:560,000 or 1:580,000, preferably 1:500,000); or (ii) greater than 1:500,000 to 1:110,041 (preferably greater than 1:500,000 to 1:250,000, e.g. a lower end of the range may be at least 1:480,000, 1:460,000, 1:440,000 or 1:420,000 and/or an upper end of the range may be less than or equal to 1:120,000, 1:140,000, 1:160,000 or 1:180,000).

The composition (or kit) may comprise:
(a) vitamin A present at a weight ratio of vitamin A to protein of: (i) 1:47,826 to 1:44,000; (ii) 1:42,308 to 1:35,032; or (iii) 1:34,375 to 1:30,899;
(b) pyridoxine present at a weight ratio of pyridoxine to protein of: (i) 1:15,714 to 1:12,088; (ii) 1:8,980 to 1:5,930; or (iii) 1:4,835 to 1:4,365;
(c) folic acid present at a weight ratio of folic acid to protein of: (i) 1:183,333 to 1:157,143; (ii) 1:146,667 to 1:112,821; or (iii) 1:107,317 to 1:104,762;
(d) cyanocobalamin present at a weight ratio of cyanocobalamin to protein of: (i) 1:8,800,000 to 1:6,769,231; (ii) 1:5,028,571 to 1:3,320,755; or (iii) 1:2,707,692 to 1:2,444,444;
(e) ascorbic acid present at a weight ratio of ascorbic acid to protein of: (i) 1:289 to 1:262; (ii) 1:157 to 1:131; or (iii) 1:110 to 1:96;
(f) cholecalciferol present at a weight ratio of cholecalciferol to protein of: (i) 1:4,888,889 to 1:3,666,667; (ii) 1:2,200,000 to 1:1,795,918; or (iii) 1:1,446,667 to 1:1,333,333;
(g) vitamin E present at a weight ratio of vitamin E to protein of: (i) 1:2,933 to 1:2,716; (ii) 1:458 to 1:417; or (iii) 1:282 to 1:268;
(h) calcium present at a weight ratio of calcium to protein of: (i) 1:50 to 1:46; (ii) 1:44 to 1:39; or (iii) 1:38 to 1:37; and/or
(i) selenium present at a weight ratio of selenium to protein of: (i) 1:666,667 to 1:516,129; or (ii) 1:484,848 to 1:390,244.

The weight ratio to protein for each of the micronutrients above preferably correspond to base/normal (i.e. where the weight ratio to protein is preceded by a (i)), medium (i.e. where the weight ratio to protein is preceded by a (ii)), or high levels of the micronutrients (i.e. where the weight ratio to protein is preceded by a (iii)), or base/normal (i.e. where the weight ratio to protein is preceded by a (i)) or high levels (i.e. where the weight ratio to protein is preceded by a (ii)) in the context of selenium. Similarly, the weight ratio to protein for each of the micronutrients above preferably correspond to either base/normal (i.e. where the weight ratio to protein is preceded by a (i)), medium (i.e. where the weight ratio to protein is preceded by a (ii)), or high need of the micronutrients (i.e. where the weight ratio to protein is preceded by a (iii)), or base/normal (i.e. where the weight ratio to protein is preceded by a (i)) or high need (i.e. where the weight ratio to protein is preceded by a (ii)) in the context of selenium. Said need is preferably a need for the indicated weight ratio to protein of the micronutrient.

The amount of micronutrients present in nutritional compositions corresponding to each nutritional reference standard may be expressed as a weight ratio to protein. The embodiments above further defining the weight ratios of the micronutrients apply equally to each of (a) to (q) below.

The composition (or kit) may comprise:
(a) vitamin A present at a weight ratio of vitamin A to protein of greater than 1:43,137 to 1:34,700; pyridoxine present at a weight ratio of pyridoxine to protein of greater than 1:10,304 to 1:5,327; folic acid present at a weight ratio of folic acid to protein of greater than 1:220,000 to 1:151,724; cyanocobalamin present at a weight ratio of cyanocobalamin to protein of greater than 1:5,770,492 to 1:2,983,051; ascorbic acid present at a weight ratio of ascorbic acid to protein of greater than 1:200 to 1:120; cholecalciferol present at a weight ratio of cholecalciferol to protein of greater than 1:1,614,679 to 1:440,000; vitamin E present at a weight ratio of vitamin E to protein of greater than 1:789 to 1:336; calcium present at a weight ratio of calcium to protein of greater than 1:55 to 1:45; and/or (preferably and) selenium present at a weight ratio of selenium to protein of greater than 1:500,000 to 1:110,041; or
(b) vitamin A present at a weight ratio of vitamin A to protein of greater than 1:34,700 to 1:14,667; pyridoxine present at a weight ratio of pyridoxine to protein of greater than 1:10,304 to 1:5,327; folic acid present at a weight ratio of folic acid to protein of greater than 1:220,000 to 1:151,724; cyanocobalamin present at a weight ratio of cyanocobalamin to protein of greater than 1:5,770,492 to 1:2,983,051; ascorbic acid present at a weight ratio of ascorbic acid to protein of greater than 1:200 to 1:120; cholecalciferol present at a weight ratio of cholecalciferol to protein of greater than 1:2,750,000 to 1:1,614,679; vitamin E present at a weight ratio of vitamin E to protein of greater than 1:789 to 1:336; calcium present at a weight ratio of calcium to protein of greater than 1:55 to 1:45; and/or (preferably and) selenium present at a weight ratio of selenium to protein of greater than 1:800,000 to 1:500,000; or
(c) vitamin A present at a weight ratio of vitamin A to protein of greater than 1:43,137 to 1:34,700; pyridoxine present at a weight ratio of pyridoxine to protein of greater than 1:5,327 to 1:440; folic acid present at a weight ratio of folic acid to protein of greater than 1:110,000 to 1:44,000; cyanocobalamin present at a weight ratio of cyanocobalamin to protein of greater than 1:2,983,051 to 1:44,000; ascorbic acid present at a weight ratio of ascorbic acid to protein of greater than 1:200 to 1:120; cholecalciferol present at a weight ratio of cholecalciferol to protein of greater than 1:1,614,679 to 1:440,000; vitamin E present at a weight ratio of vitamin E to protein of greater than 1:789 to 1:336; calcium present at a weight ratio of calcium to protein of greater than 1:45 to 1:38; and/or (preferably and) selenium present at a weight ratio of selenium to protein of greater than 1:800,000 to 1:500,000; or
(d) vitamin A present at a weight ratio of vitamin A to protein of greater than 1:34,700 to 1:14,667; pyridoxine present at a weight ratio of pyridoxine to protein of greater than 1:10,304 to 1:5,327; folic acid present at a weight ratio of folic acid to protein of greater than 1:220,000 to 1:151,724; cyanocobalamin present at a weight ratio of cyanocobalamin to protein of greater than 1:5,770,492 to 1:2,983,051; ascorbic acid present at a weight ratio of ascorbic acid to protein of greater than 1:200 to 1:120; cholecalciferol present at a weight ratio of cholecalciferol to protein of greater than 1:1,614,679 to 1:440,000; vitamin E present at a weight ratio of vitamin E to protein of greater than 1:789 to 1:336; calcium present at a weight ratio of calcium to protein of greater than 1:38 to 1:18; and/or (preferably and) selenium present at a weight ratio of selenium to protein of greater than 1:800,000 to 1:500,000; or
(e) vitamin A present at a weight ratio of vitamin A to protein of greater than 1:34,700 to 1:14,667; pyridoxine present at a weight ratio of pyridoxine to protein of greater than 1:5,327 to 1:440; folic acid present at a weight ratio of folic acid to protein of greater than 1:220,000 to 1:151,724; cyanocobalamin present at a weight ratio of cyanocobalamin to protein of greater than 1:2,983,051 to 1:44,000; ascorbic acid present at a weight ratio of ascorbic acid to protein of greater than 1:120 to 1:22; cholecalciferol present at a weight ratio of cholecalciferol to protein of greater than 1:1,614,679 to 1:440,000; vitamin E present at a weight ratio of vitamin E to protein of greater than 1:336 to 1:40; calcium present at a weight ratio of calcium to protein of greater than 1:45 to 1:38; and/or (preferably and) selenium present at a weight ratio of selenium to protein of greater than 1:500,000 to 1:110,041; or
(f) vitamin A present at a weight ratio of vitamin A to protein of greater than 1:43,137 to 1:34,700; pyridoxine present at a weight ratio of pyridoxine to protein of greater than 1:5,327 to 1:440; folic acid present at a weight ratio of folic acid to protein of greater than 1:151,724 to 1:110,000; cyanocobalamin present at a weight ratio of cyanocobalamin to protein of greater than 1:2,983,051 to 1:44,000; ascorbic acid present at a weight ratio of ascorbic acid to protein of greater than 1:200 to 1:120; cholecalciferol present at a weight ratio of cholecalciferol to protein of greater than 1:8,800,000 to 1:2,750,000; vitamin E present at a weight ratio of vitamin E to protein of greater than 1:789 to 1:336; calcium present at a weight ratio of calcium to protein of greater than 1:55 to 1:45; and/or (preferably and) selenium present at a weight ratio of selenium to protein of greater than 1:500,000 to 1:110,041; or
(g) vitamin A present at a weight ratio of vitamin A to protein of greater than 1:43,137 to 1:34,700; pyridoxine present at a weight ratio of pyridoxine to protein of greater than 1:31,429 to 1:10,304; folic acid present at a weight ratio of folic acid to protein of greater than 1:151,724 to 1:110,000; cyanocobalamin present at a weight ratio of cyanocobalamin to protein of greater than 1:17,600,000 to 1:5,770,492; ascorbic acid present at a weight ratio of ascorbic acid to protein of greater than 1:120 to 1:22; cholecalciferol present at a weight ratio of cholecalciferol to protein of greater than 1:2,750,000 to 1:1,614,679; vitamin E present at a weight ratio of vitamin E to protein of greater than 1:336 to 1:40; calcium present at a weight ratio of calcium to protein of greater than 1:45 to 1:38; and/or (preferably and) selenium present at a weight ratio of selenium to protein of greater than 1:500,000 to 1:110,041; or
(h) vitamin A present at a weight ratio of vitamin A to protein of greater than 1:43,137 to 1:34,700; pyridoxine present at a weight ratio of pyridoxine to protein of greater than 1:10,304 to 1:5,327; folic acid present at a weight ratio of folic acid to protein of greater than 1:151,724 to 1:110,000; cyanocobalamin present at a weight ratio of cyanocobalamin to protein of greater than 1:5,770,492 to 1:2,983,051; ascorbic acid present at a weight ratio of ascorbic acid to protein of greater than 1:200 to 1:120; cholecalciferol present at a weight ratio of cholecalciferol to protein of greater than 1:8,800,000 to 1:2,750,000; vitamin E present at a weight ratio of vitamin E to protein of greater than 1:789 to 1:336; calcium present at a weight ratio of calcium to protein of greater than 1:45 to 1:38; and/or (preferably and) selenium present at a weight ratio of selenium to protein of greater than 1:500,000 to 1:110,041; or
(i) vitamin A present at a weight ratio of vitamin A to protein of greater than 1:43,137 to 1:34,700; pyridoxine present at a weight ratio of pyridoxine to protein of greater than 1:10,304 to 1:5,327; folic acid present at a weight ratio of folic acid to protein of greater than 1:151,724 to 1:110,000; cyanocobalamin present at a weight ratio of cyanocobalamin to protein of greater than 1:5,770,492 to 1:2,983,051; ascorbic acid present at a weight ratio of ascorbic acid to protein of greater than 1:550 to 1:200; cholecalciferol present at a weight ratio of cholecalciferol to protein of greater than 1:2,750,000 to 1:1,614,679; vitamin E present at a weight ratio of vitamin E to protein of greater than 1:3,667 to 1:789; calcium present at a weight ratio of calcium to protein of greater than 1:45 to 1:38; and/or (preferably and) selenium present at a weight ratio of selenium to protein of greater than 1:800,000 to 1:500,000; or
(j) vitamin A present at a weight ratio of vitamin A to protein of greater than 1:34,700 to 1:14,667; pyridoxine present at a weight ratio of pyridoxine to protein of greater than 1:10,304 to 1:5,327; folic acid present at a weight ratio of folic acid to protein of greater than 1:220,000 to 1:151,724; cyanocobalamin present at a weight ratio of cyanocobalamin to protein of greater than 1:5,770,492 to 1:2,983,051; ascorbic acid present at a weight ratio of ascorbic acid to protein of greater than 1:120 to 1:22; cholecalciferol present at a weight ratio of cholecalciferol to protein of greater than 1:2,750,000 to 1:1,614,679; vitamin E present at a weight ratio of vitamin E to protein of greater than 1:336 to 1:40; calcium present at a weight ratio of calcium to protein of greater than 1:45 to 1:38; and/or (preferably and) selenium present at a weight ratio of selenium to protein of greater than 1:500,000 to 1:110,041; or
(k) vitamin A present at a weight ratio of vitamin A to protein of greater than 1:34,700 to 1:14,667; pyridoxine present at a weight ratio of pyridoxine to protein of greater than 1:10,304 to 1:5,327; folic acid present at a weight ratio of folic acid to protein of greater than 1:151,724 to 1:110,000; cyanocobalamin present at a weight ratio of cyanocobalamin to protein of greater than 1:5,770,492 to 1:2,983,051; ascorbic acid present at a weight ratio of ascorbic acid to protein of greater than 1:120 to 1:22; cholecalciferol present at a weight ratio of cholecalciferol to protein of greater than 1:2,750,000 to 1:1,614,679; vitamin E present at a weight ratio of vitamin E to protein of greater than 1:336 to 1:40; calcium present at a weight ratio of calcium to protein of greater than 1:45 to 1:38; and/or (preferably and) selenium present at a weight ratio of selenium to protein of greater than 1:800,000 to 1:500,000; or
(l) vitamin A present at a weight ratio of vitamin A to protein of greater than 1:43,137 to 1:34,700; pyridoxine present at a weight ratio of pyridoxine to protein of greater than 1:10,304 to 1:5,327; folic acid present at a weight ratio of folic acid to protein of greater than 1:151,724 to 1:110,000; cyanocobalamin present at a weight ratio of cyanocobalamin to protein greater than 1:5,770,492 to 1:2,983,051; ascorbic acid present at a weight ratio of ascorbic acid to protein of greater than 1:120 to 1:22; cholecalciferol present at a weight ratio of cholecalciferol to protein of greater than 1:8,800,000 to 1:2,750,000; vitamin E present at a weight ratio of vitamin E to protein of greater than 1:336 to 1:40; calcium present at a weight ratio of calcium to protein of greater than 1:55 to 1:45; and/or (preferably and) selenium present at a weight ratio of selenium to protein of greater than 1:500,000 to 1:110,041; or
(m) vitamin A present at a weight ratio of vitamin A to protein of greater than 1:34,700 to 1:14,667; pyridoxine present at a weight ratio of pyridoxine to protein of greater than 1:31,429 to 1:10,304; folic acid present at a weight ratio of folic acid to protein of greater than 1:110,000 to 1:44,000; cyanocobalamin present at a weight ratio of cyanocobalamin to protein of greater than 1:17,600,000 to 1:5,770,492; ascorbic acid present at a weight ratio of ascorbic acid to protein of greater than 1:200 to 1:120; cholecalciferol present at a weight ratio of cholecalciferol to protein of greater than 1:2,750,000 to 1:1,614,679; vitamin E present at a weight ratio of vitamin E to protein of greater than 1:789 to 1:336; calcium present at a weight ratio of calcium to protein of greater than 1:45 to 1:38; and/or (preferably and) selenium present at a weight ratio of selenium to protein of greater than 1:800,000 to 1:500,000; or
(n) vitamin A present at a weight ratio of vitamin A to protein of greater than 1:43,137 to 1:34,700; pyridoxine present at a weight ratio of pyridoxine to protein of greater than 1:5,327 to 1:440; folic acid present at a weight ratio of folic acid to protein of greater than 1:151,724 to 1:110,000; cyanocobalamin present at a weight ratio of cyanocobalamin to protein greater than 1:2,983,051 to 1:44,000; ascorbic acid present at a weight ratio of ascorbic acid to protein of greater than 1:120 to 1:22; cholecalciferol present at a weight ratio of cholecalciferol to protein of greater than 1:2,750,000 to 1:1,614,679; vitamin E present at a weight ratio of vitamin E to protein of greater than 1:336 to 1:40; calcium present at a weight ratio of calcium to protein of greater than 1:45 to 1:38; and/or (preferably and) selenium present at a weight ratio of selenium to protein of greater than 1:500,000 to 1:110,041; or
(o) vitamin A present at a weight ratio of vitamin A to protein of greater than 1:34,700 to 1:14,667; pyridoxine present at a weight ratio of pyridoxine to protein of greater than 1:31,429 to 1:10,304; folic acid present at a weight ratio of folic acid to protein of greater than 1:151,724 to 1:110,000; cyanocobalamin present at a weight ratio of cyanocobalamin to protein of greater than 1:17,600,000 to 1:5,770,492; ascorbic acid present at a weight ratio of ascorbic acid to protein of greater than 1:120 to 1:22; cholecalciferol present at a weight ratio of cholecalciferol to protein of greater than 1:8,800,000 to 1:2,750,000; vitamin E present at a weight ratio of vitamin E to protein of greater than 1:336 to 1:40; calcium present at a weight ratio of calcium to protein of greater than 1:55 to 1:45; and/or (preferably and) selenium present at a weight ratio of selenium to protein of greater than 1:800,000 to 1:500,000; or
(p) vitamin A present at a weight ratio of vitamin A to protein of greater than 1:43,137 to 1:34,700; pyridoxine present at a weight ratio of pyridoxine to protein of greater than 1:31,429 to 1:10,304; folic acid present at a weight ratio of folic acid to protein of greater than 1:151,724 to 1:110,000; cyanocobalamin present at a weight ratio of cyanocobalamin to protein of greater than 1:17,600,000 to 1:5,770,492; ascorbic acid present at a weight ratio of ascorbic acid to protein of greater than 1:200 to 1:120; cholecalciferol present at a weight ratio of cholecalciferol to protein of greater than 1:8,800,000 to 1:2,750,000; vitamin E present at a weight ratio of vitamin E to protein of greater than 1:789 to 1:336; calcium present at a weight ratio of calcium to protein of greater than 1:55 to 1:45; and/or (preferably and) selenium present at a weight ratio of selenium to protein of greater than 1:500,000 to 1:110,041; or
(q) vitamin A present at a weight ratio of vitamin A to protein of greater than 1:43,137 to 1:34,700; pyridoxine present at a weight ratio of pyridoxine to protein of greater than 1:10,304 to 1:5,327; folic acid present at a weight ratio of folic acid to protein of greater than 1:151,724 to 1:110,000; cyanocobalamin present at a weight ratio of cyanocobalamin to protein of greater than 1:5,770,492 to 1:2,983,051; ascorbic acid present at a weight ratio of ascorbic acid to protein of greater than 1:120 to 1:22; cholecalciferol present at a weight ratio of cholecalciferol to protein of greater than 1:1,614,679 to 1:440,000; vitamin E present at a weight ratio of vitamin E to protein of greater than 1:336 to 1:40; calcium present at a weight ratio of calcium to protein of greater than 1:38 to 1:18; and/or (preferably and) selenium present at a weight ratio of selenium to protein of greater than 1:800,000 to 1:500,000.

The composition (or kit) may comprise:
(a) vitamin A present at a weight ratio of vitamin A to protein of 1:42,308 to 1:35,032; pyridoxine present at a weight ratio of pyridoxine to protein of 1:8,980 to 1:5,930; folic acid present at a weight ratio of folic acid to protein of 1:183,333 to 1:157, 143; cyanocobalamin present at a weight ratio of cyanocobalamin to protein of 1:5,028,571 to 1:3,320,755; ascorbic acid present at a weight ratio of ascorbic acid to protein of 1:157 to 1:131; cholecalciferol present at a weight ratio of cholecalciferol to protein of 1:1,446,667 to 1:1,333,333; vitamin E present at a weight ratio of vitamin E to protein of 1:458 to 1:417; calcium present at a weight ratio of calcium to protein of: 1:50 to 1:46; and/or (preferably and) selenium present at a weight ratio of selenium to protein 1:484,848 to 1:390,244; or
(b) vitamin A present at a weight ratio of vitamin A to protein of 1:34,375 to 1:30,899; pyridoxine present at a weight ratio of pyridoxine to protein of 1:8,980 to 1:5,930; folic acid present at a weight ratio of folic acid to protein of 1:183,333 to 1:157,143; cyanocobalamin present at a weight ratio of cyanocobalamin to protein of 1:5,028,571 to 1:3,320,755; ascorbic acid present at a weight ratio of ascorbic acid to protein of 1:157 to 1:131; cholecalciferol present at a weight ratio of cholecalciferol to protein of 1:2,200,000 to 1:1,795,918; vitamin E present at a weight ratio of vitamin E to protein of 1:458 to 1:417; calcium present at a weight ratio of calcium to protein of 1:50 to 1:46; and/or (preferably and) selenium present at a weight ratio of selenium to protein of 1:666,667 to 1:516,129; or
(c) vitamin A present at a weight ratio of vitamin A to protein of 1:42,308 to 1:35,032; pyridoxine present at a weight ratio of pyridoxine to protein of 1:4,835 to 1:4,365; folic acid present at a weight ratio of folic acid to protein of 1:107,317 to 1:104,762; cyanocobalamin present at a weight ratio of cyanocobalamin to protein of 1:2,707,692 to 1:2,444,444; ascorbic acid present at a weight ratio of ascorbic acid to protein of 1:157 to 1:131; cholecalciferol present at a weight ratio of cholecalciferol to protein of 1:1,446,667 to 1:1,333,333; vitamin E present at a weight ratio of vitamin E to protein of 1:458 to 1:417; calcium present at a weight ratio of calcium to protein of 1:44 to 1:39; and/or (preferably and) selenium present at a weight ratio of selenium to protein of 1:666,667 to 1:516, 129; or
(d) vitamin A present at a weight ratio of vitamin A to protein of 1:34,375 to 1:30,899; pyridoxine present at a weight ratio of pyridoxine to protein of 1:8,980 to 1:5,930; folic acid present at a weight ratio of folic acid to protein of 1:183,333 to 1:157,143; cyanocobalamin present at a weight ratio of cyanocobalamin to protein of 1:5,028,571 to 1:3,320,755; ascorbic acid present at a weight ratio of ascorbic acid to protein of 1:157 to 1:131; cholecalciferol present at a weight ratio of cholecalciferol to protein of 1:1,446,667 to 1:1,333,333; vitamin E present at a weight ratio of vitamin E to protein of 1:458 to 1:417; calcium present at a weight ratio of calcium to protein of 1:38 to 1:37; and/or (preferably and) selenium present at a weight ratio of selenium to protein of 1:666,667 to 1:516,129; or
(e) vitamin A present at a weight ratio of vitamin A to protein of 1:34,375 to 1:30,899; pyridoxine present at a weight ratio of pyridoxine to protein of 1:4,835 to 1:4,365; folic acid present at a weight ratio of folic acid to protein of 1:183,333 to 1:157,143; cyanocobalamin present at a weight ratio of cyanocobalamin to protein of 1:2,707,692 to 1:2,444,444; ascorbic acid present at a weight ratio of ascorbic acid to protein of 1:110 to 1:96; cholecalciferol present at a weight ratio of cholecalciferol to protein of 1:1,446,667 to 1:1,333,333; vitamin E present at a weight ratio of vitamin E to protein of 1:282 to 1:268; calcium present at a weight ratio of calcium to protein of 1:44 to 1:39; and/or (preferably and) selenium present at a weight ratio of selenium to protein of 1:484,848 to 1:390,244; or
(f) vitamin A present at a weight ratio of vitamin A to protein of 1:42,308 to 1:35,032; pyridoxine present at a weight ratio of pyridoxine to protein of 1:4,835 to 1:4,365; folic acid present at a weight ratio of folic acid to protein of 1:146,667 to 1:112,821; cyanocobalamin present at a weight ratio of cyanocobalamin to protein of 1:2,707,692 to 1:2,444,444; ascorbic acid present at a weight ratio of ascorbic acid to protein of 1:157 to 1:131; cholecalciferol present at a weight ratio of cholecalciferol to protein of 1:4,888,889 to 1:3,666,667; vitamin E present at a weight ratio of vitamin E to protein of 1:458 to 1:417; calcium present at a weight ratio of calcium to protein of 1:50 to 1:46; and/or (preferably and) selenium present at a weight ratio of selenium to protein of 1:484,848 to 1:390,244; or
(g) vitamin A present at a weight ratio of vitamin A to protein of 1:42,308 to 1:35,032; pyridoxine present at a weight ratio of pyridoxine to protein of 1:15,714 to 1:12,088; folic acid present at a weight ratio of folic acid to protein of 1:146,667 to 1:112,821; cyanocobalamin present at a weight ratio of cyanocobalamin to protein of 1:8,800,000 to 1:6,769,231; ascorbic acid present at a weight ratio of ascorbic acid to protein 1:110 to 1:96; cholecalciferol present at a weight ratio of cholecalciferol to protein of 1:2,200,000 to 1:1,795,918; vitamin E present calcium to protein of 1:44 to 1:39; and/or (preferably and) selenium present at a weight ratio of selenium to protein of 1:484,848 to 1:390,244; or
(h) vitamin A present at a weight ratio of vitamin A to protein of 1:42,308 to 1:35,032; pyridoxine present at a weight ratio of pyridoxine to protein of 1:8,980 to 1:5,930; folic acid present at a weight ratio of folic acid to protein of 1:146,667 to 1:112,821; cyanocobalamin present at a weight ratio of cyanocobalamin to protein of 1:5,028,571 to 1:3,320,755; ascorbic acid present at a weight ratio of ascorbic acid to protein of 1:157 to 1:131; cholecalciferol present at a weight ratio of cholecalciferol to protein of 1:4,888,889 to 1:3,666,667; vitamin E present at a weight ratio of vitamin E to protein of 1:458 to 1:417; calcium present at a weight ratio of calcium to protein of 1:44 to 1:39; and/or (preferably and) selenium present at a weight ratio of selenium to protein of 1:484,848 to 1:390,244; or
(i) vitamin A present at a weight ratio of vitamin A to protein of 1:42,308 to 1:35,032; pyridoxine present at a weight ratio of pyridoxine to protein of 1:8,980 to 1:5,930; folic acid present at a weight ratio of folic acid to protein 1:146,667 to 1:112,821; cyanocobalamin present at a weight ratio of cyanocobalamin to protein of 1:5,028,571 to 1:3,320,755; ascorbic acid present at a weight ratio of ascorbic acid to protein of 1:289 to 1:262; cholecalciferol present at a weight ratio of cholecalciferol to protein of 1:2,200,000 to 1:1,795,918; vitamin E present at a weight ratio of vitamin E to protein of 1:2,933 to 1:2,716; calcium present at a weight ratio of calcium to protein of 1:44 to 1:39; and/or (preferably and) selenium present at a weight ratio of selenium to protein of 1:666,667 to 1:516, 129; or
(j) vitamin A present at a weight ratio of vitamin A to protein of 1:34,375 to 1:30,899; pyridoxine present at a weight ratio of pyridoxine to protein of 1:8,980 to 1:5,930; folic acid present at a weight ratio of folic acid to protein of 1:183,333 to 1:157,143; cyanocobalamin present at a weight ratio of cyanocobalamin to protein of 1:5,028,571 to 1:3,320,755; ascorbic acid present at a weight ratio of ascorbic acid to protein of 1:110 to 1:96; cholecalciferol present at a weight ratio of cholecalciferol to protein of 1:2,200,000 to 1:1,795,918; vitamin E present at a weight ratio of vitamin E to protein of 1:282 to 1:268; calcium present at a weight ratio of calcium to protein of 1:44 to 1:39; and/or (preferably and) selenium present at a weight ratio of selenium to protein of 1:484,848 to 1:390,244; or
(k) vitamin A present at a weight ratio of vitamin A to protein of 1:34,375 to 1:30,899; pyridoxine present at a weight ratio of pyridoxine to protein of 1:8,980 to 1:5,930; folic acid present at a weight ratio of folic acid to protein of 1:146,667 to 1:112,821; cyanocobalamin present at a weight ratio of cyanocobalamin to protein of 1:5,028,571 to 1:3,320,755; ascorbic acid present at a weight ratio of ascorbic acid to protein of 1:110 to 1:96; cholecalciferol present at a weight ratio of cholecalciferol to protein of 1:2,200,000 to 1:1,795,918; vitamin E present calcium to protein of 1:44 to 1:39; and/or (preferably and) selenium present at a weight ratio of selenium to protein of 1:666,667 to 1:516, 129; or
(l) vitamin A present at a weight ratio of vitamin A to protein of 1:42,308 to 1:35,032; pyridoxine present at a weight ratio of pyridoxine to protein of 1:8,980 to 1:5,930; folic acid present at a weight ratio of folic acid to protein of 1:146,667 to 1:112,821; cyanocobalamin present at a weight ratio of cyanocobalamin to protein of 1:5,028,571 to 1:3,320,755; ascorbic acid present at a weight ratio of ascorbic acid to protein of 1:110 to 1:96; cholecalciferol present at a weight ratio of cholecalciferol to protein of 1:4,888,889 to 1:3,666,667; vitamin E present at a weight ratio of vitamin E to protein of 1:282 to 1:268; calcium present at a weight ratio of calcium to protein of 1:50 to 1:46; and/or (preferably and) selenium present at a weight ratio of selenium to protein of 1:484,848 to 1:390,244; or
(m) vitamin A present at a weight ratio of vitamin A to protein of 1:34,375 to 1:30,899; pyridoxine present at a weight ratio of pyridoxine to protein of 1:15,714 to 1:12,088; folic acid present at a weight ratio of folic acid to protein of 1:107,317 to 1:104,762; cyanocobalamin present at a weight ratio of cyanocobalamin to protein of 1:8,800,000 to 1:6,769,231; ascorbic acid present at a weight ratio of ascorbic acid to protein of 1:157 to 1:131; cholecalciferol present at a weight ratio of cholecalciferol to protein of 1:2,200,000 to 1:1,795,918; or vitamin E present at a weight ratio of vitamin E to protein of 1:458 to 1:417; calcium present at a weight ratio of calcium to protein of 1:44 to 1:39; and/or (preferably and) selenium present at a weight ratio of selenium to protein of 1:666,667 to 1:516, 129; or
(n) vitamin A present at a weight ratio of vitamin A to protein 1:42,308 to 1:35,032; pyridoxine present at a weight ratio of pyridoxine to protein of 1:4,835 to 1:4,365; folic acid present at a weight ratio of folic acid to protein of 1:146,667 to 1:112,821; cyanocobalamin present at a weight ratio of cyanocobalamin to protein of 1:2,707,692 to 1:2,444,444; ascorbic acid present at a weight ratio of ascorbic acid to protein of 1:110 to 1:96; cholecalciferol present at a weight ratio of cholecalciferol to protein of 1:2,200,000 to 1:1,795,918; vitamin E present at a weight ratio of vitamin E to protein of 1:282 to 1:268; calcium present at a weight ratio of calcium to protein 1:44 to 1:39; and/or (preferably and) selenium present at a weight ratio of selenium to protein of 1:484,848 to 1:390,244; or
(o) vitamin A present at a weight ratio of vitamin A to protein of 1:34,375 to 1:30,899; pyridoxine present at a weight ratio of pyridoxine to protein of 1:15,714 to 1:12,088; folic acid present at a weight ratio of folic acid to protein of 1:146,667 to 1:112,821; cyanocobalamin present at a weight ratio of cyanocobalamin to protein of 1:8,800,000 to 1:6,769,231; ascorbic acid present at a weight ratio of ascorbic acid to protein of 1:110 to 1:96; cholecalciferol present at a weight ratio of cholecalciferol to protein of 1:4,888,889 to 1:3,666,667; vitamin E present calcium to protein of 1:50 to 1:46; and/or (preferably and) selenium present at a weight ratio of selenium to protein of 1:666,667 to 1:516,129; or
(p) vitamin A present at a weight ratio of vitamin A to protein of 1:42,308 to 1:35,032; pyridoxine present at a weight ratio of pyridoxine to protein of 1:15,714 to 1:12,088; folic acid present at a weight ratio of folic acid to protein of 1:146,667 to 1:112,821; cyanocobalamin present at a weight ratio of cyanocobalamin to protein of 1:8,800,000 to 1:6,769,231; ascorbic acid present at a weight ratio of ascorbic acid to protein of 1:157 to 1:131; cholecalciferol present at a weight ratio of cholecalciferol to protein of 1:4,888,889 to 1:3,666,667; vitamin E present at a weight ratio of vitamin E to protein of 1:458 to 1:417; calcium present at a weight ratio of calcium to protein of 1:50 to 1:46; and/or (preferably and) selenium present at a weight ratio of selenium to protein of 1:484,848 to 1:390,244; or
(q) vitamin A present at a weight ratio of vitamin A to protein of 1:42,308 to 1:35,032; pyridoxine present at a weight ratio of pyridoxine to protein of 1:8,980 to 1:5,930; folic acid present at a weight ratio of folic acid to protein of 1:146,667 to 1:112,821; cyanocobalamin present at a weight ratio of cyanocobalamin to protein of 1:5,028,571 to 1:3,320,755; ascorbic acid present at a weight ratio of ascorbic acid to protein of 1:110 to 1:96; cholecalciferol present at a weight ratio of cholecalciferol to protein of 1:1,446,667 to 1:1,333,333; vitamin E present at a weight ratio of vitamin E to protein of 1:282 to 1:268; calcium present at a weight ratio of calcium to protein of 1:38 to 1:37; and/or (preferably and) selenium present at a weight ratio of selenium to protein of 1:666,667 to 1:516,129.

In some embodiments, a composition of the invention may comprise at least one additional component. The additional component may be a stimulant, an additional macronutrient or an additional micronutrient. In one embodiment, the composition further comprises caffeine. In some embodiments, the composition comprises sodium (e.g. sodium chloride). The additional component may include gluten and/or lactose. In some embodiments, the composition further comprises at least one additive selected from: flavourings; sweeteners; emulsifying and/or stabilizing agents; binding agents; acidity regulators; and/or colourants.

In one embodiment, the composition of the invention may further comprise natural sweeteners. In one embodiment a natural sweetener may comprise steviol, erythritol, xylitol, yacon syrup and/or monk fruit sweetener, preferably steviol, more preferably steviol glycoside. These components can improve the organoleptic properties (or sensory properties, such as texture, taste, smell, sight, and/or touch) of the composition, as well the stability and shelf life of the product. The skilled person will be able to identify other constituents that are well-known in, and compatible with, nutritional compositions in general and, in particular, and will be able to determine the correct quantity for use with the embodiments of the invention.

In some embodiments, the composition provides a subject with its nutritional requirements as determined by the subject's genetic profile and/or a method of the present invention. Thus, in one embodiment one or more micronutrients and/or macronutrients are present in a nutritional composition of the invention in an amount to satisfy a subject's daily nutritional requirements as determined by the subject's genetic profile.

In some embodiments, the composition is in a form ready for consumption. For example, a dry composition of the invention may be directly consumed by a subject without any rehydration or processing of the composition.

In some embodiments, the composition may be in the form of a powder, a tablet, a bar, a confectionary product, or a granule and intended for use as a solid oral dosage form.

In one aspect of the present invention, there is provided a method for manufacturing a composition of the invention, the method comprising:
(a) admixing any of the ingredients described herein, optionally at an amount as described herein; and
(b) optionally packaging

In one aspect, the invention provides a method for manufacturing a nutritional composition, the method comprising:
(a) admixing at least two micronutrients selected from vitamin A, pyridoxine (vitamin B6), folic acid (vitamin B9), cyanocobalamin (vitamin B12), ascorbic acid (vitamin C), cholecalciferol (vitamin D3), vitamin E, calcium, and/or selenium; or
(b) admixing at least one micronutrient selected from vitamin A, pyridoxine (vitamin B6), folic acid (vitamin B9), cyanocobalamin (vitamin B12), ascorbic acid (vitamin C), cholecalciferol (vitamin D3), vitamin E, calcium, and/or selenium with a macronutrient; and
(c) optionally packaging.

The invention also provides a nutritional composition obtainable by a method of the invention.

The term “obtainable” as used herein also encompasses the term “obtained”. In one embodiment the term “obtainable” means obtained.

In a one aspect of the invention, the composition is a wet composition (e.g. a hydrated composition). Said wet composition may have been prepared from a dry composition described herein. Thus, in one aspect, the invention provides a method for preparing a wet/ready-to-eat composition, the method comprising adding an aqueous solution (preferably water or a formulation comprising water) to a composition (preferably a dry composition) of the invention.

The invention also provides a wet/ready-to-eat composition obtainable by a method of the invention.

A composition of the invention may be hydrated by any means known to the person skilled in the art, for example by addition of an aqueous solution, e.g. water or an alternative formulation comprising water, such as milk. A wet composition may have a water content of at least 5 wt. % In one embodiment a wet composition may have a water content of at least 10, 15, 20, 25, 30, 35, 40, 50, 60 or 70 wt. %. Preferably, a wet composition may have a water content of at least 80 wt. %.

A nutritional composition of the present invention may be a meal replacement composition. In other words, in some embodiments, said composition may be intended to be the sole source of nutrition for a subject. This may be the case where the composition comprises both micronutrients and macronutrients.

In one aspect, the invention provides a method for determining a subject's nutritional requirements, the method comprising:
(a) providing a subject's genetic profile, wherein the subject's genetic profile comprises a sequence of at least a region of a gene (preferably the subject's genetic profile comprises a sequence of at least a region of all copies (e.g. both copies [maternal and paternal copies]) of the gene);
(b) comparing the subject's genetic profile with a plurality of genetic reference standards, wherein each of the genetic reference standards is associated with a requirement level of a nutrient (preferably each genetic reference standard corresponds to (or comprises sequence information relating to) at least a region of all copies (e.g. both copies [maternal and paternal copies]) of the gene); and
(c) determining which of the plurality of genetic reference standards is most similar to the subject's genetic profile, thereby identifying a level of the nutrient required by the subject.

The genetic reference standards of part (b) are preferably all associated with a requirement level of the same nutrient.

A gene may affect a subject's ability to utilise a nutrient. A gene may be a gene associated with nutrient uptake and/or metabolism. The term “a gene may be a gene associated with nutrient uptake and/or metabolism” may encompass a gene that is either directly or indirectly involved in nutrient uptake and/or metabolism. In one embodiment, the gene is one that when a mutation is present (e.g. a SNP described herein) a subject's need for a nutrient and/or sensitivity to a nutrient is impacted. Preferably, a subject's need for a nutrient is increased (e.g. the subject less efficiently metabolises said nutrient and/or the subject's uptake/absorption from diet is poorer). Suitable genes may include ACE, PPARG, TCF7L2, ADRB2, ADRB3, FTO, APOC3, LPL, APOA5, CYP1A2, SOD2, CAT, GPX1, MTHFR, SLC19A1, TCN2, VDR, MCM6, HLA DQA1, and/or BCO1.

As subject's nutritional requirements may be a need for a base/normal daily amount of a nutrient (e.g. an amount that is similar to a nutritional reference value, such as the EU NRV or any other value described herein) or an elevated daily amount of a nutrient. An elevated daily amount may be a medium elevated or a highly elevated amount of the nutrient when compared to a daily nutritional reference value for a nutrient (e.g. the EU NRV or any other value described herein). Suitable base/normal, medium, and high amounts of a nutrient are described herein, for example at Tables 9-14, which define a base, medium, or high daily dose as a % of a nutritional reference value for each archetype/nutritional reference standard, Table 15, which summarises a suitable base, medium, or high daily dose as a % of a nutritional reference value, Table 16, which summarises a base, medium, or high daily dose as a % of a nutritional reference value as well as an amount of the one or more further micronutrients, Tables 17-33, which summarise for each archetype/nutritional reference standard a base, medium, or high daily dose as a % of a nutritional reference value as well as the daily amount thereof, and/or claim 7 or 8 herein). A subject's nutritional requirement may also be a sensitivity for a nutrient, e.g. a carbohydrate, in which case an increased sensitivity when compared to a baseline/normal sensitivity preferably indicates that the subject should have a lower amount (or none) of the nutrient per day.

A medium need may be a need that is higher than a base need. A high need may be a need that is higher than a base and a medium need.

A low sensitivity may be a sensitivity that is higher than a base sensitivity. A medium sensitivity may be a sensitivity that is higher than a base sensitivity. A high sensitivity may be a sensitivity that is higher than a base sensitivity and a medium sensitivity.

In one embodiment, a normal/base sensitivity may mean that a subject is not sensitive to a nutrient (e.g. lactose and/or gluten).

The terms “normal” and “base” may be used interchangeably herein.

As mentioned above, preferably, the base/normal, medium, and high needs for a micronutrient correspond to the daily unit dose, wt. %, and/or weight ratio to protein (i.e. where base/normal, medium, or high are preceded by a (i), (ii), or (iii), respectively, or for selenium where base/normal is preceded by a (i) and high is preceded by a (ii).

The term “most similar” as used herein preferably means “the same”.

A subject's genetic profile comprises a sequence of at least a region of a gene, preferably a sequence of all copies (e.g. both copies [maternal and paternal copies]) of the gene. The term “gene” as used herein may encompass translated regions, untranslated regions, promoter regions, and other regions either upstream or downstream thereof. Preferably, the term “gene” encompasses an open reading frame of the gene only. In one embodiment the subject's genetic sequence may comprise a sequence of a whole gene. However, it is preferred that the sequence is a sequence of a portion of a gene corresponding to a region with known polymorphisms, e.g. wherein each polymorphism is associated with a requirement level of a nutrient.

A “genetic reference standard” as used herein refers to known sequence information for at least a region of a gene (preferably a single genetic reference standard comprises known sequence information for at least a region of all copies (e.g. both copies [maternal and paternal copies]) of a gene). The term “genetic reference standard” as used herein encompasses sequence information recorded in a database. Said sequence information may have been obtained from a subject with a known nutrient sensitivity level and/or nutrient need level. The “genetic reference standard” may refer to sequence information obtained prior to carrying out a method of the invention. Exemplary “genetic reference standards” are presented in Table 2 at Example 1 herein. For example, for the gene BCO1, a “genetic reference standard” may correspond to the first row indicating that in some subjects there is a polymorphism (where the subject has a “T” in both copies [maternal and paternal copies] of the gene) at a region defined by SNP accession no. rs12934922 and that this is strongly associated with vitamin A need. In other words, a subject having 2 Ts (one in each gene) in the gene BCO1 at the position defined by rs12934922 requires a higher level of vitamin A in its diet. Each row for each gene may thus correspond to an independent “genetic reference standard”. Comparing a genetic profile of a subject with a genetic reference standard may be carried out using techniques known to the person skilled in the art, e.g. sequence alignment.

In one embodiment, the method of the invention may comprise the use of at least 1, 2, 3, 4, 5, 6, 7, 8, 9, 10, 11, 12, 13, 14, or 15 genetic reference standards. In one embodiment, the method of the invention may comprise the use of less than or equal to 100, 75, 50, 25, 24, 23, 22, 21, 20, 19, 18, 17, 16 or 15 genetic reference standards. In one embodiment, the method of the invention may comprise the use of 1-100, 1-70, 1-50, 1-25, 2-24, 4-22, 6-20, 8-18, 6-16, 8-14 or 10-12 genetic reference standards.

A genetic reference standard to which a subject's genetic profile is most similar may be any one or more of the genetic reference standards presented in Table 2, wherein each SNP (or insertion/deletion profile for ACE) corresponds to a reference standard. Each SNP profile (or insertion/deletion profile for ACE) is correlated with a sensitivity level for a nutrient or a level of need for a nutrient (e.g. vitamin A need). Thus, in one embodiment a genetic reference standard corresponds to a SNP profile for a given gene presented in Table 2. Thus, in one embodiment a genetic reference standard corresponds to an insertion/deletion profile for ACE presented in Table 2. In one embodiment a subject's genetic profile is most similar to a SNP profile for a given gene presented in Table 2. In one embodiment a subject's genetic profile is most similar to an insertion/deletion profile for ACE presented in Table 2.

The term “at least a region of a gene” encompasses a sequence of a single nucleotide of known position within a chromosome and/or gene. In one embodiment, the term “at least a region of a gene” means a region of at least 2, 5, 10, 50, 100, 500, 1000 or 5000 nucleotides. For example, 1-50, 1-100 or 1-1000 nucleotides. Preferably, at least a region of a gene corresponds to a region of a gene surrounding a SNP, e.g. comprising at least 5 (e.g. at least 10, 15, 20, or 50) nucleotides upstream of the SNP and 5 (e.g. at least 10, 15, 20, or 50) nucleotides downstream of the SNP.

A genetic profile may be obtainable from a sample obtainable from the subject. Typically, the sample comprises genetic material, for example DNA. In some embodiments, the sample is a cell sample (for example, a cheek swab), a saliva sample, a blood sample, or a tissue sample. The sample may be obtained by the subject itself. Having the subject obtain the sample enables the subject to obtain the sample at a time and location convenient to them, and allows for home testing and kits for home testing. The sample may have been subjected to one or more processing steps prior to use in a method of the invention.

A subject's genetic profile or a genetic reference standard herein may comprise a nucleotide sequence having at least 20%, 30%, 40%, 50%, 60%, 70%, 80%, 90%, or 95% (preferably 100%) sequence identity to a fragment of at least 5, 10, 20, 30 or 40 nucleotides of a nucleotide sequence (or information regarding a nucleotide sequence) selected from SEQ ID NOs: 4, 8, 12, 16, 20, 24, 28, 32, 36, 40, 44, 48, 52, 56, 60, 64, 68, 72, 76, 80, 84, 88, 92, 98, or 99. A subject's genetic profile or a genetic reference standard herein may comprise a nucleotide sequence having at least 20%, 30%, 40%, 50%, 60%, 70%, 80%, 90%, or 95% (preferably 100%) sequence identity to a fragment of at least 5, 10, 20, 30 or 40 nucleotides of a nucleotide sequence (or information regarding a nucleotide sequence) selected from SEQ ID NO: 100, wherein X=a nucleotide sequence having at least 20%, 30%, 40%, 50%, 60%, 70%, 80%, 90%, or 95% (preferably 100%) sequence identity to SEQ ID NO: 101 or SEQ ID NO: 102. Said fragment preferably encompasses any SNP and/or insertion and/or deletion comprised in said sequence.

A subject's genetic profile or a genetic reference standard herein may comprise a nucleotide sequence (or information regarding a nucleotide sequence) having at least 20%, 30%, 40%, 50%, 60%, 70%, 80%, 90%, or 95% (preferably 100%) sequence identity to any one of SEQ ID NOs: 4, 8, 12, 16, 20, 24, 28, 32, 36, 40, 44, 48, 52, 56, 60, 64, 68, 72, 76, 80, 84, 88, 92, 98, or 99. A subject's genetic profile or a genetic reference standard herein may comprise a nucleotide sequence (or information regarding a nucleotide sequence) having at least 20%, 30%, 40%, 50%, 60%, 70%, 80%, 90%, or 95% (preferably 100%) sequence identity to SEQ ID NO: 100, wherein X=a nucleotide sequence having at least 20%, 30%, 40%, 50%, 60%, 70%, 80%, 90%, or 95% (preferably 100%) sequence identity to SEQ ID NO: 101 or SEQ ID NO: 102.

A subject's genetic profile may comprise at least a region of at least 1, 2, 3, 4, 5, 6, 7, 8, 9, 10, 11, 12, 13, 14, or 15 genes. In one embodiment, the subject's genetic profile may comprise at least a region of less than or equal to 50, 40, 30, 25, 24, 23, 22, 21, 20, 19, 18, 17, 16 or 15 genes. In one embodiment, the subject's genetic profile may comprise at least a region of 1-50, 1-40, 1-30, 1-25, 2-24, 4-22, 6-20, 8-18, 6-16, 8-14 or 10-12 genes.

A variety of means for obtaining a sequence of at least a region of a gene are known to those skilled in the art. By way of non-limiting example, suitable methods include DNA (e.g. genomic) sequencing, real-time quantitative PCR (RT-qPCR), DNA/RNA microarray, RNA-Seq (also known as whole transcriptome shotgun sequencing), northern blotting, and/or serial analysis of gene expression (SAGE). In other words, although DNA sequencing is preferred, it is not intended that the present invention is limited to DNA sequencing. For example, a sequence of at least a region of a gene may be obtained by other methods, e.g. sequencing RNA or determining the amino acid residues in a protein (e.g. using proteomics). Such information can be used to determine the sequence of at least a region of a gene. A particularly preferred method is Kompetitive Allele Specific PCT (KASP) as described in Example 1. A technique for sequencing at least a region of a gene (e.g. KASP) may employ one or more nucleotide (e.g. primer) sequences described herein or a nucleotide sequence having at least 90% (preferably at least 95% or more preferably 100%) sequence identity thereto. A technique for sequencing at least a region of a gene (e.g. KASP) may employ one or more primer sequences shown in Example 1 (e.g. at Table 1 thereof) or a nucleotide sequence having at least 90% (preferably at least 95% or more preferably 100%) sequence identity thereto. Preferably, a technique for sequencing at least a region of a gene employs the specific combination of primers presented in Table 1 for sequencing said region (e.g. as defined by a SNPedia Accession No.). The sequences amplified by said primers may also correspond to those sequences shown in Example 1 (e.g. shown as Allele X and/or Y).

In one embodiment, where a gene is MTHFR and the SNP corresponds to SNPedia accession no. rs1801133, a method of the invention may comprise the use of a combination of primer sequences having at least 90% (preferably at least 95% or more preferably 100%) sequence identity to SEQ ID NO: 1, SEQ ID NO: 2, and SEQ ID NO: 3 to determine the subject's genetic profile and/or the corresponding genetic reference standard.

In one embodiment, where a gene is CAT and the SNP corresponds to SNPedia accession no. rs1001179, a method of the invention may comprise the use of a combination of primer sequences having at least 90% (preferably at least 95% or more preferably 100%) sequence identity to SEQ ID NO: 5, SEQ ID NO: 6, and SEQ ID NO: 7 to determine the subject's genetic profile and/or the corresponding genetic reference standard.

In one embodiment, where a gene is SOD2 and the SNP corresponds to SNPedia accession no. rs4880, a method of the invention may comprise the use of a combination of primer sequences having at least 90% (preferably at least 95% or more preferably 100%) sequence identity to SEQ ID NO: 9, SEQ ID NO: 10, and SEQ ID NO: 11 to determine the subject's genetic profile and/or the corresponding genetic reference standard.

In one embodiment, where a gene is TCF7L2 and the SNP corresponds to SNPedia accession no. rs7903146, a method of the invention may comprise the use of a combination of primer sequences having at least 90% (preferably at least 95% or more preferably 100%) sequence identity to SEQ ID NO: 13, SEQ ID NO: 14, and SEQ ID NO: 15 to determine the subject's genetic profile and/or the corresponding genetic reference standard.

In one embodiment, where a gene is HLA-DQA1 and the SNP corresponds to SNPedia accession no. rs2187668, a method of the invention may comprise the use of a combination of primer sequences having at least 90% (preferably at least 95% or more preferably 100%) sequence identity to SEQ ID NO: 17, SEQ ID NO: 18, and SEQ ID NO: 19 to determine the subject's genetic profile and/or the corresponding genetic reference standard.

In one embodiment, where a gene is PPARG and the SNP corresponds to SNPedia accession no. rs1801282, a method of the invention may comprise the use of a combination of primer sequences having at least 90% (preferably at least 95% or more preferably 100%) sequence identity to SEQ ID NO: 21, SEQ ID NO: 22, and SEQ ID NO: 23 to determine the subject's genetic profile and/or the corresponding genetic reference standard.

In one embodiment, where a gene is APOA5 and the SNP corresponds to SNPedia accession no. rs662799, a method of the invention may comprise the use of a combination of primer sequences having at least 90% (preferably at least 95% or more preferably 100%) sequence identity to SEQ ID NO: 25, SEQ ID NO: 26, and SEQ ID NO: 27 to determine the subject's genetic profile and/or the corresponding genetic reference standard.

In one embodiment, where a gene is FTO and the SNP corresponds to SNPedia accession no. rs9939609, a method of the invention may comprise the use of a combination of primer sequences having at least 90% (preferably at least 95% or more preferably 100%) sequence identity to SEQ ID NO: 29, SEQ ID NO: 30, and SEQ ID NO: 31 to determine the subject's genetic profile and/or the corresponding genetic reference standard.

In one embodiment, where a gene is VDR and the SNP corresponds to SNPedia accession no. rs1544410, a method of the invention may comprise the use of a combination of primer sequences having at least 90% (preferably at least 95% or more preferably 100%) sequence identity to SEQ ID NO: 33, SEQ ID NO: 34, and SEQ ID NO: 35 to determine the subject's genetic profile and/or the corresponding genetic reference standard.

In one embodiment, where a gene is ADRB3 and the SNP corresponds to SNPedia accession no. rs4994, a method of the invention may comprise the use of a combination of primer sequences having at least 90% (preferably at least 95% or more preferably 100%) sequence identity to SEQ ID NO: 37, SEQ ID NO: 38, and SEQ ID NO: 39 to determine the subject's genetic profile and/or the corresponding genetic reference standard.

In one embodiment, where a gene is MCM6 and the SNP corresponds to SNPedia accession no. rs4988235, a method of the invention may comprise the use of a combination of primer sequences having at least 90% (preferably at least 95% or more preferably 100%) sequence identity to SEQ ID NO: 41, SEQ ID NO: 42, and SEQ ID NO: 43 to determine the subject's genetic profile and/or the corresponding genetic reference standard.

In one embodiment, where a gene is ADRB2 and the SNP corresponds to SNPedia accession no. rs1042714, a method of the invention may comprise the use of a combination of primer sequences having at least 90% (preferably at least 95% or more preferably 100%) sequence identity to SEQ ID NO: 45, SEQ ID NO: 46, and SEQ ID NO: 47 to determine the subject's genetic profile and/or the corresponding genetic reference standard.

In one embodiment, where a gene is SLC19A1 and the SNP corresponds to SNPedia accession no. rs1051266, a method of the invention may comprise the use of a combination of primer sequences having at least 90% (preferably at least 95% or more preferably 100%) sequence identity to SEQ ID NO: 49, SEQ ID NO: 50, and SEQ ID NO: 51 to determine the subject's genetic profile and/or the corresponding genetic reference standard.

In one embodiment, where a gene is LPL and the SNP corresponds to SNPedia accession no. rs268, a method of the invention may comprise the use of a combination of primer sequences having at least 90% (preferably at least 95% or more preferably 100%) sequence identity to SEQ ID NO: 53, SEQ ID NO: 54, and SEQ ID NO: 55 to determine the subject's genetic profile and/or the corresponding genetic reference standard.

In one embodiment, where a gene is VDR and the SNP corresponds to SNPedia accession no. rs731236, a method of the invention may comprise the use of a combination of primer sequences having at least 90% (preferably at least 95% or more preferably 100%) sequence identity to SEQ ID NO: 57, SEQ ID NO: 58, and SEQ ID NO: 59 to determine the subject's genetic profile and/or the corresponding genetic reference standard.

In one embodiment, where a gene is MTHFR and the SNP corresponds to SNPedia accession no. rs1801131, a method of the invention may comprise the use of a combination of primer sequences having at least 90% (preferably at least 95% or more preferably 100%) sequence identity to SEQ ID NO: 61, SEQ ID NO: 62, and SEQ ID NO: 63 to determine the subject's genetic profile and/or the corresponding genetic reference standard.

In one embodiment, where a gene is TCN2 and the SNP corresponds to SNPedia accession no. rs1801198, a method of the invention may comprise the use of a combination of primer sequences having at least 90% (preferably at least 95% or more preferably 100%) sequence identity to SEQ ID NO: 65, SEQ ID NO: 66, and SEQ ID NO: 67 to determine the subject's genetic profile and/or the corresponding genetic reference standard.

In one embodiment, where a gene is GPX1 and the SNP corresponds to SNPedia accession no. rs1050450, a method of the invention may comprise the use of a combination of primer sequences having at least 90% (preferably at least 95% or more preferably 100%) sequence identity to SEQ ID NO: 69, SEQ ID NO: 70, and SEQ ID NO: 71 to determine the subject's genetic profile and/or the corresponding genetic reference standard.

In one embodiment, where a gene is ADRB2 and the SNP corresponds to SNPedia accession no. rs1042713, a method of the invention may comprise the use of a combination of primer sequences having at least 90% (preferably at least 95% or more preferably 100%) sequence identity to SEQ ID NO: 73, SEQ ID NO: 74, and SEQ ID NO: 75 to determine the subject's genetic profile and/or the corresponding genetic reference standard.

In one embodiment, where a gene is CYP1A2 and the SNP corresponds to SNPedia accession no. rs762551, a method of the invention may comprise the use of a combination of primer sequences having at least 90% (preferably at least 95% or more preferably 100%) sequence identity to SEQ ID NO: 77, SEQ ID NO: 78, and SEQ ID NO: 79 to determine the subject's genetic profile and/or the corresponding genetic reference standard.

In one embodiment, where a gene is APOC3 and the SNP corresponds to SNPedia accession no. rs5128, a method of the invention may comprise the use of a combination of primer sequences having at least 90% (preferably at least 95% or more preferably 100%) sequence identity to SEQ ID NO: 81, SEQ ID NO: 82, and SEQ ID NO: 83 to determine the subject's genetic profile and/or the corresponding genetic reference standard.

In one embodiment, where a gene is BCO1 and the SNP corresponds to SNPedia accession no. rs12934922, a method of the invention may comprise the use of a combination of primer sequences having at least 90% (preferably at least 95% or more preferably 100%) sequence identity to SEQ ID NO: 85, SEQ ID NO: 86, and SEQ ID NO: 87 to determine the subject's genetic profile and/or the corresponding genetic reference standard.

In one embodiment, where a gene is BCO1 and the SNP corresponds to SNPedia accession no. rs7501331, a method of the invention may comprise the use of a combination of primer sequences having at least 90% (preferably at least 95% or more preferably 100%) sequence identity to SEQ ID NO: 89, SEQ ID NO: 90, and SEQ ID NO: 91 to determine the subject's genetic profile and/or the corresponding genetic reference standard.

In one embodiment, where a gene is ACE and the SNP corresponds to SNPedia accession no. rs1799752, a method of the invention may comprise the use of a combination of primer sequences having at least 90% (preferably at least 95% or more preferably 100%) sequence identity to SEQ ID NO: 93, SEQ ID NO: 94, and SEQ ID NO: 95 to determine the subject's genetic profile and/or the corresponding genetic reference standard.

In one embodiment, where a gene is ACE and the SNP corresponds to SNPedia accession no. rs1799752, a method of the invention may comprise the use of a combination of primer sequences having at least 90% (preferably at least 95% or more preferably 100%) sequence identity to SEQ ID NO: 93, SEQ ID NO: 94, and SEQ ID NO: 96 to determine the subject's genetic profile and/or the corresponding genetic reference standard.

In one embodiment, where a gene is ACE and the SNP corresponds to SNPedia accession no. rs1799752, a method of the invention may comprise the use of a combination of primer sequences having at least 90% (preferably at least 95% or more preferably 100%) sequence identity to SEQ ID NO: 93, SEQ ID NO: 94, SEQ ID NO: 95, and SEQ ID NO: 96 to determine the subject's genetic profile and/or the corresponding genetic reference standard.

The invention may employ high-throughput techniques. For example, techniques operating at the level of transcription (e.g. transcriptomic techniques) or translation (e.g. proteomic techniques). Alternatively or additionally, the invention may employ the use of genomics, e.g. to detect the presence or absence of single nucleotide polymorphisms (SNPs), promoter sequences, gene copy number (e.g. duplications), and/or enhancer or other relevant genetic features. High-throughput techniques can be used to analyse whole genomes, proteomes and transcriptomes rapidly, providing data, including the expression levels, of all of the genes, polypeptides and transcripts in a cell. Proteomics is a technique for analysing the proteome of a cell (e.g. at a particular point in time). The proteome is different in different cell types. Typically, proteomics is carried out by mass-spectrometry (e.g. liquid chromatography and mass spectrometry (LC-MS/MS)), including tandem mass-spectrometry, and gel based techniques, including differential in-gel electrophoresis. In one embodiment, mRNA of a gene can be detected and quantified by e.g. Northern blotting or by quantitative reverse transcription PCR (RT-PCR). Single cell gene expression analysis may also be performed using commercially available systems (e.g. Fluidigm Dynamic Array).

A method of the invention preferably comprises comparing:
(i) a genetic sequence of at least a region of a first copy of a gene (e.g. maternally-derived) and a genetic sequence of at least the same region of a second copy of the gene (e.g. paternally-derived) comprised in a subject's genetic profile;
   with
(ii) a genetic reference standard comprising known sequence information for at least a region of a first copy of the gene (e.g. corresponding to a maternally-derived sequence) and the same region of a second copy of the gene (e.g. corresponding to a paternally-derived sequence).

Preferably, the subject's genetic profile comprises a plurality of genetic sequences. The plurality of genetic sequences may comprise partial or whole gene sequences for two or more different genes (preferably more than two genes). In one embodiment at least one gene is associated with nutrient uptake and/or metabolism of a first nutrient and at least one gene is associated with nutrient uptake and/or metabolism of a second different nutrient. Preferably, the subject's genetic profile comprises partial or whole gene sequences for all copies (e.g. both copies [maternal and paternal copies]) of the two or more different genes.

In some embodiments, a subject's genetic sequence(s) comprise(s) at least a region of one, two, three, four, five, six, seven, eight, nine, ten, 11, 12, 13, 14, 15, 16, 17, 18, 19, 20, 25, 30, 40, 45, 50 or more genes.

In one embodiment, a method comprises:
(a) providing a subject's genetic profile, wherein the subject's genetic profile comprises sequences of at least a region of a plurality of genes (preferably where at least one gene is associated with nutrient uptake and/or metabolism of a first nutrient and at least one gene is associated with nutrient uptake and/or metabolism of a second nutrient);
(b) comparing the subject's genetic profile with at least a first set of genetic reference standards and a second set of genetic reference standards, wherein the first set of genetic reference standards comprises at least two genetic reference standards, each associated with a different requirement level of a first nutrient, and wherein the second set of genetic reference standards comprises at least two genetic reference standards, each associated with a different requirement level of a second different nutrient; and
(c) determining which of the at least two genetic reference standards of the first set of genetic reference standards is most similar to the subject's genetic profile, thereby identifying a level of the first nutrient required by the subject, and determining which of the at least two genetic reference standards of the second set of genetic reference standards is most similar to the subject's genetic profile, thereby identifying a level of the second nutrient required by the subject.

The sets of genetic reference standards may correspond to the plurality of genes where at least a region thereof is comprised in the subject's genetic profile. For example, a set of genetic reference standards may correspond to a first gene where at least a region thereof is comprised in the subject's genetic profile, and a second set of genetic reference standards may correspond to a second gene where at least a region thereof is comprised in the subject's genetic profile.

Preferably, the subject's genetic reference standard comprises sequences of at least regions of all copies (e.g. both copies [maternal and paternal copies]) of the gene.

A set of genetic reference standards as used herein comprises at least two (e.g. three) genetic reference standards. Each genetic reference standard preferably corresponds to (or comprises sequence information relating to) all copies (e.g. both copies [maternal and paternal]) of the gene. For example, said at least two genetic reference standards preferably correspond to a single region of a gene (e.g. a SNP) and each provides different sequence information for the same region. For example, referring to Table 2 at Example 1, the three rows indicating the different sequences at BCO1 (rs12934922) may be considered a set of genetic reference standards.

In one embodiment, a method of the invention comprises a comparing step and determining step utilising at least 3, 4, 5, 6, 7, 8, 9, 10, 20, or 30 sets of genetic reference standards. In some embodiments there may be more than one set of genetic reference standards for each gene, e.g. where each set provides sequence information for a different region of the same gene. Likewise, it is preferred that there is more than one set of genetic reference standards per nutrient. In such embodiments, a scoring system may be applied to identify a level of a nutrient required by a subject which takes into account which genetic reference standard of each set is most similar to the subject's genetic profile. A suitable scoring system is described herein (e.g. in the Examples and corresponding Figures).

In one embodiment, a method further comprises selecting a nutritional composition based on the identification of the level(s) of the nutrient(s) required by the subject and/or administering a nutritional composition to the subject.

In one embodiment, a method further comprises:
(d) providing a plurality of nutritional reference standards, wherein the nutritional reference standards are different from one another, and wherein each nutritional reference standard is associated with requirement levels of a plurality of nutrients;
(e) comparing the levels of the nutrients required by the subject with the requirement levels of the same nutrients in each of the plurality of nutritional reference standards; and
(f) determining which of the plurality of nutritional reference standards is most similar to the subject's requirement levels.

A nutritional reference standard may comprise information regarding a requirement level of a plurality of nutrients (preferably micronutrients). For example, a nutritional reference standard may include information of a first micronutrient (e.g. vitamin A) and an indication that the first micronutrient (e.g. vitamin A) should be present in a high amount, while a second micronutrient (e.g. selenium) should be present in a normal/base amount. Preferably, the nutritional reference standards are a database of nutrients (e.g. micronutrients) together with a corresponding dose expressed as a daily amount or as a % of a nutritional reference value (e.g. the EU NRV)). Thus, a nutritional reference standard may define a nutrient requirement level, for example, as a daily dose amount or as a % of a nutritional reference value (e.g. the EU NRV) as provided in any one of Archetypes 1 to 17 as defined in the Examples (preferably at one of Tables 17-33). The nutrient requirement levels may be “base”, “medium”, or “high” requirements as defined in any one of Archetypes 1 to 17 as defined in the Examples at one of Tables 17-33. Preferably, the nutrient requirement levels may be daily unit dose requirements of each nutrient as defined in % s in any one of Archetypes 1 to 17. Preferably, the nutritional reference standard further includes information regarding a requirement level of one or more further micronutrients (e.g. those that do not vary between Archetypes), e.g. as defined herein, preferably as defined in Table 16 of the Examples.

In one embodiment, a nutritional reference standard is associated with at least two of the following:
(i) a medium vitamin E need when compared to a reference vitamin E daily dose of 12 mg, preferably a daily unit dose of vitamin E that is 800% of a reference vitamin E daily dose, wherein the reference vitamin E daily dose is 12 mg;
(ii) a medium vitamin A need when compared to a reference vitamin A daily dose of 800 μg, preferably a daily unit dose of vitamin A that is 130% of a reference vitamin A daily dose, wherein the reference vitamin A daily dose is 800 μg;
(iii) a medium ascorbic acid (vitamin C) need when compared to a reference ascorbic acid (vitamin C) daily dose of 80 mg, preferably a daily unit dose of ascorbic acid (vitamin C) that is 350% of a reference ascorbic acid (vitamin C) daily dose, wherein the reference ascorbic acid (vitamin C) daily dose is 80 mg;
(iv) a high selenium need when compared to a reference selenium daily dose of 55 μg, preferably a daily unit dose of selenium that is 165% of a reference selenium daily dose, wherein the reference selenium daily dose is 55 μg;
(v) a base folic acid (B9) need when compared to a reference folic acid (B9) daily dose of 200 μg, preferably a daily unit dose of folic acid (B9) that is 120% of a reference folic acid (B9) daily dose, wherein the reference folic acid (B9) daily dose is 200 μg;
(vi) a medium cyanocobalamin (B12) need when compared to a reference cyanocobalamin (B12) daily dose of 2.5 μg, preferably a daily unit dose of cyanocobalamin (B12) that is 350% of a reference cyanocobalamin (B12) daily dose, wherein the reference cyanocobalamin (B12) daily dose is 2.5 μg;
(vii) a high cholecalciferol (D3) need when compared to a reference cholecalciferol (D3) daily dose of 5 μg, preferably a daily unit dose of cholecalciferol (D3) that is 600% of a reference cholecalciferol (D3) daily dose, wherein the reference cholecalciferol (D3) daily dose is 5 μg;
(viii) a base calcium need when compared to a reference calcium daily dose of 800 mg, preferably a daily unit dose of calcium that is 110% of a reference calcium daily dose, wherein the reference calcium daily dose is 800 mg; and/or
(ix) a medium pyridoxine (B6) need when compared to a reference pyridoxine (B6) daily dose of 1.4 mg, preferably a daily unit dose of pyridoxine (B6) that is 350% of a reference pyridoxine (B6) daily dose, wherein the reference pyridoxine (B6) daily dose is 1.4 mg.

In one embodiment, a nutritional reference standard is associated with at least two of the following:
(i) a medium vitamin E need when compared to a reference vitamin E daily dose of 12 mg, preferably a daily unit dose of vitamin E that is 810% of a reference vitamin E daily dose, wherein the reference vitamin E daily dose is 12 mg;
(ii) a high vitamin A need when compared to a reference vitamin A daily dose of 800 μg, preferably a daily unit dose of vitamin A that is 160% of a reference vitamin A daily dose, wherein the reference vitamin A daily dose is 800 μg;
(iii) a medium ascorbic acid (vitamin C) need when compared to a reference ascorbic acid (vitamin C) daily dose of 80 mg, preferably a daily unit dose of ascorbic acid (vitamin C) that is 360% of a reference ascorbic acid (vitamin C) daily dose, wherein the reference ascorbic acid (vitamin C) daily dose is 80 mg;
(iv) a base selenium need when compared to a reference selenium daily dose of 55 μg, preferably a daily unit dose of selenium that is 120% of a reference selenium daily dose, wherein the reference selenium daily dose is 55 μg;
(v) a base folic acid (B9) need when compared to a reference folic acid (B9) daily dose of 200 μg, preferably a daily unit dose of folic acid (B9) that is 125% of a reference folic acid (B9) daily dose, wherein the reference folic acid (B9) daily dose is 200 μg;
(vi) a medium cyanocobalamin (B12) need when compared to a reference cyanocobalamin (B12) daily dose of 2.5 μg, preferably a daily unit dose of cyanocobalamin (B12) that is 370% of a reference cyanocobalamin (B12) daily dose, wherein the reference cyanocobalamin (B12) daily dose is 2.5 μg;
(vii) a medium cholecalciferol (D3) need when compared to a reference cholecalciferol (D3) daily dose of 5 μg, preferably a daily unit dose of cholecalciferol (D3) that is 400% of a reference cholecalciferol (D3) daily dose, wherein the reference cholecalciferol (D3) daily dose is 5 μg;
(viii) a base calcium need when compared to a reference calcium daily dose of 800 mg, preferably a daily unit dose of calcium that is 112% of a reference calcium daily dose, wherein the reference calcium daily dose is 800 mg; and/or
(ix) a medium pyridoxine (B6) need when compared to a reference pyridoxine (B6) daily dose of 1.4 mg, preferably a daily unit dose of pyridoxine (B6) that is 370% of a reference pyridoxine (B6) daily dose, wherein the reference pyridoxine (B6) daily dose is 1.4 mg.

In one embodiment, a nutritional reference standard is associated with at least two of the following:
(i) a medium vitamin E need when compared to a reference vitamin E daily dose of 12 mg, preferably a daily unit dose of vitamin E that is 820% of a reference vitamin E daily dose, wherein the reference vitamin E daily dose is 12 mg;
(ii) a medium vitamin A need when compared to a reference vitamin A daily dose of 800 μg, preferably a daily unit dose of vitamin A that is 133% of a reference vitamin A daily dose, wherein the reference vitamin A daily dose is 800 μg;
(iii) a medium ascorbic acid (vitamin C) need when compared to a reference ascorbic acid (vitamin C) daily dose of 80 mg, preferably a daily unit dose of ascorbic acid (vitamin C) that is 370% of a reference ascorbic acid (vitamin C) daily dose, wherein the reference ascorbic acid (vitamin C) daily dose is 80 mg;
(iv) a base selenium need when compared to a reference selenium daily dose of 55 μg, preferably a daily unit dose of selenium that is 125% of a reference selenium daily dose, wherein the reference selenium daily dose is 55 μg;
(v) a high folic acid (B9) need when compared to a reference folic acid (B9) daily dose of 200 μg, preferably a daily unit dose of folic acid (B9) that is 205% of a reference folic acid (B9) daily dose, wherein the reference folic acid (B9) daily dose is 200 μg;
(vi) a high cyanocobalamin (B12) need when compared to a reference cyanocobalamin (B12) daily dose of 2.5 μg, preferably a daily unit dose of cyanocobalamin (B12) that is 650% of a reference cyanocobalamin (B12) daily dose, wherein the reference cyanocobalamin (B12) daily dose is 2.5 μg;
(vii) a high cholecalciferol (D3) need when compared to a reference cholecalciferol (D3) daily dose of 5 μg, preferably a daily unit dose of cholecalciferol (D3) that is 615% of a reference cholecalciferol (D3) daily dose, wherein the reference cholecalciferol (D3) daily dose is 5 μg;
(viii) a medium calcium need when compared to a reference calcium daily dose of 800 mg, preferably a daily unit dose of calcium that is 125% of a reference calcium daily dose, wherein the reference calcium daily dose is 800 mg; and/or
(ix) a high pyridoxine (B6) need when compared to a reference pyridoxine (B6) daily dose of 1.4 mg, preferably a daily unit dose of pyridoxine (B6) that is 650% of a reference pyridoxine (B6) daily dose, wherein the reference pyridoxine (B6) daily dose is 1.4 mg.

In one embodiment, a nutritional reference standard is associated with at least two of the following:
(i) a medium vitamin E need when compared to a reference vitamin E daily dose of 12 mg, preferably a daily unit dose of vitamin E that is 830% of a reference vitamin E daily dose, wherein the reference vitamin E daily dose is 12 mg;
(ii) a high vitamin A need when compared to a reference vitamin A daily dose of 800 μg, preferably a daily unit dose of vitamin A that is 163% of a reference vitamin A daily dose, wherein the reference vitamin A daily dose is 800 μg;
(iii) a medium ascorbic acid (vitamin C) need when compared to a reference ascorbic acid (vitamin C) daily dose of 80 mg, preferably a daily unit dose of ascorbic acid (vitamin C) that is 380% of a reference ascorbic acid (vitamin C) daily dose, wherein the reference ascorbic acid (vitamin C) daily dose is 80 mg;
(iv) a base selenium need when compared to a reference selenium daily dose of 55 μg, preferably a daily unit dose of selenium that is 130% of a reference selenium daily dose, wherein the reference selenium daily dose is 55 μg;
(v) a base folic acid (B9) need when compared to a reference folic acid (B9) daily dose of 200 μg, preferably a daily unit dose of folic acid (B9) that is 130% of a reference folic acid (B9) daily dose, wherein the reference folic acid (B9) daily dose is 200 μg;
(vi) a medium cyanocobalamin (B12) need when compared to a reference cyanocobalamin (B12) daily dose of 2.5 μg, preferably a daily unit dose of cyanocobalamin (B12) that is 410% of a reference cyanocobalamin (B12) daily dose, wherein the reference cyanocobalamin (B12) daily dose is 2.5 μg;
(vii) a high cholecalciferol (D3) need when compared to a reference cholecalciferol (D3) daily dose of 5 μg, preferably a daily unit dose of cholecalciferol (D3) that is 630% of a reference cholecalciferol (D3) daily dose, wherein the reference cholecalciferol (D3) daily dose is 5 μg;
(viii) a high calcium need when compared to a reference calcium daily dose of 800 mg, preferably a daily unit dose of calcium that is 145% of a reference calcium daily dose, wherein the reference calcium daily dose is 800 mg; and/or
(ix) a medium pyridoxine (B6) need when compared to a reference pyridoxine (B6) daily dose of 1.4 mg, preferably a daily unit dose of pyridoxine (B6) that is 410% of a reference pyridoxine (B6) daily dose, wherein the reference pyridoxine (B6) daily dose is 1.4 mg.

In one embodiment, a nutritional reference standard is associated with at least two of the following:
(i) a high vitamin E need when compared to a reference vitamin E daily dose of 12 mg, preferably a daily unit dose of vitamin E that is 1300% of a reference vitamin E daily dose, wherein the reference vitamin E daily dose is 12 mg;
(ii) a high vitamin A need when compared to a reference vitamin A daily dose of 800 μg, preferably a daily unit dose of vitamin A that is 166% of a reference vitamin A daily dose, wherein the reference vitamin A daily dose is 800 μg;
(iii) a high ascorbic acid (vitamin C) need when compared to a reference ascorbic acid (vitamin C) daily dose of 80 mg, preferably a daily unit dose of ascorbic acid (vitamin C) that is 500% of a reference ascorbic acid (vitamin C) daily dose, wherein the reference ascorbic acid (vitamin C) daily dose is 80 mg;
(iv) a high selenium need when compared to a reference selenium daily dose of 55 μg, preferably a daily unit dose of selenium that is 170% of a reference selenium daily dose, wherein the reference selenium daily dose is 55 μg;
(v) a base folic acid (B9) need when compared to a reference folic acid (B9) daily dose of 200 μg, preferably a daily unit dose of folic acid (B9) that is 135% of a reference folic acid (B9) daily dose, wherein the reference folic acid (B9) daily dose is 200 μg;
(vi) a high cyanocobalamin (B12) need when compared to a reference cyanocobalamin (B12) daily dose of 2.5 μg, preferably a daily unit dose of cyanocobalamin (B12) that is 670% of a reference cyanocobalamin (B12) daily dose, wherein the reference cyanocobalamin (B12) daily dose is 2.5 μg;
(vii) a high cholecalciferol (D3) need when compared to a reference cholecalciferol (D3) daily dose of 5 μg, preferably a daily unit dose of cholecalciferol (D3) that is 645% of a reference cholecalciferol (D3) daily dose, wherein the reference cholecalciferol (D3) daily dose is 5 μg;
(viii) a medium calcium need when compared to a reference calcium daily dose of 800 mg, preferably a daily unit dose of calcium that is 127% of a reference calcium daily dose, wherein the reference calcium daily dose is 800 mg; and/or
(ix) a high pyridoxine (B6) need when compared to a reference pyridoxine (B6) daily dose of 1.4 mg, preferably a daily unit dose of pyridoxine (B6) that is 670% of a reference pyridoxine (B6) daily dose, wherein the reference pyridoxine (B6) daily dose is 1.4 mg.

In one embodiment, a nutritional reference standard is associated with at least two of the following:
(i) a medium vitamin E need when compared to a reference vitamin E daily dose of 12 mg, preferably a daily unit dose of vitamin E that is 840% of a reference vitamin E daily dose, wherein the reference vitamin E daily dose is 12 mg;
(ii) a medium vitamin A need when compared to a reference vitamin A daily dose of 800 μg, preferably a daily unit dose of vitamin A that is 136% of a reference vitamin A daily dose, wherein the reference vitamin A daily dose is 800 μg;
(iii) a medium ascorbic acid (vitamin C) need when compared to a reference ascorbic acid (vitamin C) daily dose of 80 mg, preferably a daily unit dose of ascorbic acid (vitamin C) that is 390% of a reference ascorbic acid (vitamin C) daily dose, wherein the reference ascorbic acid (vitamin C) daily dose is 80 mg;
(iv) a high selenium need when compared to a reference selenium daily dose of 55 μg, preferably a daily unit dose of selenium that is 175% of a reference selenium daily dose, wherein the reference selenium daily dose is 55 μg;
(v) a medium folic acid (B9) need when compared to a reference folic acid (B9) daily dose of 200 μg, preferably a daily unit dose of folic acid (B9) that is 150% of a reference folic acid (B9) daily dose, wherein the reference folic acid (B9) daily dose is 200 μg;
(vi) a high cyanocobalamin (B12) need when compared to a reference cyanocobalamin (B12) daily dose of 2.5 μg, preferably a daily unit dose of cyanocobalamin (B12) that is 690% of a reference cyanocobalamin (B12) daily dose, wherein the reference cyanocobalamin (B12) daily dose is 2.5 μg;
(vii) a base cholecalciferol (D3) need when compared to a reference cholecalciferol (D3) daily dose of 5 μg, preferably a daily unit dose of cholecalciferol (D3) that is 180% of a reference cholecalciferol (D3) daily dose, wherein the reference cholecalciferol (D3) daily dose is 5 μg;
(viii) a base calcium need when compared to a reference calcium daily dose of 800 mg, preferably a daily unit dose of calcium that is 114% of a reference calcium daily dose, wherein the reference calcium daily dose is 800 mg; and/or
(ix) a high pyridoxine (B6) need when compared to a reference pyridoxine (B6) daily dose of 1.4 mg, preferably a daily unit dose of pyridoxine (B6) that is 690% of a reference pyridoxine (B6) daily dose, wherein the reference pyridoxine (B6) daily dose is 1.4 mg.

In one embodiment, a nutritional reference standard is associated with at least two of the following:
(i) a high vitamin E need when compared to a reference vitamin E daily dose of 12 mg, preferably a daily unit dose of vitamin E that is 1310% of a reference vitamin E daily dose, wherein the reference vitamin E daily dose is 12 mg;
(ii) a medium vitamin A need when compared to a reference vitamin A daily dose of 800 μg, preferably a daily unit dose of vitamin A that is 139% of a reference vitamin A daily dose, wherein the reference vitamin A daily dose is 800 μg;
(iii) a high ascorbic acid (vitamin C) need when compared to a reference ascorbic acid (vitamin C) daily dose of 80 mg, preferably a daily unit dose of ascorbic acid (vitamin C) that is 510% of a reference ascorbic acid (vitamin C) daily dose, wherein the reference ascorbic acid (vitamin C) daily dose is 80 mg;
(iv) a high selenium need when compared to a reference selenium daily dose of 55 μg, preferably a daily unit dose of selenium that is 180% of a reference selenium daily dose, wherein the reference selenium daily dose is 55 μg;
(v) a medium folic acid (B9) need when compared to a reference folic acid (B9) daily dose of 200 μg, preferably a daily unit dose of folic acid (B9) that is 155% of a reference folic acid (B9) daily dose, wherein the reference folic acid (B9) daily dose is 200 μg;
(vi) a base cyanocobalamin (B12) need when compared to a reference cyanocobalamin (B12) daily dose of 2.5 μg, preferably a daily unit dose of cyanocobalamin (B12) that is 200% of a reference cyanocobalamin (B12) daily dose, wherein the reference cyanocobalamin (B12) daily dose is 2.5 μg;
(vii) a medium cholecalciferol (D3) need when compared to a reference cholecalciferol (D3) daily dose of 5 μg, preferably a daily unit dose of cholecalciferol (D3) that is 415% of a reference cholecalciferol (D3) daily dose, wherein the reference cholecalciferol (D3) daily dose is 5 μg;
(viii) a medium calcium need when compared to a reference calcium daily dose of 800 mg, preferably a daily unit dose of calcium that is 129% of a reference calcium daily dose, wherein the reference calcium daily dose is 800 mg; and/or
(ix) a base pyridoxine (B6) need when compared to a reference pyridoxine (B6) daily dose of 1.4 mg, preferably a daily unit dose of pyridoxine (B6) that is 200% of a reference pyridoxine (B6) daily dose, wherein the reference pyridoxine (B6) daily dose is 1.4 mg.

In one embodiment, a nutritional reference standard is associated with at least two of the following:
(i) a medium vitamin E need when compared to a reference vitamin E daily dose of 12 mg, preferably a daily unit dose of vitamin E that is 850% of a reference vitamin E daily dose, wherein the reference vitamin E daily dose is 12 mg;
(ii) a medium vitamin A need when compared to a reference vitamin A daily dose of 800 μg, preferably a daily unit dose of vitamin A that is 142% of a reference vitamin A daily dose, wherein the reference vitamin A daily dose is 800 μg;
(iii) a medium ascorbic acid (vitamin C) need when compared to a reference ascorbic acid (vitamin C) daily dose of 80 mg, preferably a daily unit dose of ascorbic acid (vitamin C) that is 400% of a reference ascorbic acid (vitamin C) daily dose, wherein the reference ascorbic acid (vitamin C) daily dose is 80 mg;
(iv) a high selenium need when compared to a reference selenium daily dose of 55 μg, preferably a daily unit dose of selenium that is 185% of a reference selenium daily dose, wherein the reference selenium daily dose is 55 μg;
(v) a medium folic acid (B9) need when compared to a reference folic acid (B9) daily dose of 200 μg, preferably a daily unit dose of folic acid (B9) that is 160% of a reference folic acid (B9) daily dose, wherein the reference folic acid (B9) daily dose is 200 μg;
(vi) a medium cyanocobalamin (B12) need when compared to a reference cyanocobalamin (B12) daily dose of 2.5 μg, preferably a daily unit dose of cyanocobalamin (B12) that is 430% of a reference cyanocobalamin (B12) daily dose, wherein the reference cyanocobalamin (B12) daily dose is 2.5 μg;
(vii) a base cholecalciferol (D3) need when compared to a reference cholecalciferol (D3) daily dose of 5 μg, preferably a daily unit dose of cholecalciferol (D3) that is 195% of a reference cholecalciferol (D3) daily dose, wherein the reference cholecalciferol (D3) daily dose is 5 μg;
(viii) a medium calcium need when compared to a reference calcium daily dose of 800 mg, preferably a daily unit dose of calcium that is 131% of a reference calcium daily dose, wherein the reference calcium daily dose is 800 mg; and/or
(ix) a medium pyridoxine (B6) need when compared to a reference pyridoxine (B6) daily dose of 1.4 mg, preferably a daily unit dose of pyridoxine (B6) that is 430% of a reference pyridoxine (B6) daily dose, wherein the reference pyridoxine (B6) daily dose is 1.4 mg.

In one embodiment, a nutritional reference standard is associated with at least two of the following:
(i) a base vitamin E need when compared to a reference vitamin E daily dose of 12 mg, preferably a daily unit dose of vitamin E that is 130% of a reference vitamin E daily dose, wherein the reference vitamin E daily dose is 12 mg;
(ii) a medium vitamin A need when compared to a reference vitamin A daily dose of 800 μg, preferably a daily unit dose of vitamin A that is 145% of a reference vitamin A daily dose, wherein the reference vitamin A daily dose is 800 μg;
(iii) a base ascorbic acid (vitamin C) need when compared to a reference ascorbic acid (vitamin C) daily dose of 80 mg, preferably a daily unit dose of ascorbic acid (vitamin C) that is 200% of a reference ascorbic acid (vitamin C) daily dose, wherein the reference ascorbic acid (vitamin C) daily dose is 80 mg;
(iv) a base selenium need when compared to a reference selenium daily dose of 55 μg, preferably a daily unit dose of selenium that is 135% of a reference selenium daily dose, wherein the reference selenium daily dose is 55 μg;
(v) a medium folic acid (B9) need when compared to a reference folic acid (B9) daily dose of 200 μg, preferably a daily unit dose of folic acid (B9) that is 165% of a reference folic acid (B9) daily dose, wherein the reference folic acid (B9) daily dose is 200 μg;
(vi) a medium cyanocobalamin (B12) need when compared to a reference cyanocobalamin (B12) daily dose of 2.5 μg, preferably a daily unit dose of cyanocobalamin (B12) that is 450% of a reference cyanocobalamin (B12) daily dose, wherein the reference cyanocobalamin (B12) daily dose is 2.5 μg;
(vii) a medium cholecalciferol (D3) need when compared to a reference cholecalciferol (D3) daily dose of 5 μg, preferably a daily unit dose of cholecalciferol (D3) that is 430% of a reference cholecalciferol (D3) daily dose, wherein the reference cholecalciferol (D3) daily dose is 5 μg;
(viii) a medium calcium need when compared to a reference calcium daily dose of 800 mg, preferably a daily unit dose of calcium that is 133% of a reference calcium daily dose, wherein the reference calcium daily dose is 800 mg; and/or
(ix) a medium pyridoxine (B6) need when compared to a reference pyridoxine (B6) daily dose of 1.4 mg, preferably a daily unit dose of pyridoxine (B6) that is 450% of a reference pyridoxine (B6) daily dose, wherein the reference pyridoxine (B6) daily dose is 1.4 mg.

In one embodiment, a nutritional reference standard is associated with at least two of the following:
(i) a high vitamin E need when compared to a reference vitamin E daily dose of 12 mg, preferably a daily unit dose of vitamin E that is 1320% of a reference vitamin E daily dose, wherein the reference vitamin E daily dose is 12 mg;
(ii) a high vitamin A need when compared to a reference vitamin A daily dose of 800 μg, preferably a daily unit dose of vitamin A that is 169% of a reference vitamin A daily dose, wherein the reference vitamin A daily dose is 800 μg;
(iii) a high ascorbic acid (vitamin C) need when compared to a reference ascorbic acid (vitamin C) daily dose of 80 mg, preferably a daily unit dose of ascorbic acid (vitamin C) that is 520% of a reference ascorbic acid (vitamin C) daily dose, wherein the reference ascorbic acid (vitamin C) daily dose is 80 mg;
(iv) a high selenium need when compared to a reference selenium daily dose of 55 μg, preferably a daily unit dose of selenium that is 190% of a reference selenium daily dose, wherein the reference selenium daily dose is 55 μg;
(v) a base folic acid (B9) need when compared to a reference folic acid (B9) daily dose of 200 μg, preferably a daily unit dose of folic acid (B9) that is 140% of a reference folic acid (B9) daily dose, wherein the reference folic acid (B9) daily dose is 200 μg;
(vi) a medium cyanocobalamin (B12) need when compared to a reference cyanocobalamin (B12) daily dose of 2.5 μg, preferably a daily unit dose of cyanocobalamin (B12) that is 470% of a reference cyanocobalamin (B12) daily dose, wherein the reference cyanocobalamin (B12) daily dose is 2.5 μg;
(vii) a medium cholecalciferol (D3) need when compared to a reference cholecalciferol (D3) daily dose of 5 μg, preferably a daily unit dose of cholecalciferol (D3) that is 445% of a reference cholecalciferol (D3) daily dose, wherein the reference cholecalciferol (D3) daily dose is 5 μg;
(viii) a medium calcium need when compared to a reference calcium daily dose of 800 mg, preferably a daily unit dose of calcium that is 135% of a reference calcium daily dose, wherein the reference calcium daily dose is 800 mg; and/or
(ix) a medium pyridoxine (B6) need when compared to a reference pyridoxine (B6) daily dose of 1.4 mg, preferably a daily unit dose of pyridoxine (B6) that is 470% of a reference pyridoxine (B6) daily dose, wherein the reference pyridoxine (B6) daily dose is 1.4 mg.

In one embodiment, a nutritional reference standard is associated with at least two of the following:
(i) a high vitamin E need when compared to a reference vitamin E daily dose of 12 mg, preferably a daily unit dose of vitamin E that is 1330% of a reference vitamin E daily dose, wherein the reference vitamin E daily dose is 12 mg;
(ii) a high vitamin A need when compared to a reference vitamin A daily dose of 800 μg, preferably a daily unit dose of vitamin A that is 172% of a reference vitamin A daily dose, wherein the reference vitamin A daily dose is 800 μg;
(iii) a high ascorbic acid (vitamin C) need when compared to a reference ascorbic acid (vitamin C) daily dose of 80 mg, preferably a daily unit dose of ascorbic acid (vitamin C) that is 530% of a reference ascorbic acid (vitamin C) daily dose, wherein the reference ascorbic acid (vitamin C) daily dose is 80 mg;
(iv) a base selenium need when compared to a reference selenium daily dose of 55 μg, preferably a daily unit dose of selenium that is 140% of a reference selenium daily dose, wherein the reference selenium daily dose is 55 μg;
(v) a medium folic acid (B9) need when compared to a reference folic acid (B9) daily dose of 200 μg, preferably a daily unit dose of folic acid (B9) that is 170% of a reference folic acid (B9) daily dose, wherein the reference folic acid (B9) daily dose is 200 μg;
(vi) a medium cyanocobalamin (B12) need when compared to a reference cyanocobalamin (B12) daily dose of 2.5 μg, preferably a daily unit dose of cyanocobalamin (B12) that is 490% of a reference cyanocobalamin (B12) daily dose, wherein the reference cyanocobalamin (B12) daily dose is 2.5 μg;
(vii) a medium cholecalciferol (D3) need when compared to a reference cholecalciferol (D3) daily dose of 5 μg, preferably a daily unit dose of cholecalciferol (D3) that is 460% of a reference cholecalciferol (D3) daily dose, wherein the reference cholecalciferol (D3) daily dose is 5 μg;
(viii) a medium calcium need when compared to a reference calcium daily dose of 800 mg, preferably a daily unit dose of calcium that is 137% of a reference calcium daily dose, wherein the reference calcium daily dose is 800 mg; and/or
(ix) a medium pyridoxine (B6) need when compared to a reference pyridoxine (B6) daily dose of 1.4 mg, preferably a daily unit dose of pyridoxine (B6) that is 490% of a reference pyridoxine (B6) daily dose, wherein the reference pyridoxine (B6) daily dose is 1.4 mg.

In one embodiment, a nutritional reference standard is associated with at least two of the following:
(i) a high vitamin E need when compared to a reference vitamin E daily dose of 12 mg, preferably a daily unit dose of vitamin E that is 1340% of a reference vitamin E daily dose, wherein the reference vitamin E daily dose is 12 mg;
(ii) a medium vitamin A need when compared to a reference vitamin A daily dose of 800 μg, preferably a daily unit dose of vitamin A that is 148% of a reference vitamin A daily dose, wherein the reference vitamin A daily dose is 800 μg;
(iii) a high ascorbic acid (vitamin C) need when compared to a reference ascorbic acid (vitamin C) daily dose of 80 mg, preferably a daily unit dose of ascorbic acid (vitamin C) that is 540% of a reference ascorbic acid (vitamin C) daily dose, wherein the reference ascorbic acid (vitamin C) daily dose is 80 mg;
(iv) a high selenium need when compared to a reference selenium daily dose of 55 μg, preferably a daily unit dose of selenium that is 195% of a reference selenium daily dose, wherein the reference selenium daily dose is 55 μg;
(v) a medium folic acid (B9) need when compared to a reference folic acid (B9) daily dose of 200 μg, preferably a daily unit dose of folic acid (B9) that is 175% of a reference folic acid (B9) daily dose, wherein the reference folic acid (B9) daily dose is 200 μg;
(vi) a medium cyanocobalamin (B12) need when compared to a reference cyanocobalamin (B12) daily dose of 2.5 μg, preferably a daily unit dose of cyanocobalamin (B12) that is 510% of a reference cyanocobalamin (B12) daily dose, wherein the reference cyanocobalamin (B12) daily dose is 2.5 μg;
(vii) a base cholecalciferol (D3) need when compared to a reference cholecalciferol (D3) daily dose of 5 μg, preferably a daily unit dose of cholecalciferol (D3) that is 210% of a reference cholecalciferol (D3) daily dose, wherein the reference cholecalciferol (D3) daily dose is 5 μg;
(viii) a base calcium need when compared to a reference calcium daily dose of 800 mg, preferably a daily unit dose of calcium that is 116% of a reference calcium daily dose, wherein the reference calcium daily dose is 800 mg; and/or
(ix) a medium pyridoxine (B6) need when compared to a reference pyridoxine (B6) daily dose of 1.4 mg, preferably a daily unit dose of pyridoxine (B6) that is 510% of a reference pyridoxine (B6) daily dose, wherein the reference pyridoxine (B6) daily dose is 1.4 mg.

In one embodiment, a nutritional reference standard is associated with at least two of the following:
(i) a medium vitamin E need when compared to a reference vitamin E daily dose of 12 mg, preferably a daily unit dose of vitamin E that is 870% of a reference vitamin E daily dose, wherein the reference vitamin E daily dose is 12 mg;
(ii) a high vitamin A need when compared to a reference vitamin A daily dose of 800 μg, preferably a daily unit dose of vitamin A that is 175% of a reference vitamin A daily dose, wherein the reference vitamin A daily dose is 800 μg;
(iii) a medium ascorbic acid (vitamin C) need when compared to a reference ascorbic acid (vitamin C) daily dose of 80 mg, preferably a daily unit dose of ascorbic acid (vitamin C) that is 410% of a reference ascorbic acid (vitamin C) daily dose, wherein the reference ascorbic acid (vitamin C) daily dose is 80 mg;
(iv) a base selenium need when compared to a reference selenium daily dose of 55 μg, preferably a daily unit dose of selenium that is 145% of a reference selenium daily dose, wherein the reference selenium daily dose is 55 μg;
(v) a high folic acid (B9) need when compared to a reference folic acid (B9) daily dose of 200 μg, preferably a daily unit dose of folic acid (B9) that is 210% of a reference folic acid (B9) daily dose, wherein the reference folic acid (B9) daily dose is 200 μg;
(vi) a base cyanocobalamin (B12) need when compared to a reference cyanocobalamin (B12) daily dose of 2.5 μg, preferably a daily unit dose of cyanocobalamin (B12) that is 220% of a reference cyanocobalamin (B12) daily dose, wherein the reference cyanocobalamin (B12) daily dose is 2.5 μg;
(vii) a medium cholecalciferol (D3) need when compared to a reference cholecalciferol (D3) daily dose of 5 μg, preferably a daily unit dose of cholecalciferol (D3) that is 475% of a reference cholecalciferol (D3) daily dose, wherein the reference cholecalciferol (D3) daily dose is 5 μg;
(viii) a medium calcium need when compared to a reference calcium daily dose of 800 mg, preferably a daily unit dose of calcium that is 139% of a reference calcium daily dose, wherein the reference calcium daily dose is 800 mg; and/or
(ix) a base pyridoxine (B6) need when compared to a reference pyridoxine (B6) daily dose of 1.4 mg, preferably a daily unit dose of pyridoxine (B6) that is 220% of a reference pyridoxine (B6) daily dose, wherein the reference pyridoxine (B6) daily dose is 1.4 mg.

In one embodiment, a nutritional reference standard is associated with at least two of the following:
(i) a high vitamin E need when compared to a reference vitamin E daily dose of 12 mg, preferably a daily unit dose of vitamin E that is 1350% of a reference vitamin E daily dose, wherein the reference vitamin E daily dose is 12 mg;
(ii) a medium vitamin A need when compared to a reference vitamin A daily dose of 800 μg, preferably a daily unit dose of vitamin A that is 151% of a reference vitamin A daily dose, wherein the reference vitamin A daily dose is 800 μg;
(iii) a high ascorbic acid (vitamin C) need when compared to a reference ascorbic acid (vitamin C) daily dose of 80 mg, preferably a daily unit dose of ascorbic acid (vitamin C) that is 550% of a reference ascorbic acid (vitamin C) daily dose, wherein the reference ascorbic acid (vitamin C) daily dose is 80 mg;
(iv) a high selenium need when compared to a reference selenium daily dose of 55 μg, preferably a daily unit dose of selenium that is 200% of a reference selenium daily dose, wherein the reference selenium daily dose is 55 μg;
(v) a medium folic acid (B9) need when compared to a reference folic acid (B9) daily dose of 200 μg, preferably a daily unit dose of folic acid (B9) that is 180% of a reference folic acid (B9) daily dose, wherein the reference folic acid (B9) daily dose is 200 μg;
(vi) a high cyanocobalamin (B12) need when compared to a reference cyanocobalamin (B12) daily dose of 2.5 μg, preferably a daily unit dose of cyanocobalamin (B12) that is 720% of a reference cyanocobalamin (B12) daily dose, wherein the reference cyanocobalamin (B12) daily dose is 2.5 μg;
(vii) a medium cholecalciferol (D3) need when compared to a reference cholecalciferol (D3) daily dose of 5 μg, preferably a daily unit dose of cholecalciferol (D3) that is 490% of a reference cholecalciferol (D3) daily dose, wherein the reference cholecalciferol (D3) daily dose is 5 μg;
(viii) a medium calcium need when compared to a reference calcium daily dose of 800 mg, preferably a daily unit dose of calcium that is 141% of a reference calcium daily dose, wherein the reference calcium daily dose is 800 mg; and/or
(ix) a high pyridoxine (B6) need when compared to a reference pyridoxine (B6) daily dose of 1.4 mg, preferably a daily unit dose of pyridoxine (B6) that is 720% of a reference pyridoxine (B6) daily dose, wherein the reference pyridoxine (B6) daily dose is 1.4 mg.

In one embodiment, a nutritional reference standard is associated with at least two of the following:
(i) a high vitamin E need when compared to a reference vitamin E daily dose of 12 mg, preferably a daily unit dose of vitamin E that is 1360% of a reference vitamin E daily dose, wherein the reference vitamin E daily dose is 12 mg;
(ii) a high vitamin A need when compared to a reference vitamin A daily dose of 800 μg, preferably a daily unit dose of vitamin A that is 178% of a reference vitamin A daily dose, wherein the reference vitamin A daily dose is 800 μg;
(iii) a high ascorbic acid (vitamin C) need when compared to a reference ascorbic acid (vitamin C) daily dose of 80 mg, preferably a daily unit dose of ascorbic acid (vitamin C) that is 560% of a reference ascorbic acid (vitamin C) daily dose, wherein the reference ascorbic acid (vitamin C) daily dose is 80 mg;
(iv) a base selenium need when compared to a reference selenium daily dose of 55 μg, preferably a daily unit dose of selenium that is 150% of a reference selenium daily dose, wherein the reference selenium daily dose is 55 μg;
(v) a medium folic acid (B9) need when compared to a reference folic acid (B9) daily dose of 200 μg, preferably a daily unit dose of folic acid (B9) that is 185% of a reference folic acid (B9) daily dose, wherein the reference folic acid (B9) daily dose is 200 μg;
(vi) a base cyanocobalamin (B12) need when compared to a reference cyanocobalamin (B12) daily dose of 2.5 μg, preferably a daily unit dose of cyanocobalamin (B12) that is 240% of a reference cyanocobalamin (B12) daily dose, wherein the reference cyanocobalamin (B12) daily dose is 2.5 μg;
(vii) a base cholecalciferol (D3) need when compared to a reference cholecalciferol (D3) daily dose of 5 μg, preferably a daily unit dose of cholecalciferol (D3) that is 225% of a reference cholecalciferol (D3) daily dose, wherein the reference cholecalciferol (D3) daily dose is 5 μg;
(viii) a base calcium need when compared to a reference calcium daily dose of 800 mg, preferably a daily unit dose of calcium that is 118% of a reference calcium daily dose, wherein the reference calcium daily dose is 800 mg; and/or
(ix) a base pyridoxine (B6) need when compared to a reference pyridoxine (B6) daily dose of 1.4 mg, preferably a daily unit dose of pyridoxine (B6) that is 240% of a reference pyridoxine (B6) daily dose, wherein the reference pyridoxine (B6) daily dose is 1.4 mg.

In one embodiment, a nutritional reference standard is associated with at least two of the following:
i) a medium vitamin E need when compared to a reference vitamin E daily dose of 12 mg, preferably a daily unit dose of vitamin E that is 880% of a reference vitamin E daily dose, wherein the reference vitamin E daily dose is 12 mg;
(ii) a medium vitamin A need when compared to a reference vitamin A daily dose of 800 μg, preferably a daily unit dose of vitamin A that is 154% of a reference vitamin A daily dose, wherein the reference vitamin A daily dose is 800 μg;
(iii) a medium ascorbic acid (vitamin C) need when compared to a reference ascorbic acid (vitamin C) daily dose of 80 mg, preferably a daily unit dose of ascorbic acid (vitamin C) that is 420% of a reference ascorbic acid (vitamin C) daily dose, wherein the reference ascorbic acid (vitamin C) daily dose is 80 mg;
(iv) a high selenium need when compared to a reference selenium daily dose of 55 μg, preferably a daily unit dose of selenium that is 205% of a reference selenium daily dose, wherein the reference selenium daily dose is 55 μg;
(v) a medium folic acid (B9) need when compared to a reference folic acid (B9) daily dose of 200 μg, preferably a daily unit dose of folic acid (B9) that is 190% of a reference folic acid (B9) daily dose, wherein the reference folic acid (B9) daily dose is 200 μg;
(vi) a base cyanocobalamin (B12) need when compared to a reference cyanocobalamin (B12) daily dose of 2.5 μg, preferably a daily unit dose of cyanocobalamin (B12) that is 260% of a reference cyanocobalamin (B12) daily dose, wherein the reference cyanocobalamin (B12) daily dose is 2.5 μg;
(vii) a base cholecalciferol (D3) need when compared to a reference cholecalciferol (D3) daily dose of 5 μg, preferably a daily unit dose of cholecalciferol (D3) that is 240% of a reference cholecalciferol (D3) daily dose, wherein the reference cholecalciferol (D3) daily dose is 5 μg;
(viii) a base calcium need when compared to a reference calcium daily dose of 800 mg, preferably a daily unit dose of calcium that is 120% of a reference calcium daily dose, wherein the reference calcium daily dose is 800 mg; and/or
(ix) a base pyridoxine (B6) need when compared to a reference pyridoxine (B6) daily dose of 1.4 mg, preferably a daily unit dose of pyridoxine (B6) that is 260% of a reference pyridoxine (B6) daily dose, wherein the reference pyridoxine (B6) daily dose is 1.4 mg.

In one embodiment, a nutritional reference standard is associated with at least two of the following:
(i) a high vitamin E need when compared to a reference vitamin E daily dose of 12 mg, preferably a daily unit dose of vitamin E that is 1370% of a reference vitamin E daily dose, wherein the reference vitamin E daily dose is 12 mg;
(ii) a medium vitamin A need when compared to a reference vitamin A daily dose of 800 μg, preferably a daily unit dose of vitamin A that is 157% of a reference vitamin A daily dose, wherein the reference vitamin A daily dose is 800 μg;
(iii) a high ascorbic acid (vitamin C) need when compared to a reference ascorbic acid (vitamin C) daily dose of 80 mg, preferably a daily unit dose of ascorbic acid (vitamin C) that is 570% of a reference ascorbic acid (vitamin C) daily dose, wherein the reference ascorbic acid (vitamin C) daily dose is 80 mg;
(iv) a base selenium need when compared to a reference selenium daily dose of 55 μg, preferably a daily unit dose of selenium that is 155% of a reference selenium daily dose, wherein the reference selenium daily dose is 55 μg;
(v) a medium folic acid (B9) need when compared to a reference folic acid (B9) daily dose of 200 μg, preferably a daily unit dose of folic acid (B9) that is 195% of a reference folic acid (B9) daily dose, wherein the reference folic acid (B9) daily dose is 200 μg;
(vi) a medium cyanocobalamin (B12) need when compared to a reference cyanocobalamin (B12) daily dose of 2.5 μg, preferably a daily unit dose of cyanocobalamin (B12) that is 530% of a reference cyanocobalamin (B12) daily dose, wherein the reference cyanocobalamin (B12) daily dose is 2.5 μg;
(vii) a high cholecalciferol (D3) need when compared to a reference cholecalciferol (D3) daily dose of 5 μg, preferably a daily unit dose of cholecalciferol (D3) that is 660% of a reference cholecalciferol (D3) daily dose, wherein the reference cholecalciferol (D3) daily dose is 5 μg;
(viii) a high calcium need when compared to a reference calcium daily dose of 800 mg, preferably a daily unit dose of calcium that is 147% of a reference calcium daily dose, wherein the reference calcium daily dose is 800 mg; and/or
(ix) a medium pyridoxine (B6) need when compared to a reference pyridoxine (B6) daily dose of 1.4 mg, preferably a daily unit dose of pyridoxine (B6) that is 530% of a reference pyridoxine (B6) daily dose, wherein the reference pyridoxine (B6) daily dose is 1.4 mg.

A nutritional reference standard as defined above may be associated with at least 3, 4, 5, 6, 7, or 8 (preferably all) of the one or more micronutrients as recited. Preferably, said nutritional standard is also associated with one or more further micronutrients as described herein and/or one or more macronutrients as described herein.

A method of the invention thus preferably comprises comparing the levels of the nutrients required by the subject with the nutrient level requirements presented in all (e.g. each and every one) of Archetypes 1 to 17 as presented in the Examples. A scoring system may be employed to determine to which of the nutritional reference standards (e.g. Archetypes) the subject's requirements are most similar (e.g. where more than one gene contributes to the requirement). A suitable scoring system is described herein.

In one embodiment each of the nutritional reference standards corresponds to a different nutritional composition and wherein the determining which of the plurality of nutritional reference standards is most similar to the subject's requirement levels identifies the nutritional composition corresponding to the subject's nutritional requirements. In one embodiment the method further comprises selecting the nutritional composition and/or administering the nutritional composition to the subject. A nutritional composition of the invention (e.g. selected and/or administered) may comprise at least one daily unit dose or a fraction of a daily unit dose of one or more micronutrients, wherein the daily unit dose of the one or more micronutrients is as defined at Tables 17-33 of the Examples. Optionally, the nutritional composition of the invention (e.g. selected and/or administered) may comprise at least one daily unit dose or a fraction of a daily unit dose of one or more further micronutrients, wherein the daily unit dose of the one or more further micronutrients is as defined at Table 16 of the Examples. A nutritional composition of the invention (e.g. selected and/or administered) may be admixed with one or more macronutrients prior to consumption by the subject.

The method of the invention is typically carried out in vitro (e.g. ex vivo). The method of the invention can be carried out subsequently, and at a distance from the site at which the sample is obtained.

A subject's requirement level of a nutrient may correspond to the subject's need for said nutrient and/or said subject's sensitivity to said nutrient.

In one embodiment, a gene (e.g. associated with nutrient uptake and/or metabolism) may be associated with carbohydrate sensitivity, fat sensitivity, antioxidant need, vitamin E need, folate need, vitamin B12 need, vitamin D need, calcium need, caffeine sensitivity, lactose intolerance, gluten intolerance, and/or vitamin A need.

In one embodiment, the one or more genes are selected from: ACE, PPARG, TCF7L2, ADRB2, ADRB3, FTO, APOC3, LPL, APOA5, CYP1A2, SOD2, CAT, GPX1, MTHFR, SLC19A1, TCN2, VDR, MCM6, HLA DQA1, and/or BCO1. Variants and fragments of these genes (as described herein) are also encompassed. Any one, two, three, four, five, six, seven, eight, nine, ten, fifteen, twenty or more of these genes may be utilised in a method of the invention. Preferably all of said genes are utilised in a method of the invention. The manner in which said one or more genes are associated with nutrient uptake and/or metabolism is provided in Table 2 herein.

A subject's genetic profile and/or a genetic reference standard may comprise sequence information regarding a mutation present at a position within a chromosome as defined herein, e.g. in Example 1 (preferably Table 1 thereof). In more detail, Example 1 details specific positions within chromosomes where mutations are known to occur. In one embodiment, a subject's genetic profile and/or a genetic reference standard comprises sequence information regarding mutations present at all (e.g. both) copies of a gene where mutations are known to occur. For example, an insertion/deletion in the gene ACE.

Preferably, a subject's genetic profile and/or a genetic reference standard may comprise sequence information regarding a sequence of a nucleotide present at a position within a chromosome as defined in Example 1 (preferably Table 1 thereof). In more detail, Example 1 details specific positions within chromosomes where SNPs are known to occur. In one embodiment, a subject's genetic profile and/or a genetic reference standard comprises sequence information regarding nucleotides present at all (e.g. both [maternal and paternal]) copies of a gene where SNPs are known to occur. For example, a subject's genetic profile and/or a genetic reference standard may comprise sequence information regarding whether MTHFR position 11796321 of a first chromosome 1 is a “T” or a “C” and may comprise sequence information regarding whether MTHFR position 11796321 of a second chromosome 1 is a “T” or a “C”.

The term “mutation” may be any alternation in the nucleotide sequence of a gene when compared to the reference sequence (e.g. a GenBank accession version of a gene described herein). A mutation may be a substitution, an insertion, a deletion, an inversion, or a combination thereof (e.g. a combined insertion and deletion or “INDEL”). In some embodiments, the mutation is a single nucleotide polymorphism (SNP) or an insertion/deletion mutation. A subject's genetic profile may comprise one or more SNPs and/or one or more insertion/deletion mutations. Preferably a mutation is a SNP.

In some embodiments, the method comprises the step of modifying the subject's nutritional requirements from the determined nutritional requirement based on a physiological parameter of the subject. A physiological parameter of the subject may be weight, height, BMI, percentage body fat, waist circumference, cholesterol level, and/or waist:hip ratio. Any combination of physiological parameters may be used to modify the subject's nutritional requirements.

In some embodiments, the method comprises the step of modifying the subject's nutritional requirement from the determined nutritional requirement based on a lifestyle factor of the subject. A lifestyle factor of the subject may be diet, exercise and/or physical activity, smoking status and/or alcohol consumption. Any combination of lifestyle parameters may be used to modify the subject's nutritional requirements.

In some embodiments, the method comprises the step of modifying the subject's nutritional requirement from the determined nutritional requirement based on a subject's answers to a questionnaire. A questionnaire answered by the subject may identify the current nutritional status of the subject.

In one embodiment, a method of the invention may further comprise obtaining a sample (e.g. a blood and/or stool sample) from a subject and assaying said sample. Assaying may be used to confirm whether the nutritional composition is meeting a subject's nutritional requirements. Said composition can thus be modified based on information gained via the assaying in order to further optimise the nutritional composition for the subject.

A normal need or normal sensitivity when used in the context of the invention may indicate that a particular gene sequence (e.g. SNP) is not associated with any altered need or sensitivity to a nutrient indicated.

In one embodiment, a subject's nutritional requirement is a requirement for carbohydrates. Said requirement may be determined by the subject's carbohydrate sensitivity. Carbohydrate sensitivity, may be associated with at least one gene selected from: ACE, PPARG, TCF7L2, ADRB2 and/or ADRB3 (preferably all of said genes).

The mutation in ACE may be as defined via SNPedia accession no. rs1799752. Said mutation may be at chromosome 17, position 63488529 (GRCh38). The mutation may be an insertion and/or deletion of an Alu repetitive element in an intron of the ACE gene. The Alu repetitive element may correspond to a sequence having at least 70% (e.g. at least 80%, 90% or 95%) sequence identity to SEQ ID NO: 97, 101, or 102 (preferably SEQ ID NO: 97). Preferably, the Alu repetitive element may comprise (more preferably consist of) SEQ ID NO: 97, 101, or 102, most preferably SEQ ID NO: 97. A subject may have the insertion at only one copy of ACE-such a subject may have a medium carbohydrate sensitivity. Alternatively, a subject may have the insertion at neither copy of ACE-such a subject may have a high carbohydrate sensitivity. Alternatively, a subject may have the insertion at both copies of ACE-such a subject may have a base/normal carbohydrate sensitivity. In some embodiments “deletion” or “DEL” when referring to ACE indicates that the Alu repetitive element is absent from the ACE gene. An insertion at neither copy of ACE may be assigned a point score of 2. An insertion at only one copy of ACE may be assigned a point score of 1. An insertion at both copies of ACE may be assigned a point score of 0.

The mutation in PPARG may be a SNP as defined via SNPedia accession no. rs1801282. Said SNP may be located at chromosome 3, position 12351626 (GRCh38). The subject's genetic profile and/or the genetic reference standard may comprise C:C, G:C, or G:G. C:C may be associated with high carbohydrate sensitivity. G:C may be associated with normal carbohydrate sensitivity. G:G may be associated with normal carbohydrate sensitivity. C:C may be assigned a point score of 2. G:C may be assigned a point score of 0. G:G may be assigned a point score of 0.

The mutation in TCF7L2 may be a SNP as defined via SNPedia accession no. rs7903146. Said SNP may be located at chromosome 10, position 112998590 (GRCh38). The subject's genetic profile and/or the genetic reference standard may comprise T:T, T:C, or C:C. T:T may be associated with high carbohydrate sensitivity. T:C may be associated with medium carbohydrate sensitivity. C:C may be associated with normal carbohydrate sensitivity. T:T may be assigned a point score of 2. T:C may be assigned a point score of 1. C:C may be assigned a point score of 0.

The mutation in ADRB2 may be a SNP as defined via SNPedia accession no. rs1042714. Said SNP may be located at chromosome 5, position 148826910 (GRCh38). The subject's genetic profile and/or the genetic reference standard may comprise G:G, C:G, or C:C. G:G may be associated with high carbohydrate sensitivity. C:G may be associated with medium carbohydrate sensitivity. C:C may be associated with normal carbohydrate sensitivity. G:G may be assigned a point score of 2. C:G may be assigned a point score of 1. C:C may be assigned a point score of 0.

The mutation in ADRB2 may be a SNP as defined via SNPedia accession no. rs1042713. Said SNP may be located at chromosome 5, position 148826877 (GRCh38). The subject's genetic profile and/or the genetic reference standard may comprise G:G, G:A, or A:A. G:G may be associated with high carbohydrate sensitivity. G:A may be associated with medium carbohydrate sensitivity. A:A may be associated with normal carbohydrate sensitivity. G:G may be assigned a point score of 2. G:A may be assigned a point score of 1. A:A may be assigned a point score of 0.

The mutation in ADRB3 may be a SNP as defined via SNPedia accession no. rs4994. Said SNP may be located at chromosome 8, position 37966280 (GRCh38). The subject's genetic profile and/or the genetic reference standard may comprise C:C, T:C, or T:T. C:C may be associated with high carbohydrate sensitivity. T:C may be associated with medium carbohydrate sensitivity. T:T may be associated with normal carbohydrate sensitivity. C:C may be assigned a point score of 2. T:C may be assigned a point score of 1. T:T may be assigned a point score of 0.

In one embodiment, a subject's nutritional requirement is a requirement for fat. Said requirement may be determined by the subject's fat sensitivity. Fat sensitivity, may be associated with at least one gene selected from: ADRB2, ADRB3, FTO, APOC3, LPL and/or APOA5 (preferably all of said genes).

The mutation in ADRB2 may be a SNP as defined via SNPedia accession no. rs1042713. Said SNP may be located at chromosome 5, position 148826877 (GRCh38). The subject's genetic profile and/or the genetic reference standard may comprise G:G, G:A, or A:A. G:G may be associated with high fat sensitivity. G:A may be associated with medium fat sensitivity. A:A may be associated with normal fat sensitivity. G:G may be assigned a point score of 2. G:A may be assigned a point score of 1. A:A may be assigned a point score of 0.

The mutation in ADRB3 may be a SNP as defined via SNPedia accession no. rs4994. Said SNP may be located at chromosome 8, position 37966280 (GRCh38). The subject's genetic profile and/or the genetic reference standard may comprise C:C, T:C, or T:T. C:C may be associated with high fat sensitivity. T:C may be associated with medium fat sensitivity. T:T may be associated with normal fat sensitivity. C:C may be assigned a point score of 2. T:C may be assigned a point score of 1. T:T may be assigned a point score of 0.

The mutation in FTO may be a SNP as defined via SNPedia accession no. rs9939609. Said SNP may be located at chromosome 16, position 53786615 (GRCh38). The subject's genetic profile and/or the genetic reference standard may comprise A:A, T:A, or T:T. A:A may be associated with high fat sensitivity. T:A may be associated with medium fat sensitivity. T:T may be associated with normal fat sensitivity. A:A may be assigned a point score of 2. T:A may be assigned a point score of 1. T:T may be assigned a point score of 0.

The mutation in APOC3 may be a SNP as defined via SNPedia accession no. rs5128. Said SNP may be located at chromosome 11, position 116832924 (GRCh38). The subject's genetic profile and/or the genetic reference standard may comprise C:C, G:C, or G:G. C:C may be associated with high fat sensitivity. G:C may be associated with medium fat sensitivity. G:G may be associated with normal fat sensitivity. C:C may be assigned a point score of 2. G:C may be assigned a point score of 1. G:G may be assigned a point score of 0.

The mutation in LPL may be a SNP as defined via SNPedia accession no. rs268. Said SNP may be located at chromosome 8, position 19956018 (GRCh38). The subject's genetic profile and/or the genetic reference standard may comprise G:G, G:A, or A:A. G:G may be associated with high fat sensitivity. G:A may be associated with medium fat sensitivity. A:A may be associated with normal fat sensitivity. G:G may be assigned a point score of 2. G:A may be assigned a point score of 1. A:A may be assigned a point score of 0.

The mutation in APOA5 may be a SNP as defined via SNPedia accession no. rs662799. Said SNP may be located at chromosome 11, position 116792991 (GRCh38). The subject's genetic profile and/or the genetic reference standard may comprise A:A, A:G, or G:G. A:A may be associated with high fat sensitivity. A:G may be associated with medium fat sensitivity. G:G may be associated with normal fat sensitivity. A:A may be assigned a point score of 2. A:G may be assigned a point score of 1. G:G may be assigned a point score of 0.

In one embodiment, a subject's nutritional requirement is a requirement for antioxidants. Said requirement may be determined by the subject's antioxidant need. Antioxidant need, may be associated with at least one gene selected from: SOD2, CAT, and/or GPX1 (preferably all of said genes). Antioxidant need preferably determines a subject's need for vitamin A, vitamin C, and/or vitamin E (preferably vitamin A, vitamin C, and vitamin E).

The mutation in SOD2 may be a SNP as defined via SNPedia accession no. rs4880. Said SNP may be located at chromosome 6, position 159692840 (GRCh38). The subject's genetic profile and/or the genetic reference standard may comprise C:C, C:T, or T:T. C:C may be associated with high antioxidant need. C:T may be associated with medium antioxidant need. T:T may be associated with normal antioxidant need. C:C may be assigned a point score of 2. C:T may be assigned a point score of 1. T:T may be assigned a point score of 0.

The mutation in CAT may be a SNP as defined via SNPedia accession no. rs1001179. Said SNP may be located at chromosome 11, position 34438684 (GRCh38). The subject's genetic profile and/or the genetic reference standard may comprise T:T, C:T, or C:C. T:T may be associated with high antioxidant need. C:T may be associated with medium antioxidant need.

C:C may be associated with normal antioxidant need. T:T may be assigned a point score of 2. C:T may be assigned a point score of 1. C:C may be assigned a point score of 0.

The mutation in GPX1 may be a SNP as defined via SNPedia accession no. rs1050450. Said SNP may be located at chromosome 3, position 49357401 (GRCh38). The subject's genetic profile and/or the genetic reference standard may comprise T:T, T:C, or C:C. T:T may be associated with high antioxidant need. T:C may be associated with medium antioxidant need. C:C may be associated with normal antioxidant need. T:T may be assigned a point score of 2. T:C may be assigned a point score of 1. C:C may be assigned a point score of 0.

In one embodiment, a subject's nutritional requirement is a requirement for folate (vitamin B9). Said requirement may be determined by the subject's folate (vitamin B9) need. Folate (vitamin B9) need, may be associated with at least one gene selected from: MTHFR, and/or SLC19A1 (preferably all of said genes).

The mutation in MTHFR may be a SNP as defined via SNPedia accession no. rs1801133. Said SNP may be located at chromosome 1, position 11796321 (GRCh38). The subject's genetic profile and/or the genetic reference standard may comprise T:T, C:T, or C:C. T:T may be associated with high folate (vitamin B9) need. C:T may be associated with medium folate (vitamin B9) need. C:C may be associated with normal folate (vitamin B9) need. T:T may be assigned a point score of 2. C:T may be assigned a point score of 1. C:C may be assigned a point score of 0.

The mutation in MTHFR may be a SNP as defined via SNPedia accession no. rs1801131. Said SNP may be located at chromosome 1, position 11794419 (GRCh38). The subject's genetic profile and/or the genetic reference standard may comprise C:C, C:A, or A:A. C:C may be associated with high folate (vitamin B9) need. C:A may be associated with medium folate (vitamin B9) need. A:A may be associated with normal folate (vitamin B9) need. C:C may be assigned a point score of 2. C:A may be assigned a point score of 1. A:A may be assigned a point score of 0.

The mutation in SLC19A1 may be a SNP as defined via SNPedia accession no. rs1051266. Said SNP may be located at chromosome 21, position 45537880 (GRCh38). The subject's genetic profile and/or the genetic reference standard may comprise G:G, G:A, or A:A. G:G may be associated with high folate (vitamin B9) need. G:A may be associated with normal folate (vitamin B9) need. A:A may be associated with normal folate (vitamin B9) need. G:G may be assigned a point score of 2. G:A may be assigned a point score of 0. A:A may be assigned a point score of 0.

In one embodiment, a subject's nutritional requirement is a requirement for vitamin B12. Said requirement may be determined by the subject's vitamin B12 need. Vitamin B12 need, may be associated with the gene TCN2.

The mutation in TCN2 may be a SNP as defined via SNPedia accession no. rs1801198. Said SNP may be located at chromosome 22, position 30615623 (GRCh38). The subject's genetic profile and/or the genetic reference standard may comprise G:G, G:C, or C:C. G:G may be associated with high vitamin B12 need. G:C may be associated with medium vitamin B12 need. C:C may be associated with normal vitamin B12 need. G:G may be assigned a point score of 2. G:C may be assigned a point score of 1. C:C may be assigned a point score of 0.

In one embodiment, a subject's nutritional requirement is a requirement for vitamin D. Said requirement may be determined by the subject's vitamin D need. Vitamin D need, may be associated with the gene VDR.

The mutation in VDR may be a SNP as defined via SNPedia accession no. rs731236. Said SNP may be located at chromosome 12, position 47844974 (GRCh38). The subject's genetic profile and/or the genetic reference standard may comprise C:C, T:C, or T:T. C:C may be associated with high vitamin D need. T:C may be associated with medium vitamin D need. T:T may be associated with normal vitamin D need. C:C may be assigned a point score of 2. T:C may be assigned a point score of 1. T:T may be assigned a point score of 0.

The mutation in VDR may be a SNP as defined via SNPedia accession no. rs1544410. Said SNP may be located at chromosome 12, position 47846052 (GRCh38). The subject's genetic profile and/or the genetic reference standard may comprise A:A, G:A, or G:G. A:A may be associated with high vitamin D need. G:A may be associated with normal vitamin D need. G:G may be associated with normal vitamin D need. A:A may be assigned a point score of 2. G:A may be assigned a point score of 0. G:G may be assigned a point score of 0.

In one embodiment, a subject's nutritional requirement is a requirement for calcium. Said requirement may be determined by the subject's calcium need. Calcium need, may be associated with the gene VDR.

The mutation in VDR may be a SNP as defined via SNPedia accession no. rs731236. Said SNP may be located at chromosome 12, position 47844974 (GRCh38). The subject's genetic profile and/or the genetic reference standard may comprise C:C, T:C, or T:T. C:C may be associated with high calcium need. T:C may be associated with medium calcium need. T:T may be associated with normal calcium need. C:C may be assigned a point score of 2. T:C may be assigned a point score of 1. T:T may be assigned a point score of 0.

In one embodiment, a subject's nutritional requirement corresponds to caffeine sensitivity. Caffeine sensitivity, may be associated with CYP1A2.

The mutation in CYP1A2 may be a SNP as defined via SNPedia accession no. rs762551. Said SNP may be located at chromosome 15, position 74749576 (GRCh38). The subject's genetic profile and/or the genetic reference standard may comprise C:C, C:A, or A:A. C:C may be associated with high caffeine sensitivity. C:A may be associated with medium caffeine sensitivity. A:A may be associated with normal/no caffeine sensitivity. C:C may be assigned a point score of 2. C:A may be assigned a point score of 1. A:A may be assigned a point score of 0.

Knowledge of a subject's caffeine sensitivity can be used to determine which ingredients should be present in a composition to be administered to said subject. For example, where the subject is caffeine sensitive, caffeine may be omitted from the composition of the invention. Alternatively, where the subject is caffeine insensitive (or has a normal level of caffeine sensitivity), caffeine may be present in/added to the composition of the invention.

In one embodiment, a subject's nutritional requirement corresponds to lactose sensitivity. Lactose sensitivity, may be associated with the gene MCM6.

The mutation in MCM6 may be a SNP as defined via SNPedia accession no. rs4988235. Said SNP may be located at chromosome 2, position 135851076 (GRCh38). The subject's genetic profile and/or the genetic reference standard may comprise C:C, C:T, or T:T. C:C may be associated with medium lactose sensitivity. C:T may be associated with normal/no lactose sensitivity. T:T may be associated with normal/no lactose sensitivity. C:C may be assigned a point score of 1. C:T may be assigned a point score of 0. T:T may be assigned a point score of 0.

Knowledge of a subject's lactose sensitivity can be used to determine which ingredients should be present in a composition to be administered to said subject. For example, where the subject is lactose sensitive, lactose may be omitted from the composition of the invention. Alternatively, where the subject is lactose insensitive (or has a normal level of lactose sensitivity), lactose may be present in/added to the composition of the invention.

In one embodiment, a subject's nutritional requirement corresponds to gluten sensitivity and/or insensitivity (e.g. wherein the subject has coeliac disease). Gluten sensitivity and/or the presence of coeliac disease, may be associated with the gene HLA-DQA1.

The mutation in HLA-DQA1 may be a SNP as defined via SNPedia accession no. rs2187668. Said SNP may be located at chromosome 6, position 32638107 (GRCh38). The subject's genetic profile and/or the genetic reference standard may comprise T:T, T:C, or C:C. T:T may be associated with medium gluten sensitivity (optionally and the presence of coeliac disease). T:C may be associated with medium gluten sensitivity (optionally and the presence of coeliac disease). C:C may be associated with normal gluten sensitivity (optionally and the absence of coeliac disease). T:T may be assigned a point score of 1. T:C may be assigned a point score of 1. C:C may be assigned a point score of 0.

Knowledge of a subject's gluten sensitivity and/or presence/absence of coeliac disease can be used to determine which ingredients should be present in a composition to be administered to said subject. For example, where the subject is gluten sensitive and/or coeliac disease is present, gluten may be omitted from the composition of the invention. Alternatively, where the subject is gluten insensitive (or has a normal level of gluten sensitivity) and/or coeliac disease is absent, gluten may be present in/added to the composition of the invention.

In one embodiment, a subject's nutritional requirement is a requirement for vitamin A. Said requirement may be determined by the subject's vitamin A need. Vitamin A need, may be associated with the gene BCO1.

The mutation in BCO1 may be a SNP as defined via SNPedia accession no. rs12934922. Said SNP may be located at chromosome 16, position 81268089 (GRCh38). The subject's genetic profile and/or the genetic reference standard may comprise T:T, T:A, or A:A. T:T may be associated with high vitamin A need. T:A may be associated with medium vitamin A need. A:A may be associated with normal vitamin A need. T:T may be assigned a point score of 2. T:A may be assigned a point score of 1. A:A may be assigned a point score of 0.

The mutation in BCO1 may be a SNP as defined via SNPedia accession no. rs7501331. Said SNP may be located at chromosome 16, position 81280891 (GRCh38). The subject's genetic profile and/or the genetic reference standard may comprise C:C, T:C, or T:T. C:C may be associated with high vitamin A need. T:C may be associated with medium vitamin A need. T:T may be associated with normal vitamin A need. C:C may be assigned a point score of 2. T:C may be assigned a point score of 1. T:T may be assigned a point score of 0.

In one embodiment, vitamin A need may be determined by the gene BCO1 and/or the antioxidant genes CAT, SOD2, and GPX1. In one embodiment, where vitamin A need may be determined by the gene BCO1 and the antioxidant genes CAT, SOD2 and GPX1, vitamin A need is dictated by the gene BCO1. Thus, preferably where a vitamin A need provided by the antioxidant genes disagrees with a vitamin A need provided by BCO1, the vitamin A need is determined based on BCO1 only.

In one embodiment, a subject's nutritional requirement is a requirement for vitamin B6. Said requirement may be determined by the subject's vitamin B6 need. Vitamin B6 need, may be associated with the gene TCN2.

The mutation in TCN2 may be a SNP as defined via SNPedia accession no. rs1801198. Said SNP may be located at chromosome 22, position 30615623 (GRCh38). The subject's genetic profile and/or the genetic reference standard may comprise G:G, G:C, or C:C. G:G may be associated with high vitamin B6 need. G:C may be associated with medium vitamin B6 need. C:C may be associated with normal vitamin B6 need. G:G may be assigned a point score of 2. G:C may be assigned a point score of 1. C:C may be assigned a point score of 0.

The mutation in GPX1 may be a SNP as defined via SNPedia accession no. rs1050450. Said SNP may be located at chromosome 3, position 49357401 (GRCh38). The subject's genetic profile and/or the genetic reference standard may comprise T:T, T:C, or C:C. T:T may be associated with high selenium need. T:C may be associated with high selenium need. C:C may be associated with normal selenium need. T:T may be assigned a point score of 2. T:C may be assigned a point score of 2. C:C may be assigned a point score of 0.

A subject may have an increased need or sensitivity when said subject has homozygous alleles of a SNP and/or insertion/deletion that negatively affects the subject's ability to utilise a nutrient (e.g. where the negative affect is poor uptake/absorption and/or metabolism of the nutrient) when compared to a subject that is heterozygous for a SNP and/or insertion/deletion that negatively affects the subject's ability to utilise the nutrient. Likewise, subjects that are either homozygous or heterozygous for a SNP and/or insertion/deletion that negatively affects the subject's ability to utilise a nutrient may have an increased need or sensitivity when compared to a subject that does not have the SNP and/or insertion/deletion that negatively affects the subject's ability to utilise a nutrient (e.g. is homozygous for a fully functional copy of the gene that does not comprise the SNP and/or insertion/deletion).

More than one gene may contribute to a requirement for a nutrient or group thereof (e.g. nutrient need or sensitivity). A weighted score may be applied which takes into consideration the relative contribution of each gene to the requirement for the nutrient or group thereof.

The weighted score may be calculated by multiplying the number of points of a point score associated with a particular genetic reference standard to which the subject's genetic profile is most similar by the % importance. Said % importance scores are provided in Table 2 for each gene (e.g. SNP thereof).

Where two or more genes (e.g. SNPs thereof) contribute to a nutrient need or sensitivity, the combined effect of the genes (e.g. SNPs thereof) may be determined by adding up the weighted scores for each of the genes defining the nutrient need or sensitivity. A base, medium or high nutrient need or sensitivity may be assigned based on the weighted score.

Of course, the same approach may be applied where there is one gene defining a nutrient need or sensitivity. However, given the relative importance of the gene will be 100%, the weighted score will be equal to the point score associated with a particular genetic reference standard to which the subject's genetic profile is most similar.

In one embodiment, where a subject's nutritional requirement is a requirement for vitamin A, a weighted score of 0 corresponds to a base need. In one embodiment, where a subject's nutritional requirement is a requirement for vitamin A, a weighted score of >0-<1.5 corresponds to a medium need. In one embodiment, where a subject's nutritional requirement is a requirement for vitamin A, a weighted score of 1.5+ corresponds to a high need.

In one embodiment, where a subject's nutritional requirement is a requirement for antioxidants, a weighted score of 0 corresponds to a base need. In one embodiment, where a subject's nutritional requirement is a requirement for antioxidants, a weighted score of >0-<0.7 corresponds to a medium need. In one embodiment, where a subject's nutritional requirement is a requirement for antioxidants, a weighted score of 0.7+ corresponds to a high need.

In one embodiment, where a subject's nutritional requirement is a requirement for selenium, a weighted score of 0 corresponds to a base need. In one embodiment, where a subject's nutritional requirement is a requirement for selenium, a weighted score of >0 corresponds to a high need.

In one embodiment, where a subject's nutritional requirement is a requirement for vitamin B6, a weighted score of 0 corresponds to a base need. In one embodiment, where a subject's nutritional requirement is a requirement for vitamin B6, a weighted score of >0-<2 corresponds to a medium need. In one embodiment, where a subject's nutritional requirement is a requirement for vitamin B6, a score of 2 corresponds to a high need.

In one embodiment, where a subject's nutritional requirement is a requirement for folate (vitamin B9), a weighted score of 0 corresponds to a base need. In one embodiment, where a subject's nutritional requirement is a requirement for folate (vitamin B9), a weighted score of >0-<0.7 corresponds to a medium need. In one embodiment, where a subject's nutritional requirement is a requirement for folate (vitamin B9), a weighted score of 0.7+ corresponds to a high need.

In one embodiment, where a subject's nutritional requirement is a requirement for vitamin B12, a weighted score of 0 corresponds to a base need. In one embodiment, where a subject's nutritional requirement is a requirement for vitamin B12, a weighted score of >0-<2 corresponds to a medium need. In one embodiment, where a subject's nutritional requirement is a requirement for vitamin B12, a weighted score of 2 corresponds to a high need.

In one embodiment, where a subject's nutritional requirement is a requirement for vitamin D, a weighted score of 0 corresponds to a base need. In one embodiment, where a subject's nutritional requirement is a requirement for vitamin D, a weighted score of >0-<1.5 corresponds to a medium need. In one embodiment, where a subject's nutritional requirement is a requirement for vitamin D, a weighted score of 1.5+ corresponds to a high need.

In one embodiment, where a subject's nutritional requirement is a requirement for calcium, a weighted score of 0 corresponds to a base need. In one embodiment, where a subject's nutritional requirement is a requirement for calcium, a weighted score of >0-<2 corresponds to a medium need. In one embodiment, where a subject's nutritional requirement is a requirement for calcium, a weighted score of 2 corresponds to a high need.

In one embodiment, where a subject's nutritional requirement is a requirement for carbohydrates, a weighted score of >0-<0.67 corresponds to a base/normal sensitivity. In one embodiment, where a subject's nutritional requirement is a requirement for carbohydrates, a weighted score of 0.67-<1 corresponds to a medium sensitivity. In one embodiment, where a subject's nutritional requirement is a requirement for carbohydrates, a weighted score of 1+ corresponds to a high sensitivity.

In one embodiment, where a subject's nutritional requirement corresponds to caffeine sensitivity, a weighted score of 0 corresponds to a base/normal sensitivity. In one embodiment, where a subject's nutritional requirement corresponds to caffeine sensitivity, a weighted score of >0-<2 corresponds to a medium sensitivity. In one embodiment, where a subject's nutritional requirement corresponds to caffeine sensitivity, a weighted score of 2 corresponds to a high sensitivity.

In one embodiment, where a subject's nutritional requirement corresponds to lactose sensitivity, a weighted score of 0 corresponds to a base/normal sensitivity. In one embodiment, where a subject's nutritional requirement corresponds to lactose sensitivity, a weighted score of >0 corresponds to a medium sensitivity.

In one embodiment, where a subject's nutritional requirement is a requirement for fat, a weighted score of >0-<0.7 corresponds to a base/normal sensitivity. In one embodiment, where a subject's nutritional requirement is a requirement for fat, a weighted score of 0.7-<1 corresponds to a medium sensitivity. In one embodiment, where a subject's nutritional requirement is a requirement for fat, a weighted score of 1+ corresponds to a high sensitivity.

In one embodiment, where a subject's nutritional requirement corresponds to gluten sensitivity and/or insensitivity (e.g. wherein the subject has coeliac disease), a weighted score of 0 corresponds to a base/normal sensitivity. In one embodiment, where a subject's nutritional requirement corresponds to gluten sensitivity and/or insensitivity (e.g. wherein the subject has coeliac disease), a weighted score of >0 corresponds to a medium sensitivity.

Herein where a SNP is presented separated by a colon “e.g. C:C”, the nucleotide before the colon is preferably the nucleotide present at the indicated position on a first chromosome and the nucleotide after the colon is preferably the nucleotide present at the indicated position on a second chromosome. Thus, sequence information is provided for both the maternally-derived and paternally-derived copies of said chromosome. The nucleotide before the colon may correspond to either the maternally-derived copy or the paternally-derived copy, however, where the nucleotide before the colon corresponds to the maternally-derived copy, the nucleotide after the colon corresponds to the paternally-derived copy and vice versa.

An “rs” number (or Reference SNP cluster ID) is an accession number used to refer to specific SNPs. Details around said SNPs can be found by searching https://www.snpedia.com/index.php/SNPedia for the Reference SNP cluster ID. In the event that the SNP accession number entry is modified over time, it is intended that the SNP version referred to herein is the version that is current as of 18 Oct. 2021. Further SNP information referred to can be obtained at the following website: https://www.ncbi.nlm.nih.gov/snp/.

Exemplary sequences for each of the genes referred to herein can be obtained at https://www.ncbi.nlm.nih.gov/gene/using the indicated accession numbers (version numbers are indicated by the “.X” value), as accessed on 18 Oct. 2021: angiotensin I converting enzyme (ACE; NG_011648.1); peroxisome proliferator activated receptor gamma (PPARG; NG_011749.1); transcription factor 7 like 2 (TCF7L2, NG_012631.1); adrenoceptor beta 2 (ADRB2; NG_016421.2); adrenoceptor beta 3 (ADRB3; NG_011936.1); FTO alpha-ketoglutarate dependent dioxygenase (FTO; NG_012969.1); apolipoprotein C3 (APOC3; NG_008949.1); lipoprotein lipase (LPL; NG_008855.2); apolipoprotein A5 (APOA5; NG_015894.2); cytochrome P450 family 1 subfamily A member 2 (CYP1A2; NG_061543.1); superoxide dismutase 2 (SOD2; NG_008729.3); catalase (CAT; NG_013339.2); glutathione peroxidase 1 (GPX1; NG_012264.1); methylenetetrahydrofolate reductase (MTHFR; NG_013351.1); solute carrier family 19 member 1 (SLC19A1; NG_028278.2); transcobalamin 2 (TCN2; NG_007263.1); vitamin D receptor (VDR; NG_008731.1); minichromosome maintenance complex component (MCM6; NG_008958.1); major histocompatibility complex, class II, DQ alpha 1 (HLA-DQA1; NG_032876.1); and beta-carotene oxygenase 1 (BCO1; NG_012171.1).

In one aspect the invention provides a use of a nutritional composition or kit according to the invention to provide a subject with its daily micronutrient requirements, wherein a daily unit dose of the one or more micronutrients comprised in the composition is administered to the subject per day. In a related aspect, the invention provides a method of providing a subject with its daily micronutrient requirements, the method comprising administering a daily unit dose of the one or more micronutrients comprised in the composition according to the invention to the subject per day.

The nutritional composition or kit is preferably administered by the subject to itself. The composition may be administered in any manner and/or in any amount suitable to achieve the daily unit dose per day. For example, the composition may be administered in one or more servings per day. Where administered in a single serving, the amount administered at the serving corresponds to the daily unit dose of the one or more micronutrients. Where administered in two or more servings, the amount administered at each serving corresponds to less than the daily unit dose of the one or more micronutrients. For example, where administered in two servings, the amount administered at each serving is 50% of the daily unit dose of the one or more micronutrients.

In one embodiment, the use or method of the invention further provides the subject with a daily unit dose of one or more further micronutrients as described herein. Thus, the composition preferably comprises all micronutrients required by the subject per day.

In some embodiments, the use or method of the invention further provides the subject with a daily unit dose of one or more macronutrients as described herein. However, it is preferred that the use or method provides the subject with a fraction of a daily unit dose of the one or more macronutrients.

The nutritional composition comprising one or more micronutrients (and optionally one or more further micronutrients) may be admixed with one or more macronutrients prior to administration.

The nutritional composition is preferably hydrated prior to administration.

A subject may be administered the composition (or kit) for at least 2, 10, 20, 50, 100, 200, 500 or 1000 days. Preferably, the subject is administered the composition (or kit) daily.

Embodiments related to the various compositions of the invention are intended to be applied equally to the kits, methods, or uses, and vice versa.

### Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22 (22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264 (4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8 (5) CABIOS 501-509 (1992); Gibbs sampling, see, e.g., C. E. Lawrence et al., Detecting Subtle Sequence Signals: A Gibbs Sampling Strategy for Multiple Alignment, 262 (5131) Science 208-214 (1993); Align-M, see, e.g., Ivo Van Walle et al., Align-M-A New Algorithm for Multiple Alignment of Highly Divergent Sequences, 20 (9) Bioinformatics: 1428-1435 (2004).

Thus, percent sequence identity is determined by conventional methods. See, for example, Altschul et al., Bull. Math. Bio. 48:603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

ALIGNMENT SCORES FOR DETERMINING SEQUENCE IDENTITY

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

The percent identity is then calculated as:

$$\frac{Total number of identical matches}{[ \begin{matrix}length of the longer sequence plus the \\ number of gaps introduced into the longer \\ sequence in order to align the two sequences\end{matrix} ]} \times 100$$

Substantially homologous polypeptides are characterized as having one or more amino acid substitutions, deletions or additions. These changes are preferably of a minor nature, that is conservative amino acid substitutions (see below) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

### Conservative Amino Acid Substitutions

Basic: arginine
   lysine
   histidine
Acidic: glutamic acid
   aspartic acid
   glutamine
Polar: asparagine
Hydrophobic: leucine
   isoleucine
   valine
Aromatic: phenylalanine
   tryptophan
   tyrosine
Small: glycine
   alanine
   serine
   threonine
   methionine

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins.

For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244:1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

Amino acids are referred to herein using the name of the amino acid, the three letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “a micronutrient” includes a plurality of such candidate agents and reference to “the micronutrient” includes reference to one or more micronutrients and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

### BRIEF DESCRIPTION OF THE DRAWINGS

Embodiments of the invention will now be described, by way of example only, with reference to the following Figures and Examples.

FIG. 1 shows a process of determining a subject's nutritional requirements based on their genetic profile. (A) Step 1: genes are mapped to the nutrients they effect and assigned a weighting to indicate the relative level of influence on nutrient requirement; (B) Step 2: each potential expression of a gene is awarded at point (e.g. 0, 1 or 2 points). The higher the score, the greater the impact (e.g. on need); (C) Step 3: subject DNA results are compared against the table and a “1” inserted against the genetic profile observed (column 6); a weighted score is calculated for the gene as indicated (column 7) and an overall score is calculated for the nutrient category; (D) Step 4: the subject's scores for each nutrient category are compared against a score range indicative of a final nutrient requirement and a “1” placed against the appropriate requirement level.

FIG. 2 shows how a subject is assigned to a particular archetype. The scores from FIG. 1D are compared against all archetypes, where each archetype has been similarly scored. For example, where an archetype is representative of a high (increased) vitamin A requirement, a “1” is scored against that requirement level for the archetype. Whenever the subject's requirement and the archetype requirement are the same, 1 point is scored. The archetype that has scored the most points across all nutrients tested is considered to be the archetype that best fits the subject's genetically-determined nutritional requirements.

### EXAMPLES

### Example 1

### Genetic Analysis & Weight Scoring for a Population of Subjects

The DNA from samples provided by 181 people was extracted by LGC and their genetic (SNP or for ACE insertion/deletion) profile was determined using LGC's proprietary Kompetitive Allele Specific PCT (KASP) technology as carried out according to LGC's standard operating procedures. LGC's protocols for running KASP genotyping can be obtained from https://www.biosearchtech.com/products/pcr-kits-and-reagents/genotyping-assays/kasp-genotyping-chemistry. Briefly, DNA from samples provided by subjects was extracted and parallel samples (one per SNP (or insertion/deletion for ACE) were each mixed with a SNP (or insertion/deletion for ACE)-specific Triton X-100-free Assay mix (LGC KASP-TF Master Mix [as developed and manufactured by LGC]) each containing three (or four where indicated) assay-specific non-labelled oligos: two allele-specific forward primers and one common reverse primer. The table below shows the sequence of the allele-specific forward primers (Primer 1 and Primer 2, respectively) and common reverse primers (Common Primer) for each SNP (or insertion/deletion for ACE) tested. The columns headed “Allele X” and “Allele Y” of the table presents each possible SNP (or insertion/deletion profile for ACE), with Primer 1 being specific for “Allele X” and Primer 2 being specific for “Allele Y”. The allele-specific primers each harboured a unique tail sequence that corresponds with a universal FRET (fluorescence resonant energy transfer) cassette; one labelled with FAM™ dye and the other with HEX™ dye. The LGC KASP-TF Master Mix contained the universal FRET cassettes, ROX™ passive reference dye, taq polymerase, free nucleotides and MgCl₂ in an optimised buffer solution. As part of LGC's protocols in this instance, the relevant allele-specific primer binds to the template and polymerises, thus attaching the tail sequence to the newly synthesised strand during the thermal cycling process. The complement of the allele-specific tail sequence is then generated during subsequent rounds of PCR, enabling the fluor labelled part of the FRET cassette to bind to the DNA. The fluor is no longer quenched and emits fluorescence. Competitive allele-specific PCR achieves bi-allelic discrimination through the competitive binding of the two allele-specific forward primers. If the genotype at a given SNP is homozygous, only one of the two possible fluorescent signals will be generated. If the genotype is heterozygous, a mixed fluorescent signal will be generated.

The sequences of primers, are shown for each region in the table below (Table 1), together with an exemplary context sequence around the SNP or insertion/deletion.

TABLE 1

| SNPedia | | | | | | | |
|---|---|---|---|---|---|---|---|
| Access | | Primer 1 | Primer 2 | Common | | | Context |
| ion No. | Location | (Allele X) | (Allele Y) | Primer | Allel X | Allele Y | sequence |
| rs1801133 | Gene: | AAAGCT | AAAGC | TTGAG | T | C | GAGCTTTGAGGCT |
| | MTHFR | GCGTGA | TGCGT | GCTGA | | | GACCTGAAGCACT |
| | Chromosome: | TGATGA | GATGA | CCTGA | | | TGAAGGAGAAGGT |
| | 1 | AATCGA | TGAAA | AGCAC | | | GTCTGCGGGAG[X] |
| | Position: | (SEQ ID | TCGG | TTGA | | | CGATTTCATCAT |
| | 11796321 | NO: 1) | (SEQ ID | (SEQ ID | | | CACGCAGCTTTTC |
| | (GRCh38) | | NO: 2) | NO: 3) | | | TTTGAGGCTGACA |
| | | | | | | | CATTCTTCCGCT |
| | | | | | | | (SEQ ID NO: 4) |
| | | | | | | | X = C or T |
| rs1001179* | Gene: CAT | CCGCCC | CGCCC | TCCCG | T | C | GCGGCCTGAAGGA |
| | Chromosome: | TGGGTT | TGGGT | CTCTG | | | TGCTGATAACCGG |
| | 11 | CGGCTA | TCGGC | GCCCA | | | GAGCCCCGCCCTG |
| | Position: | TT | TATC | GCAAT | | | GGTTCGGCTAT[X] |
| | 34438684 | (SEQ ID | (SEQ ID | T | | | CCGGGCACCCCG |
| | (GRCh38) | NO: 5) | NO: 6) | (SEQ ID | | | GGCCGGCGGGGCG |
| | | 5) | | NO: 7) | | | AGGCTCTCCAATT |
| | | | | | | | GCTGGGCCAGAG |
| | | | | | | | (SEQ ID NO: 8) |
| | | | | | | | X = C or T |
| rs4880 | Gene: | GGAGCC | GGAGC | CCGGG | T | C | GACCGGGCTGTGC |
| | SOD2 | CAGATA | CCAGA | CTGTG | | | TTTCTCGTCTTCA |
| | Chromosome: | CCCCAA | TACCC | CTTTC | | | GCACCAGCAGGCA |
| | 6 | AA | CAAAG | TCGTC | | | GCTGGCTCCGG[X] |
| | Position: | (SEQ | (SEQ | TT | | | TTTGGGGTATCT |
| | 159692840 | ID NO: | ID | (SEQ | | | GGGCTCCAGGCAG |
| | (GRCh38) | 9) | NO: | ID | | | AAGCACAGCCTCC |
| | | | 10) | NO: | | | CCGACCTGCCCT |
| | | | | 11) | | | (SEQ ID NO: 12) |
| | | | | | | | X = C or T |
| rs7903146 | Gene: | AATTAG | CAATT | GGGTG | C | T | GCACAGCTGTTAT |
| | TCF7L2 | AGAGCT | AGAGA | CCTCA | | | TTACTGAACAATT |
| | Chromosome: | AAGCAC | GCTAA | TACGG | | | AGAGAGCTAAGCA |
| | 10 | TTTTTA | GCACT | CAATT | | | CTTTTTAGATA[X] |
| | Position: | GATAC | TTTTA | AAATT | | | TATATAATTTAA |
| | 112998590 | (SEQ ID | GATAT | ATAT | | | TTGCCGTATGAGG |
| | (GRCh38) | NO: 13) | (SEQ ID | (SEQ ID | | | CACCCTTAGTTTT |
| | | | NO: 14) | NO: 15) | | | CAGACGAGAAAC |
| | | | | | | | (SEQ ID NO: 16) |
| | | | | | | | X = C or T |
| rs2187668** | Gene: | CATTTT | CAYTC | GACAC | C | T | AGTCCAGCAGGCT |
| | HLA-DQA1 | ACCACA | ATTTT | ATATG | | | GAATGCCTTCAAC |
| | Chromosome: | TGGTCC | ACCAC | AGGCA | | | AYTCATTTTACCA |
| | 6 | TCAC | ATGGT | GCTGA | | | CATGGTCCTCA[X] |
| | Position: | (SEQ ID | CCTCA | GAGTA | | | TTACTCTCAGCT |
| | 32638107 | NO: 17) | T | (SEQ ID | | | GCCTCATATGTGT |
| | (GRCh38) | | (SEQ ID | NO: 19) | | | CACCTCACAARTA |
| | | | NO: 18) | | | | ATCAAATAAAAT |
| | | | | | | | (SEQ ID NO: 20) |
| | | | | | | | X = C or T |
| rs1801282 | Gene: | ATCAGT | ATCAG | CCCTA | C | G | CCCTATTCCATGC |
| | PPARG | GAAGGA | TGAAG | TTCCA | | | TGTTATGGGTGAA |
| | Chromosome: | ATCGCT | GAATC | TGCTG | | | ACTCTGGGAGATT |
| | 3 | TTCTGG | GCTTT | TTATG | | | CTCCTATTGAC[X] |
| | Position: | (SEQ ID | CTGC | GGTGA | | | CAGAAAGCGATT |
| | 12351626 | NO: 21) | (SEQ ID | A | | | CCTTCACTGATAC |
| | (GRCh38) | | NO: 22) | (SEQ ID | | | ACTGTCTGCAAAC |
| | | | | NO: 23) | | | ATATCACAAGGT |
| | | | | | | | (SEQ ID NO: 24) |
| | | | | | | | X = C or G |
| rs662799 | Gene: | CCAGGA | CCCAG | CAGAG | G | A | AAGAGGCATCTGG |
| | APOA5 | ACTGGA | GAACT | AAGAT | | | GCCAGNGACTCTG |
| | Chromosome: | GCGAAA | GGAGC | CTGAG | | | AGCCCCAGGAACT |
| | 11 | GTG | GAAAG | CATTT | | | GGAGCGAAAGT[X] |
| | Position: | (SEQ ID | TA | GGGCT | | | AGATTTGCCCCA |
| | 116792991 | NO: 25) | (SEQ ID | T | | | TGAGGANAAGNTG |
| | (GRCh38) | | NO: 26) | (SEQ ID | | | AACTCCACTCNCA |
| | | | | NO: 27) | | | GGGCCTCTGAGG |
| | | | | | | | (SEQ ID NO: 28) |
| | | | | | | | X = A or G |
| rs9939609 | Gene: FTO | ACAGAG | ACAGA | CTGAA | A | T | TTAGAATGTCTGA |
| | Chromosome: | ACTATC | GACTA | TTATT | | | ATTATTATTCTAG |
| | 16 | CAAGTG | TCCAA | ATTCT | | | GTTCCTTGCGACT |
| | Position: | CATCAC | GTGCA | AGGTT | | | GCTGTGAATTT[X] |
| | 53786615 | T | TCACA | CCTTG | | | GTGATGCACTTG |
| | (GRCh38) | (SEQ ID | (SEQ ID | CGACT | | | GATAGTCTCTGTT |
| | | NO: 29) | NO: 30) | (SEQ ID | | | ACTCTAAAGTTTT |
| | | | | NO: 31) | | | AATAGGTAACAG |
| | | | | | | | (SEQ ID NO: 32) |
| | | | | | | | X = A or T |
| rs1544410 | Gene: VDR | CAGAGC | CAGAG | ATTCT | A | G | ATTCTGAGGAACT |
| | Chromosome: | CTGAGT | CCTGA | GAGGA | | | AGATAAGCAGGGT |
| | 12 | ATTGGG | GTATT | ACTAG | | | TCCTGGGGCCACA |
| | Position: | AATGT | GGGAA | ATAAG | | | GACAGGCCTGC[X] |
| | 47846052 | (SEQ ID | TGC | CAGGG | | | CATTCCCAATAC |
| | (GRCh38) | NO: 33) | (SEQ ID | TT | | | TCAGGCTCTGCTC |
| | | | NO: 34) | (SEQ ID | | | TTGCGTGAACTGG |
| | | | | NO: 35) | | | GCTCAACATTCC |
| | | | | | | | (SEQ ID NO: 36) |
| | | | | | | | X = A or G |
| rs4994 | Gene: | GTCATC | GGTCA | GGTCA | C | T | CGGTGCTGGCCAC |
| | ADRB3 | GTGGCC | TCGTG | TGGTC | | | CGTGGGAGGCAAC |
| | Chromosome: | ATCGCC | GCCAT | TGGAG | | | CTGCTGGTCATCG |
| | 8 | C | CGCCT | TCTCG | | | TGGCCATCGCC[X] |
| | Position: | (SEQ ID | (SEQ ID | GA | | | GGACTCCGAGAC |
| | 37966280 | NO: 37) | NO: 38) | (SEQ ID | | | TCCAGACCATGAC |
| | (GRCh38) | | | NO: 39) | | | CAACGTGTTCGTG |
| | | | | | | | ACTTCGCTGGCC |
| | | | | | | | (SEQ ID NO: 40) |
| | | | | | | | X = C or T |
| 8235 | Gene: | AGAGTT | GAGTT | CCCTA | T | C | ACAATGTACTAGT |
| | MCM6 | CCTTTG | CCTTT | CAATG | | | AGGCCTCTGCNCT |
| | Chromosome: | AGGCCA | GAGGC | TACTA | | | GGCAATACAGATA |
| | 2 | GGGA | CAGGG | GTAGG | | | AGATAANGTAG[X] |
| | Position: | (SEQ ID | G | CCTCT | | | CCCTGGCCTCAA |
| | 135851076 | NO: 41) | (SEQ ID | (SEQ ID | | | AGGAACTCTCCTC |
| | (GRCh38) | | NO: 42) | NO: 43) | | | CTTAGGTTGCATT |
| | | | | | | | TGTATAATGTTT |
| | | | | | | | (SEQ ID NO: 44) |
| | | | | | | | X = C or T |
| rs1042714 | Gene: | CACCCA | CACCC | GAAGC | G | C | TCTTGCTGGCACC |
| | ADRB2 | CACCTC | ACACC | CATGC | | | CANTNGAAGCCAT |
| | Chromosome: | GTCCCT | TCGTC | GCCGG | | | GCGCCGGACCANG |
| | 5 | TTC | CCTTT | ACCA | | | ACGTCACGCAG[X] |
| | Position: | (SEQ ID | G | (SEQ ID | | | AAAGGGACGAGG |
| | 148826910 | NO: 45) | (SEQ ID | NO: 47) | | | TGTGGGTGGTGGG |
| | (GRCh38) | | NO: 46) | | | | CATGGGCATCGTC |
| | | | | | | | ATGTCTCTCATC |
| | | | | | | | (SEQ ID NO: 48) |
| | | | | | | | X = C or G |
| rs1051266 | Gene: | GAAGCA | AAGCA | GACCC | A | G | GCAGGTGCCCGTG |
| | SLC19A1 | AAGGTA | AAGGT | CGAGC | | | GAACCTGGGCCTG |
| | Chromosome: | GCACAC | AGCAC | TCCGG | | | ACCCCGAGCTCCG |
| | 21 | GAGGT | ACGAG | TCCT | | | GTCCTGGCGGC[X] |
| | Position: | (SEQ ID | GC | (SEQ ID | | | CCTCGTGTGCTA |
| | 45537880 | NO: 49) | (SEQ ID | NO: 51) | | | CCTTTGCTTCTAC |
| | (GRCh38) | | NO: 50) | | | | GGCTTCATGGCGC |
| | | | | | | | AGATACGGCCAG |
| | | | | | | | (SEQ ID NO: 52) |
| | | | | | | | X = A or G |
| rs268 | Gene: LPL | GCAACA | CAACA | TTTTG | A | G | CTGCTTGAGTTGT |
| | Chromosome: | ATCTGG | ATCTG | CTGCT | | | AGAAAGAACCGCT |
| | 8 | GCTATG | GGCTA | TCTTT | | | GCAACAATCTGGG |
| | Position: | AGATCA | TGAGA | TGGCT | | | CTATGAGATCA[X] |
| | 19956018 | A | TCAG | CTGAC | | | TAAAGTCAGAGC |
| | (GRCh38) | (SEQ ID | (SEQ ID | TTTA | | | CAAAAGAAGCAGC |
| | | NO: 53) | NO: 54) | (SEQ ID | | | AAAATGTACCTGA |
| | | | | NO: 55) | | | AGACTCGTTCTC |
| | | | | | | | (SEQ ID NO: 56) |
| | | | | | | | X = A or G |
| rs731236 | Gene: VDR | CGGTCC | GCGGT | GTGCA | C | T | TCTATCCCCGTGC |
| | Chromosome: | TGGATG | CCTGG | GGACG | | | CCACAGATCGTCC |
| | 12 | GCCTCG | ATGGC | CCGRG | | | TGGGGTGCAGGAC |
| | Position: | (SEQ ID | CTCA | CTGAT | | | GCCGRGCTGAT[X] |
| | 47844974 | NO: 57) | (SEQ ID | (SEQ ID | | | GAGGCCATCCAG |
| | (GRCh38) | | NO: 58) | NO: 59) | | | GACCGCCTGTCCA |
| | | | | | | | ACAYACTGCAGAC |
| | | | | | | | GTAYATCCGSTG |
| | | | | | | | (SEQ ID NO: 60) |
| | | | | | | | X = C or T |
| rs1801131 | Gene: | GGGAKG | GGAKG | AGGTA | A | C | CCCCAAGGAGGAG |
| | MTHFR | AGCTGA | AGCTG | AAGAA | | | CTGCTGAAGATGT |
| | Chromosome: | CCAGTG | ACCAG | CRAAG | | | GGGGGGAKGAGCT |
| | 1 | AAGA | TGAAG | ACTTC | | | GACCAGTGAAG[X] |
| | Position: | (SEQ ID | C | AAAGA | | | AAGTGTCTTTGA |
| | 11794419 | NO: 61) | (SEQ ID | CACTT | | | AGTCTTYGTTCTT |
| | (GRCh38) | | NO: 62) | (SEQ ID | | | TACCTCTCGGGAG |
| | | | | NO: 63) | | | AACCAAACCGGA |
| | | | | | | | (SEQ ID NO: 64) |
| | | | | | | | X = A or C |
| rs1801198 | Gene: | GTTCCC | CTGTT | CCTCA | C | G | CTCTCTCTCTTCC |
| | TCN2 | AGTTCT | CCCAG | CTCTA | | | TCACTCTATCACC |
| | Chromosome: | GCCCCA | TTCTG | TCACC | | | AGTTCCTCATGAC |
| | 22 | G | CCCCA | AGTTC | | | TTCCCCCATGC[X] |
| | Position: | (SEQ ID | C | CTCAT | | | TGGGGCAGAACT |
| | 30615623 | NO: 65) | (SEQ ID | (SEQ ID | | | GGGAACAGCATGT |
| | (GRCh38) | | NO: 66) | NO: 67) | | | CTCAAGGCGAGGG |
| | | | | | | | TTGCTTTGCTGG |
| | | | | | | | (SEQ ID NO: 68) |
| | | | | | | | X = C or G |
| rs1050450 | Gene: | CGCCCT | GCGCC | GACAT | C | T | CCAGACCATTGAC |
| | GPX1 | AGGCAC | CTAGG | CGAAG | | | ATCGAGCCTGACA |
| | Chromosome: | AGCTGG | CACAG | CCCTG | | | TCGAAGCCCTGCT |
| | 3 | (SEQ ID | CTGA | CTGTC | | | GTCTCAAGGGC[X] |
| | Position: | NO: 69) | (SEQ ID | TCAA | | | CAGCTGTGCCTA |
| | 49357401 | | NO: 70) | (SEQ ID | | | GGGCGCCCCTCCT |
| | (GRCh38) | | | NO: 71) | | | ACCCCGGCTGCTT |
| | | | | | | | GGCAGTTGCAGT |
| | | | | | | | (SEQ ID NO: 72) |
| | | | | | | | X = C or T |
| rs1042713 | Gene: | GCCTTC | CCTTC | CGTGG | A | G | GCGCCATGGGGCA |
| | ADRB2 | TTGCTG | TTGCT | TCCGG | | | ACCCGGGAACGGC |
| | Chromosome: | GCACCC | GGCAC | CGCAT | | | AGCGCCTTCTTGC |
| | 5 | AATA | CCAAT | GGCTT | | | TGGCACCCAAT[X] |
| | Position: | (SEQ ID | G | (SEQ ID | | | GAAGCCATGCGC |
| | 148826877 | NO: 73) | (SEQ ID | NO: 75) | | | CGGACCACGACGT |
| | (GRCh38) | | NO: 74) | | | | CACGCAGGAAAGG |
| | | | | | | | GACGAGGTGTGG |
| | | | | | | | (SEQ ID NO: 76) |
| | | | | | | | X = A or G |
| rs762551 | Gene: | CCATCT | CCATC | CAGCT | A | C | CCTTTCCAGCTCT |
| | CYP1A2 | ACCATG | TACCA | CTCAG | | | CAGATTCTGTGAT |
| | Chromosome: | CGTCCT | TGCGT | ATTCT | | | GCTCAAAGGGTGA |
| | 15 | GT | CCTGG | GTGAT | | | GCTCTGTGGGC[X] |
| | Position: | (SEQ ID | (SEQ ID | GCTCA | | | CAGGACGCATGG |
| | 74749576 | NO: 77) | NO: 78) | A | | | TAGATGGAGCTTA |
| | (GRCh38) | | | (SEQ ID | | | GTCTTTCTGGTAT |
| | | | | NO: 79) | | | CCAGCTGGGAGC |
| | | | | | | | (SEQ ID NO: 80) |
| | | | | | | | X = A or C |
| rs5128 | Gene: | ATTGCA | GCAGG | CCCAA | C | G | TTTAAGCAACCTA |
| | APOC3 | GGACCC | ACCCA | GTCCA | | | CAGGGGCAGCCCT |
| | Chromosome: | AAGGAG | AGGAG | CCTGC | | | GGAGATTGCAGGA |
| | 11 | CTC | CTG | CTATC | | | CCCAAGGAGCT[X] |
| | Position: | (SEQ | (SEQ | CAT | | | GCAGGATGGATA |
| | 116832924 | ID NO: | ID | (SEQ | | | GGCAGGTGGACTT |
| | (GRCh38) | 81) | NO: | ID | | | GGGGTATTGAGGT |
| | | | 82) | NO: | | | CTCAGGCAGCCA |
| | | | | 83) | | | (SEQ ID NO: 84) |
| | | | | | | | X = C or G |
| rs12934922 | Gene: | GGCAGG | GGCAG | ATATT | A | T | CAGCCTTTCAGGT |
| | BCO1 | AGGCCC | GAGGC | CTCAA | | | TGGATATTCTCAA |
| | Chromosome: | AGCTCA | CCAGC | GATGG | | | GATGGCAACCGCA |
| | 16 | TT | TCATA | CAACC | | | TACATCCGGAG[X] |
| | Position: | (SEQ | (SEQ | GCATA | | | ATGAGCTGGGCC |
| | 81268089 | ID NO: | ID | CAT | | | TCCTGCCTGGCTT |
| | (GRCh38) | 85) | NO: | (SEQ | | | TCCACAGGGAGGA |
| | | | 86) | ID | | | GAAGGTGAGGTC |
| | | | | NO: | | | (SEQ ID NO: |
| | | | | 87) | | | 88) |
| | | | | | | | X = A or T |
| rs7501331 | Gene: | GTGGGC | AGTGG | CAGGG | C | T | AACTGAATTTTTT |
| | BCO1 | ACAAAT | GCACA | CCGTG | | | CAGAATGCAGAAG |
| | Chromosome: | TTAATC | AATTT | GCTGT | | | TGGGCACAAATTT |
| | me: | AAAGTG | AATCA | TGTAG | | | AATCAAAGTGG[X] |
| | 16 | GC | AAGTG | AT | | | ATCTACAACAGC |
| | Position: | (SEQ | GT | (SEQ | | | CACGGCCCTGAAG |
| | 81280891 | ID NO: | (SEQ | ID | | | GAAGAAGATGGCC |
| | (GRCh38) | 89) | ID | NO: | | | AAGTCTACTGCC |
| | | | NO: | 91) | | | (SEQ ID NO : 92) |
| | | | | | | | X = C or T |
| rs1799752 | Gene: ACE | CCTGCT | CCTGC | AGCTC | | ATACAGTCACTT | TGCTGGAGACCAC |
| | Chromosome: | GCCTAT | TGCCT | AGAGA | | TTTTTTTTTTTT | TCCCATCCTTTCT |
| | 17 | ACAGTC | ATACA | ATTTC | | TGAGACGGAGTC | CCCATTTCTCTAG |
| | Position: | ACTTTT | GTCAC | AGAGC | | TCGCTCTGTCGC | ACCTGCTGCCT[/ |
| | 63488529 | A | TTTTT | TGGAA | | CCAGGCTGGAGT | ATACAGTCACTTT |
| | (GRCh38) | (SEQ ID | (SEQ ID | TAAA | | GCAGTGGCGGGA | TTTTTTTTTTTTG |
| | | NO: 93) | NO: 94) | (SEQ ID | | TCTCGGCTCACT | AGACGGAGTCTCG |
| | | | | NO: 95) | | GCAACGTCCGCC | CTCTGTCGCCCAG |
| | | | | and/or | | TCCCGGGTTCAC | GCTGGAGTGCAGT |
| | | | | GACAG | | GCCATTCTCCTG | GGCGGGATCTCGG |
| | | | | AGCGA | | CCTCAGCCTCCC | CTCACTGCAACGT |
| | | | | GACTC | | AAGTAGCTGGGA | CCGCCTCCCGGGT |
| | | | | CGTCT | | CCACAGCGCCCG | TCACGCCATTCTC |
| | | | | CAA | | CCACTACGCCCG | CTGCCTCAGCCTC |
| | | | | (SEQ ID | | GCTAATTTTTTG | CCAAGTAGCTGGG |
| | | | | NO: 96) | | TATTTTTAGTAG | ACCACAGCGCCCG |
| | | | | | | AGACGGGGTTTC | CCACTACGCCCGG |
| | | | | | | ACCGTTTTAGCC | CTAATTTTTTGTA |
| | | | | | | GGGATGGTCTCG | TTTTTAGTAGAGA |
| | | | | | | ATCTCCTGACCT | CGGGGTTTCACCG |
| | | | | | | CGTGATCCGCCC | TTTTAGCCGGGAT |
| | | | | | | GCCTCGGCCTCC | GGTCTCGATCTCC |
| | | | | | | CAAAGTGCTGGG | TGACCTCGTGATC |
| | | | | | | ATTACAGGCGTG | CGCCCGCCTCGGC |
| | | | | | | (SEQ NO: | CTCCCAAAGTGCT |
| | | | | | | 97) *** | GGGATTACAGGCG |
| | | | | | | | TG] ATACAGTCAC |
| | | | | | | | TTTTATGTGGTTT |
| | | | | | | | CGCCAATTTTATT |
| | | | | | | | CCAGCTCTGAAAT |
| | | | | | | | T (SEQ ID NO: |
| | | | | | | | 98 = insertion |
| | | | | | | | present) |
| | | | | | | | TGCTGGAGACCAC |
| | | | | | | | TCCCATCCTTTCT |
| | | | | | | | CCCATTTCTCTAG |
| | | | | | | | ACCTGCTGCCTAT |
| | | | | | | | ACAGTCACTTTTA |
| | | | | | | | TGTGGTTTCGCCA |
| | | | | | | | ATTTTATTCCAGC |
| | | | | | | | TCTGAAATT |
| | | | | | | | (SEQ ID NO: 99 |
| | | | | | | | insertion |
| | | | | | | | absent) |

(NB, R, Y, and K in the above sequences correspond to the IUPAC nucleotide code, where R = A or G, Y =
C or T, K = G or T, and N = one or more nucleotides with any base/unknown.)
*The SNP is shown with alleles X as T and allele Y as C, as the above primers amplify the opposite strand
to that shown on SNPedia (where the SNP is thus reported as A or G).
**The SNP is shown with allele X as C and allele Y as T, as the above primers amplify the opposite strand
to that shown on SNPedia (where the SNP is thus reported as G or A).
***Other insertioins/deletions (or insertions and deletions) may occur in the ACE gene, have a similar effect
on a subject's nutrient requirements, and be detected by the above-mentioned primers, indeed others are
reported by SNPedia. For example:
TGCAGGTGTCTGCAGCATGTGGCCCCAGGCCGGGGACTCTGTAAGCCACTGCTGGAGAGCCACTCCCATCCTTTC
TCCCATTTCTCTAGACCTGCTGCCT[X]ATGTGGTTTCGCCAATTTTATTCCAGCTCTGAAATTCTCTGAGCTCC
CCTTACAAGCAGAGGTGAGCTAAGGGCTGGAGCTCAAGGC (Consensus sequence 2-SEQ ID NO: 100)
X = ATACAGTCACTTTT (SEQ ID NO: 101) or X = ATACAGTCACTTTTTTTTTTTTTTTGAGACGGAGTCTCGCTCTGTCGCCCATACAGTCACTTTT
(SEQ ID NO: 102)

The table below (Table 2) shows the gene/regions with each possible sequence having an ‘rs’ (SNPedia) accession number (DEL=deletion; INS=insertion). The association of each SNP (or insertion/deletion profile for ACE) with nutrient uptake/metabolism is presented below, together with a weighting score for each.

TABLE 2

| | | Impor- | | | |
|---|---|---|---|---|---|
| | | tance | | | |
| CATEGORY | GENE | (Wgt) | EXPRESSION | Points | Effect |
| Carb | ACE | ~16.7% | DEL:DEL | 2 | Strong Association |
| Sensitivity | rs1799752 | | | | (High sensitivity) |
| | | | INS:DEL | 1 | Medium Association |
| | | | | | (Medium sensitivity) |
| | | | INS:INS | 0 | No Association |
| | | | | | (Normal sensitivity) |
| | PPARG | ~16.7% | C:C | 2 | Strong Association |
| | rs1801282 | | | | (High sensitivity) |
| | | | G:C | 0 | No Association |
| | | | | | (Normal sensitivity) |
| | | | G:G | 0 | No Association |
| | | | | | (Normal sensitivity) |
| | TCF7L2 | ~16.7% | T:T | 2 | Strong Association |
| | rs7903146 | | | | (High sensitivity) |
| | | | T:C | 1 | Medium Association |
| | | | | | (Medium sensitivity) |
| | | | C:C | 0 | No Association |
| | | | | | (Normal sensitivity) |
| | ADRB2 | ~16.7% | G:G | 2 | Strong Association |
| | rs1042714 | | | | (High sensitivity) |
| | | | C:G | 1 | Medium Association |
| | | | | | (Medium sensitivity) |
| | | | C:C | 0 | No Association |
| | | | | | (Normal sensitivity) |
| | | ~16.7% | G:G | 2 | Strong Association |
| | | | | | (High sensitivity) |
| | ADRB2 | | G:A | 1 | Medium Association |
| | rs1042713 | | | | (Medium sensitivity) |
| | | | A:A | 0 | No Association |
| | | | | | (Normal sensitivity) |
| | ADRB3 | ~16.7% | C:C | 2 | Strong Association |
| | rs4994 | | | | (High sensitivity) |
| | | | T:C | 1 | Medium Association |
| | | | | | (Medium sensitivity) |
| | | | T:T | 0 | No Association |
| | | | | | (Normal sensitivity) |
| | Total % | 100% | | | |
| Fat Sensitivity | ADRB2 | ~16.7% | G:G | 2 | Strong Association |
| | rs1042713 | | | | (High sensitivity) |
| | | | G:A | 1 | Medium Association |
| | | | | | (Medium sensitivity) |
| | | | A:A | 0 | No Association |
| | | | | | (Normal sensitivity) |
| | ADRB3 | ~16.7% | C:C | 2 | Strong Association |
| | rs4994 | | | | (High sensitivity) |
| | | | T:C | 1 | Medium Association |
| | | | | | (Medium sensitivity) |
| | | | T:T | 0 | No Association |
| | | | | | (Normal sensitivity) |
| | FTO | ~16.7% | A:A | 2 | Strong Association |
| | rs9939609 | | | | (High sensitivity) |
| | | | T:A | 1 | Medium Association |
| | | | | | (Medium sensitivity) |
| | | | T:T | 0 | No Association |
| | | | | | (Normal sensitivity) |
| | APOC3 | ~16.7% | C:C | 2 | Strong Association |
| | rs5128 | | | | (High sensitivity) |
| | | | G:C | 1 | Medium Association |
| | | | | | (Medium sensitivity) |
| | | | G:G | 0 | No Association |
| | | | | | (Normal sensitivity) |
| | LPL | ~16.7% | G:G | 2 | Strong Association |
| | rs268 | | | | (High sensitivity) |
| | | | G:A | 1 | Medium Association |
| | | | | | (Medium sensitivity) |
| | | | A:A | 0 | No Association |
| | | | | | (Normal sensitivity) |
| | APOA5 | ~16.7% | A:A | 2 | Strong Association |
| | rs662799 | | | | (High sensitivity) |
| | | | A:G | 1 | Medium Association |
| | | | | | (Medium sensitivity) |
| | | | G:G | 0 | No Association |
| | | | | | (Normal sensitivity) |
| | Total % | 100% | | | |
| Antioxidants | SOD2 | ~33.3% | C:C | 2 | Strong Association |
| | rs4880 | | | | (High antioxidant |
| | | | | | need) |
| | | | C:T | 1 | Medium Association |
| | | | | | (Medium antioxidant |
| | | | | | need) |
| | | | T:T | 0 | No Association |
| | | | | | (Normal antioxidant |
| | | | | | need) |
| | CAT | ~33.3% | T:T | 2 | Strong Association |
| | rs1001179 | | | | (High antioxidant |
| | | | | | need) |
| | | | C:T | 1 | Medium Association |
| | | | | | (Medium antioxidant |
| | | | | | need) |
| | | | C:C | 0 | No Association |
| | | | | | (Normal antioxidant |
| | | | | | need) |
| | GPX1 | ~33.3% | T:T | 2 | Strong Association |
| | rs1050450 | | | | (High antioxidant |
| | | | | | need) |
| | | | T:C | 1 | Medium Association |
| | | | | | (Medium antioxidant |
| | | | | | need) |
| | | | C:C | 0 | No Association |
| | | | | | (Normal antioxidant |
| | | | | | need) |
| | Total % | 100% | | | |
| Folate | MTHFR | ~33.3% | T:T | 2 | Strong Association |
| (Vit B9) | rs1801133 | | | | (High need) |
| | | | C:T | 1 | Medium Association |
| | | | | | (Medium need) |
| | | | C:C | 0 | No Association |
| | | | | | (Normal need) |
| | MTHFR | ~33.3% | C:C | 2 | Strong Association |
| | rs1801131 | | | | (High need) |
| | | | C:A | 1 | Medium Association |
| | | | | | (Medium need) |
| | | | A:A | 0 | No Association |
| | | | | | (Normal need) |
| | SLC19A1 | ~33.3% | G:G | 2 | Strong Association |
| | rs1051266 | | | | (High need) |
| | | | G:A | 0 | No Association |
| | | | | | (Normal need) |
| | | | A:A | 0 | No Association |
| | | | | | (Normal need) |
| | Total % | 100% | | | |
| Vitamin B12 | TCN2 | 100% | G:G | 2 | Strong Association |
| | rs1801198 | | | | (High need) |
| | | | G:C | 1 | Medium Association |
| | | | | | (Medium need) |
| | | | C:C | 0 | No Association |
| | | | | | (Normal need) |
| | Total % | 100% | | | |
| Vitamin D | VDR | 50% | C:C | 2 | Strong Association |
| | rs731236 | | | | (High need) |
| | | | T:C | 1 | Medium Association |
| | | | | | (Medium need) |
| | | | T:T | 0 | No Association |
| | | | | | (Normal need) |
| | VDR | 50% | A:A | 2 | Strong Association |
| | rs1544410 | | | | (High need) |
| | | | G:A | 0 | No Association |
| | | | | | (Normal need) |
| | | | G:G | 0 | No Association |
| | | | | | (Normal need) |
| | Total % | 100% | | | |
| Calcium | VDR | 100% | C:C | 2 | Strong Association |
| | rs731236 | | | | (High need) |
| | | | T:C | 1 | Medium Association |
| | | | | | (Medium need) |
| | | | T:T | 0 | No Association |
| | | | | | (Normal need) |
| | Total % | 100% | | | |
| Caffeine | CYP1A2 | 100% | C:C | 2 | Strong Association |
| Sensitivity | rs762551 | | | | (High sensitivity) |
| | | | C:A | 1 | Medium Association |
| | | | | | (Medium sensitivity) |
| | | | A:A | 0 | No Association |
| | | | | | (Normal/no sensitivity) |
| | Total % | 100% | | | |
| Lactose | MCM6 | 100% | C:C | 1 | Medium Association |
| | rs4988235 | | | | (Medium sensitivity) |
| | | | C:T | 0 | No Association |
| | | | | | (Normal/no sensitivity) |
| | | | T:T | 0 | No Association |
| | | | | | (Normal/no sensitivity) |
| | Total % | 100% | | | |
| Coeliac/ | HLA | 100% | T:T | 1 | Medium Association |
| Gluten | DQA1 | | | | (Medium sensitivity) |
| | rs2187668 | | T:C | 1 | Medium Association |
| | | | | | (Medium sensitivity) |
| | | | C:C | 0 | No Association |
| | | | | | (Normal/no sensitivity) |
| | Total % | 100% | | | |
| Vitamin A | BCO1 | 50% | T:T | 2 | Strong Association |
| | rs12934922 | | | | (High need) |
| | | | T:A | 1 | Medium Association |
| | | | | | (Medium need) |
| | | | A:A | 0 | No Association |
| | | | | | (Normal need) |
| | BCO1 | 50% | C:C | 2 | Strong Association |
| | rs7501331 | | | | (High need) |
| | | | T:C | 1 | Medium Association |
| | | | | | (Medium need) |
| | | | T:T | 0 | No Association |
| | | | | | (Normal need) |
| | Total % | 100% | | | |
| Vitamin B6 | TCN2 | 100% | G:G | 2 | Strong Association |
| | rs1801198 | | | | (High need) |
| | | | G:C | 1 | Medium Association |
| | | | | | (Medium need) |
| | | | C:C | 0 | No Association |
| | | | | | (Normal need) |
| | Total % | 100% | | | |
| Selenium | GPX1 | 100% | T:T | 2 | Strong Association |
| | rs1050450 | | | | (High selenium need) |
| | | | T:C | 2 | Strong Association |
| | | | | | (High selenium need) |
| | | | C:C | 0 | No Association |
| | | | | | (Normal selenium |
| | | | | | need) |
| | Total % | 100% | | | |

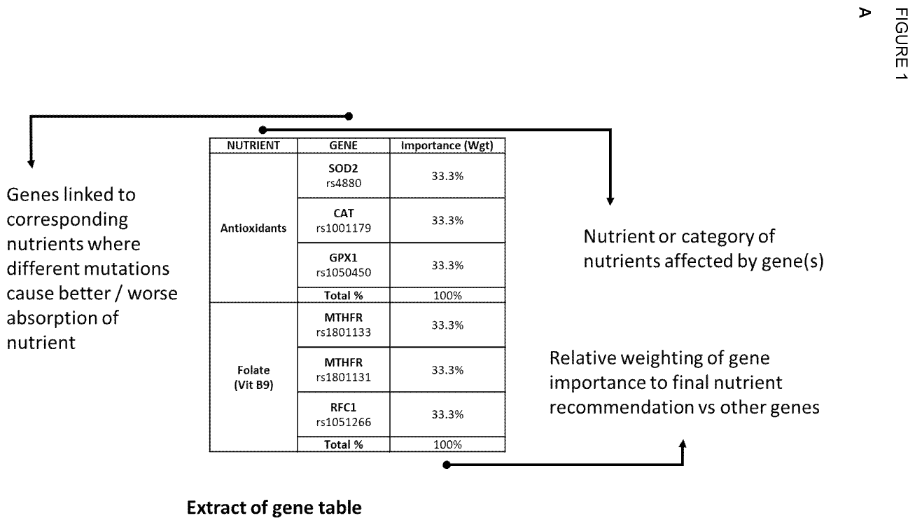

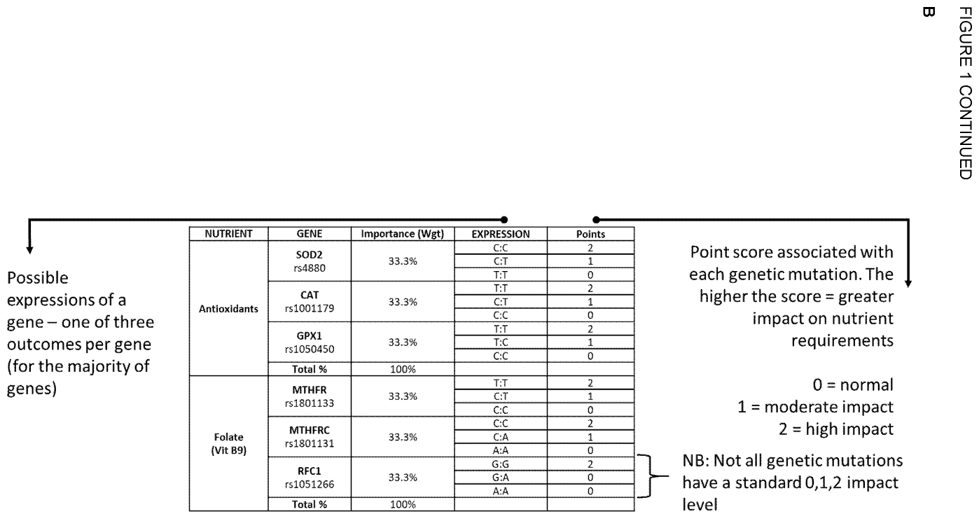

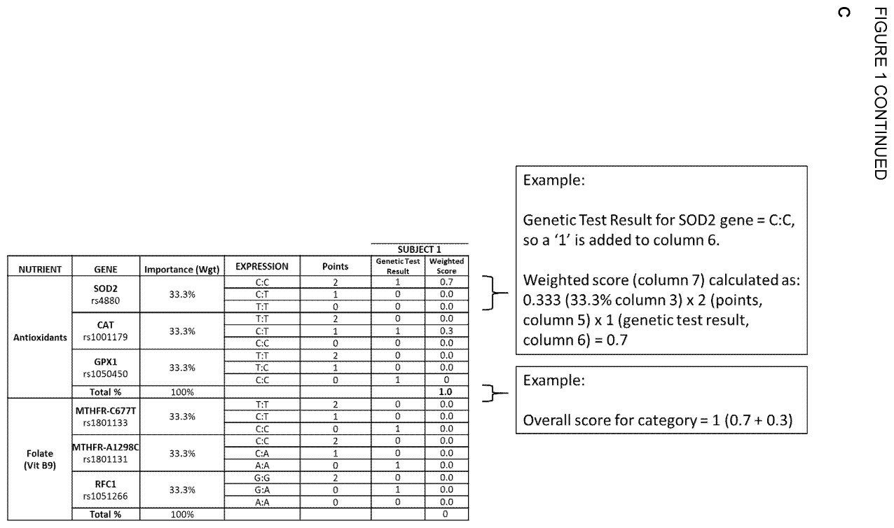

In more detail, the higher score values above are associated with a stronger association between the sequence and the corresponding effect and vice versa. The score thus indicates the level of influence of a particular SNP profile (or insertion/deletion for ACE1) on nutrient need (see FIGS. 1A and B).

The subjects' sequences (genetic profile) were compared to the table above and a ‘Genetic Test Result’ value of 1 was placed in the row corresponding to the particular sequence in the table above. A row in the above table relating to a specific SNP (e.g. C:C for BCO1 rs7501331, which has a strong association with vitamin A) or insertion or deletion profile for ACE1 may be considered a genetic reference standard. A combination of each possible SNP for a gene (e.g. C:C, T:C, and T:T for BCO1 rs7501331) may be considered a set of genetic reference standards.

A weighted score was then calculated for each gene. This was calculated as follows:

$$Weighted score = importance \% \times point score \times genetic test result$$

The total weighted score for a category (e.g. antioxidants) was calculated by adding together the weighted scores for each gene (e.g. SOD2, CAT, and GPX1) (see FIG. 1C).

The total weighted scores were then compared to the tables below for each nutrient, and a nutritional requirement assigned based on the score. FIG. 1D explains the process in more detail.

TABLE 3
Vitamin A need

| Base | Medium | High |
|---|---|---|
| 0 | >0-<1.5 | 1.5+ |

TABLE 4

| | |
|---|---|
| Antioxidants | Selenium |
| Vitamins A, C, and E Need | Need |

| Base | Medium | High | Base | High |
|---|---|---|---|---|
| 0 | >0-<0.7 | 0.7+ | 0 | >0 |

TABLE 5

| Vitamin | | | Folate | | | Vitamin | | |
|---|---|---|---|---|---|---|---|---|
| B6 Need | | | (Vit B9) Need | | | B12 Need | | |
| Base | Medium | High | Base | Medium | High | Base | Medium | High |
| 0 | >0-<2 | 2 | 0 | >0-<0.7 | 0.7+ | 0 | >0-<2 | 2 |

TABLE 6

| Vitamin D Need | | | Calcium Need | | |
|---|---|---|---|---|---|
| Base | Medium | High | Base | Med | High |
| 0 | >0-<1.5 | 1.5+ | 0 | >0-<2 | 2 |

TABLE 7

| Carb | | | Caffeine | | | Lactose | |
|---|---|---|---|---|---|---|---|
| Sensitivity | | | Sensitivity | | | Sensitivity | |
| Base/ | | | Base/ | | | Base/ | |
| Normal | Med | High | Normal | Med | High | Normal | Med |
| >0-<0.67 | 0.67-<1 | 1+ | 0 | >0-<2 | 2 | 0 | >0 |

TABLE 8

| | |
|---|---|
| Fat Sensitivity | Gluten Sensitivity |

| Base/Normal | Med | High | Base/Normal | Med |
|---|---|---|---|---|
| >0-<0.7 | 0.7-<1 | 1+ | 0 | >0 |

Reference to medium, and high in the tables above refers to the extent of the increase over the base/normal (e.g. EU NRV) level of the nutrient.

Although vitamin A need was provided by the antioxidant gene category, this need was modified by vitamin A need dictated by the gene BCO1. Thus, where a vitamin A need determined by the antioxidant genes (CAT, SOD2, and GPX1) contradicted that determined by BCO1, it was determined that the composition should include an amount of vitamin A consistent with the need as determined by BCO1 only.

### Example 2

### Generation of Archetypes & Definition of the Nutritional Requirements Thereof

A process was devised in which correlations (and strengths thereof) between the various nutritional requirements were determined, based on a panel of genes from 181 customer samples.

The correlations were then ranked from strongest to weakest. The total number of subjects were divided based on different nutritional requirements to create a segmentation map where only branches with a significantly large group were followed through into sub-branches and outliers were ignored. Each branch of the map represented a set of unique nutritional requirements that were used as the baseline for creating the archetypes. Each branch had the requirement for either a Normal (e.g. EU NRV) level, Medium or High increase amount for each nutrient, depending on the underlying genetic variation. The Normal, Medium and High levels for each individual nutrient were determined by an internal panel of experts in nutrigenetics, nutrigenomics, nutrition and microbiology and through a combination of scientific literature review, nutritional guideline review and expert opinion.

The base/normal level of each nutrient was determined with reference to the European Food Safety Authority (EFSA) Dietary Reference Value (DRV) guidelines for Adults and setting each nutrient at 100% (see https://www.efsa.europa.eu/en/topics/topic/dietary-reference-values).

Medium and high levels were based on known proteomic and transcriptomic analysis of different genetic studies (e.g. see Khalid M. Al-Batayneh et al., Homologous G776G Variant of Transcobalamin-II Gene is Linked to Vitamin B12 Deficiency, International Journal for Vitamin and Nutrition Research (2020) 90:1-2, 151-155).

Notably, each of the SNPs (and/or insertion/deletion for the ACE gene) have been well characterised (e.g. see SNPedia details under each accession no.). Care was taken to ensure that no nutrient level exceeded 50% of the tolerable upper limit for that nutrient. Using the determined Normal, Medium and High levels for each nutrient, each segment was converted into an archetype of 30 nutrients, each with a unique nutritional profile (i.e. no two archetypes have exactly the same level of nutrient for every nutrient). Each archetype was then applied to a broader dataset of 934 customer genetic profiles. Archetypes with the lowest number of allocated subjects were then eliminated sequentially (as after each elimination subjects were reallocated to the next most suitable archetype by the algorithm, changing the lowest performers). Archetype nutrient levels were then adjusted to achieve economically viable subject distribution whilst maintaining high accuracy of fit to subject needs (100%=perfect archetype match to customer needs). 17 micronutrient archetypes were successfully derived, achieving a minimum fit of 65% and minimum subject share of 3%. Archetypes were then validated for individual uniqueness to ensure no archetypes overlapped.

Each archetype may be considered to be a nutritional reference standard. In conclusion, using a normal distribution curve, archetypes were prepared that captured the nutritional requirements of a majority of subjects tested and excluded outliers.

### Example 3

### Generation of Exemplary Nutritional Compositions for Each Archetype

A number of micronutrients were found to differ between archetypes, these were vitamin E, vitamin A, ascorbic acid (vitamin C), selenium, folic acid, cyanocobalamin (vitamin B12), cholecalciferol (vitamin D3), calcium, and pyridoxine.

As explained above, the % change in nutrients was based on the requirement to compensate for subjects' nutrient processing and metabolising efficiencies/inefficiencies based on their genetic profile. The “normal” processing that is assumed in nutritional guidelines (e.g. EU Nutrient Reference Values (NRVs)), was therefore proportionately increased to account for each subjects' processing and metabolising efficiencies/inefficiencies to ensure they get a sufficient supply of essential nutrients. The levels required for each archetype and their deviation from the EU NRVs are presented in the tables below for the key variable micronutrients.

TABLE 9

| | | Archetype 1 | | Archetype 2 | | Archetype 3 | |
|---|---|---|---|---|---|---|---|
| | EU NRV | Require- | | Require- | | Require- | |
| Nutrient | (per 70 g) | ment | % NRV | ment | % NRV | ment | % NRV |
| Vitamin E | 12 | Med | 800% | Med | 810% | Med | 820% |
| (mg) | | | | | | | |
| Vit A (ug) | 800 | Med | 130% | High | 160% | Med | 133% |
| Ascorbic | 80 | Med | 350% | Med | 360% | Med | 370% |
| Acid (Vit | | | | | | | |
| C) (mg) | | | | | | | |
| Selenium | 55 | High | 165% | Base | 120% | Base | 125% |
| (ug) | | | | | | | |
| Folic Acid | 200 | Base | 120% | Base | 125% | High | 205% |
| (B9) (ug) | | | | | | | |
| Cyanocobalamin | 2.5 | Med | 350% | Med | 370% | High | 650% |
| (B12) (ug) | | | | | | | |
| Cholecalciferol | 5 | High | 600% | Med | 400% | High | 615% |
| (D3) (ug) | | | | | | | |
| Calcium | 800 | Base | 110% | Base | 112% | Med | 125% |
| (mg) | | | | | | | |
| Pyridoxine | 1.4 | Med | 350% | Med | 370% | High | 650% |
| (B6) (mg) | | | | | | | |

TABLE 10

| | | Archetype 4 | | Archetype 5 | | Archetype 6 | |
|---|---|---|---|---|---|---|---|
| | EU NRV | Require- | | Require- | | Require- | |
| Nutrient | (per 70 g) | ment | % NRV | ment | % NRV | ment | % NRV |
| Vitamin E | 12 | Med | 830% | High | 1300% | Med | 840% |
| (mg) | | | | | | | |
| Vit A (ug) | 800 | High | 163% | High | 166% | Med | 136% |
| Ascorbic | 80 | Med | 380% | High | 500% | Med | 390% |
| Acid (Vit | | | | | | | |
| C) (mg) | | | | | | | |
| Selenium | 55 | Base | 130% | High | 170% | High | 175% |
| (ug) | | | | | | | |
| Folic Acid | 200 | Base | 130% | Base | 135% | Med | 150% |
| B9) (ug) | | | | | | | |
| Cyanocobalamin | 2.5 | Med | 410% | High | 670% | High | 690% |
| (B12) (ug) | | | | | | | |
| Cholecalciferol | 5 | High | 630% | High | 645% | Base | 180% |
| (D3) (ug) | | | | | | | |
| Calcium | 800 | High | 145% | Med | 127% | Base | 114% |
| (mg) | | | | | | | |
| Pyridoxine | 1.4 | Med | 410% | High | 670% | High | 690% |
| (B6) (mg) | | | | | | | |

TABLE 11

| | | Archetype 7 | | Archetype 8 | | Archetype 9 | |
|---|---|---|---|---|---|---|---|
| | EU NRV | Require- | | Require- | | Require- | |
| Nutrient | (per 70 g) | ment | % NRV | ment | % NRV | ment | % NRV |
| Vitamin E | 12 | High | 1310% | Med | 850% | Base | 130% |
| (mg) | | | | | | | |
| Vit A (ug) | 800 | Med | 139% | Med | 142% | Med | 145% |
| Ascorbic | 80 | High | 510% | Med | 400% | Base | 200% |
| Acid (Vit | | | | | | | |
| C) (mg) | | | | | | | |
| Selenium | 55 | High | 180% | High | 185% | Base | 135% |
| (ug) | | | | | | | |
| Folic Acid | 200 | Med | 155% | Med | 160% | Med | 165% |
| (B9) (ug) | | | | | | | |
| Cyanocobalamin | 2.5 | Base | 200% | Med | 430% | Med | 450% |
| (B12) (ug) | | | | | | | |
| Cholecalciferol | 5 | Med | 415% | Base | 195% | Med | 430% |
| (D3) (ug) | | | | | | | |
| Calcium | 800 | Med | 129% | Med | 131% | Med | 133% |
| (mg) | | | | | | | |
| Pyridoxine | 1.4 | Base | 200% | Med | 430% | Med | 450% |
| (B6) (mg) | | | | | | | |

TABLE 12

| | | Archetype 10 | | Archetype 11 | | Archetype 12 | |
|---|---|---|---|---|---|---|---|
| | EU NRV | Require- | | Require- | | Require- | |
| Nutrient | (per 70 g) | ment | % NRV | ment | % NRV | ment | % NRV |
| Vitamin E | 12 | High | 1320% | High | 1330% | High | 1340% |
| (mg) | | | | | | | |
| Vit A (ug) | 800 | High | 169% | High | 172% | Med | 148% |
| Ascorbic | 80 | High | 520% | High | 530% | High | 540% |
| Acid (Vit | | | | | | | |
| C) (mg) | | | | | | | |
| Selenium | 55 | High | 190% | Base | 140% | High | 195% |
| (ug) | | | | | | | |
| Folic Acid | 200 | Base | 140% | Med | 170% | Med | 175% |
| (B9) (ug) | | | | | | | |
| Cyanocobalamin | 2.5 | Med | 470% | Med | 490% | Med | 510% |
| (B12) (ug) | | | | | | | |
| Cholecalciferol | 5 | Med | 445% | Med | 460% | Base | 210% |
| (D3) (ug) | | | | | | | |
| Calcium | 800 | Med | 135% | Med | 137% | Base | 116% |
| (mg) | | | | | | | |
| Pyridoxine | 1.4 | Med | 470% | Med | 490% | Med | 510% |
| (B6) (mg) | | | | | | | |

TABLE 13

| | | Archetype 13 | | Archetype 14 | | Archetype 15 | |
|---|---|---|---|---|---|---|---|
| | EU NRV | Require- | | Require- | | Require- | |
| Nutrient | (per 70 g) | ment | % NRV | ment | % NRV | ment | % NRV |
| Vitamin E | 12 | Med | 870% | High | 1350% | High | 1360% |
| (mg) | | | | | | | |
| Vit A (ug) | 800 | High | 175% | Med | 151% | High | 178% |
| Ascorbic | 80 | Med | 410% | High | 550% | High | 560% |
| Acid (Vit | | | | | | | |
| C) (mg) | | | | | | | |
| Selenium | 55 | Base | 145% | High | 200% | Base | 150% |
| (ug) | | | | | | | |
| Folic Acid | 200 | High | 210% | Med | 180% | Med | 185% |
| (B9) (ug) | | | | | | | |
| Cyanocobalamin | 2.5 | Base | 220% | High | 720% | Base | 240% |
| (B12) (ug) | | | | | | | |
| Cholecalciferol | 5 | Med | 475% | Med | 490% | Base | 225% |
| (D3) (ug) | | | | | | | |
| Calcium | 800 | Med | 139% | Med | 141% | Base | 118% |
| (mg) | | | | | | | |
| Pyridoxine | 1.4 | Base | 220% | High | 720% | Base | 240% |
| (B6) (mg) | | | | | | | |

TABLE 14

| | EU NRV | Archetype 16 | | Archetype 17 | |
|---|---|---|---|---|---|
| Nutrient | (per 70 g) | Requirement | % NRV | Requirement | % NRV |
| Vitamin E (mg) | 12 | Med | 880% | High | 1370% |
| Vit A (ug) | 800 | Med | 154% | Med | 157% |
| Ascorbic Acid | 80 | Med | 420% | High | 570% |
| (Vit C) (mg) | | | | | |
| Selenium (ug) | 55 | High | 205% | Base | 155% |
| Folic Acid (B9) | 200 | Med | 190% | Med | 195% |
| (ug) | | | | | |
| Cyanocobalamin | 2.5 | Base | 260% | Med | 530% |
| (B12) (ug) | | | | | |
| Cholecalciferol | 5 | Base | 240% | High | 660% |
| (D3) (ug) | | | | | |
| Calcium (mg) | 800 | Base | 120% | High | 147% |
| Pyridoxine (B6) | 1.4 | Base | 260% | Med | 530% |
| (mg) | | | | | |

A summary is provided in the table below:

TABLE 15

| | | ARCHETYPE NUTRIENT RANGES AS % NRV | | | | | |
|---|---|---|---|---|---|---|---|
| | NRV | Base | | Med | | High | |
| Range | (per 70 g) | Bottom | Top | Bottom | Top | Bottom | Top |
| Vit A (ug) | 800 | 115% | 125% | 130% | 157% | 160% | 178% |
| Pyridoxine | 1.4 | 200% | 260% | 350% | 530% | 650% | 720% |
| (B6) (mg) | | | | | | | |
| Folic Acid | 200 | 120% | 140% | 150% | 195% | 205% | 210% |
| (B9) (ug) | | | | | | | |
| Cyanocobalamin | 2.5 | 200% | 260% | 350% | 530% | 650% | 720% |
| (B12) (ug) | | | | | | | |
| Acorbic Acid | 80 | 200% | 200% | 350% | 420% | 500% | 570% |
| (Vit C) (mg) | | | | | | | |
| Cholecalciferol | 5 | 180% | 240% | 400% | 490% | 600% | 660% |
| (D3) (ug) | | | | | | | |
| Vitamin E (mg) | 12 | 130% | 130% | 800% | 880% | 1300% | 1370% |
| Calcium (mg) | 800 | 110% | 120% | 125% | 141% | 145% | 147% |

| | | | | | | |
|---|---|---|---|---|---|---|
| Selenium (ug) | 55 | 120% | 155% | N/A | 165% | 205% |

### Base Micronutrient Composition

Based on the above, complete nutritional compositions were prepared tailored to suit the nutritional needs of each archetype. The “base” composition below shows the amount of each non-varying micronutrient and highlights those that vary between archetypes (“variable”).

TABLE 16

| | | | |
|---|---|---|---|
| | EU NRV | | Amount |
| Nutrient | (per day) | % NRV | per day |

| | | |
|---|---|---|
| Vitamin A (RE) (ug) | 800 | VARIABLE |

| | | | |
|---|---|---|---|
| Thiamin (B1) (mg) | 1.1 | 108% | 1.2 |
| Riboflavin (B2) (mg) | 1.4 | 100% | 1.4 |
| Nicotinic acid (B3) (mg) | 16 | 112% | 17.9 |
| Pantothenic Acid (B5) (mg) | 6 | 100% | 6.0 |

| | | |
|---|---|---|
| Pyridoxine (B6) (mg) | 1.4 | VARIABLE |

| | | | |
|---|---|---|---|
| Biotin (B7) (ug) | 50 | 100% | 50.0 |

| | | |
|---|---|---|
| Folic Acid (B9) (ug) | 200 | VARIABLE |

| | | | |
|---|---|---|---|
| PABA (B10) (mg) | — * | — | 25.0 |

| | | |
|---|---|---|
| Cyanocobalamin (B12) (ug) | 2.5 | VARIABLE |
| Ascorbic Acid (Vit C) (mg) | 80 | VARIABLE |
| Cholecalciferol (D3) (ug) | 5 | VARIABLE |
| Vitamin E (d-a-TE) (mg) | 12 | VARIABLE |

| | | | |
|---|---|---|---|
| Vitamin K1 (ug) | 75 | 187% | 140.3 |
| Inositol (mg) | — * | — | 30.0 |
| Choline (Vitamin J) (mg) | 400 | 137% | 548.0 |
| Chloride (mg) | 800 | 100% | 800.0 |
| Chromium (ug) | 40 | 100% | 40.0 |

| | | |
|---|---|---|
| Calcium (mg) | 800 | VARIABLE |
| Selenium (ug) | 55 | VARIABLE |

| | | | |
|---|---|---|---|
| Phosphorus (mg) | 700 | 100% | 700.0 |
| lodine (ug) | 150 | 100% | 150.0 |
| Iron (ug) | 14 | 100% | 14.0 |
| Molybdenum (ug) | 50 | 100% | 50.0 |
| Manganese (ug) | 2 | 116% | 2.3 |
| Magnesium (mg) | 375 | 100% | 375.0 |
| Fluoride (mg) | 3.5 | 100% | 3.5 |
| Potassium (mg) | 2,000 | 15% | 599.9 |
| Copper (mg) | 1 | 100% | 1.0 |
| Zinc (mg) | 10 | 110% | 11.0 |

* EU NRV not established

Subsequent tables show the amounts of the ‘variable’ micronutrient compositions for each archetype corresponding to that determined using the methodology described in Example 2. The micronutrient nutritional compositions for each archetype further included the above non-varying (base) micronutrients.

TABLE 17
Archetype 1 Variable Micronutrient Composition

| | EU NRV | | Amount |
|---|---|---|---|
| Nutrient | (per day) | % NRV | per day |
| Vitamin A (RE) (ug) | 800 | 130 | 1040 |
| Pyridoxine (B6) (mg) | 1.4 | 350 | 5 |
| Folic Acid (B9) (ug) | 200 | 120 | 240 |
| Cyanocobalamin (B12) (ug) | 2.5 | 350 | 9 |
| Ascorbic Acid (Vit C) (mg) | 80 | 350 | 280 |
| Cholecalciferol (D3) (ug) | 5 | 600 | 30 |
| Vitamin E (d-a-TE) (mg) | 12 | 800 | 96 |
| Calcium (mg) | 800 | 110 | 880 |
| Selenium (ug) | 55 | 165 | 91 |

TABLE 18
Archetype 2 Variable Micronutrient Composition

| | EU NRV | | Amount |
|---|---|---|---|
| Nutrient | (per day) | % NRV | per day |
| Vitamin A (RE) (ug) | 800 | 160 | 1280 |
| Pyridoxine (B6) (mg) | 1.4 | 370 | 5 |
| Folic Acid (B9) (ug) | 200 | 125 | 250 |
| Cyanocobalamin (B12) (ug) | 2.5 | 370 | 9 |
| Ascorbic Acid (Vit C) (mg) | 80 | 360 | 288 |
| Cholecalciferol (D3) (ug) | 5 | 400 | 20 |
| Vitamin E (d-a-TE) (mg) | 12 | 810 | 97 |
| Calcium (mg) | 800 | 112 | 896 |
| Selenium (ug) | 55 | 120 | 66 |

TABLE 19
Archetype 3 Variable Micronutrient Composition

| | EU NRV | | Amount |
|---|---|---|---|
| Nutrient | (per day) | % NRV | per day |
| Vitamin A (RE) (ug) | 800 | 133 | 1064 |
| Pyridoxine (B6) (mg) | 1.4 | 650 | 9 |
| Folic Acid (B9) (ug) | 200 | 205 | 410 |
| Cyanocobalamin (B12) (ug) | 2.5 | 650 | 16 |
| Ascorbic Acid (Vit C) (mg) | 80 | 370 | 296 |
| Cholecalciferol (D3) (ug) | 5 | 615 | 31 |
| Vitamin E (d-a-TE) (mg) | 12 | 820 | 98 |
| Calcium (mg) | 800 | 125 | 1000 |
| Selenium (ug) | 55 | 125 | 69 |

TABLE 20
Archetype 4 Variable Micronutrient Composition

| | EU NRV | | Amount |
|---|---|---|---|
| Nutrient | (per day) | % NRV | per day |
| Vitamin A (RE) (ug) | 800 | 163 | 1304 |
| Pyridoxine (B6) (mg) | 1.4 | 410 | 6 |
| Folic Acid (B9) (ug) | 200 | 130 | 260 |
| Cyanocobalamin (B12) (ug) | 2.5 | 410 | 10 |
| Ascorbic Acid (Vit C) (mg) | 80 | 380 | 304 |
| Cholecalciferol (D3) (ug) | 5 | 630 | 32 |
| Vitamin E (d-a-TE) (mg) | 12 | 830 | 100 |
| Calcium (mg) | 800 | 145 | 1160 |
| Selenium (ug) | 55 | 130 | 72 |

TABLE 21
Archetype 5 Variable Micronutrient Composition

| | EU NRV | | Amount |
|---|---|---|---|
| Nutrient | (per day) | % NRV | per day |
| Vitamin A (RE) (ug) | 800 | 166 | 1328 |
| Pyridoxine (B6) (mg) | 1.4 | 670 | 9 |
| Folic Acid (B9) (ug) | 200 | 135 | 270 |
| Cyanocobalamin (B12) (ug) | 2.5 | 670 | 17 |
| Ascorbic Acid (Vit C) (mg) | 80 | 500 | 400 |
| Cholecalciferol (D3) (ug) | 5 | 645 | 32 |
| Vitamin E (d-a-TE) (mg) | 12 | 1300 | 156 |
| Calcium (mg) | 800 | 127 | 1016 |
| Selenium (ug) | 55 | 170 | 94 |

TABLE 22
Archetype 6 Variable Micronutrient Composition

| | EU NRV | | Amount |
|---|---|---|---|
| Nutrient | (per day) | % NRV | per day |
| Vitamin A (RE) (ug) | 800 | 136 | 1088 |
| Pyridoxine (B6) (mg) | 1.4 | 690 | 10 |
| Folic Acid (B9) (ug) | 200 | 150 | 300 |
| Cyanocobalamin (B12) (ug) | 2.5 | 690 | 17 |
| Ascorbic Acid (Vit C) (mg) | 80 | 390 | 312 |
| Cholecalciferol (D3) (ug) | 5 | 180 | 9 |
| Vitamin E (d-a-TE) (mg) | 12 | 840 | 101 |
| Calcium (mg) | 800 | 114 | 912 |
| Selenium (ug) | 55 | 175 | 96 |

TABLE 23
Archetype 7 Variable Micronutrient Composition

| | EU NRV | | Amount |
|---|---|---|---|
| Nutrient | (per day) | % NRV | per day |
| Vitamin A (RE) (ug) | 800 | 139 | 1112 |
| Pyridoxine (B6) (mg) | 1.4 | 200 | 3 |
| Folic Acid (B9) (ug) | 200 | 155 | 310 |
| Cyanocobalamin (B12) (ug) | 2.5 | 200 | 5 |
| Ascorbic Acid (Vit C) (mg) | 80 | 510 | 408 |
| Cholecalciferol (D3) (ug) | 5 | 415 | 21 |
| Vitamin E (d-a-TE) (mg) | 12 | 1310 | 157 |
| Calcium (mg) | 800 | 129 | 1032 |
| Selenium (ug) | 55 | 180 | 99 |

TABLE 24
Archetype 8 Variable Micronutrient Composition

| | EU NRV | | Amount |
|---|---|---|---|
| Nutrient | (per day) | % NRV | per day |
| Vitamin A (RE) (ug) | 800 | 142 | 1136 |
| Pyridoxine (B6) (mg) | 1.4 | 430 | 6 |
| Folic Acid (B9) (ug) | 200 | 160 | 320 |
| Cyanocobalamin (B12) (ug) | 2.5 | 430 | 11 |
| Ascorbic Acid (Vit C) (mg) | 80 | 400 | 320 |
| Cholecalciferol (D3) (ug) | 5 | 195 | 10 |
| Vitamin E (d-a-TE) (mg) | 12 | 850 | 102 |
| Calcium (mg) | 800 | 131 | 1048 |
| Selenium (ug) | 55 | 185 | 102 |

TABLE 25
Archetype 9 Variable Micronutrient Composition

| | EU NRV | | Amount |
|---|---|---|---|
| Nutrient | (per day) | % NRV | per day |
| Vitamin A (RE) (ug) | 800 | 145 | 1160 |
| Pyridoxine (B6) (mg) | 1.4 | 450 | 6 |
| Folic Acid (B9) (ug) | 200 | 165 | 330 |
| Cyanocobalamin (B12) (ug) | 2.5 | 450 | 11 |
| Ascorbic Acid (Vit C) (mg) | 80 | 200 | 160 |
| Cholecalciferol (D3) (ug) | 5 | 430 | 22 |
| Vitamin E (d-a-TE) (mg) | 12 | 130 | 16 |
| Calcium (mg) | 800 | 133 | 1064 |
| Selenium (ug) | 55 | 135 | 74 |

TABLE 26
Archetype 10 Variable Micronutrient Composition

| | EU NRV | | Amount |
|---|---|---|---|
| Nutrient | (per day) | % NRV | per day |
| Vitamin A (RE) (ug) | 800 | 169 | 1352 |
| Pyridoxine (B6) (mg) | 1.4 | 470 | 7 |
| Folic Acid (B9) (ug) | 200 | 140 | 280 |
| Cyanocobalamin (B12) (ug) | 2.5 | 470 | 12 |
| Ascorbic Acid (Vit C) (mg) | 80 | 520 | 416 |
| Cholecalciferol (D3) (ug) | 5 | 445 | 22 |
| Vitamin E (d-a-TE) (mg) | 12 | 1320 | 158 |
| Calcium (mg) | 800 | 135 | 1080 |
| Selenium (ug) | 55 | 190 | 105 |

TABLE 27
Archetype 11 Variable Micronutrient Composition

| | EU NRV | | Amount |
|---|---|---|---|
| Nutrient | (per day) | % NRV | per day |
| Vitamin A (RE) (ug) | 800 | 172 | 1376 |
| Pyridoxine (B6) (mg) | 1.4 | 490 | 7 |
| Folic Acid (B9) (ug) | 200 | 170 | 340 |
| Cyanocobalamin (B12) (ug) | 2.5 | 490 | 12 |
| Ascorbic Acid (Vit C) (mg) | 80 | 530 | 424 |
| Cholecalciferol (D3) (ug) | 5 | 460 | 23 |
| Vitamin E (d-a-TE) (mg) | 12 | 1330 | 160 |
| Calcium (mg) | 800 | 137 | 1096 |
| Selenium (ug) | 55 | 140 | 77 |

TABLE 28
Archetype 12 Variable Micronutrient Composition

| | EU NRV | | Amount |
|---|---|---|---|
| Nutrient | (per day) | % NRV | per day |
| Vitamin A (RE) (ug) | 800 | 148 | 1184 |
| Pyridoxine (B6) (mg) | 1.4 | 510 | 7 |
| Folic Acid (B9) (ug) | 200 | 175 | 350 |
| Cyanocobalamin (B12) (ug) | 2.5 | 510 | 13 |
| Ascorbic Acid (Vit C) (mg) | 80 | 540 | 432 |
| Cholecalciferol (D3) (ug) | 5 | 210 | 11 |
| Vitamin E (d-a-TE) (mg) | 12 | 1340 | 161 |
| Calcium (mg) | 800 | 116 | 928 |
| Selenium (ug) | 55 | 195 | 107 |

TABLE 29
Archetype 13 Variable Micronutrient Composition

| | EU NRV | | Amount |
|---|---|---|---|
| Nutrient | (per day) | % NRV | per day |
| Vitamin A (RE) (ug) | 800 | 175 | 1400 |
| Pyridoxine (B6) (mg) | 1.4 | 220 | 3 |
| Folic Acid (B9) (ug) | 200 | 210 | 420 |
| Cyanocobalamin (B12) (ug) | 2.5 | 220 | 6 |
| Ascorbic Acid (Vit C) (mg) | 80 | 410 | 328 |
| Cholecalciferol (D3) (ug) | 5 | 475 | 24 |
| Vitamin E (d-a-TE) (mg) | 12 | 870 | 104 |
| Calcium (mg) | 800 | 139 | 1112 |
| Selenium (ug) | 55 | 145 | 80 |

TABLE 30
Archetype 14 Variable Micronutrient Composition

| | EU NRV | | Amount |
|---|---|---|---|
| Nutrient | (per day) | % NRV | per day |
| Vitamin A (RE) (ug) | 800 | 151 | 1208 |
| Pyridoxine (B6) (mg) | 1.4 | 720 | 10 |
| Folic Acid (B9) (ug) | 200 | 180 | 360 |
| Cyanocobalamin (B12) (ug) | 2.5 | 720 | 18 |
| Ascorbic Acid (Vit C) (mg) | 80 | 550 | 440 |
| Cholecalciferol (D3) (ug) | 5 | 490 | 25 |
| Vitamin E (d-a-TE) (mg) | 12 | 1350 | 162 |
| Calcium (mg) | 800 | 141 | 1128 |
| Selenium (ug) | 55 | 200 | 110 |

TABLE 31
Archetype 15 Variable Micronutrient Composition

| | EU NRV | | Amount |
|---|---|---|---|
| Nutrient | (per day) | % NRV | per day |
| Vitamin A (RE) (ug) | 800 | 178 | 1424 |
| Pyridoxine (B6) (mg) | 1.4 | 240 | 3 |
| Folic Acid (B9) (ug) | 200 | 185 | 370 |
| Cyanocobalamin (B12) (ug) | 2.5 | 240 | 6 |
| Ascorbic Acid (Vit C) (mg) | 80 | 560 | 448 |
| Cholecalciferol (D3) (ug) | 5 | 225 | 11 |
| Vitamin E (d-a-TE) (mg) | 12 | 1360 | 163 |
| Calcium (mg) | 800 | 118 | 944 |
| Selenium (ug) | 55 | 150 | 83 |

TABLE 32
Archetype 16 Variable Micronutrient Composition

| | EU NRV | | Amount |
|---|---|---|---|
| Nutrient | (per day) | % NRV | per day |
| Vitamin A (RE) (ug) | 800 | 154 | 1232 |
| Pyridoxine (B6) (mg) | 1.4 | 260 | 4 |
| Folic Acid (B9) (ug) | 200 | 190 | 380 |
| Cyanocobalamin (B12) (ug) | 2.5 | 260 | 7 |
| Ascorbic Acid (Vit C) (mg) | 80 | 420 | 336 |
| Cholecalciferol (D3) (ug) | 5 | 240 | 12 |
| Vitamin E (d-a-TE) (mg) | 12 | 880 | 106 |
| Calcium (mg) | 800 | 120 | 960 |
| Selenium (ug) | 55 | 205 | 113 |

TABLE 33
Archetype 17 Variable Micronutrient Composition

| | EU NRV | | Amount |
|---|---|---|---|
| Nutrient | (per day) | % NRV | per day |
| Vitamin A (RE) (ug) | 800 | 157 | 1256 |
| Pyridoxine (B6) (mg) | 1.4 | 530 | 7 |
| Folic Acid (B9) (ug) | 200 | 195 | 390 |
| Cyanocobalamin (B12) (ug) | 2.5 | 530 | 13 |
| Ascorbic Acid (Vit C) (mg) | 80 | 570 | 456 |
| Cholecalciferol (D3) (ug) | 5 | 660 | 33 |
| Vitamin E (d-a-TE) (mg) | 12 | 1370 | 164 |
| Calcium (mg) | 800 | 147 | 1176 |
| Selenium (ug) | 55 | 155 | 85 |

### Example 4

### Addition of a Macronutrient Component

Macronutrients are added to the micronutrient compositions of Example 3 to provide a complete nutritional composition.

The macronutrients are prepared in four different types:
1. Low fat and low carbohydrates;
2. High fat and low carbohydrates;
3. Medium fat and medium carbohydrates; and
4. Low fat and high carbohydrates.

### 1) Low Fat & Low Carbohydrates

A single serving size of 35 g of a complete nutritional composition is prepared by adding 2.2 g of an micronutrient composition corresponding to archetype 1, 2, 6, or 9, or 2.3 g of a micronutrient composition corresponding to archetype 3, 4, 8, 12, 13, 15, or 16, or 2.4 g of a micronutrient composition corresponding to archetype 5, 7, 10, 11, or 14, or 2.5 g of a micronutrient composition corresponding to archetype 17 (respectively) to:
22 g of protein;
1.1 g of carbohydrate;
4.1 g of fats; and
2.4 g of fibre.

Fillers and flavourings (e.g. salt) are added to bring the total to 35 g. The amount of micronutrient composition corresponds to 50% of a subject's daily requirement (i.e. 50% of that shown in Example 3). It is intended that 2×35 g of composition (i.e. 70 g total) are consumed daily for a subject to meet its optimal micronutrient requirements, together with desired macronutrient requirements.

### 2) High Fat & Low Carbohydrates

A single serving size of 50 g of a complete nutritional composition is prepared by adding 2.2 g of an micronutrient composition corresponding to archetype 1, 2, 6, or 9, or 2.3 g of a micronutrient composition corresponding to archetype 3, 4, 8, 12, 13, 15, or 16, or 2.4 g of a micronutrient composition corresponding to archetype 5, 7, 10, 11, or 14, or 2.5 g of a micronutrient composition corresponding to archetype 17 (respectively) to:
22 g of protein;
1.1 g of carbohydrate;
14.1 g of fats; and
6.2 g of fibre.

Fillers and flavourings (e.g. salt) are added to bring the total to 50 g. The amount of micronutrient composition corresponds to 50% of a subject's daily requirement. It is intended that 2×50 g of composition (i.e. 100 g total) are consumed daily for a subject to meet its optimal micronutrient requirements, together with desired macronutrient requirements.

### 3) Medium Fat & Medium Carbohydrates

A single serving size of 50 g of a complete nutritional composition is prepared by adding 2.2 g of an micronutrient composition corresponding to archetype 1, 2, 6, or 9, or 2.3 g of a micronutrient composition corresponding to archetype 3, 4, 8, 12, 13, 15, or 16, or 2.4 g of a micronutrient composition corresponding to archetype 5, 7, 10, 11, or 14, or 2.5 g of a micronutrient composition corresponding to archetype 17 (respectively) to:
23 g of protein;
7.8 g of carbohydrate;
7.9 g of fats; and
4.6 g of fibre.

Fillers and flavourings (e.g. salt) are added to bring the total to 50 g. The amount of micronutrient composition corresponds to 50% of a subject's daily requirement. It is intended that 2×50 g of composition (i.e. 100 g total) are consumed daily for a subject to meet its optimal micronutrient requirements, together with desired macronutrient requirements.

### 4) Low Fat & High Carbohydrates

A single serving size of 50 g of a complete nutritional composition is prepared by adding 2.2 g of an micronutrient composition corresponding to archetype 1, 2, 6, or 9, or 2.3 g of a micronutrient composition corresponding to archetype 3, 4, 8, 12, 13, 15, or 16, or 2.4 g of a micronutrient composition corresponding to archetype 5, 7, 10, 11, or 14, or 2.5 g of a micronutrient composition corresponding to archetype 17 (respectively) to:
23 g of protein;
11.1 g of carbohydrate;
5.0 g of fats; and
3.4 g of fibre.

Fillers and flavourings (e.g. salt) are added to bring the total to 50 g. The amount of micronutrient composition corresponds to 50% of a subject's daily requirement. It is intended that 2×50 g of composition (i.e. 100 g total) are consumed daily for a subject to meet its optimal micronutrient requirements, together with desired macronutrient requirements.

### Example 5

### Further Customisation of a Nutritional Composition Based on a Subject's Goals

The micronutrient nutritional compositions of the invention are further tailored based on a subject's particular lifestyle and/or goals.

Exemplary goals may be as follows:
1. Wellbeing—requires a low fat/low carb macronutrient composition;
2. Slimming/weight loss—requires a higher energy dependency on fats over carbs (high fat/low carb);
3. Achieving lean muscle—requires a higher energy dependency on fats over carbs (high fat/low carb);
4. Increasing endurance performance—requires balanced energy dependency to give a steady access to energy between slow burn carbs and fats (medium fat/medium carb); or

5. Increasing power performance—requires higher carbs to give access to rapid energy sources (high carb/low fat).

To achieve 1-5 above, instead of the optimal macronutrient composition for each archetype shown in Example 4, a different macronutrient composition from Example 4 is employed.

### Example 6

### Method for Determining a Subject's Nutritional Requirements

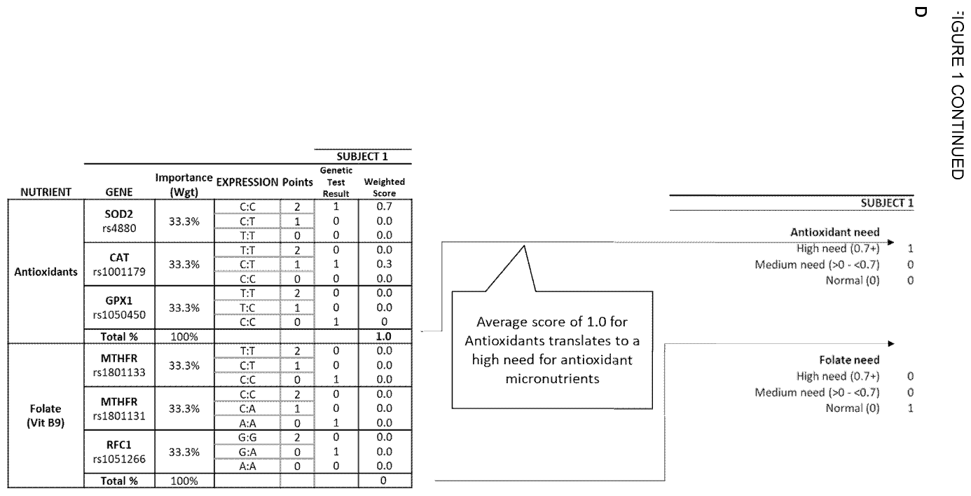

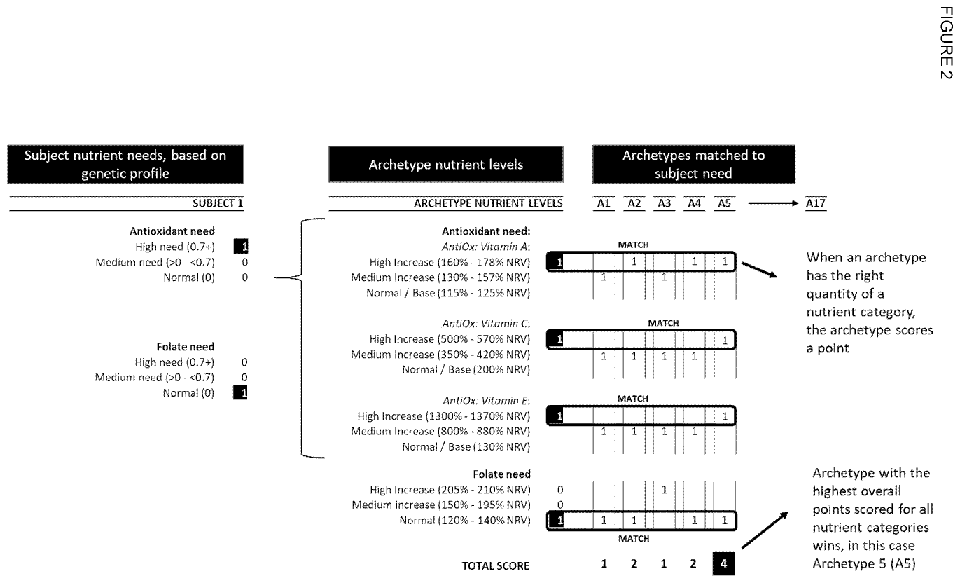

A subject's genetic profile is determined, total weighted scores calculated, and nutritional requirements determined in accordance with Example 1. Each of the subject's nutrient requirements (scored 1 in each category) are compared with the same nutrient requirements for each archetype (nutritional reference standard) and the highest scoring (i.e. most similar) archetype is considered to be the archetype of best fit for the subject. The nutritional composition corresponding to said archetype is considered to be the optimal composition for the subject. The process is explained in more detail in FIG. 2. The subject is assigned to Archetype 5 and consumes 70 g of a nutritional composition per day comprising the Archetype 5 micronutrient composition plus low carb and low fat macronutrient composition. The composition is prepared in single serving sizes by admixing 2.4 g of micronutrient composition with 32.6 g of a macronutrient composition (comprising 22 g of protein, 1.1 g of carbohydrate, 4.1 g of fats, and 2.4 g of fibre (plus fillers and/or flavourings)) to provide a 35 g serving size.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in in biochemistry, biotechnology, food technology, formulation chemistry or related fields are intended to be within the scope of the following claims.

1. A nutritional composition comprising one or more micronutrients, wherein an amount of the one or more micronutrients in the composition is based on a subject's nutritional requirement for the one or more micronutrients, wherein the subject's nutritional requirement for the one or more micronutrients has been determined using the subject's genetic profile, and wherein the one or more micronutrients comprise vitamin A, pyridoxine (vitamin B6), folic acid (vitamin B9), cyanocobalamin (vitamin B12), ascorbic acid (vitamin C), cholecalciferol (vitamin D3), vitamin E, calcium, and/or selenium.

2. A nutritional composition comprising one or more micronutrients, wherein the one or more micronutrients comprise vitamin A, pyridoxine (vitamin B6), folic acid (vitamin B9), cyanocobalamin (vitamin B12), ascorbic acid (vitamin C), cholecalciferol (vitamin D3), vitamin E, calcium, and/or selenium.

3. The composition according to claim 1 or 2, wherein the composition comprises at least a daily unit dose (e.g. two or more daily unit doses) of the one or more micronutrients or a fraction of a daily unit dose of the one or more micronutrients.

4. A kit comprising a nutritional composition, wherein the nutritional composition comprises one or more micronutrients, wherein the kit comprises:
(a) a container comprising at least a daily unit dose (e.g. two or more daily unit doses) of the one or more micronutrients; or
(b) a plurality of containers each comprising a fraction of the daily unit dose of the one or more micronutrients; and
(c) optionally instructions for use of the same;
wherein the kit comprises at least one daily unit dose of the one or more micronutrients, and wherein the one or more micronutrients comprise vitamin A, pyridoxine (vitamin B6), folic acid (vitamin B9), cyanocobalamin (vitamin B12), ascorbic acid (vitamin C), cholecalciferol (vitamin D3), vitamin E, calcium, and/or selenium.

5. The kit according to claim 4, wherein an amount of the one or more micronutrients (preferably all) in the composition is based on a subject's nutritional requirement for the one or more micronutrients (preferably all), wherein the subject's nutritional requirement for the one or more micronutrients (preferably all) has been determined using the subject's genetic profile.

6. The composition or kit according to any one of claims 3-5, wherein the daily unit dose of the one or more micronutrients comprises:
(a) vitamin A present at greater than 100% to 375% of a reference vitamin A daily dose, wherein the reference vitamin A daily dose is 800 μg;
(b) pyridoxine present at greater than 100% to 7143% of a reference pyridoxine daily dose, wherein the reference pyridoxine daily dose is 1.4 mg;
(c) folic acid present at greater than 100% to 500% of a reference folic acid daily dose, wherein the reference folic acid daily dose is 200 μg;
(d) cyanocobalamin present at greater than 100% to 40000% of a reference cyanocobalamin daily dose, wherein the reference cyanocobalamin daily dose is 2.5 μg;
(e) ascorbic acid present at greater than 100% to 2500% of a reference ascorbic acid daily dose, wherein the reference ascorbic acid daily dose is 80 mg;
(f) cholecalciferol present at greater than 100% to 2000% of a reference cholecalciferol daily dose, wherein the reference cholecalciferol daily dose is 5 μg;
(g) vitamin E present at greater than 100% to 9167% of a reference vitamin E daily dose, wherein the reference vitamin E daily dose is 12 mg;
(h) calcium present at greater than 100% to 313% of a reference calcium daily dose, wherein the reference calcium daily dose is 800 mg; and/or
(i) selenium present at greater than 100% to 727% of a reference selenium daily dose, wherein the reference selenium daily dose is 55 μg.

7. The composition or kit according to any one of claims 3-6, wherein the daily unit dose of the one or more micronutrients comprises:
(a) vitamin A present at: (i) greater than 100% to 127.5% of a reference vitamin A daily dose; (ii) greater than 127.5% to 158.5% of a reference vitamin A daily dose; or (iii) greater than 158.5% to 375% of a reference vitamin A daily dose, wherein the reference vitamin A daily dose is 800 μg;
(b) pyridoxine present at: (i) greater than 100% to 305% of a reference pyridoxine daily dose; (ii) greater than 305% to 590% of a reference pyridoxine daily dose; or (iii) greater than 590% to 7143% of a reference pyridoxine daily dose, wherein the reference pyridoxine daily dose is 1.4 mg;
(c) folic acid present at: (i) greater than 100% to 145% of a reference folic acid daily dose; (ii) greater than 145% to 200% of a reference folic acid daily dose; or (iii) greater than 200% to 500% of a reference folic acid daily dose, wherein the reference folic acid daily dose is 200 μg;
(d) cyanocobalamin present at: (i) greater than 100% to 305% of a reference cyanocobalamin daily dose; (ii) greater than 305% to 590% of a reference cyanocobalamin daily dose; or (iii) greater than 590% to 40000% of a reference cyanocobalamin daily dose, wherein the reference cyanocobalamin daily dose is 2.5 μg;
(e) ascorbic acid present at: (i) greater than 100% to 275% of a reference ascorbic acid daily dose; (ii) greater than 275% to 460% of a reference ascorbic acid daily dose; or (iii) greater than 460% to 2500% of a reference ascorbic acid daily dose, wherein the reference ascorbic acid daily dose is 80 mg;
(f) cholecalciferol present at: (i) greater than 100% to 320% of a reference cholecalciferol daily dose; (ii) greater than 320% to 545% of a reference cholecalciferol daily dose; or (iii) greater than 545% to 2000% of a reference cholecalciferol daily dose, wherein the reference cholecalciferol daily dose is 5 μg;
(g) vitamin E present at: (i) greater than 100% to 465% of a reference vitamin E daily dose; (ii) greater than 465% to 1090% of a reference vitamin E daily dose; or (iii) greater than 1090% to 9167% of a reference vitamin E daily dose, wherein the reference vitamin E daily dose is 12 mg;
(h) calcium present at: (i) greater than 100% to 122.5% of a reference calcium daily dose; (ii) greater than 122.5% to 143% of a reference calcium daily dose; or (iii) greater than 143% to 313% of a reference calcium daily dose, wherein the reference calcium daily dose is 800 mg; and/or
(i) selenium present at: (i) greater than 100% to 160% of a reference selenium daily dose; or (ii) greater than 160% to 727% of a reference selenium daily dose, wherein the reference selenium daily dose is 55 μg.

8. The composition or kit according to any one of claims 3-7, wherein the daily unit dose of the one or more micronutrients comprises:
(a) vitamin A present at: (i) 115% to 125% of a reference vitamin A daily dose; (ii) 130% to 157% of a reference vitamin A daily dose; or (iii) 160% to 178% of a reference vitamin A daily dose, wherein the reference vitamin A daily dose is 800 μg;
(b) pyridoxine present at: (i) 200% to 260% of a reference pyridoxine daily dose; (ii) 350% to 530% of a reference pyridoxine daily dose; or (iii) 650% to 720% of a reference pyridoxine daily dose, wherein the reference pyridoxine daily dose is 1.4 mg;
(c) folic acid present at: (i) 120% to 140% of a reference folic acid daily dose; (ii) 150% to 195% of a reference folic acid daily dose; or (iii) 205% to 210% of a reference folic acid daily dose, wherein the reference folic acid daily dose is 200 μg;
(d) cyanocobalamin present at: (i) 200% to 260% of a reference cyanocobalamin daily dose; (ii) 350% to 530% of a reference cyanocobalamin daily dose; or (iii) 650% to 720% of a reference cyanocobalamin daily dose, wherein the reference cyanocobalamin daily dose is 2.5 μg;
(e) ascorbic acid present at: (i) 190% to 210% (preferably 200%) of a reference ascorbic acid daily dose; (ii) 350% to 420% of a reference ascorbic acid daily dose; or (iii) 500% to 570% of a reference ascorbic acid daily dose, wherein the reference ascorbic acid daily dose is 80 mg;
(f) cholecalciferol present at: (i) 180% to 240% of a reference cholecalciferol daily dose; (ii) 400% to 490% of a reference cholecalciferol daily dose; or (iii) 600% to 660% of a reference cholecalciferol daily dose, wherein the reference cholecalciferol daily dose is 5 μg;
(g) vitamin E present at: (i) 125% to 135% (preferably 130%) of a reference vitamin E daily dose; (ii) 800% to 880% of a reference vitamin E daily dose; or (iii) 1300% to 1370% of a reference vitamin E daily dose, wherein the reference vitamin E daily dose is 12 mg;
(h) calcium present at: (i) 110% to 120% of a reference calcium daily dose; (ii) 125% to 141% of a reference calcium daily dose; or (iii) 145% to 147% of a reference calcium daily dose, wherein the reference calcium daily dose is 800 mg; and/or
(i) selenium present at: (i) 120% to 155% of a reference selenium daily dose; or (ii) 165% to 205% of a reference selenium daily dose, wherein the reference selenium daily dose is 55 μg.

9. The composition or kit according to any one of claims 3-8, wherein the daily unit dose of the one or more micronutrients comprises:
(a) vitamin A present at greater than 127.5% to 158.5% of a reference vitamin A daily dose, wherein the reference vitamin A daily dose is 800 μg; pyridoxine present at greater than 305% to 590% of a reference pyridoxine daily dose, wherein the reference pyridoxine daily dose is 1.4 mg; folic acid present at greater than 100% to 145% of a reference folic acid daily dose, wherein the reference folic acid daily dose is 200 μg; cyanocobalamin present at greater than 305% to 590% of a reference cyanocobalamin daily dose, wherein the reference cyanocobalamin daily dose is 2.5 μg; ascorbic acid present at greater than 275% to 460% of a reference ascorbic acid daily dose, wherein the reference ascorbic acid daily dose is 80 mg; cholecalciferol present at greater than 545% to 2000% of a reference cholecalciferol daily dose, wherein the reference cholecalciferol daily dose is 5 μg; vitamin E present at greater than 465% to 1090% of a reference vitamin E daily dose, wherein the reference vitamin E daily dose is 12 mg; calcium present at greater than 100% to 122.5% of a reference calcium daily dose, wherein the reference calcium daily dose is 800 mg; and/or selenium present at greater than 160% to 727% of a reference selenium daily dose, wherein the reference selenium daily dose is 55 μg; or
(b) vitamin A present at greater than 158.5% to 375% of a reference vitamin A daily dose, wherein the reference vitamin A daily dose is 800 μg; pyridoxine present at greater than 305% to 590% of a reference pyridoxine daily dose, wherein the reference pyridoxine daily dose is 1.4 mg; folic acid present at greater than 100% to 145% of a reference folic acid daily dose, wherein the reference folic acid daily dose is 200 μg; cyanocobalamin present at greater than 305% to 590% of a reference cyanocobalamin daily dose, wherein the reference cyanocobalamin daily dose is 2.5 μg; ascorbic acid present at greater than 275% to 460% of a reference ascorbic acid daily dose, wherein the reference ascorbic acid daily dose is 80 mg; cholecalciferol present at greater than 320% to 545% of a reference cholecalciferol daily dose, wherein the reference cholecalciferol daily dose is 5 μg; vitamin E present at greater than 465% to 1090% of a reference vitamin E daily dose, wherein the reference vitamin E daily dose is 12 mg; calcium present at greater than 100% to 122.5% of a reference calcium daily dose, wherein the reference calcium daily dose is 800 mg; and/or selenium present at greater than 100% to 160% of a reference selenium daily dose, wherein the reference selenium daily dose is 55 μg; or
(c) vitamin A present at greater than 127.5% to 158.5% of a reference vitamin A daily dose, wherein the reference vitamin A daily dose is 800 μg; pyridoxine present at greater than 590% to 7143% of a reference pyridoxine daily dose, wherein the reference pyridoxine daily dose is 1.4 mg; folic acid present at greater than 200% to 500% of a reference folic acid daily dose, wherein the reference folic acid daily dose is 200 μg; cyanocobalamin present at greater than 590% to 40000% of a reference cyanocobalamin daily dose, wherein the reference cyanocobalamin daily dose is 2.5 μg; ascorbic acid present at greater than 275% to 460% of a reference ascorbic acid daily dose, wherein the reference ascorbic acid daily dose is 80 mg; cholecalciferol present at greater than 545% to 2000% of a reference cholecalciferol daily dose, wherein the reference cholecalciferol daily dose is 5 μg; vitamin E present at greater than 465% to 1090% of a reference vitamin E daily dose, wherein the reference vitamin E daily dose is 12 mg; calcium present at greater than 122.5% to 143% of a reference calcium daily dose, wherein the reference calcium daily dose is 800 mg; and/or selenium present at greater than 100% to 160% of a reference selenium daily dose, wherein the reference selenium daily dose is 55 μg; or
(d) vitamin A present at greater than 158.5% to 375% of a reference vitamin A daily dose, wherein the reference vitamin A daily dose is 800 μg; pyridoxine present at greater than 305% to 590% of a reference pyridoxine daily dose, wherein the reference pyridoxine daily dose is 1.4 mg; folic acid present at greater than 100% to 145% of a reference folic acid daily dose, wherein the reference folic acid daily dose is 200 μg; cyanocobalamin present at greater than 305% to 590% of a reference cyanocobalamin daily dose, wherein the reference cyanocobalamin daily dose is 2.5 μg; ascorbic acid present at greater than 275% to 460% of a reference ascorbic acid daily dose, wherein the reference ascorbic acid daily dose is 80 mg; cholecalciferol present at greater than 545% to 2000% of a reference cholecalciferol daily dose, wherein the reference cholecalciferol daily dose is 5 μg; vitamin E present at greater than 465% to 1090% of a reference vitamin E daily dose, wherein the reference vitamin E daily dose is 12 mg; calcium present at greater than 143% to 313% of a reference calcium daily dose, wherein the reference calcium daily dose is 800 mg; and/or selenium present at greater than 100% to 160% of a reference selenium daily dose, wherein the reference selenium daily dose is 55 μg; or
(e) vitamin A present at greater than 158.5% to 375% of a reference vitamin A daily dose, wherein the reference vitamin A daily dose is 800 μg; pyridoxine present at greater than 590% to 7143% of a reference pyridoxine daily dose, wherein the reference pyridoxine daily dose is 1.4 mg; folic acid present at greater than 100% to 145% of a reference folic acid daily dose, wherein the reference folic acid daily dose is 200 μg; cyanocobalamin present at greater than 590% to 40000% of a reference cyanocobalamin daily dose, wherein the reference cyanocobalamin daily dose is 2.5 μg; ascorbic acid present at greater than 460% to 2500% of a reference ascorbic acid daily dose wherein the reference ascorbic acid daily dose is 80 mg; cholecalciferol present at greater than 545% to 2000% of a reference cholecalciferol daily dose, wherein the reference cholecalciferol daily dose is 5 μg; vitamin E present at greater than 1090% to 9167% of a reference vitamin E daily dose, wherein the reference vitamin E daily dose is 12 mg; calcium present at greater than 122.5% to 143% of a reference calcium daily dose, wherein the reference calcium daily dose is 800 mg; and/or selenium present at greater than 160% to 727% of a reference selenium daily dose, wherein the reference selenium daily dose is 55 μg; or
(f) vitamin A present at greater than 127.5% to 158.5% of a reference vitamin A daily dose, wherein the reference vitamin A daily dose is 800 μg; pyridoxine present at greater than 590% to 7143% of a reference pyridoxine daily dose, wherein the reference pyridoxine daily dose is 1.4 mg; folic acid present at greater than 145% to 200% of a reference folic acid daily dose, wherein the reference folic acid daily dose is 200 μg; cyanocobalamin present at greater than 590% to 40000% of a reference cyanocobalamin daily dose, wherein the reference cyanocobalamin daily dose is 2.5 μg; ascorbic acid present at greater than 275% to 460% of a reference ascorbic acid daily dose, wherein the reference ascorbic acid daily dose is 80 mg; cholecalciferol present at greater than 100% to 320% of a reference cholecalciferol daily dose, wherein the reference cholecalciferol daily dose is 5 μg; vitamin E present at greater than 465% to 1090% of a reference vitamin E daily dose, wherein the reference vitamin E daily dose is 12 mg; calcium present at greater than 100% to 122.5% of a reference calcium daily dose, wherein the reference calcium daily dose is 800 mg; and/or selenium present at greater than 160% to 727% of a reference selenium daily dose, wherein the reference selenium daily dose is 55 μg; or
(g) vitamin A present at greater than 127.5% to 158.5% of a reference vitamin A daily dose, wherein the reference vitamin A daily dose is 800 μg; pyridoxine present greater than 100% to 305% of a reference pyridoxine daily dose, wherein the reference pyridoxine daily dose is 1.4 mg; folic acid present at greater than 145% to 200% of a reference folic acid daily dose, wherein the reference folic acid daily dose is 200 μg; cyanocobalamin present at greater than 100% to 305% of a reference cyanocobalamin daily dose, wherein the reference cyanocobalamin daily dose is 2.5 μg; ascorbic acid present at greater than 460% to 2500% of a reference ascorbic acid daily dose wherein the reference ascorbic acid daily dose is 80 mg; cholecalciferol present at greater than 320% to 545% of a reference cholecalciferol daily dose, wherein the reference cholecalciferol daily dose is 5 μg; vitamin E present at greater than 1090% to 9167% of a reference vitamin E daily dose, wherein the reference vitamin E daily dose is 12 mg; calcium present at greater than 122.5% to 143% of a reference calcium daily dose, wherein the reference calcium daily dose is 800 mg; and/or selenium present at greater than 160% to 727% of a reference selenium daily dose, wherein the reference selenium daily dose is 55 μg; or
(h) vitamin A present at greater than 127.5% to 158.5% of a reference vitamin A daily dose, wherein the reference vitamin A daily dose is 800 μg; pyridoxine present at greater than 305% to 590% of a reference pyridoxine daily dose, wherein the reference pyridoxine daily dose is 1.4 mg; folic acid present at greater than 145% to 200% of a reference folic acid daily dose, wherein the reference folic acid daily dose is 200 μg; cyanocobalamin present at greater than 305% to 590% of a reference cyanocobalamin daily dose, wherein the reference cyanocobalamin daily dose is 2.5 μg; ascorbic acid present at greater than 275% to 460% of a reference ascorbic acid daily dose, wherein the reference ascorbic acid daily dose is 80 mg; cholecalciferol present at greater than 100% to 320% of a reference cholecalciferol daily dose, wherein the reference cholecalciferol daily dose is 5 μg; vitamin E present at greater than 465% to 1090% of a reference vitamin E daily dose, wherein the reference vitamin E daily dose is 12 mg; calcium present at greater than 122.5% to 143% of a reference calcium daily dose, wherein the reference calcium daily dose is 800 mg; and/or selenium present at greater than 160% to 727% of a reference selenium daily dose, wherein the reference selenium daily dose is 55 μg; or
(i) vitamin A present at greater than 127.5% to 158.5% of a reference vitamin A daily dose, wherein the reference vitamin A daily dose is 800 μg; pyridoxine present at greater than 305% to 590% of a reference pyridoxine daily dose, wherein the reference pyridoxine daily dose is 1.4 mg; folic acid present at greater than 145% to 200% of a reference folic acid daily dose, wherein the reference folic acid daily dose is 200 μg; cyanocobalamin present at greater than 305% to 590% of a reference cyanocobalamin daily dose, wherein the reference cyanocobalamin daily dose is 2.5 μg; ascorbic acid present at greater than 100% to 275% of a reference ascorbic acid daily dose, wherein the reference ascorbic acid daily dose is 80 mg; cholecalciferol present at greater than 320% to 545% of a reference cholecalciferol daily dose, wherein the reference cholecalciferol daily dose is 5 μg; vitamin E present at greater than 100% to 465% of a reference vitamin E daily dose, wherein the reference vitamin E daily dose is 12 mg; calcium present at greater than 122.5% to 143% of a reference calcium daily dose, wherein the reference calcium daily dose is 800 mg; and/or selenium present at greater than 100% to 160% of a reference selenium daily dose, wherein the reference selenium daily dose is 55 μg; or
(j) vitamin A present at greater than 158.5% to 375% of a reference vitamin A daily dose, wherein the reference vitamin A daily dose is 800 μg; pyridoxine present at greater than 305% to 590% of a reference pyridoxine daily dose, wherein the reference pyridoxine daily dose is 1.4 mg; folic acid present at greater than 100% to 145% of a reference folic acid daily dose, wherein the reference folic acid daily dose is 200 μg; cyanocobalamin present at greater than 305% to 590% of a reference cyanocobalamin daily dose, wherein the reference cyanocobalamin daily dose is 2.5 μg; ascorbic acid present at greater than 460% to 2500% of a reference ascorbic acid daily dose wherein the reference ascorbic acid daily dose is 80 mg; cholecalciferol present at greater than 320% to 545% of a reference cholecalciferol daily dose, wherein the reference cholecalciferol daily dose is 5 μg; vitamin E present at greater than 1090% to 9167% of a reference vitamin E daily dose, wherein the reference vitamin E daily dose is 12 mg; calcium present at greater than 122.5% to 143% of a reference calcium daily dose, wherein the reference calcium daily dose is 800 mg; and/or selenium present at greater than 160% to 727% of a reference selenium daily dose, wherein the reference selenium daily dose is 55 μg; or
(k) vitamin A present at greater than 158.5% to 375% of a reference vitamin A daily dose, wherein the reference vitamin A daily dose is 800 μg; pyridoxine present at greater than 305% to 590% of a reference pyridoxine daily dose, wherein the reference pyridoxine daily dose is 1.4 mg; folic acid present at greater than 145% to 200% of a reference folic acid daily dose, wherein the reference folic acid daily dose is 200 μg; cyanocobalamin present at greater than 305% to 590% of a reference cyanocobalamin daily dose, wherein the reference cyanocobalamin daily dose is 2.5 μg; ascorbic acid present at greater than 460% to 2500% of a reference ascorbic acid daily dose wherein the reference ascorbic acid daily dose is 80 mg; cholecalciferol present at greater than 320% to 545% of a reference cholecalciferol daily dose, wherein the reference cholecalciferol daily dose is 5 μg; vitamin E present at greater than 1090% to 9167% of a reference vitamin E daily dose, wherein the reference vitamin E daily dose is 12 mg; calcium present at greater than 122.5% to 143% of a reference calcium daily dose, wherein the reference calcium daily dose is 800 mg; and/or selenium present at greater than 100% to 160% of a reference selenium daily dose, wherein the reference selenium daily dose is 55 μg; or
(l) vitamin A present at greater than 127.5% to 158.5% of a reference vitamin A daily dose, wherein the reference vitamin A daily dose is 800 μg; pyridoxine present at greater than 305% to 590% of a reference pyridoxine daily dose, wherein the reference pyridoxine daily dose is 1.4 mg; folic acid present at greater than 145% to 200% of a reference folic acid daily dose, wherein the reference folic acid daily dose is 200 μg; cyanocobalamin present at greater than 305% to 590% of a reference cyanocobalamin daily dose, wherein the reference cyanocobalamin daily dose is 2.5 μg; ascorbic acid present at greater than 460% to 2500% of a reference ascorbic acid daily dose wherein the reference ascorbic acid daily dose is 80 mg; cholecalciferol present at greater than 100% to 320% of a reference cholecalciferol daily dose, wherein the reference cholecalciferol daily dose is 5 μg; vitamin E present at greater than 1090% to 9167% of a reference vitamin E daily dose, wherein the reference vitamin E daily dose is 12 mg; calcium present at greater than 100% to 122.5% of a reference calcium daily dose, wherein the reference calcium daily dose is 800 mg; and/or selenium present at greater than 160% to 727% of a reference selenium daily dose, wherein the reference selenium daily dose is 55 μg; or
(m) vitamin A present at greater than 158.5% to 375% of a reference vitamin A daily dose, wherein the reference vitamin A daily dose is 800 μg; pyridoxine present at greater than 100% to 305% of a reference pyridoxine daily dose, wherein the reference pyridoxine daily dose is 1.4 mg; folic acid present at greater than 200% to 500% of a reference folic acid daily dose, wherein the reference folic acid daily dose is 200 μg; cyanocobalamin present at greater than 100% to 305% of a reference cyanocobalamin daily dose, wherein the reference cyanocobalamin daily dose is 2.5 μg; ascorbic acid present at greater than 275% to 460% of a reference ascorbic acid daily dose, wherein the reference ascorbic acid daily dose is 80 mg; cholecalciferol present at greater than 320% to 545% of a reference cholecalciferol daily dose, wherein the reference cholecalciferol daily dose is 5 μg; vitamin E present at greater than 465% to 1090% of a reference vitamin E daily dose, wherein the reference vitamin E daily dose is 12 mg; calcium present at greater than 122.5% to 143% of a reference calcium daily dose, wherein the reference calcium daily dose is 800 mg; and/or selenium present at greater than 100% to 160% of a reference selenium daily dose, wherein the reference selenium daily dose is 55 μg; or
(n) vitamin A present at greater than 127.5% to 158.5% of a reference vitamin A daily dose, wherein the reference vitamin A daily dose is 800 μg; pyridoxine present at greater than 590% to 7143% of a reference pyridoxine daily dose, wherein the reference pyridoxine daily dose is 1.4 mg; folic acid present greater than 145% to 200% of a reference folic acid daily dose, wherein the reference folic acid daily dose is 200 μg; cyanocobalamin present at greater than 590% to 40000% of a reference cyanocobalamin daily dose, wherein the reference cyanocobalamin daily dose is 2.5 μg; ascorbic acid present at greater than 460% to 2500% of a reference ascorbic acid daily dose wherein the reference ascorbic acid daily dose is 80 mg; cholecalciferol present at greater than 320% to 545% of a reference cholecalciferol daily dose, wherein the reference cholecalciferol daily dose is 5 μg; vitamin E present at greater than 1090% to 9167% of a reference vitamin E daily dose, wherein the reference vitamin E daily dose is 12 mg; calcium present at greater than 122.5% to 143% of a reference calcium daily dose, wherein the reference calcium daily dose is 800 mg; and/or selenium present at greater than 160% to 727% of a reference selenium daily dose, wherein the reference selenium daily dose is 55 μg; or
(o) vitamin A present at greater than 158.5% to 375% of a reference vitamin A daily dose, wherein the reference vitamin A daily dose is 800 μg; pyridoxine present at greater than 100% to 305% of a reference pyridoxine daily dose, wherein the reference pyridoxine daily dose is 1.4 mg; folic acid present at greater than 145% to 200% of a reference folic acid daily dose, wherein the reference folic acid daily dose is 200 μg; cyanocobalamin present at greater than 100% to 305% of a reference cyanocobalamin daily dose, wherein the reference cyanocobalamin daily dose is 2.5 μg; ascorbic acid present at greater than 460% to 2500% of a reference ascorbic acid daily dose, wherein the reference ascorbic acid daily dose is 80 mg; cholecalciferol present at greater than 100% to 320% of a reference cholecalciferol daily dose, wherein the reference cholecalciferol daily dose is 5 μg; vitamin E present at greater than 1090% to 9167% of a reference vitamin E daily dose, wherein the reference vitamin E daily dose is 12 mg; calcium present at greater than 100% to 122.5% of a reference calcium daily dose, wherein the reference calcium daily dose is 800 mg; and/or selenium present at greater than 100% to 160% of a reference selenium daily dose, wherein the reference selenium daily dose is 55 μg; or
(p) vitamin A present at greater than 127.5% to 158.5% of a reference vitamin A daily dose, wherein the reference vitamin A daily dose is 800 μg; pyridoxine present at greater than 100% to 305% of a reference pyridoxine daily dose, wherein the reference pyridoxine daily dose is 1.4 mg; folic acid present at greater than 145% to 200% of a reference folic acid daily dose, wherein the reference folic acid daily dose is 200 μg; cyanocobalamin present at greater than 100% to 305% of a reference cyanocobalamin daily dose, wherein the reference cyanocobalamin daily dose is 2.5 μg; ascorbic acid present at greater than 275% to 460% of a reference ascorbic acid daily dose, wherein the reference ascorbic acid daily dose is 80 mg; cholecalciferol present at greater than 100% to 320% of a reference cholecalciferol daily dose, wherein the reference cholecalciferol daily dose is 5 μg; vitamin E present at greater than 465% to 1090% of a reference vitamin E daily dose, wherein the reference vitamin E daily dose is 12 mg; calcium present at greater than 100% to 122.5% of a reference calcium daily dose, wherein the reference calcium daily dose is 800 mg; and/or selenium present at greater than 160% to 727% of a reference selenium daily dose, wherein the reference selenium daily dose is 55 μg; or
(q) vitamin A present at greater than 127.5% to 158.5% of a reference vitamin A daily dose, wherein the reference vitamin A daily dose is 800 μg; pyridoxine present at greater than 305% to 590% of a reference pyridoxine daily dose, wherein the reference pyridoxine daily dose is 1.4 mg; folic acid present at greater than 145% to 200% of a reference folic acid daily dose, wherein the reference folic acid daily dose is 200 μg; cyanocobalamin present at greater than 305% to 590% of a reference cyanocobalamin daily dose, wherein the reference cyanocobalamin daily dose is 2.5 μg; ascorbic acid present at greater than 460% to 2500% of a reference ascorbic acid daily dose, wherein the reference ascorbic acid daily dose is 80 mg; cholecalciferol present at greater than 545% to 2000% of a reference cholecalciferol daily dose, wherein the reference cholecalciferol daily dose is 5 μg; vitamin E present at greater than 1090% to 9167% of a reference vitamin E daily dose, wherein the reference vitamin E daily dose is 12 mg; calcium present at greater than 143% to 313% of a reference calcium daily dose, wherein the reference calcium daily dose is 800 mg; and/or selenium present at greater than 100% to 160% of a reference selenium daily dose, wherein the reference selenium daily dose is 55 μg.

10. The composition or kit according to any one of claims 3-9, wherein the daily unit dose of the one or more micronutrients comprises:
(a) vitamin A present at 130% to 157% (preferably 130%) of a reference vitamin A daily dose, wherein the reference vitamin A daily dose is 800 μg; pyridoxine present at 350% to 530% (preferably 350%) of a reference pyridoxine daily dose, wherein the reference pyridoxine daily dose is 1.4 mg; folic acid present at 120% to 140% (preferably 120%) of a reference folic acid daily dose, wherein the reference folic acid daily dose is 200 μg; cyanocobalamin present at 350% to 530% (preferably 350%) of a reference cyanocobalamin daily dose, wherein the reference cyanocobalamin daily dose is 2.5 μg; ascorbic acid present at 350% to 420% (preferably 350%) of a reference ascorbic acid daily dose, wherein the reference ascorbic acid daily dose is 80 mg; cholecalciferol present at 600% to 660% (preferably 600%) of a reference cholecalciferol daily dose, wherein the reference cholecalciferol daily dose is 5 μg; vitamin E present at 800% to 880% (preferably 800%) of a reference calcium present at 110% to 120% (preferably 110%) of a reference calcium selenium present at 165% to 205% (preferably 165%) of a reference selenium daily dose, wherein the reference selenium daily dose is 55 μg; or
(b) vitamin A present at 160% to 178% (preferably 160%) of a reference vitamin A daily dose, wherein the reference vitamin A daily dose is 800 μg; pyridoxine present at 350% to 530% (preferably 370%) of a reference pyridoxine daily dose, wherein the reference pyridoxine daily dose is 1.4 mg; folic acid present at 120% to 140% (preferably 125%) of a reference folic acid daily dose, wherein the reference folic acid daily dose is 200 μg; cyanocobalamin present at 350% to 530% (preferably 370%) of a reference cyanocobalamin daily dose, wherein the reference cyanocobalamin daily dose is 2.5 μg; ascorbic acid present at 350% to 420% (preferably 360%) of a reference ascorbic acid daily dose, wherein the reference ascorbic acid daily dose is 80 mg; cholecalciferol present at 400% to 490% (preferably 400%) of a reference cholecalciferol daily dose, wherein the reference cholecalciferol daily dose is 5 μg; vitamin E present at 800% to 880% (preferably 810%) of a reference vitamin E daily dose, wherein the reference vitamin E daily dose is 12 mg; calcium present at 110% to 120% (preferably 112%) of a reference calcium daily dose, wherein the reference calcium daily dose is 800 mg; and/or selenium present at 120% to 155% (preferably 120%) of a reference selenium daily dose, wherein the reference selenium daily dose is 55 μg; or
(c) vitamin A present at 130% to 157% (preferably 133%) of a reference vitamin A daily dose, wherein the reference vitamin A daily dose is 800 μg; pyridoxine present at 650% to 720% (preferably 650%) of a reference pyridoxine daily dose, wherein the reference pyridoxine daily dose is 1.4 mg; folic acid present at 205% to 210% (preferably 205%) of a reference folic acid daily dose, wherein the reference folic acid daily dose is 200 μg; cyanocobalamin present at 650% to 720% (preferably 650%) of a reference cyanocobalamin daily dose, wherein the reference cyanocobalamin daily dose is 2.5 μg; ascorbic acid present at 350% to 420% (preferably 370%) of a reference ascorbic acid daily dose, wherein the reference ascorbic acid daily dose is 80 mg; cholecalciferol present at 600% to 660% (preferably 615%) of a reference cholecalciferol daily dose, wherein the reference cholecalciferol daily dose is 5 μg; vitamin E present at 800% to 880% (preferably 820%) of a reference calcium present at 125% to 141% (preferably 125%) of a reference calcium daily dose, wherein the reference calcium daily dose is 800 μg; and/or selenium present at 120% to 155% (preferably 125%) of a reference selenium daily dose, wherein the reference selenium daily dose is 55 μg; or
(d) vitamin A present at 160% to 178% (preferably 163%) of a reference vitamin A daily dose, wherein the reference vitamin A daily dose is 800 μg; pyridoxine present at 350% to 530% (preferably 410%) of a reference pyridoxine daily dose, wherein the reference pyridoxine daily dose is 1.4 mg; folic acid present at 120% to 140% (preferably 130%) of a reference folic acid daily dose, wherein the reference folic acid daily dose is 200 μg; cyanocobalamin present at 350% to 530% (preferably 410%) of a reference cyanocobalamin daily dose, wherein the reference cyanocobalamin daily dose is 2.5 μg; ascorbic acid present at 350% to 420% (preferably 380%) of a reference ascorbic acid daily dose, wherein the reference ascorbic acid daily dose is 80 mg; cholecalciferol present at 600% to 660% (preferably 630%) of a reference cholecalciferol daily dose, wherein the reference cholecalciferol daily dose is 5 μg; vitamin E present at 800% to 880% (preferably 830%) of a reference vitamin E daily dose, wherein the reference vitamin E daily dose is 12 mg; calcium present at 145% to 147% (preferably 145%) of a reference calcium daily dose, wherein the reference calcium daily dose is 800 mg; and/or selenium present at 120% to 155% (preferably 130%) of a reference selenium daily dose, wherein the reference selenium daily dose is 55 μg; or
(e) vitamin A present at 160% to 178% (preferably 166%) of a reference vitamin A daily dose, wherein the reference vitamin A daily dose is 800 μg; pyridoxine present at 650% to 720% (preferably 670%) of a reference pyridoxine daily dose, wherein the reference pyridoxine daily dose is 1.4 mg; folic acid present at 120% to 140% (preferably 135%) of a reference folic acid daily dose, wherein the reference folic acid daily dose is 200 μg; cyanocobalamin present at 650% to 720% (preferably 670%) of a reference cyanocobalamin daily dose, wherein the reference cyanocobalamin daily dose is 2.5 μg; ascorbic acid present at 500% to 570% (preferably 500%) of a reference ascorbic acid daily dose, wherein the reference ascorbic acid daily dose is 80 mg; cholecalciferol present at: 600% to 660% (preferably 645%) of a reference cholecalciferol daily dose, wherein the reference cholecalciferol daily dose is 5 μg; vitamin E present at 1300% to 1370% (preferably 1300%) of a reference calcium present at 125% to 141% (preferably 127%) of a reference calcium daily dose, wherein the reference calcium daily dose is 800 μg; and/or selenium present at 165% to 205% (preferably 170%) of a reference selenium daily dose, wherein the reference selenium daily dose is 55 μg; or
(f) vitamin A present at 130% to 157% (preferably 136%) of a reference vitamin A daily dose, wherein the reference vitamin A daily dose is 800 μg; pyridoxine present at 650% to 720% (preferably 690%) of a reference pyridoxine daily dose, wherein the reference pyridoxine daily dose is 1.4 mg; folic acid present at 150% to 195% (preferably 150%) of a reference folic acid daily dose, wherein the reference folic acid daily dose is 200 μg; cyanocobalamin present at 650% to 720% (preferably 690%) of a reference cyanocobalamin daily dose, wherein the reference cyanocobalamin daily dose is 2.5 μg; ascorbic acid present at 350% to 420% (preferably 390%) of a reference ascorbic acid daily dose, wherein the reference ascorbic acid daily dose is 80 mg; cholecalciferol present at 180% to 240% (preferably 180%) of a reference cholecalciferol daily dose, wherein the reference cholecalciferol daily dose is 5 μg; vitamin E present at 800% to 880% (preferably 840%) of a reference vitamin E daily dose, wherein the reference vitamin E daily dose is 12 mg; calcium present at 110% to 120% (preferably 114%) of a reference calcium daily dose, wherein the reference calcium daily dose is 800 mg; and/or selenium present at 165% to 205% (preferably 175%) of a reference selenium daily dose, wherein the reference selenium daily dose is 55 μg; or
(g) vitamin A present at 130% to 157% (preferably 139%) of a reference vitamin A daily dose, wherein the reference vitamin A daily dose is 800 μg; pyridoxine present at 200% to 260% (preferably 200%) of a reference pyridoxine daily dose, wherein the reference pyridoxine daily dose is 1.4 mg; folic acid present at 150% to 195% (preferably 155%) of a reference folic acid daily dose, wherein the reference folic acid daily dose is 200 μg; cyanocobalamin present at 200% to 260% (preferably 200%) of a reference cyanocobalamin daily dose, wherein the reference cyanocobalamin daily dose is 2.5 μg; ascorbic acid present at 500% to 570% (preferably 510%) of a reference ascorbic acid daily dose, wherein the reference ascorbic acid daily dose is 80 mg; cholecalciferol present at 400% to 490% (preferably 415%) of a reference cholecalciferol daily dose, wherein the reference cholecalciferol daily dose is 5 μg; vitamin E present at 1300% to 1370% (preferably 1310%) of a reference calcium present at 125% to 141% (preferably 129%) of a reference calcium daily dose, wherein the reference calcium daily dose is 800 μg; and/or selenium present at 165% to 205% (preferably 180%) of a reference selenium daily dose, wherein the reference selenium daily dose is 55 μg; or
(h) vitamin A present at 130% to 157% (preferably 142%) of a reference vitamin A daily dose, wherein the reference vitamin A daily dose is 800 μg; pyridoxine present at 350% to 530% (preferably 430%) of a reference pyridoxine daily dose, wherein the reference pyridoxine daily dose is 1.4 mg; folic acid present at 150% to 195% (preferably 160%) of a reference folic acid daily dose, wherein the reference folic acid daily dose is 200 μg; cyanocobalamin present at 350% to 530% (preferably 430%) of a reference cyanocobalamin daily dose, wherein the reference cyanocobalamin daily dose is 2.5 μg; ascorbic acid present at 350% to 420% (preferably 400%) of a reference ascorbic acid daily dose, wherein the reference ascorbic acid daily dose is 80 mg; cholecalciferol present at 180% to 240% (preferably 195%) of a reference cholecalciferol daily dose, wherein the reference cholecalciferol daily dose is 5 μg; vitamin E present at 800% to 880% (preferably 850%) of a reference vitamin E daily dose, wherein the reference vitamin E daily dose is 12 mg; calcium present at 125% to 141% (preferably 131%) of a reference calcium daily dose, wherein the reference calcium daily dose is 800 mg; and/or selenium present at 165% to 205% (preferably 185%) of a reference selenium daily dose, wherein the reference selenium daily dose is 55 μg; or
(i) vitamin A present at 130% to 157% (preferably 145%) of a reference vitamin A daily dose, wherein the reference vitamin A daily dose is 800 μg; pyridoxine present at 350% to 530% (preferably 450%) of a reference pyridoxine daily dose, wherein the reference pyridoxine daily dose is 1.4 mg; folic acid present at 150% to 195% (preferably 165%) of a reference folic acid daily dose, wherein the reference folic acid daily dose is 200 μg; cyanocobalamin present at 350% to 530% (preferably 450%) of a reference cyanocobalamin daily dose, wherein the reference cyanocobalamin daily dose is 2.5 μg; ascorbic acid present at 190% to 210% (preferably 200%) of a reference ascorbic acid daily dose, wherein the reference ascorbic acid daily dose is 80 mg; cholecalciferol present at 400% to 490% (preferably 430%) of a reference cholecalciferol daily dose, wherein the reference cholecalciferol daily dose is 5 μg; vitamin E present at 125% to 135% (preferably 130%) of a reference calcium present at 125% to 141% (preferably 133%) of a reference calcium daily dose, wherein the reference calcium daily dose is 800 μg; and/or selenium present at 120% to 155% (preferably 135%) of a reference selenium daily dose, wherein the reference selenium daily dose is 55 μg; or
(j) vitamin A present at 160% to 178% (preferably 169%) of a reference vitamin A daily dose, wherein the reference vitamin A daily dose is 800 μg; pyridoxine present at 350% to 530% (preferably 470%) of a reference pyridoxine daily dose, wherein the reference pyridoxine daily dose is 1.4 mg; folic acid present at 120% to 140% (preferably 140%) of a reference folic acid daily dose, wherein the reference folic acid daily dose is 200 μg; cyanocobalamin present at 350% to 530% (preferably 470%) of a reference cyanocobalamin daily dose, wherein the reference cyanocobalamin daily dose is 2.5 μg; ascorbic acid present at 500% to 570% (preferably 520%) of a reference ascorbic acid daily dose, wherein the reference ascorbic acid daily dose is 80 mg; cholecalciferol present at 400% to 490% (preferably 445%) of a reference cholecalciferol daily dose, wherein the reference cholecalciferol daily dose is 5 μg; vitamin E present at 1300% to 1370% (preferably 1320%) of a reference vitamin E daily dose, wherein the reference vitamin E daily dose is 12 mg; calcium present at 125% to 141% (preferably 135%) of a reference calcium daily dose, wherein the reference calcium daily dose is 800 mg; and/or selenium present at 165% to 205% (preferably 190%) of a reference selenium daily dose, wherein the reference selenium daily dose is 55 μg; or
(k) vitamin A present at 160% to 178% (preferably 172%) of a reference vitamin A daily dose, wherein the reference vitamin A daily dose is 800 μg; pyridoxine present at 350% to 530% (preferably 490%) of a reference pyridoxine daily dose, wherein the reference pyridoxine daily dose is 1.4 mg; folic acid present at 150% to 195% (preferably 170%) of a reference folic acid daily dose, wherein the reference folic acid daily dose is 200 μg; cyanocobalamin present at 350% to 530% (preferably 490%) of a reference cyanocobalamin daily dose, wherein the reference cyanocobalamin daily dose is 2.5 μg; ascorbic acid present at 500% to 570% (preferably 530%) of a reference ascorbic acid daily dose wherein the reference ascorbic acid daily dose is 80 mg; cholecalciferol present at 400% to 490% (preferably 460%) of a reference cholecalciferol daily dose, wherein the reference cholecalciferol daily dose is 5 μg; vitamin E present at 1300% to 1370% (preferably 1330%) of a reference calcium present at 125% to 141% (preferably 137%) of a reference calcium daily dose, wherein the reference calcium daily dose is 800 μg; and/or selenium present at 120% to 155% (preferably 140%) of a reference selenium daily dose, wherein the reference selenium daily dose is 55 μg; or
(l) vitamin A present at 130% to 157% (preferably 148%) of a reference vitamin A daily dose, wherein the reference vitamin A daily dose is 800 μg; pyridoxine present at 350% to 530% (preferably 510%) of a reference pyridoxine daily dose, wherein the reference pyridoxine daily dose is 1.4 mg; folic acid present at 150% to 195% (preferably 175%) of a reference folic acid daily dose, wherein the reference folic acid daily dose is 200 μg; cyanocobalamin present at 350% to 530% (preferably 510%) of a reference cyanocobalamin daily dose, wherein the reference cyanocobalamin daily dose is 2.5 μg; ascorbic acid present at 500% to 570% (preferably 540%) of a reference ascorbic acid daily dose wherein the reference ascorbic acid daily dose is 80 mg; cholecalciferol present at 180% to 240% (preferably 210%) of a reference cholecalciferol daily dose, wherein the reference cholecalciferol daily dose is 5 μg; vitamin E present 1300% to 1370% (preferably 1340%) of a reference vitamin E daily dose, wherein the reference vitamin E daily dose is 12 mg; calcium present at 110% to 120% (preferably 116%) of a reference calcium daily dose, wherein the reference calcium daily dose is 800 mg; and/or selenium present at 165% to 205% (preferably 195%) of a reference selenium daily dose, wherein the reference selenium daily dose is 55 μg; or
(m) vitamin A present at 160% to 178% (preferably 175%) of a reference vitamin A daily dose, wherein the reference vitamin A daily dose is 800 μg; pyridoxine present at 200% to 260% (preferably 220%) of a reference pyridoxine daily dose, wherein the reference pyridoxine daily dose is 1.4 mg; folic acid present at 205% to 210% (preferably 210%) of a reference folic acid daily dose, wherein the reference folic acid daily dose is 200 μg; cyanocobalamin present at 200% to 260% (preferably 220%) of a reference cyanocobalamin daily dose, wherein the reference cyanocobalamin daily dose is 2.5 μg; ascorbic acid present at 350% to 420% (preferably 410%) of a reference ascorbic acid daily dose, wherein the reference ascorbic acid daily dose is 80 mg; cholecalciferol present at 400% to 490% (preferably 475%) of a reference cholecalciferol daily dose, wherein the reference cholecalciferol daily dose is 5 μg; vitamin E present at 800% to 880% (preferably 870%) of a reference calcium present at 125% to 141% (preferably 139%) of a reference calcium daily dose, wherein the reference calcium daily dose is 800 μg; and/or selenium present at 120% to 155% (preferably 145%) of a reference selenium daily dose, wherein the reference selenium daily dose is 55 μg; or
(n) vitamin A present at 130% to 157% (preferably 151%) of a reference vitamin A daily dose, wherein the reference vitamin A daily dose is 800 μg; pyridoxine present at 650% to 720% (preferably 720%) of a reference pyridoxine daily dose, wherein the reference pyridoxine daily dose is 1.4 mg; folic acid present at 150% to 195% (preferably 180%) of a reference folic acid daily dose, wherein the reference folic acid daily dose is 200 μg; cyanocobalamin present at 650% to 720% (preferably 720%) of a reference cyanocobalamin daily dose, wherein the reference cyanocobalamin daily dose is 2.5 μg; ascorbic acid present at 500% to 570% (preferably 550%) of a reference ascorbic acid daily dose, wherein the reference ascorbic acid daily dose is 80 mg; cholecalciferol present at 400% to 490% (preferably 490%) of a reference cholecalciferol daily dose, wherein the reference cholecalciferol daily dose is 5 μg; vitamin E present at 1300% to 1370% (preferably 1350%) of a reference vitamin E daily dose, wherein the reference vitamin E daily dose is 12 mg; calcium present at 125% to 141% (preferably 141%) of a reference calcium daily dose, wherein the reference calcium daily dose is 800 mg; and/or selenium present at 165% to 205% (preferably 200%) of a reference selenium daily dose, wherein the reference selenium daily dose is 55 μg; or
(o) vitamin A present at 160% to 178% (preferably 178%) of a reference vitamin A daily dose, wherein the reference vitamin A daily dose is 800 μg; pyridoxine present at 200% to 260% (preferably 240%) of a reference pyridoxine daily dose, wherein the reference pyridoxine daily dose is 1.4 mg; folic acid present at 150% to 195% (preferably 185%) of a reference folic acid daily dose, wherein the reference folic acid daily dose is 200 μg; cyanocobalamin present at 200% to 260% (preferably 240%) of a reference cyanocobalamin daily dose, wherein the reference cyanocobalamin daily dose is 2.5 μg; ascorbic acid present at 500% to 570% (preferably 560%) of a reference ascorbic acid daily dose, wherein the reference ascorbic acid daily dose is 80 mg; cholecalciferol present at 180% to 240% (preferably 225%) of a reference cholecalciferol daily dose, wherein the reference cholecalciferol daily dose is 5 μg; vitamin E present at 1300% to 1370% (preferably 1360%) of a reference calcium present at 110% to 120% (preferably 118%) of a reference calcium daily dose, wherein the reference calcium daily dose is 800 μg; and/or selenium present at 120% to 155% (preferably 150%) of a reference selenium daily dose, wherein the reference selenium daily dose is 55 μg; or
(p) vitamin A present at 130% to 157% (preferably 154%) of a reference vitamin A daily dose, wherein the reference vitamin A daily dose is 800 μg; pyridoxine present at 200% to 260% (preferably 260%) of a reference pyridoxine daily dose, wherein the reference pyridoxine daily dose is 1.4 mg; folic acid present at 150% to 195% (preferably 190%) of a reference folic acid daily dose, wherein the reference folic acid daily dose is 200 μg; cyanocobalamin present at 200% to 260% (preferably 260%) of a reference cyanocobalamin daily dose, wherein the reference cyanocobalamin daily dose is 2.5 μg; ascorbic acid present at 350% to 420% (preferably 420%) of a reference ascorbic acid daily dose, wherein the reference ascorbic acid daily dose is 80 mg; cholecalciferol present at 180% to 240% (preferably 240%) of a reference cholecalciferol daily dose, wherein the reference cholecalciferol daily dose is 5 μg; vitamin E present at 800% to 880% (preferably 880%) of a reference vitamin E daily dose, wherein the reference vitamin E daily dose is 12 mg; calcium present at 110% to 120% (preferably 120%) of a reference calcium daily dose, wherein the reference calcium daily dose is 800 mg; and/or selenium present at 165% to 205% (preferably 205%) of a reference selenium daily dose, wherein the reference selenium daily dose is 55 μg; or
(q) vitamin A present at 130% to 157% (preferably 157%) of a reference vitamin A daily dose, wherein the reference vitamin A daily dose is 800 μg; pyridoxine present at 350% to 530% (preferably 530%) of a reference pyridoxine daily dose, wherein the reference pyridoxine daily dose is 1.4 mg; folic acid present at 150% to 195% (preferably 195%) of a reference folic acid daily dose, wherein the reference folic acid daily dose is 200 μg; cyanocobalamin present at 350% to 530% (preferably 530%) of a reference cyanocobalamin daily dose, wherein the reference cyanocobalamin daily dose is 2.5 μg; ascorbic acid present at 500% to 570% (preferably 570%) of a reference ascorbic acid daily dose, wherein the reference ascorbic acid daily dose is 80 mg; cholecalciferol present at 600% to 660% (preferably 660%) of a reference cholecalciferol daily dose, wherein the reference cholecalciferol daily dose is 5 μg; vitamin E present at 1300% to 1370% (preferably 1370%) of a reference calcium present at 145% to 147% (preferably 147%) of a reference calcium daily dose, wherein the reference calcium daily dose is 800 μg; and/or selenium present at 120% to 155% (preferably 155%) of a reference selenium daily dose, wherein the reference selenium daily dose is 55 μg.

11. The composition according to any one of claims 3-10, wherein the fraction of the daily unit dose of the one or more micronutrients is 50% of the daily unit dose of the one or more micronutrients.

12. The composition or kit according to any one of the preceding claims, wherein:
(a) vitamin A is present at greater than 0.017 wt. % to 0.065 wt. % of the total micronutrients present in the composition;
(b) pyridoxine is present at greater than 0.030 wt. % to 2.174 wt. % of the total micronutrients present in the composition;
(c) folic acid is present at greater than 0.004 wt. % to 0.022 wt. % of the total micronutrients present in the composition;
(d) cyanocobalamin is present at greater than 0.00005 wt. % to 0.02174 wt. % of the total micronutrients present in the composition;
(e) ascorbic acid is present at greater than 1.739 wt. % to 43.478 wt. % of the total micronutrients present in the composition;
(f) cholecalciferol is present at greater than 0.00011 wt. % to 0.00217 wt. % of the total micronutrients present in the composition;
(g) vitamin E is present at greater than 0.26 wt. % to 23.91 wt. % of the total micronutrients present in the composition;
(h) calcium is present at greater than 17.39 wt. % to 54.43 wt. % of the total micronutrients present in the composition; and/or
(i) selenium is present at greater than 0.001 wt. % to 0.009 wt. % of the total micronutrients present in the composition.

13. The composition or kit according to any one of the preceding claims, wherein:
(a) vitamin A is present at: (i) greater than 0.017 wt. % to 0.022 wt. % of the total micronutrients present in the composition; (ii) greater than 0.022 wt. % to 0.028 wt. % of the total micronutrients present in the composition; or (iii) greater than 0.028 wt. % to 0.065 wt. % of the total micronutrients present in the composition;
(b) pyridoxine is present at: (i) greater than 0.030 wt. % to 0.093 wt. % of the total micronutrients present in the composition; (ii) greater than 0.093 wt. % to 0.180 wt. % of the total micronutrients present in the composition; or (iii) greater than 0.180 wt. % to 2.174 wt. % of the total micronutrients present in the composition;
(c) folic acid is present at: (i) greater than 0.004 wt. % to 0.006 wt. % of the total micronutrients present in the composition; (ii) greater than 0.006 wt. % to 0.009 wt. % of the total micronutrients present in the composition; or (iii) greater than 0.009 wt. % to 0.022 wt. % of the total micronutrients present in the composition;
(d) cyanocobalamin is present at: (i) greater than 0.00005 wt. % to 0.00017 wt. % of the total micronutrients present in the composition; (ii) greater than 0.00017 wt. % to 0.00032 wt. % of the total micronutrients present in the composition; or (iii) greater than 0.00032 wt. % to 0.02174 wt. % of the total micronutrients present in the composition;
(e) ascorbic acid is present at: (i) greater than 1.739 wt. % to 4.783 wt. % of the total micronutrients present in the composition; (ii) greater than 4.783 wt. % to 8.000 wt. % of the total micronutrients present in the composition; or (iii) greater than 8.000 wt. % to 43.478 wt. % of the total micronutrients present in the composition;
(f) cholecalciferol is present at: (i) greater than 0.00011 wt. % to 0.00035 wt. % of the total micronutrients present in the composition; (ii) greater than 0.00035 wt. % to 0.00059 wt. % of the total micronutrients present in the composition; or (iii) greater than 0.00059 wt. % to 0.00217 wt. % of the total micronutrients present in the composition;
(g) vitamin E is present at: (i) greater than 0.26 wt. % to 1.21 wt. % of the total micronutrients present in the composition; (ii) greater than 1.21 wt. % to 2.84 wt. % of the total micronutrients present in the composition; or (iii) greater than 2.84 wt. % to 23.91 wt. % of the total micronutrients present in the composition;
(h) calcium is present at: (i) greater than 17.39 wt. % to 21.30 wt. % of the total micronutrients present in the composition; (ii) greater than 21.30 wt. % to 24.87 wt. % of the total micronutrients present in the composition; or (iii) greater than 24.87 wt. % to 54.43 wt. % of the total micronutrients present in the composition; and/or
(i) selenium is present at: (i) greater than 0.001 wt. % to 0.002 wt. % of the total micronutrients present in the composition; or (ii) greater than 0.002 wt. % to 0.009 wt. % of the total micronutrients present in the composition.

14. The composition or kit according to any one of the preceding claims, wherein:
(a) vitamin A is present at: (i) 0.020 wt. % to 0.022 wt. % of the total micronutrients present in the composition; (ii) 0.023 wt. % to 0.027 wt. % of the total micronutrients present in the composition; or (iii) 0.028 wt. % to 0.031 wt. % of the total micronutrients present in the composition;
(b) pyridoxine is present at: (i) 0.061 wt. % to 0.079 wt. % of the total micronutrients present in the composition; (ii) 0.107 wt. % to 0.161 wt. % of the total micronutrients present in the composition; or (iii) 0.198 wt. % to 0.219 wt. % of the total micronutrients present in the composition;
(c) folic acid is present at: (i) 0.0052 wt. % to 0.0061 wt. % of the total micronutrients present in the composition; (ii) 0.0065 wt. % to 0.0085 wt. % of the total micronutrients present in the composition; or (iii) 0.0089 wt. % to 0.0091 wt. % of the total micronutrients present in the composition;
(d) cyanocobalamin is present at: (i) 0.00011 wt. % to 0.00014 wt. % of the total micronutrients present in the composition; (ii) 0.00019 wt. % to 0.00029 wt. % of the total micronutrients present in the composition; or (iii) 0.00035 wt. % to 0.00039 wt. % of the total micronutrients present in the composition;
(e) ascorbic acid is present at: (i) 3.304 wt. % to 3.652 wt. % of the total micronutrients present in the composition; (ii) 6.087 wt. % to 7.304 wt. % of the total micronutrients present in the composition; or (iii) 8.696 wt. % to 9.913 wt. % of the total micronutrients present in the composition;
(f) cholecalciferol is present at: (i) 0.00020 wt. % to 0.00026 wt. % of the total micronutrients present in the composition; (ii) 0.00043 wt. % to 0.00053 wt. % of the total micronutrients present in the composition; or (iii) 0.00065 wt. % to 0.00072 wt. % of the total micronutrients present in the composition;
(g) vitamin E is present at: (i) 0.33 wt. % to 0.35 wt. % of the total micronutrients present in the composition; (ii) 2.09 wt. % to 2.30 wt. % of the total micronutrients present in the composition; or (iii) 3.39 wt. % to 3.57 wt. % of the total micronutrients present in the composition;
(h) calcium is present at: (i) 19.13 wt. % to 20.87 wt. % of the total micronutrients present in the composition; (ii) 21.74 wt. % to 24.52 wt. % of the total micronutrients present in the composition; or (iii) 25.22 wt. % to 25.57 wt. % of the total micronutrients present in the composition; and/or
(i) selenium is present at: (i) 0.0014 wt. % to 0.0019 wt. % of the total micronutrients present in the composition; or (ii) 0.0020 wt. % to 0.0025 wt. % of the total micronutrients present in the composition.

15. The composition or kit according to any one of the preceding claims, wherein:
(a) vitamin A is present at greater than 0.022 wt. % to 0.028 wt. % of the total micronutrients present in the composition; pyridoxine is present at greater than 0.093 wt. % to 0.180 wt. % of the total micronutrients present in the composition; folic acid is present at greater than 0.004 wt. % to 0.006 wt. % of the total micronutrients present in the composition; cyanocobalamin is present at greater than 0.00017 wt. % to 0.00032 wt. % of the total micronutrients present in the composition; ascorbic acid is present at greater than 4.783 wt. % to 8.000 wt. % of the total micronutrients present in the composition; cholecalciferol is present at greater than 0.00059 wt. % to 0.00217 wt. % of the total micronutrients present in the composition; vitamin E is present at greater than 1.21 wt. % to 2.84 wt. % of the total micronutrients present in the composition; calcium is present at greater than 17.39 wt. % to 21.30 wt. % of the total micronutrients present in the composition; and/or selenium is present at greater than 0.002 wt. % to 0.009 wt. % of the total micronutrients present in the composition; or
(b) vitamin A is present at greater than 0.028 wt. % to 0.065 wt. % of the total micronutrients present in the composition; pyridoxine is present at greater than 0.093 wt. % to 0.180 wt. % of the total micronutrients present in the composition; folic acid is present at greater than 0.004 wt. % to 0.006 wt. % of the total micronutrients present in the composition; cyanocobalamin is present at greater than 0.00017 wt. % to 0.00032 wt. % of the total micronutrients present in the composition; ascorbic acid is present at greater than 4.783 wt. % to 8.000 wt. % of the total micronutrients present in the composition; cholecalciferol is present at greater than 0.00035 wt. % to 0.00059 wt. % of the total micronutrients present in the composition; vitamin E is present at greater than 1.21 wt. % to 2.84 wt. % of the total micronutrients present in the composition; calcium is present at greater than 17.39 wt. % to 21.30 wt. % of the total micronutrients present in the composition; and/or selenium is present at greater than 0.001 wt. % to 0.002 wt. % of the total micronutrients present in the composition; or
(c) vitamin A is present at greater than 0.022 wt. % to 0.028 wt. % of the total micronutrients present in the composition; pyridoxine is present at greater than 0.180 wt. % to 2.174 wt. % of the total micronutrients present in the composition; folic acid is present at greater than 0.009 wt. % to 0.022 wt. % of the total micronutrients present in the composition; cyanocobalamin is present at greater than 0.00032 wt. % to 0.02174 wt. % of the total micronutrients present in the composition; ascorbic acid is present at greater than 4.783 wt. % to 8.000 wt. % of the total micronutrients present in the composition; cholecalciferol is present at greater than 0.00059 wt. % to 0.00217 wt. % of the total micronutrients present in the composition; vitamin E is present at greater than 1.21 wt. % to 2.84 wt. % of the total micronutrients present in the composition; calcium is present at greater than 21.30 wt. % to 24.87 wt. % of the total micronutrients present in the composition; and/or selenium is present at greater than 0.001 wt. % to 0.002 wt. % of the total micronutrients present in the composition; or
(d) vitamin A is present at greater than 0.028 wt. % to 0.065 wt. % of the total micronutrients present in the composition; pyridoxine is present at greater than 0.093 wt. % to 0.180 wt. % of the total micronutrients present in the composition; folic acid is present at greater than 0.004 wt. % to 0.006 wt. % of the total micronutrients present in the composition; cyanocobalamin is present at greater than 0.00017 wt. % to 0.00032 wt. % of the total micronutrients present in the composition; ascorbic acid is present at greater than 4.783 wt. % to 8.000 wt. % of the total micronutrients present in the composition; cholecalciferol is present at greater than 0.00059 wt. % to 0.00217 wt. % of the total micronutrients present in the composition; vitamin E is present at greater than 1.21 wt. % to 2.84 wt. % of the total micronutrients present in the composition; calcium is present at greater than 24.87 wt. % to 54.43 wt. % of the total micronutrients present in the composition; and/or selenium is present at greater than 0.001 wt. % to 0.002 wt. % of the total micronutrients present in the composition; or
(e) vitamin A is present at greater than 0.028 wt. % to 0.065 wt. % of the total micronutrients present in the composition; pyridoxine is present at greater than 0.180 wt. % to 2.174 wt. % of the total micronutrients present in the composition; folic acid is present at greater than 0.004 wt. % to 0.006 wt. % of the total micronutrients present in the composition; cyanocobalamin is present at greater than 0.00032 wt. % to 0.02174 wt. % of the total micronutrients present in the composition; ascorbic acid is present at greater than 8.000 wt. % to 43.478 wt. % of the total micronutrients present in the composition; cholecalciferol is present at greater than 0.00059 wt. % to 0.00217 wt. % of the total micronutrients present in the composition; vitamin E is present at greater than 2.84 wt. % to 23.91 wt. % of the total micronutrients present in the composition; calcium is present at greater than 21.30 wt. % to 24.87 wt. % of the total micronutrients present in the composition; and/or selenium is present at greater than 0.002 wt. % to 0.009 wt. % of the total micronutrients present in the composition; or
(f) vitamin A is present at greater than 0.022 wt. % to 0.028 wt. % of the total micronutrients present in the composition; pyridoxine is present at greater than 0.180 wt. % to 2.174 wt. % of the total micronutrients present in the composition; folic acid is present at greater than 0.006 wt. % to 0.009 wt. % of the total micronutrients present in the composition; cyanocobalamin is present at greater than 0.00032 wt. % to 0.02174 wt. % of the total micronutrients present in the composition; ascorbic acid is present at greater than 4.783 wt. % to 8.000 wt. % of the total micronutrients present in the composition; cholecalciferol is present at greater than 0.00011 wt. % to 0.00035 wt. % of the total micronutrients present in the composition; vitamin E is present at greater than 1.21 wt. % to 2.84 wt. % of the total micronutrients present in the composition; calcium is present at greater than 17.39 wt. % to 21.30 wt. % of the total micronutrients present in the composition; and/or selenium is present at greater than 0.002 wt. % to 0.009 wt. % of the total micronutrients present in the composition; or
(g) vitamin A is present at greater than 0.022 wt. % to 0.028 wt. % of the total micronutrients present in the composition; pyridoxine is present at greater than 0.030 wt. % to 0.093 wt. % of the total micronutrients present in the composition; folic acid is present at greater than 0.006 wt. % to 0.009 wt. % of the total micronutrients present in the composition; cyanocobalamin is present at greater than 0.00005 wt. % to 0.00017 wt. % of the total micronutrients present in the composition; ascorbic acid is present at greater than 8.000 wt. % to 43.478 wt. % of the total micronutrients present in the composition; cholecalciferol is present at greater than 0.00035 wt. % to 0.00059 wt. % of the total micronutrients present in the composition; vitamin E is present at greater than 2.84 wt. % to 23.91 wt. % of the total micronutrients present in the composition; calcium is present at greater than 21.30 wt. % to 24.87 wt. % of the total micronutrients present in the composition; and/or selenium is present at greater than 0.002 wt. % to 0.009 wt. % of the total micronutrients present in the composition; or
(h) vitamin A is present at greater than 0.022 wt. % to 0.028 wt. % of the total micronutrients present in the composition; pyridoxine is present at greater than 0.093 wt. % to 0.180 wt. % of the total micronutrients present in the composition; folic acid is present at greater than 0.006 wt. % to 0.009 wt. % of the total micronutrients present in the composition; cyanocobalamin is present at greater than 0.00017 wt. % to 0.00032 wt. % of the total micronutrients present in the composition; ascorbic acid is present at greater than 4.783 wt. % to 8.000 wt. % of the total micronutrients present in the composition; cholecalciferol is present at greater than 0.00011 wt. % to 0.00035 wt. % of the total micronutrients present in the composition; vitamin E is present at greater than 1.21 wt. % to 2.84 wt. % of the total micronutrients present in the composition; calcium is present at greater than 21.30 wt. % to 24.87 wt. % of the total micronutrients present in the composition; and/or selenium is present at greater than 0.002 wt. % to 0.009 wt. % of the total micronutrients present in the composition; or
(i) vitamin A is present at greater than 0.022 wt. % to 0.028 wt. % of the total micronutrients present in the composition; pyridoxine is present at greater than 0.093 wt. % to 0.180 wt. % of the total micronutrients present in the composition; folic acid is present at greater than 0.006 wt. % to 0.009 wt. % of the total micronutrients present in the composition; cyanocobalamin is present at greater than 0.00017 wt. % to 0.00032 wt. % of the total micronutrients present in the composition; ascorbic acid is present at greater than 1.739 wt. % to 4.783 wt. % of the total micronutrients present in the composition; cholecalciferol is present at greater than 0.00035 wt. % to 0.00059 wt. % of the total micronutrients present in the composition; vitamin E is present at greater than 0.26 wt. % to 1.21 wt. % of the total micronutrients present in the composition; calcium is present at greater than 21.30 wt. % to 24.87 wt. % of the total micronutrients present in the composition; and/or selenium is present at greater than 0.001 wt. % to 0.002 wt. % of the total micronutrients present in the composition; or
(j) vitamin A is present at greater than 0.028 wt. % to 0.065 wt. % of the total micronutrients present in the composition; pyridoxine is present at greater than 0.093 wt. % to 0.180 wt. % of the total micronutrients present in the composition; folic acid is present at greater than 0.004 wt. % to 0.006 wt. % of the total micronutrients present in the composition; cyanocobalamin is present at greater than 0.00017 wt. % to 0.00032 wt. % of the total micronutrients present in the composition; ascorbic acid is present at greater than 8.000 wt. % to 43.478 wt. % of the total micronutrients present in the composition; cholecalciferol is present at greater than 0.00035 wt. % to 0.00059 wt. % of the total micronutrients present in the composition; vitamin E is present at greater than 2.84 wt. % to 23.91 wt. % of the total micronutrients present in the composition; calcium is present at greater than 21.30 wt. % to 24.87 wt. % of the total micronutrients present in the composition; and/or selenium is present at greater than 0.002 wt. % to 0.009 wt. % of the total micronutrients present in the composition; or
(k) vitamin A is present at greater than 0.028 wt. % to 0.065 wt. % of the total micronutrients present in the composition; pyridoxine is present at greater than 0.093 wt. % to 0.180 wt. % of the total micronutrients present in the composition; folic acid is present at greater than 0.006 wt. % to 0.009 wt. % of the total micronutrients present in the composition; cyanocobalamin is present at greater than 0.00017 wt. % to 0.00032 wt. % of the total micronutrients present in the composition; ascorbic acid is present at greater than 8.000 wt. % to 43.478 wt. % of the total micronutrients present in the composition; cholecalciferol is present at greater than 0.00035 wt. % to 0.00059 wt. % of the total micronutrients present in the composition; vitamin E is present at greater than 2.84 wt. % to 23.91 wt. % of the total micronutrients present in the composition; calcium is present at greater than 21.30 wt. % to 24.87 wt. % of the total micronutrients present in the composition; and/or selenium is present at greater than 0.001 wt. % to 0.002 wt. % of the total micronutrients present in the composition; or
(l) vitamin A is present at greater than 0.022 wt. % to 0.028 wt. % of the total micronutrients present in the composition; pyridoxine is present at greater than 0.093 wt. % to 0.180 wt. % of the total micronutrients present in the composition; folic acid is present at greater than 0.006 wt. % to 0.009 wt. % of the total micronutrients present in the composition; cyanocobalamin is present at greater than 0.00017 wt. % to 0.00032 wt. % of the total micronutrients present in the composition; ascorbic acid is present at greater than 8.000 wt. % to 43.478 wt. % of the total micronutrients present in the composition; cholecalciferol is present at greater than 0.00011 wt. % to 0.00035 wt. % of the total micronutrients present in the composition; vitamin E is present at greater than 2.84 wt. % to 23.91 wt. % of the total micronutrients present in the composition; calcium is present at greater than 17.39 wt. % to 21.30 wt. % of the total micronutrients present in the composition; and/or selenium is present at greater than 0.002 wt. % to 0.009 wt. % of the total micronutrients present in the composition; or
(m) vitamin A is present at greater than 0.028 wt. % to 0.065 wt. % of the total micronutrients present in the composition; pyridoxine is present at greater than 0.030 wt. % to 0.093 wt. % of the total micronutrients present in the composition; folic acid is present at greater than 0.009 wt. % to 0.022 wt. % of the total micronutrients present in the composition; cyanocobalamin is present at greater than 0.00005 wt. % to 0.00017 wt. % of the total micronutrients present in the composition; ascorbic acid is present at greater than 4.783 wt. % to 8.000 wt. % of the total micronutrients present in the composition; cholecalciferol is present at greater than 0.00035 wt. % to 0.00059 wt. % of the total micronutrients present in the composition; vitamin E is present at greater than 1.21 wt. % to 2.84 wt. % of the total micronutrients present in the composition; calcium is present at greater than 21.30 wt. % to 24.87 wt. % of the total micronutrients present in the composition; and/or selenium is present at greater than 0.001 wt. % to 0.002 wt. % of the total micronutrients present in the composition; or
(n) vitamin A is present at greater than 0.022 wt. % to 0.028 wt. % of the total micronutrients present in the composition; pyridoxine is present at greater than 0.180 wt. % to 2.174 wt. % of the total micronutrients present in the composition; folic acid is present at greater than 0.006 wt. % to 0.009 wt. % of the total micronutrients present in the composition; cyanocobalamin is present at greater than 0.00032 wt. % to 0.02174 wt. % of the total micronutrients present in the composition; ascorbic acid is present at greater than 8.000 wt. % to 43.478 wt. % of the total micronutrients present in the composition; cholecalciferol is present at greater than 0.00035 wt. % to 0.00059 wt. % of the total micronutrients present in the composition; vitamin E is present at greater than 2.84 wt. % to 23.91 wt. % of the total micronutrients present in the composition; calcium is present at greater than 21.30 wt. % to 24.87 wt. % of the total micronutrients present in the composition; and/or selenium is present at greater than 0.002 wt. % to 0.009 wt. % of the total micronutrients present in the composition; or
(o) vitamin A is present at greater than 0.028 wt. % to 0.065 wt. % of the total micronutrients present in the composition; pyridoxine is present at greater than 0.030 wt. % to 0.093 wt. % of the total micronutrients present in the composition; folic acid is present at greater than 0.006 wt. % to 0.009 wt. % of the total micronutrients present in the composition; cyanocobalamin is present at greater than 0.00005 wt. % to 0.00017 wt. % of the total micronutrients present in the composition; ascorbic acid is present at greater than 8.000 wt. % to 43.478 wt. % of the total micronutrients present in the composition; cholecalciferol is present at greater than 0.00011 wt. % to 0.00035 wt. % of the total micronutrients present in the composition; vitamin E is present at greater than 2.84 wt. % to 23.91 wt. % of the total micronutrients present in the composition; calcium is present at greater than 17.39 wt. % to 21.30 wt. % of the total micronutrients present in the composition; and/or selenium is present at greater than 0.001 wt. % to 0.002 wt. % of the total micronutrients present in the composition; or
(p) vitamin A is present at greater than 0.022 wt. % to 0.028 wt. % of the total micronutrients present in the composition; pyridoxine is present at greater than 0.030 wt. % to 0.093 wt. % of the total micronutrients present in the composition; folic acid is present at greater than 0.006 wt. % to 0.009 wt. % of the total micronutrients present in the composition; cyanocobalamin is present at greater than 0.00005 wt. % to 0.00017 wt. % of the total micronutrients present in the composition; ascorbic acid is present at greater than 4.783 wt. % to 8.000 wt. % of the total micronutrients present in the composition; cholecalciferol is present at greater than 0.00011 wt. % to 0.00035 wt. % of the total micronutrients present in the composition; vitamin E is present at greater than 1.21 wt. % to 2.84 wt. % of the total micronutrients present in the composition; calcium is present at greater than 17.39 wt. % to 21.30 wt. % of the total micronutrients present in the composition; and/or selenium is present at greater than 0.002 wt. % to 0.009 wt. % of the total micronutrients present in the composition; or
(q) vitamin A is present at greater than 0.022 wt. % to 0.028 wt. % of the total micronutrients present in the composition; pyridoxine is present at greater than 0.093 wt. % to 0.180 wt. % of the total micronutrients present in the composition; folic acid is present at greater than 0.006 wt. % to 0.009 wt. % of the total micronutrients present in the composition; cyanocobalamin is present at greater than 0.00017 wt. % to 0.00032 wt. % of the total micronutrients present in the composition; ascorbic acid is present at greater than 8.000 wt. % to 43.478 wt. % of the total micronutrients present in the composition; cholecalciferol is present at greater than 0.00059 wt. % to 0.00217 wt. % of the total micronutrients present in the composition; vitamin E is present at greater than 2.84 wt. % to 23.91 wt. % of the total micronutrients present in the composition; calcium is present at greater than 24.87 wt. % to 54.43 wt. % of the total micronutrients present in the composition; and/or selenium is present at greater than 0.001 wt. % to 0.002 wt. % of the total micronutrients present in the composition.

16. The composition or kit according to any one of the preceding claims, wherein:
(a) vitamin A is present at 0.023 wt. % to 0.027 wt. % of the total micronutrients present in the composition; pyridoxine is present at 0.107 wt. % to 0.161 wt. % of the total micronutrients present in the composition; folic acid is present at 0.0052 wt. % to 0.0061 wt. % of the total micronutrients present in the composition; cyanocobalamin is present at 0.00019 wt. % to 0.00029 wt. % of the total micronutrients present in the composition; ascorbic acid is present at 6.087 wt. % to 7.304 wt. % of the total micronutrients present in the composition; cholecalciferol is present at 0.00065 wt. % to 0.00072 wt. % of the total micronutrients present in the composition; vitamin E is present at 2.09 wt. % to 2.30 wt. % of the total micronutrients present in the composition; calcium is present at 19.13 wt. % to 20.87 wt. % of the total micronutrients present in the composition; and/or selenium is present at 0.0020 wt. % to 0.0025 wt. % of the total micronutrients present in the composition; or
(b) vitamin A is present at 0.028 wt. % to 0.031 wt. % of the total micronutrients present in the composition; pyridoxine is present at 0.107 wt. % to 0.161 wt. % of the total micronutrients present in the composition; folic acid is present at 0.0052 wt. % to 0.0061 wt. % of the total micronutrients present in the composition; cyanocobalamin is present at 0.00019 wt. % to 0.00029 wt. % of the total micronutrients present in the composition; ascorbic acid is present at 6.087 wt. % to 7.304 wt. % of the total micronutrients present in the composition; cholecalciferol is present at 0.00043 wt. % to 0.00053 wt. % of the total micronutrients present in the composition; vitamin E is present at 2.09 wt. % to 2.30 wt. % of the total micronutrients present in the composition; calcium is present at 19.13 wt. % to 20.87 wt. % of the total micronutrients present in the composition; and/or selenium is present at 0.0014 wt. % to 0.0019 wt. % of the total micronutrients present in the composition; or
(c) vitamin A is present at 0.023 wt. % to 0.027 wt. % of the total micronutrients present in the composition; pyridoxine is present at 0.198 wt. % to 0.219 wt. % of the total micronutrients present in the composition; folic acid is present at 0.0089 wt. % to 0.0091 wt. % of the total micronutrients present in the composition; cyanocobalamin is present at 0.00035 wt. % to 0.00039 wt. % of the total micronutrients present in the composition; ascorbic acid is present at 6.087 wt. % to 7.304 wt. % of the total micronutrients present in the composition; cholecalciferol is present at 0.00065 wt. % to 0.00072 wt. % of the total micronutrients present in the composition; vitamin E is present at 2.09 wt. % to 2.30 wt. % of the total micronutrients present in the composition; calcium is present at 21.74 wt. % to 24.52 wt. % of the total micronutrients present in the composition; and/or selenium is present at 0.0014 wt. % to 0.0019 wt. % of the total micronutrients present in the composition; or
(d) vitamin A is present at 0.028 wt. % to 0.031 wt. % of the total micronutrients present in the composition; pyridoxine is present at 0.107 wt. % to 0.161 wt. % of the total micronutrients present in the composition; folic acid is present at 0.0052 wt. % to 0.0061 wt. % of the total micronutrients present in the composition; cyanocobalamin is present at 0.00019 wt. % to 0.00029 wt. % of the total micronutrients present in the composition; ascorbic acid is present at 6.087 wt. % to 7.304 wt. % of the total micronutrients present in the composition; cholecalciferol is present at 0.00065 wt. % to 0.00072 wt. % of the total micronutrients present in the composition; vitamin E is present at 2.09 wt. % to 2.30 wt. % of the total micronutrients present in the composition; calcium is present at 25.22 wt. % to 25.57 wt. % of the total micronutrients present in the composition; and/or selenium is present at 0.0014 wt. % to 0.0019 wt. % of the total micronutrients present in the composition; or
(e) vitamin A is present at 0.028 wt. % to 0.031 wt. % of the total micronutrients present in the composition; pyridoxine is present at 0.198 wt. % to 0.219 wt. % of the total micronutrients present in the composition; folic acid is present at 0.0052 wt. % to 0.0061 wt. % of the total micronutrients present in the composition; cyanocobalamin is present at 0.00035 wt. % to 0.00039 wt. % of the total micronutrients present in the composition; ascorbic acid is present at 8.696 wt. % to 9.913 wt. % of the total micronutrients present in the composition; cholecalciferol is present at 0.00065 wt. % to 0.00072 wt. % of the total micronutrients present in the composition; vitamin E is present at 3.39 wt. % to 3.57 wt. % of the total micronutrients present in the composition; calcium is present at 21.74 wt. % to 24.52 wt. % of the total micronutrients present in the composition; and/or selenium is present at 0.0020 wt. % to 0.0025 wt. % of the total micronutrients present in the composition; or
(f) vitamin A is present at 0.023 wt. % to 0.027 wt. % of the total micronutrients present in the composition; pyridoxine is present at 0.198 wt. % to 0.219 wt. % of the total micronutrients present in the composition; folic acid is present at 0.0065 wt. % to 0.0085 wt. % of the total micronutrients present in the composition; cyanocobalamin is present at 0.00035 wt. % to 0.00039 wt. % of the total micronutrients present in the composition; ascorbic acid is present at 6.087 wt. % to 7.304 wt. % of the total micronutrients present in the composition; cholecalciferol is present at 0.00020 wt. % to 0.00026 wt. % of the total micronutrients present in the composition; vitamin E is present at 2.09 wt. % to 2.30 wt. % of the total micronutrients present in the composition; calcium is present at 19.13 wt. % to 20.87 wt. % of the total micronutrients present in the composition; and/or selenium is present at 0.0020 wt. % to 0.0025 wt. % of the total micronutrients present in the composition; or
(g) vitamin A is present at 0.023 wt. % to 0.027 wt. % of the total micronutrients present in the composition; pyridoxine is present at 0.061 wt. % to 0.079 wt. % of the total micronutrients present in the composition; folic acid is present at 0.0065 wt. % to 0.0085 wt. % of the total micronutrients present in the composition; cyanocobalamin is present at 0.00011 wt. % to 0.00014 wt. % of the total micronutrients present in the composition; ascorbic acid is present at 8.696 wt. % to 9.913 wt. % of the total micronutrients present in the composition; cholecalciferol is present at 0.00043 wt. % to 0.00053 wt. % of the total micronutrients present in the composition; vitamin E is present at 3.39 wt. % to 3.57 wt. % of the total micronutrients present in the composition; calcium is present at 21.74 wt. % to 24.52 wt. % of the total micronutrients present in the composition; and/or selenium is present at 0.0020 wt. % to 0.0025 wt. % of the total micronutrients present in the composition; or
(h) vitamin A is present at 0.023 wt. % to 0.027 wt. % of the total micronutrients present in the composition; pyridoxine is present at 0.107 wt. % to 0.161 wt. % of the total micronutrients present in the composition; folic acid is present at 0.0065 wt. % to 0.0085 wt. % of the total micronutrients present in the composition; cyanocobalamin is present at 0.00019 wt. % to 0.00029 wt. % of the total micronutrients present in the composition; ascorbic acid is present at 6.087 wt. % to 7.304 wt. % of the total micronutrients present in the composition; cholecalciferol is present at 0.00020 wt. % to 0.00026 wt. % of the total micronutrients present in the composition; vitamin E is present at 2.09 wt. % to 2.30 wt. % of the total micronutrients present in the composition; calcium is present at 21.74 wt. % to 24.52 wt. % of the total micronutrients present in the composition; and/or selenium is present at 0.0020 wt. % to 0.0025 wt. % of the total micronutrients present in the composition; or
(i) vitamin A is present at 0.023 wt. % to 0.027 wt. % of the total micronutrients present in the composition; pyridoxine is present at 0.107 wt. % to 0.161 wt. % of the total micronutrients present in the composition; folic acid is present at 0.0065 wt. % to 0.0085 wt. % of the total micronutrients present in the composition; cyanocobalamin is present at 0.00019 wt. % to 0.00029 wt. % of the total micronutrients present in the composition; ascorbic acid is present at 3.304 wt. % to 3.652 wt. % of the total micronutrients present in the composition; cholecalciferol is present at 0.00043 wt. % to 0.00053 wt. % of the total micronutrients present in the composition; vitamin E is present at 0.33 wt. % to 0.35 wt. % of the total micronutrients present in the composition; calcium is present at 21.74 wt. % to 24.52 wt. % of the total micronutrients present in the composition; and/or selenium is present at 0.0014 wt. % to 0.0019 wt. % of the total micronutrients present in the composition; or
(j) vitamin A is present at 0.028 wt. % to 0.031 wt. % of the total micronutrients present in the composition; pyridoxine is present at 0.107 wt. % to 0.161 wt. % of the total micronutrients present in the composition; folic acid is present at 0.0052 wt. % to 0.0061 wt. % of the total micronutrients present in the composition; cyanocobalamin is present at 0.00019 wt. % to 0.00029 wt. % of the total micronutrients present in the composition; ascorbic acid is present at 8.696 wt. % to 9.913 wt. % of the total micronutrients present in the composition; cholecalciferol is present at 0.00043 wt. % to 0.00053 wt. % of the total micronutrients present in the composition; vitamin E is present at 3.39 wt. % to 3.57 wt. % of the total micronutrients present in the composition; calcium is present at: 21.74 wt. % to 24.52 wt. % of the total micronutrients present in the composition; and/or selenium is present at 0.0020 wt. % to 0.0025 wt. % of the total micronutrients present in the composition; or
(k) vitamin A is present at 0.028 wt. % to 0.031 wt. % of the total micronutrients present in the composition; pyridoxine is present at 0.107 wt. % to 0.161 wt. % of the total micronutrients present in the composition; folic acid is present at 0.0065 wt. % to 0.0085 wt. % of the total micronutrients present in the composition; cyanocobalamin is present at 0.00019 wt. % to 0.00029 wt. % of the total micronutrients present in the composition; ascorbic acid is present at 8.696 wt. % to 9.913 wt. % of the total micronutrients present in the composition; cholecalciferol is present at 0.00043 wt. % to 0.00053 wt. % of the total micronutrients present in the composition; vitamin E is present at 3.39 wt. % to 3.57 wt. % of the total micronutrients present in the composition; calcium is present at 21.74 wt. % to 24.52 wt. % of the total micronutrients present in the composition; and/or selenium is present at 0.0014 wt. % to 0.0019 wt. % of the total micronutrients present in the composition; or
(l) vitamin A is present at 0.023 wt. % to 0.027 wt. % of the total micronutrients present in the composition; pyridoxine is present at 0.107 wt. % to 0.161 wt. % of the total micronutrients present in the composition; folic acid is present at 0.0065 wt. % to 0.0085 wt. % of the total micronutrients present in the composition; cyanocobalamin is present at 0.00019 wt. % to 0.00029 wt. % of the total micronutrients present in the composition; ascorbic acid is present at 8.696 wt. % to 9.913 wt. % of the total micronutrients present in the composition; cholecalciferol is present at 0.00020 wt. % to 0.00026 wt. % of the total micronutrients present in the composition; vitamin E is present at 3.39 wt. % to 3.57 wt. % of the total micronutrients present in the composition; calcium is present at 19.13 wt. % to 20.87 wt. % of the total micronutrients present in the composition; and/or selenium is present at 0.0020 wt. % to 0.0025 wt. % of the total micronutrients present in the composition; or
(m) vitamin A is present at 0.028 wt. % to 0.031 wt. % of the total micronutrients present in the composition; pyridoxine is present at 0.061 wt. % to 0.079 wt. % of the total micronutrients present in the composition; folic acid is present at 0.0089 wt. % to 0.0091 wt. % of the total micronutrients present in the composition; cyanocobalamin is present at 0.00011 wt. % to 0.00014 wt. % of the total micronutrients present in the composition; ascorbic acid is present at 6.087 wt. % to 7.304 wt. % of the total micronutrients present in the composition; cholecalciferol is present at 0.00043 wt. % to 0.00053 wt. % of the total micronutrients present in the composition; vitamin E is present at 2.09 wt. % to 2.30 wt. % of the total micronutrients present in the composition; calcium is present at 21.74 wt. % to 24.52 wt. % of the total micronutrients present in the composition; and/or selenium is present at 0.0014 wt. % to 0.0019 wt. % of the total micronutrients present in the composition; or
(n) vitamin A is present at 0.023 wt. % to 0.027 wt. % of the total micronutrients present in the composition; pyridoxine is present at 0.198 wt. % to 0.219 wt. % of the total micronutrients present in the composition; folic acid is present at 0.0065 wt. % to 0.0085 wt. % of the total micronutrients present in the composition; cyanocobalamin is present at 0.00035 wt. % to 0.00039 wt. % of the total micronutrients present in the composition; ascorbic acid is present at 8.696 wt. % to 9.913 wt. % of the total micronutrients present in the composition; cholecalciferol is present at 0.00043 wt. % to 0.00053 wt. % of the total micronutrients present in the composition; vitamin E is present at 3.39 wt. % to 3.57 wt. % of the total micronutrients present in the composition; calcium is present at 21.74 wt. % to 24.52 wt. % of the total micronutrients present in the composition; and/or selenium is present at 0.0020 wt. % to 0.0025 wt. % of the total micronutrients present in the composition; or
(o) vitamin A is present at 0.028 wt. % to 0.031 wt. % of the total micronutrients present in the composition; pyridoxine is present at 0.061 wt. % to 0.079 wt. % of the total micronutrients present in the composition; folic acid is present at 0.0065 wt. % to 0.0085 wt. % of the total micronutrients present in the composition; cyanocobalamin is present at 0.00011 wt. % to 0.00014 wt. % of the total micronutrients present in the composition; ascorbic acid is present at 8.696 wt. % to 9.913 wt. % of the total micronutrients present in the composition; cholecalciferol is present at 0.00020 wt. % to 0.00026 wt. % of the total micronutrients present in the composition; vitamin E is present at 3.39 wt. % to 3.57 wt. % of the total micronutrients present in the composition; calcium is present at 19.13 wt. % to 20.87 wt. % of the total micronutrients present in the composition; and/or selenium is present at 0.0014 wt. % to 0.0019 wt. % of the total micronutrients present in the composition; or
(p) vitamin A is present at 0.023 wt. % to 0.027 wt. % of the total micronutrients present in the composition; pyridoxine is present at 0.061 wt. % to 0.079 wt. % of the total micronutrients present in the composition; folic acid is present at 0.0065 wt. % to 0.0085 wt. % of the total micronutrients present in the composition; cyanocobalamin is present at 0.00011 wt. % to 0.00014 wt. % of the total micronutrients present in the composition; ascorbic acid is present at 6.087 wt. % to 7.304 wt. % of the total micronutrients present in the composition; cholecalciferol is present at 0.00020 wt. % to 0.00026 wt. % of the total micronutrients present in the composition; vitamin E is present at 2.09 wt. % to 2.30 wt. % of the total micronutrients present in the composition; calcium is present at 19.13 wt. % to 20.87 wt. % of the total micronutrients present in the composition; and/or selenium is present at 0.0020 wt. % to 0.0025 wt. % of the total micronutrients present in the composition; or
(q) vitamin A is present at 0.023 wt. % to 0.027 wt. % of the total micronutrients present in the composition; pyridoxine is present at 0.107 wt. % to 0.161 wt. % of the total micronutrients present in the composition; folic acid is present at 0.0065 wt. % to 0.0085 wt. % of the total micronutrients present in the composition; cyanocobalamin is present at 0.00019 wt. % to 0.00029 wt. % of the total micronutrients present in the composition; ascorbic acid is present at 8.696 wt. % to 9.913 wt. % of the total micronutrients present in the composition; cholecalciferol is present at 0.00065 wt. % to 0.00072 wt. % of the total micronutrients present in the composition; vitamin E is present at 3.39 wt. % to 3.57 wt. % of the total micronutrients present in the composition; calcium is present at 25.22 wt. % to 25.57 wt. % of the total micronutrients present in the composition; and/or selenium is present at 0.0014 wt. % to 0.0019 wt. % of the total micronutrients present in the composition.

17. The composition or kit according to any one of the preceding claims, wherein the composition further comprises one or more macronutrients selected from: a protein, a carbohydrate, a fat, and a fibre.

18. The composition or kit according to any one of the preceding claims, wherein protein is present at 40-80 wt. %.

19. The composition or kit according to claim 17 or 18, wherein the composition comprises protein and wherein the composition comprises:
(a) vitamin A present at a weight ratio of vitamin A to protein of greater than 1:55,000 to 1:14,667;
(b) pyridoxine present at a weight ratio of pyridoxine to protein of greater than 1:31,429 to 1:440;
(c) folic acid present at a weight ratio of folic acid to protein of greater than 1:220,000 to 1:44,000;
(d) cyanocobalamin present at a weight ratio of cyanocobalamin to protein of greater than 1:17,600,000 to 1:44,000;
(e) ascorbic acid present at a weight ratio of ascorbic acid to protein of greater than 1:550 to 1:22;
(f) cholecalciferol present at a weight ratio of cholecalciferol to protein of greater than 1:8,800,000 to 1:440,000;
(g) vitamin E present at a weight ratio of vitamin E to protein of greater than 1:3,667 to 1:40;
(h) calcium present at a weight ratio of calcium to protein of greater than 1:55 to 1:18; and/or
(i) selenium present at a weight ratio of selenium to protein of greater than 1:800,000 to 1:110,041.

20. The composition or kit according to claim 17-19, wherein the composition comprises protein and wherein the composition comprises:
(a) vitamin A present at a weight ratio of vitamin A to protein of: (i) greater than 1:55,000 to 1:43,137; (ii) greater than 1:43, 137 to 1:34,700; or (iii) greater than 1:34,700 to 1:14,667;
(b) pyridoxine present at a weight ratio of pyridoxine to protein of: (i) greater than 1:31,429 to 1:10,304; (ii) greater than 1:10,304 to 1:5,327; or (iii) greater than 1:5,327 to 1:440;
(c) folic acid present at a weight ratio of folic acid to protein of: (i) greater than 1:220,000 to 1:151,724; (ii) greater than 1:151,724 to 1:110,000; or (iii) greater than 1:110,000 to 1:44,000;
(d) cyanocobalamin present at a weight ratio of cyanocobalamin to protein of: (i) greater than 1:17,600,000 to 1:5,770,492; (ii) greater than 1:5,770,492 to 1:2,983,051; or (iii) greater than 1:2,983,051 to 1:44,000;
(e) ascorbic acid present at a weight ratio of ascorbic acid to protein of: (i) greater than 1:550 to 1:200; (ii) greater than 1:200 to 1:120; or (iii) greater than 1:120 to 1:22;
(f) cholecalciferol present at a weight ratio of cholecalciferol to protein of: (i) greater than 1:8,800,000 to 1:2,750,000; (ii) greater than 1:2,750,000 to 1:1,614,679; or (iii) greater than 1:1,614,679 to 1:440,000;
(g) vitamin E present at a weight ratio of vitamin E to protein of: (i) greater than 1:3,667 to 1:789; (ii) greater than 1:789 to 1:336; or (iii) greater than 1:336 to 1:40;
(h) calcium present at a weight ratio of calcium to protein of: (i) greater than 1:55 to 1:45; (ii) greater than 1:45 to 1:38; or (iii) greater than 1:38 to 1:18; and/or
(i) selenium present at a weight ratio of selenium to protein of: (i) greater than 1:800,000 to 1:500,000; or (ii) greater than 1:500,000 to 1:110,041.

21. The composition or kit according to any one of claims 17-20, wherein the composition comprises protein and wherein the composition comprises:
(a) vitamin A present at a weight ratio of vitamin A to protein of: (i) 1:47,826 to 1:44,000; (ii) 1:42,308 to 1:35,032; or (iii) 1:34,375 to 1:30,899;
(b) pyridoxine present at a weight ratio of pyridoxine to protein of: (i) 1:15,714 to 1:12,088; (ii) 1:8,980 to 1:5,930; or (iii) 1:4,835 to 1:4,365;
(c) folic acid present at a weight ratio of folic acid to protein of: (i) 1:183,333 to 1:157, 143; (ii) 1:146,667 to 1:112,821; or (iii) 1:107,317 to 1:104,762;
(d) cyanocobalamin present at a weight ratio of cyanocobalamin to protein of: (i) 1:8,800,000 to 1:6,769,231; (ii) 1:5,028,571 to 1:3,320,755; or (iii) 1:2,707,692 to 1:2,444,444;
(e) ascorbic acid present at a weight ratio of ascorbic acid to protein of: (i) 1:289 to 1:262; (ii) 1:157 to 1:131; or (iii) 1:110 to 1:96;
(f) cholecalciferol present at a weight ratio of cholecalciferol to protein of: (i) 1:4,888,889 to 1:3,666,667; (ii) 1:2,200,000 to 1:1,795,918; or (iii) 1:1,446,667 to 1:1,333,333;
(g) vitamin E present at a weight ratio of vitamin E to protein of: (i) 1:2,933 to 1:2,716; (ii) 1:458 to 1:417; or (iii) 1:282 to 1:268;
(h) calcium present at a weight ratio of calcium to protein of: (i) 1:50 to 1:46; (ii) 1:44 to 1:39; or (iii) 1:38 to 1:37; and/or
(i) selenium present at a weight ratio of selenium to protein of: (i) 1:666,667 to 1:516, 129; or (ii) 1:484,848 to 1:390,244.

22. The composition or kit according to any one of claims 17-21, wherein the composition comprises protein and wherein the composition comprises:
(a) vitamin A present at a weight ratio of vitamin A to protein of greater than 1:43,137 to 1:34,700; pyridoxine present at a weight ratio of pyridoxine to protein of greater than 1:10,304 to 1:5,327; folic acid present at a weight ratio of folic acid to protein of greater than 1:220,000 to 1:151,724; cyanocobalamin present at a weight ratio of cyanocobalamin to protein of greater than 1:5,770,492 to 1:2,983,051; ascorbic acid present at a weight ratio of ascorbic acid to protein of greater than 1:200 to 1:120; cholecalciferol present at a weight ratio of cholecalciferol to protein of greater than 1:1,614,679 to 1:440,000; vitamin E present at a weight ratio of vitamin E to protein of greater than 1:789 to 1:336; calcium present at a weight ratio of calcium to protein of greater than 1:55 to 1:45; and/or selenium present at a weight ratio of selenium to protein of greater than 1:500,000 to 1:110,041; or
(b) vitamin A present at a weight ratio of vitamin A to protein of greater than 1:34,700 to 1:14,667; pyridoxine present at a weight ratio of pyridoxine to protein of greater than 1:10,304 to 1:5,327; folic acid present at a weight ratio of folic acid to protein of greater than 1:220,000 to 1:151,724; cyanocobalamin present at a weight ratio of cyanocobalamin to protein of greater than 1:5,770,492 to 1:2,983,051; ascorbic acid present at a weight ratio of ascorbic acid to protein of greater than 1:200 to 1:120; cholecalciferol present at a weight ratio of cholecalciferol to protein of greater than 1:2,750,000 to 1:1,614,679; vitamin E present at a weight ratio of vitamin E to protein of greater than 1:789 to 1:336; calcium present at a weight ratio of calcium to protein of greater than 1:55 to 1:45; and/or selenium present at a weight ratio of selenium to protein of greater than 1:800,000 to 1:500,000; or
(c) vitamin A present at a weight ratio of vitamin A to protein of greater than 1:43,137 to 1:34,700; pyridoxine present at a weight ratio of pyridoxine to protein of greater than 1:5,327 to 1:440; folic acid present at a weight ratio of folic acid to protein of greater than 1:110,000 to 1:44,000; cyanocobalamin present at a weight ratio of cyanocobalamin to protein of greater than 1:2,983,051 to 1:44,000; ascorbic acid present at a weight ratio of ascorbic acid to protein of greater than 1:200 to 1:120; cholecalciferol present at a weight ratio of cholecalciferol to protein of greater than 1:1,614,679 to 1:440,000; vitamin E present at a weight ratio of vitamin E to protein of greater than 1:789 to 1:336; calcium present at a weight ratio of calcium to protein of greater than 1:45 to 1:38; and/or selenium present at a weight ratio of selenium to protein of greater than 1:800,000 to 1:500,000; or
(d) vitamin A present at a weight ratio of vitamin A to protein of greater than 1:34,700 to 1:14,667; pyridoxine present at a weight ratio of pyridoxine to protein of greater than 1:10,304 to 1:5,327; folic acid present at a weight ratio of folic acid to protein of greater than 1:220,000 to 1:151,724; cyanocobalamin present at a weight ratio of cyanocobalamin to protein of greater than 1:5,770,492 to 1:2,983,051; ascorbic acid present at a weight ratio of ascorbic acid to protein of greater than 1:200 to 1:120; cholecalciferol present at a weight ratio of cholecalciferol to protein of greater than 1:1,614,679 to 1:440,000; vitamin E present at a weight ratio of vitamin E to protein of greater than 1:789 to 1:336; calcium present at a weight ratio of calcium to protein of greater than 1:38 to 1:18; and/or selenium present at a weight ratio of selenium to protein of greater than 1:800,000 to 1:500,000; or
(e) vitamin A present at a weight ratio of vitamin A to protein of greater than 1:34,700 to 1:14,667; pyridoxine present at a weight ratio of pyridoxine to protein of greater than 1:5,327 to 1:440; folic acid present at a weight ratio of folic acid to protein of greater than 1:220,000 to 1:151,724; cyanocobalamin present at a weight ratio of cyanocobalamin to protein of greater than 1:2,983,051 to 1:44,000; ascorbic acid present at a weight ratio of ascorbic acid to protein of greater than 1:120 to 1:22; cholecalciferol present at a weight ratio of cholecalciferol to protein of greater than 1:1,614,679 to 1:440,000; vitamin E present at a weight ratio of vitamin E to protein of greater than 1:336 to 1:40; calcium present at a weight ratio of calcium to protein of greater than 1:45 to 1:38; and/or selenium present at a weight ratio of selenium to protein of greater than 1:500,000 to 1:110,041; or
(f) vitamin A present at a weight ratio of vitamin A to protein of greater than 1:43,137 to 1:34,700; pyridoxine present at a weight ratio of pyridoxine to protein of greater than 1:5,327 to 1:440; folic acid present at a weight ratio of folic acid to protein of greater than 1:151,724 to 1:110,000; cyanocobalamin present at a weight ratio of cyanocobalamin to protein of greater than 1:2,983,051 to 1:44,000; ascorbic acid present at a weight ratio of ascorbic acid to protein of greater than 1:200 to 1:120; cholecalciferol present at a weight ratio of cholecalciferol to protein of greater than 1:8,800,000 to 1:2,750,000; vitamin E present at a weight ratio of vitamin E to protein of greater than 1:789 to 1:336; calcium present at a weight ratio of calcium to protein of greater than 1:55 to 1:45; and/or selenium present at a weight ratio of selenium to protein of greater than 1:500,000 to 1:110,041; or
(g) vitamin A present at a weight ratio of vitamin A to protein of greater than 1:43,137 to 1:34,700; pyridoxine present at a weight ratio of pyridoxine to protein of greater than 1:31,429 to 1:10,304; folic acid present at a weight ratio of folic acid to protein of greater than 1:151,724 to 1:110,000; cyanocobalamin present at a weight ratio of cyanocobalamin to protein of greater than 1:17,600,000 to 1:5,770,492; ascorbic acid present at a weight ratio of ascorbic acid to protein of greater than 1:120 to 1:22; cholecalciferol present at a weight ratio of cholecalciferol to protein of greater than 1:2,750,000 to greater than 1:336 to 1:40; calcium present at a weight ratio of calcium to protein of greater than 1:45 to 1:38; and/or selenium present at a weight ratio of selenium to protein of greater than 1:500,000 to 1:110,041; or
(h) vitamin A present at a weight ratio of vitamin A to protein of greater than 1:43,137 to 1:34,700; pyridoxine present at a weight ratio of pyridoxine to protein of greater than 1:10,304 to 1:5,327; folic acid present at a weight ratio of folic acid to protein of greater than 1:151,724 to 1:110,000; cyanocobalamin present at a weight ratio of cyanocobalamin to protein of greater than 1:5,770,492 to 1:2,983,051; ascorbic acid present at a weight ratio of ascorbic acid to protein of greater than 1:200 to 1:120; cholecalciferol present at a weight ratio of cholecalciferol to protein of greater than 1:8,800,000 to 1:2,750,000; vitamin E present at a weight ratio of vitamin E to protein of greater than 1:789 to 1:336; calcium present at a weight ratio of calcium to protein of greater than 1:45 to 1:38; and/or selenium present at a weight ratio of selenium to protein of greater than 1:500,000 to 1:110,041; or
(i) vitamin A present at a weight ratio of vitamin A to protein of greater than 1:43,137 to 1:34,700; pyridoxine present at a weight ratio of pyridoxine to protein of greater than 1:10,304 to 1:5,327; folic acid present at a weight ratio of folic acid to protein of greater than 1:151,724 to 1:110,000; cyanocobalamin present at a weight ratio of cyanocobalamin to protein of greater than 1:5,770,492 to 1:2,983,051; ascorbic acid present at a weight ratio of ascorbic acid to protein of greater than 1:550 to 1:200; cholecalciferol present at a weight ratio of cholecalciferol to protein of greater than 1:2,750,000 to 1:1,614,679; vitamin E present at a weight ratio of vitamin E to protein of greater than 1:3,667 to 1:789; calcium present at a weight ratio of calcium to protein of greater than 1:45 to 1:38; and/or selenium present at a weight ratio of selenium to protein of greater than 1:800,000 to 1:500,000; or
(j) vitamin A present at a weight ratio of vitamin A to protein of greater than 1:34,700 to 1:14,667; pyridoxine present at a weight ratio of pyridoxine to protein of greater than 1:10,304 to 1:5,327; folic acid present at a weight ratio of folic acid to protein of greater than 1:220,000 to 1:151,724; cyanocobalamin present at a weight ratio of cyanocobalamin to protein of greater than 1:5,770,492 to 1:2,983,051; ascorbic acid present at a weight ratio of ascorbic acid to protein of greater than 1:120 to 1:22; cholecalciferol present at a weight ratio of cholecalciferol to protein of greater than 1:2,750,000 to greater than 1:336 to 1:40; calcium present at a weight ratio of calcium to protein of greater than 1:45 to 1:38; and/or selenium present at a weight ratio of selenium to protein of greater than 1:500,000 to 1:110,041; or
(k) vitamin A present at a weight ratio of vitamin A to protein of greater than 1:34,700 to 1:14,667; pyridoxine present at a weight ratio of pyridoxine to protein of greater than 1:10,304 to 1:5,327; folic acid present at a weight ratio of folic acid to protein of greater than 1:151,724 to 1:110,000; cyanocobalamin present at a weight ratio of cyanocobalamin to protein of greater than 1:5,770,492 to 1:2,983,051; ascorbic acid present at a weight ratio of ascorbic acid to protein of greater than 1:120 to 1:22; cholecalciferol present at a weight ratio of cholecalciferol to protein of greater than 1:2,750,000 to 1:1,614,679; vitamin E present at a weight ratio of vitamin E to protein of greater than 1:336 to 1:40; calcium present at a weight ratio of calcium to protein of greater than 1:45 to 1:38; and/or selenium present at a weight ratio of selenium to protein of greater than 1:800,000 to 1:500,000; or
(l) vitamin A present at a weight ratio of vitamin A to protein of greater than 1:43, 137 to 1:34,700; pyridoxine present at a weight ratio of pyridoxine to protein of greater than 1:10,304 to 1:5,327; folic acid present at a weight ratio of folic acid to protein of greater than 1:151,724 to 1:110,000; cyanocobalamin present at a weight ratio of cyanocobalamin to protein greater than 1:5,770,492 to 1:2,983,051; ascorbic acid present at a weight ratio of ascorbic acid to protein of greater than 1:120 to 1:22; cholecalciferol present at a weight ratio of cholecalciferol to protein of greater than 1:8,800,000 to 1:2,750,000; vitamin E present at a weight ratio of vitamin E to protein of greater than 1:336 to 1:40; calcium present at a weight ratio of calcium to protein of greater than 1:55 to 1:45; and/or selenium present at a weight ratio of selenium to protein of greater than 1:500,000 to 1:110,041; or
(m) vitamin A present at a weight ratio of vitamin A to protein of greater than 1:34,700 to 1:14,667; pyridoxine present at a weight ratio of pyridoxine to protein of greater than 1:31,429 to 1:10,304; folic acid present at a weight ratio of folic acid to protein of greater than 1:110,000 to 1:44,000; cyanocobalamin present at a weight ratio of cyanocobalamin to protein of greater than 1:17,600,000 to 1:5,770,492; ascorbic acid present at a weight ratio of ascorbic acid to protein of greater than 1:200 to 1:120; cholecalciferol present at a weight ratio of cholecalciferol to protein of greater than 1:2,750,000 to greater than 1:789 to 1:336; calcium present at a weight ratio of calcium to protein of greater than 1:45 to 1:38; and/or selenium present at a weight ratio of selenium to protein of greater than 1:800,000 to 1:500,000; or
(n) vitamin A present at a weight ratio of vitamin A to protein of greater than 1:43,137 to 1:34,700; pyridoxine present at a weight ratio of pyridoxine to protein of greater than 1:5,327 to 1:440; folic acid present at a weight ratio of folic acid to protein of greater than 1:151,724 to 1:110,000; cyanocobalamin present at a weight ratio of cyanocobalamin to protein greater than 1:2,983,051 to 1:44,000; ascorbic acid present at a weight ratio of ascorbic acid to protein of greater than 1:120 to 1:22; cholecalciferol present at a weight ratio of cholecalciferol to protein of greater than 1:2,750,000 to 1:1,614,679; vitamin E present at a weight ratio of vitamin E to protein of greater than 1:336 to 1:40; calcium present at a weight ratio of calcium to protein of greater than 1:45 to 1:38; and/or selenium present at a weight ratio of selenium to protein of greater than 1:500,000 to 1:110,041; or
(o) vitamin A present at a weight ratio of vitamin A to protein of greater than 1:34,700 to 1:14,667; pyridoxine present at a weight ratio of pyridoxine to protein of greater than 1:31,429 to 1:10,304; folic acid present at a weight ratio of folic acid to protein of greater than 1:151,724 to 1:110,000; cyanocobalamin present at a weight ratio of cyanocobalamin to protein of greater than 1:17,600,000 to 1:5,770,492; ascorbic acid present at a weight ratio of ascorbic acid to protein of greater than 1:120 to 1:22; cholecalciferol present at a weight ratio of cholecalciferol to protein of greater than 1:8,800,000 to 1:2,750,000; vitamin E present at a weight ratio of vitamin E to protein of greater than 1:336 to 1:40; calcium present at a weight ratio of calcium to protein of greater than 1:55 to 1:45; and/or selenium present at a weight ratio of selenium to protein of greater than 1:800,000 to 1:500,000; or
(p) vitamin A present at a weight ratio of vitamin A to protein of greater than 1:43,137 to 1:34,700; pyridoxine present at a weight ratio of pyridoxine to protein of greater than 1:31,429 to 1:10,304; folic acid present at a weight ratio of folic acid to protein of greater than 1:151,724 to 1:110,000; cyanocobalamin present at a weight ratio of cyanocobalamin to protein of greater than 1:17,600,000 to 1:5,770,492; ascorbic acid present at a weight ratio of ascorbic acid to protein of greater than 1:200 to 1:120; cholecalciferol present at a weight ratio of cholecalciferol to protein of greater than 1:8,800,000 to 1:2,750,000; vitamin E present at a weight ratio of vitamin E to protein of greater than 1:789 to 1:336; calcium present at a weight ratio of calcium to protein of greater than 1:55 to 1:45; and/or selenium present at a weight ratio of selenium to protein of greater than 1:500,000 to 1:110,041; or
(q) vitamin A present at a weight ratio of vitamin A to protein of greater than 1:43,137 to 1:34,700; pyridoxine present at a weight ratio of pyridoxine to protein of greater than 1:10,304 to 1:5,327; folic acid present at a weight ratio of folic acid to protein of greater than 1:151,724 to 1:110,000; cyanocobalamin present at a weight ratio of cyanocobalamin to protein of greater than 1:5,770,492 to 1:2,983,051; ascorbic acid present at a weight ratio of ascorbic acid to protein of greater than 1:120 to 1:22; cholecalciferol present at a weight ratio of cholecalciferol to protein of greater than 1:1,614,679 to 1:440,000; vitamin E present at a weight ratio of vitamin E to protein of greater than 1:336 to 1:40; calcium present at a weight ratio of calcium to protein of greater than 1:38 to 1:18; and/or selenium present at a weight ratio of selenium to protein of greater than 1:800,000 to 1:500,000.

23. The composition or kit according to any one of claims 17-22, wherein the composition comprises protein and wherein the composition comprises:
(a) vitamin A present at a weight ratio of vitamin A to protein of 1:42,308 to 1:35,032; pyridoxine present at a weight ratio of pyridoxine to protein of 1:8,980 to 1:5,930; folic acid present at a weight ratio of folic acid to protein of 1:183,333 to 1:157,143; cyanocobalamin present at a weight ratio of cyanocobalamin to protein of 1:5,028,571 to 1:3,320,755; ascorbic acid present at a weight ratio of ascorbic acid to protein of 1:157 to 1:131; cholecalciferol present at a weight ratio of cholecalciferol to protein of 1:1,446,667 to 1:1,333,333; vitamin E present at a weight ratio of vitamin E to protein of 1:458 to 1:417; calcium present at a weight ratio of calcium to protein of: 1:50 to 1:46; and/or selenium present at a weight ratio of selenium to protein 1:484,848 to 1:390,244; or
(b) vitamin A present at a weight ratio of vitamin A to protein of 1:34,375 to 1:30,899; pyridoxine present at a weight ratio of pyridoxine to protein of 1:8,980 to 1:5,930; folic acid present at a weight ratio of folic acid to protein of 1:183,333 to 1:157,143; cyanocobalamin present at a weight ratio of cyanocobalamin to protein of 1:5,028,571 to 1:3,320,755; ascorbic acid present at a weight ratio of ascorbic acid to protein of 1:157 to 1:131; cholecalciferol present at a weight ratio of cholecalciferol to protein of 1:2,200,000 to 1:1,795,918; vitamin E present at a weight ratio of vitamin E to protein of 1:458 to 1:417; calcium present at a weight ratio of calcium to protein of 1:50 to 1:46; and/or selenium present at a weight ratio of selenium to protein of 1:666,667 to 1:516, 129; or
(c) vitamin A present at a weight ratio of vitamin A to protein of 1:42,308 to 1:35,032; pyridoxine present at a weight ratio of pyridoxine to protein of 1:4,835 to 1:4,365; folic acid present at a weight ratio of folic acid to protein of 1:107,317 to 1:104,762; cyanocobalamin present at a weight ratio of cyanocobalamin to protein of 1:2,707,692 to 1:2,444,444; ascorbic acid present at a weight ratio of ascorbic acid to protein of 1:157 to 1:131; cholecalciferol present at a weight ratio of cholecalciferol to protein of 1:1,446,667 to 1:1,333,333; vitamin E present at a weight ratio of vitamin E to protein of 1:458 to 1:417; calcium present at a weight ratio of calcium to protein of 1:44 to 1:39; and/or selenium present at a weight ratio of selenium to protein of 1:666,667 to 1:516, 129; or
(d) vitamin A present at a weight ratio of vitamin A to protein of 1:34,375 to 1:30,899; pyridoxine present at a weight ratio of pyridoxine to protein of 1:8,980 to 1:5,930; folic acid present at a weight ratio of folic acid to protein of 1:183,333 to 1:157,143; cyanocobalamin present at a weight ratio of cyanocobalamin to protein of 1:5,028,571 to 1:3,320,755; ascorbic acid present at a weight ratio of ascorbic acid to protein of 1:157 to 1:131; cholecalciferol present at a weight ratio of cholecalciferol to protein of 1:1,446,667 to 1:1,333,333; vitamin E present at a weight ratio of vitamin E to protein of 1:458 to 1:417; calcium present at a weight ratio of calcium to protein of 1:38 to 1:37; and/or selenium present at a weight ratio of selenium to protein of 1:666,667 to 1:516, 129; or
(e) vitamin A present at a weight ratio of vitamin A to protein of 1:34,375 to 1:30,899; pyridoxine present at a weight ratio of pyridoxine to protein of 1:4,835 to 1:4,365; folic acid present at a weight ratio of folic acid to protein of 1:183,333 to 1:157,143; cyanocobalamin present at a weight ratio of cyanocobalamin to protein of 1:2,707,692 to 1:2,444,444; ascorbic acid present at a weight ratio of ascorbic acid to protein of 1:110 to 1:96; cholecalciferol present at a weight ratio of cholecalciferol to protein of 1:1,446,667 to 1:1,333,333; vitamin E present at a weight ratio of vitamin E to protein of 1:282 to 1:268; calcium present at a weight ratio of calcium to protein of 1:44 to 1:39; and/or selenium present at a weight ratio of selenium to protein of 1:484,848 to 1:390,244; or
(f) vitamin A present at a weight ratio of vitamin A to protein of 1:42,308 to 1:35,032; pyridoxine present at a weight ratio of pyridoxine to protein of 1:4,835 to 1:4,365; folic acid present at a weight ratio of folic acid to protein of 1:146,667 to 1:112,821; cyanocobalamin present at a weight ratio of cyanocobalamin to protein of 1:2,707,692 to 1:2,444,444; ascorbic acid present at a weight ratio of ascorbic acid to protein of 1:157 to 1:131; cholecalciferol present at a weight ratio of cholecalciferol to protein of 1:4,888,889 to 1:3,666,667; vitamin E present at a weight ratio of vitamin E to protein of 1:458 to 1:417; calcium present at a weight ratio of calcium to protein of 1:50 to 1:46; and/or selenium present at a weight ratio of selenium to protein of 1:484,848 to 1:390,244; or
(g) vitamin A present at a weight ratio of vitamin A to protein of 1:42,308 to 1:35,032; pyridoxine present at a weight ratio of pyridoxine to protein of 1:15,714 to 1:12,088; folic acid present at a weight ratio of folic acid to protein of 1:146,667 to 1:112,821; cyanocobalamin present at a weight ratio of cyanocobalamin to protein of 1:8,800,000 to 1:6,769,231; ascorbic acid present at a weight ratio of ascorbic acid to protein 1:110 to 1:96; cholecalciferol present at a weight ratio of cholecalciferol to protein of 1:2,200,000 to 1:1,795,918; vitamin E present at a weight ratio of vitamin E to protein of 1:282 to 1:268; calcium present at a weight ratio of calcium to protein of 1:44 to 1:39; and/or selenium present at a weight ratio of selenium to protein of 1:484,848 to 1:390,244; or
(h) vitamin A present at a weight ratio of vitamin A to protein of 1:42,308 to 1:35,032; pyridoxine present at a weight ratio of pyridoxine to protein of 1:8,980 to 1:5,930; folic acid present at a weight ratio of folic acid to protein of 1:146,667 to 1:112,821; cyanocobalamin present at a weight ratio of cyanocobalamin to protein of 1:5,028,571 to 1:3,320,755; ascorbic acid present at a weight ratio of ascorbic acid to protein of 1:157 to 1:131; cholecalciferol present at a weight ratio of cholecalciferol to protein of 1:4,888,889 to 1:3,666,667; vitamin E present at a weight ratio of vitamin E to protein of 1:458 to 1:417; calcium present at a weight ratio of calcium to protein of 1:44 to 1:39; and/or selenium present at a weight ratio of selenium to protein of 1:484,848 to 1:390,244; or
(i) vitamin A present at a weight ratio of vitamin A to protein of 1:42,308 to 1:35,032; pyridoxine present at a weight ratio of pyridoxine to protein of 1:8,980 to 1:5,930; folic acid present at a weight ratio of folic acid to protein 1:146,667 to 1:112,821; cyanocobalamin present at a weight ratio of cyanocobalamin to protein of 1:5,028,571 to 1:3,320,755; ascorbic acid present at a weight ratio of ascorbic acid to protein of 1:289 to 1:262; cholecalciferol present at a weight ratio of cholecalciferol to protein of 1:2,200,000 to 1:1,795,918; vitamin E present at a weight ratio of vitamin E to protein of 1:2,933 to 1:2,716; calcium present at a weight ratio of calcium to protein of 1:44 to 1:39; and/or selenium present at a weight ratio of selenium to protein of 1:666,667 to 1:516, 129; or
(j) vitamin A present at a weight ratio of vitamin A to protein of 1:34,375 to 1:30,899; pyridoxine present at a weight ratio of pyridoxine to protein of 1:8,980 to 1:5,930; folic acid present at a weight ratio of folic acid to protein of 1:183,333 to 1:157,143; cyanocobalamin present at a weight ratio of cyanocobalamin to protein of 1:5,028,571 to 1:3,320,755; ascorbic acid present at a weight ratio of ascorbic acid to protein of 1:110 to 1:96; cholecalciferol present at a weight ratio of cholecalciferol to protein of 1:2,200,000 to 1:1,795,918; vitamin E present at a weight ratio of vitamin E to protein of 1:282 to 1:268; calcium present at a weight ratio of calcium to protein of 1:44 to 1:39; and/or selenium present at a weight ratio of selenium to protein of 1:484,848 to 1:390,244; or
(k) vitamin A present at a weight ratio of vitamin A to protein of 1:34,375 to 1:30,899; pyridoxine present at a weight ratio of pyridoxine to protein of 1:8,980 to 1:5,930; folic acid present at a weight ratio of folic acid to protein of 1:146,667 to 1:112,821; cyanocobalamin present at a weight ratio of cyanocobalamin to protein of 1:5,028,571 to 1:3,320,755; ascorbic acid present at a weight ratio of ascorbic acid to protein of 1:110 to 1:96; cholecalciferol present at a weight ratio of cholecalciferol to protein of 1:2,200,000 to 1:1,795,918; vitamin E present at a weight ratio of vitamin E to protein of 1:282 to 1:268; calcium present at a weight ratio of calcium to protein of 1:44 to 1:39; and/or selenium present at a weight ratio of selenium to protein of 1:666,667 to 1:516, 129; or
(l) vitamin A present at a weight ratio of vitamin A to protein of 1:42,308 to 1:35,032; pyridoxine present at a weight ratio of pyridoxine to protein of 1:8,980 to 1:5,930; folic acid present at a weight ratio of folic acid to protein of 1:146,667 to 1:112,821; cyanocobalamin present at a weight ratio of cyanocobalamin to protein of 1:5,028,571 to 1:3,320,755; ascorbic acid present at a weight ratio of ascorbic acid to protein of 1:110 to 1:96; cholecalciferol present at a weight ratio of cholecalciferol to protein of 1:4,888,889 to 1:3,666,667; vitamin E present at a weight ratio of vitamin E to protein of 1:282 to 1:268; calcium present at a weight ratio of calcium to protein of 1:50 to 1:46; and/or selenium present at a weight ratio of selenium to protein of 1:484,848 to 1:390,244; or
(m) vitamin A present at a weight ratio of vitamin A to protein of 1:34,375 to 1:30,899; pyridoxine present at a weight ratio of pyridoxine to protein of 1:15,714 to 1:12,088; folic acid present at a weight ratio of folic acid to protein of 1:107,317 to 1:104,762; cyanocobalamin present at a weight ratio of cyanocobalamin to protein of 1:8,800,000 to 1:6,769,231; ascorbic acid present at a weight ratio of ascorbic acid to protein of 1:157 to 1:131; cholecalciferol present at a weight ratio of cholecalciferol to protein of 1:2,200,000 to 1:1,795,918; or vitamin E present at a weight ratio of vitamin E to protein of 1:458 to 1:417; calcium present at a weight ratio of calcium to protein of 1:44 to 1:39; and/or selenium present at a weight ratio of selenium to protein of 1:666,667 to 1:516, 129; or
(n) vitamin A present at a weight ratio of vitamin A to protein 1:42,308 to 1:35,032; pyridoxine present at a weight ratio of pyridoxine to protein of 1:4,835 to 1:4,365; folic acid present at a weight ratio of folic acid to protein of 1:146,667 to 1:112,821; cyanocobalamin present at a weight ratio of cyanocobalamin to protein of 1:2,707,692 to 1:2,444,444; ascorbic acid present at a weight ratio of ascorbic acid to protein of 1:110 to 1:96; cholecalciferol present at a weight ratio of cholecalciferol to protein of 1:2,200,000 to 1:1,795,918; vitamin E present at a weight ratio of vitamin E to protein of 1:282 to 1:268; calcium present at a weight ratio of calcium to protein 1:44 to 1:39; and/or selenium present at a weight ratio of selenium to protein of 1:484,848 to 1:390,244; or
(o) vitamin A present at a weight ratio of vitamin A to protein of 1:34,375 to 1:30,899; pyridoxine present at a weight ratio of pyridoxine to protein of 1:15,714 to 1:12,088; folic acid present at a weight ratio of folic acid to protein of 1:146,667 to 1:112,821; cyanocobalamin present at a weight ratio of cyanocobalamin to protein of 1:8,800,000 to 1:6,769,231; ascorbic acid present at a weight ratio of ascorbic acid to protein of 1:110 to 1:96; cholecalciferol present at a weight ratio of cholecalciferol to protein of 1:4,888,889 to 1:3,666,667; vitamin E present at a weight ratio of vitamin E to protein of 1:282 to 1:268; calcium present at a weight ratio of calcium to protein of 1:50 to 1:46; and/or selenium present at a weight ratio of selenium to protein of 1:666,667 to 1:516, 129; or
(p) vitamin A present at a weight ratio of vitamin A to protein of 1:42,308 to 1:35,032; pyridoxine present at a weight ratio of pyridoxine to protein of 1:15,714 to 1:12,088; folic acid present at a weight ratio of folic acid to protein of 1:146,667 to 1:112,821; cyanocobalamin present at a weight ratio of cyanocobalamin to protein of 1:8,800,000 to 1:6,769,231; ascorbic acid present at a weight ratio of ascorbic acid to protein of 1:157 to 1:131; cholecalciferol present at a weight ratio of cholecalciferol to protein of 1:4,888,889 to 1:3,666,667; vitamin E present at a weight ratio of vitamin E to protein of 1:458 to 1:417; calcium present at a weight ratio of calcium to protein of 1:50 to 1:46; and/or selenium present at a weight ratio of selenium to protein of 1:484,848 to 1:390,244; or
(q) vitamin A present at a weight ratio of vitamin A to protein of 1:42,308 to 1:35,032; pyridoxine present at a weight ratio of pyridoxine to protein of 1:8,980 to 1:5,930; folic acid present at a weight ratio of folic acid to protein of 1:146,667 to 1:112,821; cyanocobalamin present at a weight ratio of cyanocobalamin to protein of 1:5,028,571 to 1:3,320,755; ascorbic acid present at a weight ratio of ascorbic acid to protein of 1:110 to 1:96; cholecalciferol present at a weight ratio of cholecalciferol to protein of 1:1,446,667 to 1:1,333,333; vitamin E present at a weight ratio of vitamin E to protein of 1:282 to 1:268; calcium present at a weight ratio of calcium to protein of 1:38 to 1:37; and/or selenium present at a weight ratio of selenium to protein of 1:666,667 to 1:516, 129.

24. The composition or kit according to any of the preceding claims, wherein:
(a) the composition comprises additional nutritional components, preferably a stimulant, an additional macronutrient and/or an additional micronutrient; and/or
(b) wherein the composition comprises at least one additive, preferably an additive selected from: a flavouring; a sweetener; an emulsifying and/or stabilizing agent; a binding agent; an acidity regulator; and/or a colourant; and/or
(c) wherein the composition provides a subject with their nutritional requirements as determined by the subject's genetic profile.

25. The composition or kit according to any of the preceding claims, wherein the composition is in a form ready for consumption.

26. The composition or kit according to any of the preceding claims, wherein the composition is in the form of a powder, a tablet, a bar, a confectionary product, or a granule.

27. A method for manufacturing a nutritional composition, the method comprising:
(a) admixing at least two micronutrients selected from vitamin A, pyridoxine (vitamin B6), folic acid (vitamin B9), cyanocobalamin (vitamin B12), ascorbic acid (vitamin C), cholecalciferol (vitamin D3), vitamin E, calcium, and/or selenium; or
(b) admixing at least one micronutrient selected from vitamin A, pyridoxine (vitamin B6), folic acid (vitamin B9), cyanocobalamin (vitamin B12), ascorbic acid (vitamin C), cholecalciferol (vitamin D3), vitamin E, calcium, and/or selenium with a macronutrient; and
(c) optionally packaging.

28. A method for determining a subject's nutritional requirements, the method comprising:
(a) providing a subject's genetic profile, wherein the subject's genetic profile comprises a sequence of at least a region of a gene;
(b) comparing the subject's genetic profile with a plurality of genetic reference standards, wherein each of the genetic reference standards is associated with a requirement level of a nutrient; and
(c) determining which of the plurality of genetic reference standards is most similar to the subject's genetic profile, thereby identifying a level of the nutrient required by the subject.

29. The method according to claim 28, wherein the method comprises:
(a) providing a subject's genetic profile, wherein the subject's genetic profile comprises sequences of at least a region of a plurality of genes;
(b) comparing the subject's genetic profile with at least a first set of genetic reference standards and a second set of genetic reference standards, wherein the first set of genetic reference standards comprises at least two (preferably three) genetic reference standards, each associated with a different requirement level of a first nutrient, and wherein the second set of genetic reference standards comprises at least two (preferably three) genetic reference standards, each associated with a different requirement level of a second different nutrient; and
(c) determining which of the at least two (preferably three) genetic reference standards of the first set of genetic reference standards is most similar to the subject's genetic profile, thereby identifying a level of the first nutrient required by the subject, and determining which of the at least two (preferably three) genetic reference standards of the second set of genetic reference standards is most similar to the subject's genetic profile, thereby identifying a level of the second nutrient required by the subject.

30. The method according to claim 28 or 29, further comprising selecting a nutritional composition based on the identification of the level(s) of the nutrient(s) required by the subject and/or administering a nutritional composition to the subject.

31. The method according to claim 29 or 30, wherein the method further comprises:
(d) providing a plurality of nutritional reference standards, wherein the nutritional reference standards are different from one another, and wherein each nutritional reference standard is associated with requirement levels of a plurality of nutrients;
(e) comparing the levels of the nutrients required by the subject with the requirement levels of the same nutrients in each of the plurality of nutritional reference standards; and
(f) determining which of the plurality of nutritional reference standards is most similar to the subject's requirement levels.

32. The method according to claim 31, wherein each of the nutritional reference standards corresponds to a different nutritional composition and wherein the determining which of the plurality of nutritional reference standards is most similar to the subject's requirement levels identifies the nutritional composition corresponding to the subject's nutritional requirements.

33. The method according to claim 32, further comprising selecting the nutritional composition and/or administering the nutritional composition to the subject.

34. The method according to any one of claims 28-33, wherein the nutritional requirements of the subject are further modified from the determined nutritional requirements based on:
(a) the subject's current BMI;
(b) the subject's current diet;
(c) the subject's current health status; and/or
(d) the subject's current fitness status.

35. The method according to any one of claims 28-34, wherein the subject's nutritional requirements are daily nutritional requirements.

36. The method according to any one of claims 28-35, wherein the subject's genetic profile is obtained by DNA sequencing.

37. The method according to any one of claims 28-36, wherein the nutrient is a micronutrient.

38. The method according to any one of claims 28-37, wherein the gene is: ACE, PPARG, TCF7L2, ADRB2, ADRB3, FTO, APOC3, LPL, APOA5, CYP1A2, SOD2, CAT, GPX1, MTHFR, SLC19A1, TCN2, VDR, MCM6, HLA DQA1, and/or BCO1.

39. The method according to any one of claims 31-38, wherein the nutritional reference standard corresponds to one or more of Archetypes 1-17 as defined in Tables 9-14.

40. The method according to any one of claims 31-39, wherein the nutritional reference standard corresponds to one or more of Tables 17-33.

41. The method according to any one of claims 31-40, wherein:
(a) carbohydrate sensitivity is associated with at least one gene selected from: ACE, PPARG, TCF7L2, ADRB2 and/or ADRB3;
(b) fat sensitivity is associated with at least one gene selected from: ADRB2, ADRB3 APOC3, LPL, APOA5 and/or FTO;
(c) antioxidant need is associated with at least one gene selected from: SOD2, CAT and/or GPX1;
(d) folic acid need is associated with at least one gene selected from: MTHFR and/or SLC19A1;
(e) vitamin B12 need is associated with: TCN2;
(f) vitamin D need is associated with: VDR;
(g) calcium need is associated with: VDR;
(h) caffeine sensitivity is associated with: CYP1A2;
(i) lactose sensitivity is associated with: MCM6;
(j) gluten sensitivity is associated with: HLA DQA1; and/or
(k) vitamin A need is associated with: BCO1.

42. Use of a nutritional composition or kit according to any one of claims 1-26 to provide a subject with its daily micronutrient requirements, wherein at least a daily unit dose of the one or more micronutrients comprised in the composition is administered to the subject per day.

43. A method of providing a subject with its daily micronutrient requirements, the method comprising administering at least a daily unit dose of the one or more micronutrients comprised in the composition according to any one of claims 1-26 to the subject per day.
